SEQUENCE LISTING (645 sequences)

SEQ ID NO: 1 - DNA, 63 bp, Artificial Sequence
  Synthetic
atngaatnnaanngaatggannnnaanggaatngaatnnaatggaatggaanngantngaatg

SEQ ID NO: 2 - DNA, 75 bp, Artificial Sequence
  Synthetic
aannggaatnnaatngaatnnaanngaatggannnnaanggaatngaatnnaatggaatggaanngantngaatg

SEQ ID NO: 3 - DNA, 85 bp, Artificial Sequence
  Synthetic
aaanagaatcaannggaatnnaatngaatnnaanngaatggannnnaanggaatngaatnnaatggaatggaanngantngaatg

SEQ ID NO: 4 - DNA, 13 bp, Artificial Sequence
  Synthetic
nnatcnntaaang

SEQ ID NO: 5 - DNA, 13 bp, Artificial Sequence
  Synthetic
aaatcaataaatg

SEQ ID NO: 6 - DNA, 12 bp, Artificial Sequence
  Synthetic
tagccggtgacc

SEQ ID NO: 7 - DNA, 75 bp, Artificial Sequence
  Synthetic
aatcgaatggaatcaacatcaaacggaaaaaaacggaattatcgaatggaatcgaagagaatcatcgaatggacc

SEQ ID NO: 8 - DNA, 78 bp, Artificial Sequence
  Synthetic
tggaatcgaatggaatcaacatcaaacggaaaaaaacggaattatcgaatggaatcgaagagaatcatcgaatggacc

SEQ ID NO: 9 - DNA, 103 bp, Artificial Sequence
  Synthetic
agcattcatatcttgcagtgttgggaaagagtgagaggttgtgatgtcaagaaggataggtcagaagtggaaggtatgggggattgtgcctgctgtcatggct

SEQ ID NO: 10 - DNA, 148 bp, Artificial Sequence
  Synthetic
ggaacgaaatcgaatggaacggaatagaatagactcgaatgtaatggattgctatgtaattgattcgaatggaatggaatcgaatggaatgcaatccaatggaatggaatgcaatgcaatgaatggaatggaatggaatggaatggaa

SEQ ID NO: 11 - DNA, 147 bp, Artificial Sequence
  Synthetic
ggaacgaaatcgaatggaacggaatagaatagactcgaatgtaatggattgctatgtaattgattcgaatggaatggaatcgaatggaatgcaatccaatggaatggaatgcaatgcaatgaatggaatggaatggaatggaatgga

SEQ ID NO: 12 - DNA, 86 bp, Artificial Sequence
  Synthetic
tacgcaaatcgataaatgtaatccagcatataaacagaaccaaagacaaaaaccacatgattatctcaatagatgcagaaaaggcc

SEQ ID NO: 13 - DNA, 105 bp, Artificial Sequence
  Synthetic
actcgaatgcaatcaacatcaaacggaatcaaacggaattatcgaatggaatcgaagagaatcatcgaacggactcgaatggaatcatctaatggaatggaatgg

SEQ ID NO: 14 - DNA, 81 bp, Artificial Sequence
  Synthetic
gaaattccaattaaaatgaaatcgacttatcttaacaaatatagcaatgctgacaacacttctccggatatgggtactgct

SEQ ID NO: 15 - DNA, 91 bp, Artificial Sequence
  Synthetic
aaggaaaagtaaaaggaacttaacaccttcaagaaaagacagacaaataacaaaacagcagtttgatagaatgagatatcaggggatggca

SEQ ID NO: 16 - DNA, 63 bp, Artificial Sequence
  Synthetic
atcaacatcaaacggaaaaaacggaattatcgaatggaatcgaagagaatcatcgaacggacc

SEQ ID NO: 17 - DNA, 139 bp, Artificial Sequence
  Synthetic
aaagaaagacagagaacaaacgtaattcaagatgactgattacatatccaagaacattagatggtcaaagactttaagaaggaatacattcaaaggcaaaaagtcacttactgattttggtggagtttgccacatggac

SEQ ID NO: 18 - DNA, 79 bp, Artificial Sequence
  Synthetic
aagggaattgaatagaatgaatccgaatggaatggaatggaatggaatggaatggaatggaatggaatggaatggaatg

SEQ ID NO: 19 - DNA, 84 bp, Artificial Sequence
  Synthetic
gaatggaatcgaatcaaattaaatcaaatggaatgcaatagaagggaatacaatggaatagaatggaatggaatggaatggact

SEQ ID NO: 20 - DNA, 104 bp, Artificial Sequence
  Synthetic
acagcaagagagaaataaaacgacaagaaaactacaaaatgcctatcaatagttactttaaatatcagtggaccaaatcagtgaaacaaaagacacagagtggc

SEQ ID NO: 21 - DNA, 80 bp, Artificial Sequence
  Synthetic
tagcaggaaacagcaaactcaaattaagtaatttcaagagcgtatcatcaatgaactattttcaaagatgtgggcaagat

SEQ ID NO: 22 - DNA, 78 bp, Artificial Sequence
  Synthetic
aaacggaattatcaaatggaatcgaagagaatcatcgaacggactcgaatggaatcatctaatggaatggaatggaag

SEQ ID NO: 23 - DNA, 136 bp, Artificial Sequence
  Synthetic
gaatgaaatgaaatcaaatngaatgtacatgaatggaatagaaaagaatgcatctttctcgaacggaagtgcattgaatggaaaggaatctactggaatggattcgaatggaatggaangggatggaatggtatgg

SEQ ID NO: 24 - DNA, 97 bp, Artificial Sequence
  Synthetic
aatggactcgaatgaaatcatcatcaaacggaatcgaatggaatcattgaatggaaaggatgggatcatcatggaatggaaacgaatggaatcactg

SEQ ID NO: 25 - DNA, 104 bp, Artificial Sequence
  Synthetic
aatggaatcattgaatggaatggaatggaatcatcaaagaaaggaatcgaagggaatcatcgaatggaatcaaacggaatcatcgaatggaatggaatggaatg

SEQ ID NO: 26 - DNA, 73 bp, Artificial Sequence
  Synthetic
agcagaagaaataactgaaatcagagtgaaactgaatcaaattgagatgcaaaaatacatacgaaatggccag

SEQ ID NO: 27 - DNA, 79 bp, Artificial Sequence
  Synthetic
agttaatccgaatagaatggaatggaatgcaatggaacggaatggaacggaatggaatggaatggaatggaatggaatg

SEQ ID NO: 28 - DNA, 165 bp, Artificial Sequence
  Synthetic
atggaatcaacatcaaacggaatcaaacggaattatcgaatggaatcgaagagaatcatcgaacggattcgaatggaatcatctaatggaatggaatggaagaatccatggactcgaatgcaatcatcagcgaatggaatcgaatggaatcatcgaatggactcg

SEQ ID NO: 29 - DNA, 84 bp, Artificial Sequence
  Synthetic
aaaggaatggactggaacaaaatgaaatcgaacggtaggaatcgtacagaacggacagaaatggaacggcatggaatgcactcg

SEQ ID NO: 30 - DNA, 66 bp, Artificial Sequence
  Synthetic
aaatcaacaacaaacggaaaaaaaaggaattatcgaatggaatcaaagagaatcatcgaatggacc

SEQ ID NO: 31 - DNA, 86 bp, Artificial Sequence
  Synthetic
aaatgaacaaaactagaggaatgacattacctgacttcaaattatactacagagctatagtaaccaaaacagcatggtacaggcat

SEQ ID NO: 32 - DNA, 69 bp, Artificial Sequence
  Synthetic
gtaatggaatggaatggaaaggaatcgaaacgaaaggaatggagacagatggaatggaatggaacagag

SEQ ID NO: 33 - DNA, 74 bp, Artificial Sequence
  Synthetic
atcgaatggaatcaacatcaaacggaaaaaaacggaattatcgaatggaatcgaagagaatcatcgaatggacc

SEQ ID NO: 34 - DNA, 57 bp, Artificial Sequence
  Synthetic
caatcagagcggacacaaacaaattgcatgggaagaatcaatatcgtgaaaatggcc

SEQ ID NO: 35 - DNA, 80 bp, Artificial Sequence
  Synthetic
agacctttctcagaagacacacaaattgccaacaggtatatgaaaaaatgttcaatatcactaatcatcagggcgatgcc

SEQ ID NO: 36 - DNA, 106 bp, Artificial Sequence
  Synthetic
catggaatcgaatggaattatcatcgaatggaatcgaatggtaccaacaccaaacggaaaaaaacggaattatcgaatggaatcgaagagaatcttcgaacggacc

SEQ ID NO: 37 - DNA, 86 bp, Artificial Sequence
  Synthetic
gaacgatttatcactgaaaattaatactcatgcaagtagtaaacgaatgtaatgaccatgataaggagacggacggtggtgatagt

SEQ ID NO: 38 - DNA, 80 bp, Artificial Sequence
  Synthetic
aaagatcaangnncaaaaatcagcagcatttctataaaccaacaatgtccaggctgagagngaaatcaagaaancaattc

SEQ ID NO: 39 - DNA, 121 bp, Artificial Sequence
  Synthetic
acacacataccaacagaacatgacaaaagaacaaaaccagccgcatgcatactcgatggagacaaaggtaacactgcagaatggtgaaggaagaacagtcattttaatgacagtgttggct

SEQ ID NO: 40 - DNA, 70 bp, Artificial Sequence
  Synthetic
aatggaatcaacatcaaacggaaaaaaacggaattatcgaatggaatcgaagagaatcatcgaatggacc

SEQ ID NO: 41 - DNA, 99 bp, Artificial Sequence
  Synthetic
atcaaaaggaacggaatggaatggaatggaatggaatggaatggaatggaatggaatgaaatcaacccgaatggaatggattggcatagagtggaatgg

SEQ ID NO: 42 - DNA, 90 bp, Artificial Sequence
  Synthetic
taaagaaaaacaaacaaacagaaatcaatgaaaatcccattcaaaggtcagcaacctcaaagactgaaggtagataagcccacaaggatg

SEQ ID NO: 43 - DNA, 55 bp, Artificial Sequence
  Synthetic
aaacggaaaaaaacggaattatcgaatggaatcgaatagaatcatcgaatggacc

SEQ ID NO: 44 - DNA, 96 bp, Artificial Sequence
  Synthetic
ggaatcaactcgattgcaatggaatgcaatggaaaggaatggaatgcaattaaagcgaatagaatggaatggaatggaatggaacggaatggaatg

SEQ ID NO: 45 - DNA, 83 bp, Artificial Sequence
  Synthetic
gaagaagaaaaaacatggatatacaatgtcaacagaaatcaaggagaaacggaatttcaccaatcaatttagtgatctgggtt

SEQ ID NO: 46 - DNA, 132 bp, Artificial Sequence
  Synthetic
tggaatcatctaatggaatggaatggaataatccatggactcgaatgcaatcatcataaaatggaatcgaatggaatcaacatcaaatggaatcaaatgggatcattgaacggaattgaatggaatcgtcat

SEQ ID NO: 47 - DNA, 105 bp, Artificial Sequence
  Synthetic
tgaacagagaattggacaaaacgcacaaagtaaagaaaaagaatgaagcaacaaaagcagagatttattgaaaacaaaagtacacaccacacagggtgggagtgg

SEQ ID NO: 48 - DNA, 67 bp, Artificial Sequence
  Synthetic
ggaatcaacatcaaacggaaaaaaacggaattatcgaatggaatcgaagagaatcatcgaatggacc

SEQ ID NO: 49 - DNA, 98 bp, Artificial Sequence
  Synthetic
aacacgactttgagaagagtaagtgattgttaattaaagcaagagaattattgatgtatcacagtcatgagaaatattggaaggaatatggtccatac

SEQ ID NO: 50 - DNA, 80 bp, Artificial Sequence
  Synthetic
tgaaaagaagaatgaccataagcaagcagatgaaaaacaaaacagaatttttacagacgtcttggactgatatcttgggc

SEQ ID NO: 51 - DNA, 80 bp, Artificial Sequence
  Synthetic
aatcaataaatgtaaaccagcatataaacagaaccaacgacaaaaaccacatgattatctcaatagatgcagaaaaggcc

SEQ ID NO: 52 - DNA, 95 bp, Artificial Sequence
  Synthetic
caacatcaaacggaatcaaacggaattatcgaatggaatcgaagagaatcatcgaatggactcgaatggaatcatctaatggaatggaatggaag

SEQ ID NO: 53 - DNA, 85 bp, Artificial Sequence
  Synthetic
aatggaagggaatggaatggaatcgaatcgaatggaacagaattcaatggaatggaatggaatggaatggaatcgaatggaatgg

SEQ ID NO: 54 - DNA, 90 bp, Artificial Sequence
  Synthetic
aaagacttaaacataagacctaaaaccataaaaaccacagaagaaaacataggcaatgccattcaggacataggcatgggcaaagacttc

SEQ ID NO: 55 - DNA, 80 bp, Artificial Sequence
  Synthetic
agacttgaaaagcacagacaacgaaagcaaaaatggacaaatggaatcacatcaagctaaaaggttttgcatggcaaagg

SEQ ID NO: 56 - DNA, 88 bp, Artificial Sequence
  Synthetic
agcaacttcagcaaagtctcaggatacaaaatcaatgtgcaaaaatcacaagcattcttatacaccaacaacagacaaacagagagcc

SEQ ID NO: 57 - DNA, 147 bp, Artificial Sequence
  Synthetic
tgaatgctatagagcagtaaaaacaaataaatgaactacattacagctacttacaaccatatgaaagaatataaccataacaatgatgagtggacaaaagctaagtgtgaaagaatgcatagtgctacagcagccaacatttacagc

SEQ ID NO: 58 - DNA, 100 bp, Artificial Sequence
  Synthetic
aacaaaattgaacaacatgcaaagaaacataaacgaagcaatgaaagtgtgcagatccactgaaatgaaagtgctgtccagagtgggagccagctcgaga

SEQ ID NO: 59 - DNA, 136 bp, Artificial Sequence
  Synthetic
tggaattatcgtcgaatagaatcgaatggtatcaacatcaaacggaaaaaaacggaattatcgaatggaatcgaagagaatcatcgaacggactcgaatggaatcatctaatggaatggaatggaataatccatgg

SEQ ID NO: 60 - DNA, 94 bp, Artificial Sequence
  Synthetic
agataagtggatgaacagatggacagatggatggatggatggatggatggatggatgcctggaagaaagaagaatggatagtaagctgggtata

SEQ ID NO: 61 - DNA, 55 bp, Artificial Sequence
  Synthetic
aatcaaagaattgaatcgaatggaatcatctaatgtactcgaatggaatcaccat

SEQ ID NO: 62 - DNA, 79 bp, Artificial Sequence
  Synthetic
aatggaatcgaacggaatcatcatcaaacggaaccgaatggaatcattgaatggaatcaaaggcaatcatggtcgaatg

SEQ ID NO: 63 - DNA, 82 bp, Artificial Sequence
  Synthetic
aggaatctataatacagctgtttatagccaagcactaaatcatatgatacagaaaacaaatgcagatggtttgaagggtggg

SEQ ID NO: 64 - DNA, 54 bp, Artificial Sequence
  Synthetic
aacggaaaaaaacggaattatcgaatggaatcgaagagaatcatcgaatggacc

SEQ ID NO: 65 - DNA, 124 bp, Artificial Sequence
  Synthetic
tgagaaaatgatggaaaagaggaataanacgaaacaaaaccacaggaacacaggtgcatgtgaatgtgcacagacaaagatacagggcggactgggaaggaagtttctgcaccagaatttgggg

SEQ ID NO: 66 - DNA, 79 bp, Artificial Sequence
  Synthetic
aatggaatcgaagagaatggaaacaaatggaatggaattgaatggaatggaattgaatggaatgggaaggaatggagtg

SEQ ID NO: 67 - DNA, 76 bp, Artificial Sequence
  Synthetic
aatgtcaagtggaatcgagtggaatcatcgaaagaaatcgaatggaatcgaagggaatcattggatgggctcaaat

SEQ ID NO: 68 - DNA, 84 bp, Artificial Sequence
  Synthetic
aaacaatggaagataatggaaagatatcgaatggaatagaatggaatggaatggactcaaatggaatggactttaatggaatgg

SEQ ID NO: 69 - DNA, 84 bp, Artificial Sequence
  Synthetic
gaacaatcaatggaagcagaaacaaataaaccaaggtgtgcatcaaggaatacattcacgcatgatggctgtatgagtaaaatg

SEQ ID NO: 70 - DNA, 85 bp, Artificial Sequence
  Synthetic
aaaccgaatggaatggaatggacgcaaaatgaatggaatggaagtcaatggactcgaaatgaatggaatggaatggaatggaatg

SEQ ID NO: 71 - DNA, 68 bp, Artificial Sequence
  Synthetic
aggatacaaaatcaaagtgcaaaaatcacaagcattcttatacaccaataacagacaaacagagagcc

SEQ ID NO: 72 - DNA, 77 bp, Artificial Sequence
  Synthetic
ggaatcgaatggaatcaacatcaaacggaaaaaaacagaattatcgtatggaatcgaatagaatcatcgaatggacc

SEQ ID NO: 73 - DNA, 96 bp, Artificial Sequence
  Synthetic
caacccgagtggaataaaatggaatggaatggaatgaaatggaatggatcggaatggaatccaatggaatcaactggaatggaatggaatggaatg

SEQ ID NO: 74 - DNA, 88 bp, Artificial Sequence
  Synthetic
tatcatcgaatggaatcgaatggaatcaacatcaaacggaaaaaaacggaattatcgaatggaatcgaagagaatcatcgaatggacc

SEQ ID NO: 75 - DNA, 88 bp, Artificial Sequence
  Synthetic
cggaataatcattgaacggaatcgaatggaatcatcatcggatggaaacgaatggaatcatcatcgaatggaaatgaaaggagtcatc

SEQ ID NO: 76 - DNA, 83 bp, Artificial Sequence
  Synthetic
caacacacagagattaaaacaaacaaacaaacaatccagccctgacatttatgagtttacagactggtggagaggcagagaag

SEQ ID NO: 77 - DNA, 107 bp, Artificial Sequence
  Synthetic
ggaatggaatgaacacgaatgtaatgcaacccaatagaatggaatcgaatggcatggaatataaagaaatggaatcgaagagaatggaaacaaatggaatggaattg

SEQ ID NO: 78 - DNA, 71 bp, Artificial Sequence
  Synthetic
cactacaaaccacgctcaaggcaataaaagaacacaaacaaatggaaaaacattccatgctcatggatggg

SEQ ID NO: 79 - DNA, 75 bp, Artificial Sequence
  Synthetic
aatcgaatggaattaacatcaaacggaaaaaaacggaattatcgaatggaatcgaagagaatcatcgaatggacc

SEQ ID NO: 80 - DNA, 82 bp, Artificial Sequence
  Synthetic
tggaaaagaatcaaattgaatggcatcgaacggaatgggatggaatggaatagacccagatgtaatggactcgaatggaatg

SEQ ID NO: 81 - DNA, 81 bp, Artificial Sequence
  Synthetic
aatcagtctagatcttaaaggaacaccagagggagtatttaaatgtgcccaataagcaagaattatggtgatgtggaagta

SEQ ID NO: 82 - DNA, 74 bp, Artificial Sequence
  Synthetic
ccataacacaattaaaaacaacctaaatgtctaatagaagaacactgttcagaccgggcatggtggcttatacc

SEQ ID NO: 83 - DNA, 95 bp, Artificial Sequence
  Synthetic
gactaatattcagaatatacaaggaactcaaacaactcaacagtagaaaaaaaaacctgaatagacatttctcaaaagaagacatacaaatggcc

SEQ ID NO: 84 - DNA, 98 bp, Artificial Sequence
  Synthetic
aacagaccataaataaacacagaagacacacgagtgtaaagtcagtgccccgctgcgaattaaatcggggtgatgtgatggcgagtgagtgggtagtt

SEQ ID NO: 85 - DNA, 138 bp, Artificial Sequence
  Synthetic
atcattgaatgcaatcacatggaatcatcacagaatggaatcgtacggaatcatcatcgaatggaattgaatggaatcatcaattggactcgaatggaaacatcaaatggaatcgattggaagtgtcgaatggactcg

SEQ ID NO: 86 - DNA, 67 bp, Artificial Sequence
  Synthetic
ggtccattcgatgattctcttcgattccattcgataattccgttttttcccgtttgatgttgattcc

SEQ ID NO: 87 - DNA, 91 bp, Artificial Sequence
  Synthetic
agcaacttcagtaaagtgtcaggatacaaaatcaatgtgcaaaaatcacaagcattcttatacatcaataacagacaaacagagagccaaa

SEQ ID NO: 88 - DNA, 88 bp, Artificial Sequence
  Synthetic
agcaacttcagcaaagtctcaggatacaaaatcaatgtgcaaaaatcacaagcattcctatacaccaacaacagacaaacagagagcc

SEQ ID NO: 89 - DNA, 86 bp, Artificial Sequence
  Synthetic
gaataatcattgaacggaatcgaatggaatcatcatcggatggaaacgaatggaatcatcatcgaatggaaatgaaaggagtcatc

SEQ ID NO: 90 - DNA, 66 bp, Artificial Sequence
  Synthetic
taatcatcttcgaattgaaaacaaagcaatcattaaatgtactctaacggaatcatcgaatggacc

SEQ ID NO: 91 - DNA, 77 bp, Artificial Sequence
  Synthetic
ggaatcgaatggaatcaacatcaaacggaaaaaaacggaattatcgaatggaatcgaagagaatcatcgaatggacc

SEQ ID NO: 92 - DNA, 113 bp, Artificial Sequence
  Synthetic
gatcagcttagaatacaatggaacagaacagattagaacaatgtgattttattaggggccacagcactgttgactcaagtacaagttctgactcatgtagaactaacactttt

SEQ ID NO: 93 - DNA, 86 bp, Artificial Sequence
  Synthetic
agagaaaagatgatcatgtaaccattgaaaagacaatgtacaaaactaatactaatcacacaggaccagaaagcaatttagaccat

SEQ ID NO: 94 - DNA, 79 bp, Artificial Sequence
  Synthetic
aatggaatcgaatggaatcaacatcaaacggaaaaaacggaattatcgaatggaatcaaagagaatcatcgaatggacc

SEQ ID NO: 95 - DNA, 96 bp, Artificial Sequence
  Synthetic
aatggaattatcatcgaatggaatcgaatggaatcaacatcaaacggaaaaaaacggaattatcgaatggaatcgaagagaatcatcgaatggacc

SEQ ID NO: 96 - DNA, 149 bp, Artificial Sequence
  Synthetic
gtcaacacaggaccaacataggaccaacacagggtcaacacaggaccaacataggaccaacacagggtcaacacaagaccaacatgggaccaacacagggtcaacataggaccaacatgggaccaacacagggtcaacacaggaccaac

SEQ ID NO: 97 - DNA, 109 bp, Artificial Sequence
  Synthetic
tatagttgaatgaacacacatacacacacacatgccacaaaacaaaaacaaagttatcctcacacacaggatagaaaccaaaccaaatcccaacacatggcaagatgat

SEQ ID NO: 98 - DNA, 79 bp, Artificial Sequence
  Synthetic
gaatcaactcgattgcaatcgaatggaatggaatggtattaacagaatagaatggaatggaatggaatggaacggaacg

SEQ ID NO: 99 - DNA, 86 bp, Artificial Sequence
  Synthetic
aatggaatggaataatcgacggacccgaatgcaatcatcatcgtacagaatcgaatggaatcatcgaatggactggaatggaatgg

SEQ ID NO: 100 - DNA, 106 bp, Artificial Sequence
  Synthetic
aatacaaaccactgctcaacgaaataaaagaggatacaaacaaatggaagaacattctatgctcatgggtaggatgaattcatatcgtgaaaatggccatactgcc

SEQ ID NO: 101 - DNA, 142 bp, Artificial Sequence
  Synthetic
aaacacgcaaacacacacacaagcacactaccacacaagcggacacacatgcaaacacgcgaacacacacacatatacacacaagcacattacaaaacacaagcaaacaccagcagacacacaaacacacaaacatacatgg

SEQ ID NO: 102 - DNA, 76 bp, Artificial Sequence
  Synthetic
aatcgaacggaatcaacatcaaacggaaaaaaaacggaattatcgaatggaatcgaagagaatcatcgaatggacc

SEQ ID NO: 103 - DNA, 88 bp, Artificial Sequence
  Synthetic
acacatttcaaggaaggaaacaagaacagacagaaacacaacatacttcatgaaaccacattttagcatcctggccgagtattcatca

SEQ ID NO: 104 - DNA, 67 bp, Artificial Sequence
  Synthetic
ggatacaaaatcaatgtacaaaaatcacaagcattcttatacaccaataacagacaaacagagagcc

SEQ ID NO: 105 - DNA, 81 bp, Artificial Sequence
  Synthetic
taattgattcgaatggaatggaatagaatggaattgaatggaatggaccataatggattggactttaatagaaagggcatg

SEQ ID NO: 106 - DNA, 88 bp, Artificial Sequence
  Synthetic
agcaacttcagcaaagtctcaggatacaaaatcaatgtacaaaagtcacaagcattcttatacaccaacaaaagacaaacagagagcc

SEQ ID NO: 107 - DNA, 68 bp, Artificial Sequence
  Synthetic
acatcaaacggaaaaaaaaaacaaaacggaattatcgaatggaatcgaagagaatcatcgaatggacc

SEQ ID NO: 108 - DNA, 92 bp, Artificial Sequence
  Synthetic
acatctcacttttagtaatgaacagatcattcagacagaaaattagcaaagaaacatcagagttaaactacactctaaaccaaatggaccta

SEQ ID NO: 109 - DNA, 88 bp, Artificial Sequence
  Synthetic
gaagaaagcattcattcaagacatctaactcgttgatataatgcatacagttcaaaatgattacactatcattacatctagggctttc

SEQ ID NO: 110 - DNA, 83 bp, Artificial Sequence
  Synthetic
gcaaaagaaacaatcagtagagtaaacagacaactcatagaatgcaagaaaatcatcgcaatctgtacatccaacaaagggct

SEQ ID NO: 111 - DNA, 62 bp, Artificial Sequence
  Synthetic
acacacacattcaaagcagcaatatttacaacagccaaaaggtggaaacaattgagcaattg

SEQ ID NO: 112 - DNA, 87 bp, Artificial Sequence
  Synthetic
atcatcgaatagaatcgaatggtatcaacaccaaacggaaaaaaacggaattatcgaatggaatcgaagagaatcttcgaacggacc

SEQ ID NO: 113 - DNA, 95 bp, Artificial Sequence
  Synthetic
tgaaaatacaaatgaccatgcaagtaattccgcagggagagagcggatatgaacaaacagaagaaatcagatgggatagtgctggcgggaagtca

SEQ ID NO: 114 - DNA, 92 bp, Artificial Sequence
  Synthetic
aatcgaaaggaatgtcatcgaatggaatggactcaaatggaatagaatcggatggaatggcatcgaatggaatggaatggaattggatggac

SEQ ID NO: 115 - DNA, 138 bp, Artificial Sequence
  Synthetic
aacatgaacagtggaacaatcagtgaaccaatacaagggttaaataagctagcaattaaaagctgtatcactggtctaaagatagaagatcaagtagaaaatcagcgcaagaggaaagatatacgaaaactaatggcc

SEQ ID NO: 116 - DNA, 123 bp, Artificial Sequence
  Synthetic
cgaatggaatcattatggaatggaatgaaatggaataatcaaatggaattgaatggaatcatcgaatggaatcgaacaaaatcctctttgaatggaataagatggaatcaccaaatggaattg

SEQ ID NO: 117 - DNA, 100 bp, Artificial Sequence
  Synthetic
aaacggaatcaaacggaattatcgaatggaatcgaagagaatcatcgaacggactcgaatggaatcatctaatggaatggaatggaagaatccatggact

SEQ ID NO: 118 - DNA, 96 bp, Artificial Sequence
  Synthetic
gctagttcaacatatgcaaatcaataaacgtaatccatcacataaacagaaccaatgacaaaaaccacgattatctcaatagatgcagaaaaggcc

SEQ ID NO: 119 - DNA, 61 bp, Artificial Sequence
  Synthetic
accaatcaagaaaacaatgcaacccacagagaatggacaaaagcaaggcaggacaatggct

SEQ ID NO: 120 - DNA, 74 bp, Artificial Sequence
  Synthetic
atcgaatggaatcaacatcagacggaaaaaaacggaattatcaaatggaatcgaagagaatcatcgaatggacc

SEQ ID NO: 121 - DNA, 101 bp, Artificial Sequence
  Synthetic
atggaatcaacatcaaacggaaaaaaaaacggaattatcgaatggaatcgaagagaatcatcgaatggaccagaatggaatcatctaatggaatggaatgg

SEQ ID NO: 122 - DNA, 88 bp, Artificial Sequence
  Synthetic
aatggaatcatcatcgaatggaatcgaatggaatcatggaatggaatcaaatggaatcaaatggaatcgaatggaatggaatggaatg

SEQ ID NO: 123 - DNA, 87 bp, Artificial Sequence
  Synthetic
aacggaatcaaacggaattaccgaatggaatcgaatagaatcatcgaacggactcgaatggaatcatctaatggaatggaatggaag

SEQ ID NO: 124 - DNA, 97 bp, Artificial Sequence
  Synthetic
aaacggaatcaaacggaattatcgaatggaatcgaaaagaatcatcgaacggactcgaatggaatcatctaatggaatggaatggaagaatccatgg

SEQ ID NO: 125 - DNA, 88 bp, Artificial Sequence
  Synthetic
agcaacttcagcaaagtctcaggatacaaaatcaatgtacaaaaatcacaagcattcttatacaccaataacagacaaacagagagcc

SEQ ID NO: 126 - DNA, 71 bp, Artificial Sequence
  Synthetic
gaatgatacggantannnngnaatggaacgaaatgaaatggaatggaatggaatggaatggaatggaatgg

SEQ ID NO: 127 - DNA, 89 bp, Artificial Sequence
  Synthetic
aatggactcgaatggattaatcattgaacggaatcgaatggaatcatcatcggatggtaatgaatggaatcatcatcgaatggaatcgg

SEQ ID NO: 128 - DNA, 101 bp, Artificial Sequence
  Synthetic
gaatggaatcgaaaggaatgtcatcgaatggaatggaatggaacggaatggaatcgaatggaatggactcgaatggaatagaatcgaatgcaatggcatcg

SEQ ID NO: 129 - DNA, 69 bp, Artificial Sequence
  Synthetic
gaatagaatagaatggaatcatcgaatggaatcgaatggaatcatcatgatatggaattgagtggaatc

SEQ ID NO: 130 - DNA, 107 bp, Artificial Sequence
  Synthetic
taagccgataagcaacttcagcaaagtctcaggagacaaaatcaatgtgcaaaaaatcacaagcattcttatacactaataacagacaaacagagagccaaatcatg

SEQ ID NO: 131 - DNA, 88 bp, Artificial Sequence
  Synthetic
agcaacttcagcaaagtctcaggatacaaaatcaatgtgcaaaaatcaaaagcattcttatgcaccaataacagacacagagccaaat

SEQ ID NO: 132 - DNA, 81 bp, Artificial Sequence
  Synthetic
aggaaagttttcaatatgagaaagatacaaaccaacagaataagcaaactggataaacagaaaatacagagagagccaagg

SEQ ID NO: 133 - DNA, 79 bp, Artificial Sequence
  Synthetic
aatggaatggaacgcaattgaatggaatggaatggaacggaatcaacctgagtcaaatggaatggaatggaatggaatg

SEQ ID NO: 134 - DNA, 96 bp, Artificial Sequence
  Synthetic
aggaaaatgcaaatcagaacgactataacacaccatctcaaactcgttaggatggctattatcaaaaagtcaagagataacaaatgtgggcaaggg

SEQ ID NO: 135 - DNA, 82 bp, Artificial Sequence
  Synthetic
ggaacgaaatcgaatggaacggaatagaatagactcgaatgtcatggattgctatgtaattgattggaatggaatggaatcg

SEQ ID NO: 136 - DNA, 96 bp, Artificial Sequence
  Synthetic
gaattgaaaggaatgtattggaataaaatggaatcgaataggttgaaataccataggttcgaattgaatggaatgggagggacaccaatggaattg

SEQ ID NO: 137 - DNA, 106 bp, Artificial Sequence
  Synthetic
aacaaaacaaaaacccaactcaataacaagaagacaaacaacccaatttaaaatgagcaaagaacttgataaacatgtctccaaagaagatacggccaaagagcac

SEQ ID NO: 138 - DNA, 83 bp, Artificial Sequence
  Synthetic
atggttaaaactcaacaatgaaaacacaaacagcgcaatttaaaaatgggcaaaatgacaggccagacccagtggctcatgcg

SEQ ID NO: 139 - DNA, 89 bp, Artificial Sequence
  Synthetic
aagcaacttcagcaaagtctcgggatacaaaatcaatgtgcaaaaatcacaagcattcttatacaccactaacagacaaatggagagtc

SEQ ID NO: 140 - DNA, 113 bp, Artificial Sequence
  Synthetic
gaatggaatcaacatcaaacggaaaaaaacggaattatcgaatggaatcgaagagaatcatcgaatggaccagaatggaatcatctaatggaatggaatggaataatccatgg

SEQ ID NO: 141 - DNA, 83 bp, Artificial Sequence
  Synthetic
tagaaggaatttgatacatgctcagaaatacaggcaaaggaagtaggtgcctgccagtgaacacaggggaactatggctccta

SEQ ID NO: 142 - DNA, 77 bp, Artificial Sequence; identical to SEQ ID NO: 91
  Synthetic

SEQ ID NO: 143 - DNA, 95 bp, Artificial Sequence
  Synthetic
aactaagacaacagattgatttacactactattttcacacagccaaaaatatcactatggcaatcgtcaaaaggtcaattcaaagatgggacagt

SEQ ID NO: 144 - DNA, 77 bp, Artificial Sequence
  Synthetic
aaaagcaattggactgattttaaatatacgtggcaacaaggataaactgctaatgatgggtttgcaaatacagatcg

SEQ ID NO: 145 - DNA, 70 bp, Artificial Sequence
  Synthetic
aatggaatcaacatcgaacggaaaaaaacggaattatcgaatggaatcgaagagaatcatcgaatggacc

SEQ ID NO: 146 - DNA, 108 bp, Artificial Sequence
  Synthetic
tgcaagataacacattttagttgacaccattgaaaacagttttaaccaagaatattagaaccaatgaagcagagaaatcaaaagggtggatggaactgccaaaggatg

SEQ ID NO: 147 - DNA, 97 bp, Artificial Sequence
  Synthetic
tagaacagaattgaatggaatggcatcaaatggaatggaaacgaaaggaatggaattgaatggactcaaatgttatggaatcaaagggaatggactc

SEQ ID NO: 148 - DNA, 93 bp, Artificial Sequence
  Synthetic
aagagaatcatcgaatggaatcgaatggaatcaacatcaaacggaaaaaaacggaattatcgaatggaatcgaagagaatcatcgaatggacc

SEQ ID NO: 149 - DNA, 64 bp, Artificial Sequence
  Synthetic
atcaacatcaaacggaaaaaaacggaattatcgaatggaatcgaagagaatcatcgaatggacc

SEQ ID NO: 150 - DNA, 66 bp, Artificial Sequence
  Synthetic
gaatcaacatcaaacggaaaaaaaccgaattatcgaatggaatcgaagagaatcatcgaatggacc

SEQ ID NO: 151 - DNA, 106 bp, Artificial Sequence
  Synthetic
atcaacatcaaacggaatcaaacggaattatcgaatggaatcgaagagaatcatcaaatggactcgaatggaatcatctaatggaatggaatggaagaatccatgg

SEQ ID NO: 152 - DNA, 99 bp, Artificial Sequence
  Synthetic
aaacagttcaaaaattattgcaacaaaatgagagagatgagtttatcttgcaaactaatggatggtagcagtgacagtggcaaaacgtggtttgattct

SEQ ID NO: 153 - DNA, 89 bp, Artificial Sequence
  Synthetic
atcgaatggaatcattgaatggaaaggaatggaatcatcatggaatggaaacgaatggaatcactgaatggactcgaatgggatcatca

SEQ ID NO: 154 - DNA, 90 bp, Artificial Sequence
  Synthetic
attcagcctttaaaaaaagaagacagtcctgtcatttgtgacaatatgaatgaaacagacatcacattaaatgaaatgagccaggcgcag

SEQ ID NO: 155 - DNA, 83 bp, Artificial Sequence
  Synthetic
aggagaatagcagtagaatgacaaaattagattttcacatgaaacttgatgacagtgtaggaaatggactgaaaggacaagac

SEQ ID NO: 156 - DNA, 87 bp, Artificial Sequence
  Synthetic
aacccacaaagacaacagaagaaaagacaacagtagacaaggatgtcaaccacattttggaagagacaagtaatcaaacacatggca

SEQ ID NO: 157 - DNA, 83 bp, Artificial Sequence
  Synthetic
gaaaatgaacaatatgaacaaacaaacaaaattactacccttacgaaagtacgtgcattctagtatggtgacaaaaaggaaag

SEQ ID NO: 158 - DNA, 157 bp, Artificial Sequence
  Synthetic
aacatcaaacggaatcaaacggaattatcgaatggaatcgaagagaatcatcgaacggactcgaatggaatcatctaatggaatggaatggaagaatccatggactcgaatgcaatcatcatcgaatgaaatcgaatggaatcatcgaatggactcg

SEQ ID NO: 159 - DNA, 75 bp, Artificial Sequence
  Synthetic
accaacataagacaaagaaacatccagcagctgcctatggcaaaagattacaatgtgtcaaacaagagggcaatg

SEQ ID NO: 160 - DNA, 72 bp, Artificial Sequence
  Synthetic
atggaattcaatggaatggacatgantgnaatgnacttcaatggaatggnatcnaatggaatgnaattcant

SEQ ID NO: 161 - DNA, 79 bp, Artificial Sequence
  Synthetic
tatgactttcacaaattacagaaaaagacacccatttgacaagggaactgaaggtggtgaagacatactggcaggctac

SEQ ID NO: 162 - DNA, 85 bp, Artificial Sequence
  Synthetic
aatggaaaggaatcgaatggaagggaatgaaattgaatcaacaggaatggaagggaatagaatagacggcaatggaatggactcg

SEQ ID NO: 163 - DNA, 113 bp, Artificial Sequence
  Synthetic
agcctatcaaaaagtgggctaagaatatgaatacacaattctcaaaagaagatatacaaatgggcaacaaacatatgaaaacatactcaacatcactaatgatcagggaaatg

SEQ ID NO: 164 - DNA, 88 bp, Artificial Sequence
  Synthetic
agcaacttcagcaaagtatcaggatacaaaatcaatgtacaaaaatcccaagcattcttatacaccaacaacagacaaacagagagcc

SEQ ID NO: 165 - DNA, 87 bp, Artificial Sequence
  Synthetic
aaagacaatatacaaatggccaataagcacatgaaaagacgctcaacatccttagtcgttaaggcaatgcaaatcaaaaccacaatg

SEQ ID NO: 166 - DNA, 88 bp, Artificial Sequence
  Synthetic
agcaacttcagcaaagtctcaggatacaaaatcgatgtgcaaaaatcacaagcattcttatacaccaacaacagataaacagagagcc

SEQ ID NO: 167 - DNA, 106 bp, Artificial Sequence
  Synthetic
aacggaaaaaaaacggaattatcgaatggaatcgaagagaatcatcgaatggaccagaatggaatcatctaatggaatggaatggaataatccatggactcgaatg

SEQ ID NO: 168 - DNA, 70 bp, Artificial Sequence
  Synthetic
aacagcaatagacacaaagtcagcacttacagtacaaaaactaatggcaaaagcacatgaagtgggacat

SEQ ID NO: 169 - DNA, 102 bp, Artificial Sequence
  Synthetic
ggaatcaaacggaattatcgaatggaatcgaagagaatcatagaacggactcaaatggaatcatctaatggaatggaatgggagaatccatggactcgaatg

SEQ ID NO: 170 - DNA, 70 bp, Artificial Sequence
  Synthetic
aatggaatcaatatcaaacggaaaaaaacggaattatcgaatggaatcgaagagaatcatcgaatggacc

SEQ ID NO: 171 - DNA, 87 bp, Artificial Sequence
  Synthetic
aaaatgatcatgagaaaattcagcaacaaaaccatgaaattgcaaagatattacttttgggatggaacagagctggaaggcaaagag

SEQ ID NO: 172 - DNA, 96 bp, Artificial Sequence
  Synthetic
aacggaatcaaacggaattatcgaatggaatcgaaaagaatcatcgaacggactcgaatggaatcatctaatggaatggaatggaagaatccatgg

SEQ ID NO: 173 - DNA, 90 bp, Artificial Sequence
  Synthetic
aaacggaattatcgaanggaatcaaagagaatcatcgaannnnnacgaatggaatcatataatggaatggaatggaataatccatggacc

SEQ ID NO: 174 - DNA, 85 bp, Artificial Sequence
  Synthetic
aatggaatcgaatggattgatatcaaatggaatggaatggaagggaatggaatggaatggaattgaaccaaatgtaatggatttg

SEQ ID NO: 175 - DNA, 89 bp, Artificial Sequence
  Synthetic
taaaagacggaacagatagaaagcagaaaggaaaggtgaattgcattaccactattcatactgccacacacatgacattaggccaagtc

SEQ ID NO: 176 - DNA, 62 bp, Artificial Sequence
  Synthetic
acaaacaatccaattcgaaaatgggcaagatatttcaccaaagacatgagctgatatttcac

SEQ ID NO: 177 - DNA, 79 bp, Artificial Sequence
  Synthetic
aatggaatcgaatggaacaatcaaatggactccaatggagtcatctaatggaatcgagtggaatcatcgaatggactcg

SEQ ID NO: 178 - DNA, 102 bp, Artificial Sequence
  Synthetic
taacacataaacaaacacagagacaaaatctccgagatgttaatctgctccagcaatacagaacaatttctattaccaacagaatgcttaatttttctgcct

SEQ ID NO: 179 - DNA, 77 bp, Artificial Sequence
  Synthetic
ggaatcgaatggaatcaacatcaaacggaaaaaaacggaattatcgaatggaatcaaagagaatcatcgaatggacc

SEQ ID NO: 180 - DNA, 51 bp, Artificial Sequence
  Synthetic
agaatggaaaggaatcgaaacgaaaggaatggagacagatggaatggaatg

SEQ ID NO: 181 - DNA, 71 bp, Artificial Sequence
  Synthetic
gaatggaatggaaaggaatcgaaacgaaaggaatggagacagatggaatggaatggaacagagagcaatgg

SEQ ID NO: 182 - DNA, 89 bp, Artificial Sequence
  Synthetic
gaatcatcataaaatggaatcgaatggaatcaacatcaaatggaatcaaatggtctcgaatggaatcatcttcaaatggaatggaatgg

SEQ ID NO: 183 - DNA, 127 bp, Artificial Sequence
  Synthetic
aacaacaatgacaaacaaacaacaacgacaaagacatttatttggttcacaaatctccagggtgtacaagaagcatggtgccagcatctgctcagcttctgatgagggctctgggaagcttttactc

SEQ ID NO: 184 - DNA, 96 bp, Artificial Sequence
  Synthetic
aacggactcgaacggaatataatggaatggaatggattcgaaaggaatggaatggaatggacaggaaaagaattgaatgggattggaatggaatcg

SEQ ID NO: 185 - DNA, 85 bp, Artificial Sequence
  Synthetic
aggaaataaaagaagacacaaacaaatggaagaacattccatgcttatggatagggagaatcagtatcgtgaaaatggccatact

SEQ ID NO: 186 - DNA, 112 bp, Artificial Sequence
  Synthetic
aacatcaaacgaaatcaaacggaattatcaaattgaatcgaagagaatcatcgaattgccacgaatgcaatcatctaatggtatggaatggaataatccatggacccagatg

SEQ ID NO: 187 - DNA, 94 bp, Artificial Sequence
  Synthetic
agaaattaacagcaaaagaaggatgcagtgcaactcaggacaacacatacaattcaagcaacaaatgtatagtggctgggcaccaaggatacag

SEQ ID NO: 188 - DNA, 82 bp, Artificial Sequence
  Synthetic
gcaataaaatcgactcagatagagaagaatgcaatggaatggaatggaatggaatggaatgggatggaatggtatggaatgg

SEQ ID NO: 189 - DNA, 82 bp, Artificial Sequence
  Synthetic
ccacataaaacaaaactacaagacaatgataaagttcacaacattaacacaatcagtaatggaaaagcctagtcaatggcag

SEQ ID NO: 190 - DNA, 91 bp, Artificial Sequence
  Synthetic
ggacaacatacacaaatcagtcaagatacatcatttcaacagaatgaaagacaaaaaccatttgatcacttcaatcgatgatgaaaaagca

SEQ ID NO: 191 - DNA, 72 bp, Artificial Sequence
  Synthetic
gaaatcatcatcaaacggaatcgaatggaatcattgaatggaatggaatggaatcatcatggaatggaaacg

SEQ ID NO: 192 - DNA, 157 bp, Artificial Sequence
  Synthetic
tggaatggantggaatgnaatcnaatcnnntggtaatgaatcaaatggaatcaaatcgaatggnaataatggaatcnannggaaacgaatggnatcgaattgcactgattctactgacttcgaggaaaatgaaatgaaatgcggtgaagtggaatgg

SEQ ID NO: 193 - DNA, 88 bp, Artificial Sequence
  Synthetic
agcaacttcagcaaagtctcaggatacaaaatcaatgggaaaaaatcacaagcattcctatacatcaataacagacaaacagagagcc

SEQ ID NO: 194 - DNA, 71 bp, Artificial Sequence
  Synthetic
gaatgttatgaaatcaactcgaacggaatgcaatagaatggaatggaatggaatggaatggaatggaatgg

SEQ ID NO: 195 - DNA, 102 bp, Artificial Sequence
  Synthetic
agtagaattgcaattgcaaatttcacacatatactcacacacaagtacacacatccacttttacaactaaaaaaactagcacccaggacaggtgcagtggct

SEQ ID NO: 196 - DNA, 68 bp, Artificial Sequence
  Synthetic
ggaatcaacatcaaacggaaaaaaaacggaattatcgaatggaatcgaagagaatcatcgaatggacc

SEQ ID NO: 197 - DNA, 69 bp, Artificial Sequence
  Synthetic
ggaataatcatcatcaaacagaaccaaatggaatcattgaatggaatcaaaggcaatcatggtcgaatg

SEQ ID NO: 198 - DNA, 101 bp, Artificial Sequence
  Synthetic
actcaggaaaaataacgaatccaactcacaggagaaagaagtacaaaccagaaaccaatttcaaattacaaggaccagaatactcatgttggctggccagt

SEQ ID NO: 199 - DNA, 83 bp, Artificial Sequence
  Synthetic
aaacgcacaaacaaagcaaggaaagaatgaagcaacaaaagcagagatttattgaaaatgaaaaatacactccacagggtggg

SEQ ID NO: 200 - DNA, 108 bp, Artificial Sequence
  Synthetic
gcatagaatcgaatggaattatcattgaatggaatcgaatggaatcaacatcaaacggaaaaaaacggaattatcgaatggaatcgaagagaatcatcgaatggaccc

SEQ ID NO: 201 - DNA, 82 bp, Artificial Sequence
  Synthetic
aatggaatcgaanagaatcatcgaacggactcgaatggaatcatctaatggaatggaatggaataatccatggacccgaatg

SEQ ID NO: 202 - DNA, 99 bp, Artificial Sequence
  Synthetic
aaatgaatcgaatggaattgaatggaatcaaatagaacaaatggaatcgaaatgaatcaaatggaatcgaatcgaatggaattgaatggcatggaattg

SEQ ID NO: 203 - DNA, 131 bp, Artificial Sequence
  Synthetic
ntcacaatcacacaacacattgcacatgnnnannatgcactcacaatacacacacaacacatacacaacacacatgcaatacaacacaaaacgcaacacaacatatacacnacacacagcacacanatgcc

SEQ ID NO: 204 - DNA, 74 bp, Artificial Sequence
  Synthetic
gaatggaatcaaatcgaatgaaatggaatggaatagaaaggaatggaatgaaatggaatggaaaggattcgaat

SEQ ID NO: 205 - DNA, 87 bp, Artificial Sequence
  Synthetic
aaagacttaaacgttagacctaaaaccataaaaaccctagaggaaaacctaggcattaccattcaggacttaggcatgggcaaggac

SEQ ID NO: 206 - DNA, 86 bp, Artificial Sequence
  Synthetic
gtttacagtcaagtgtacaaacagaatataagcaaacaaaagagaacatatacttacaaactatgctaagtgccatgaaggaaaag

SEQ ID NO: 207 - DNA, 108 bp, Artificial Sequence
  Synthetic
aaagtccaaagatgaacaaaatatccagaaggaaaacaaatgcacttggggagtgggaaagaaaaccaagactgagcaatgcgtcaagctcagacgtgcctcactacg

SEQ ID NO: 208 - DNA, 97 bp, Artificial Sequence
  Synthetic
aaacggaatcaaacggaattatcgaatggagtcgaaaagaatcatcgaacggactcgaatggaatcatctaatggaatggaatggaagaatccatgg

SEQ ID NO: 209 - DNA, 94 bp, Artificial Sequence
  Synthetic
aattgattcgaaattaatggaattgaatggaatgcaatcaaatggaatggaatgtaatgcaatggaatgtaatagaatggaaagcaatggaatg

SEQ ID NO: 210 - DNA, 79 bp, Artificial Sequence
  Synthetic
tacagaacacatgactcaacaacagcagaaagcatattcttttcaaatgcacatgaaacattatcatgatggaccaaat

SEQ ID NO: 211 - DNA, 75 bp, Artificial Sequence
  Synthetic
taagacacatagaaaacataaagcaaaatggcagatgtaaatgcaacctatcaatcaaaacattacgaatggctt

SEQ ID NO: 212 - DNA, 65 bp, Artificial Sequence
  Synthetic
ggaacaaaatgaaatcgaacggtaggaatcatacagaacagaaagaaatggaacggaatggaatg

SEQ ID NO: 213 - DNA, 68 bp, Artificial Sequence
  Synthetic
aacggaaaaaacggaattatcgaatggaatcgaagagaatcatcgaatggaatcgaatggagtcatcg

SEQ ID NO: 214 - DNA, 93 bp, Artificial Sequence
  Synthetic
aacatacgaaaatcaataaacgtaatccagcatataaacagaaccaaagacaaaaaccacatgattatctcaatagatgcagaaaaggccttt

SEQ ID NO: 215 - DNA, 75 bp, Artificial Sequence
  Synthetic
aatcgaacggaatcaacatcaaacggaaaaaaacggaattatcgaatggaatcgaagagaatcatcgaatggacc

SEQ ID NO: 216 - DNA, 87 bp, Artificial Sequence
  Synthetic
agaatggaatgcaatagaatggaatgcaatggaatggagtcatccgtaatggaatggaaaggaatgcaatggaatggaatggaatgg

SEQ ID NO: 217 - DNA, 98 bp, Artificial Sequence
  Synthetic
ggaataaaacggactcaatagtaatggattgcaatgtaattgattcgatttcgaatggaatcgcatggaatgtaatggaatggaatggaatggaaggc

SEQ ID NO: 218 - DNA, 88 bp, Artificial Sequence
  Synthetic
agcaacttcagcaaagtctcaggatacaaaatcaatgtacaaaaatcacaagcattcttatacaccaacaacagacaaacagagagcc

SEQ ID NO: 219 - DNA, 68 bp, Artificial Sequence
  Synthetic
taagcagagaaaatatcaacacgaaaataatgcaaggagaaaaatacagaacaatccaaaatgtggcc

SEQ ID NO: 220 - DNA, 70 bp, Artificial Sequence
  Synthetic
aatggaatcaacatcaaacggaaaaaaacggaattatcgtatggaatcgaaaagaattatcgaatggacc

SEQ ID NO: 221 - DNA, 57 bp, Artificial Sequence
  Synthetic
tcaaacggaaaaaaacggaattatcgaatggaatcgaagagaatcatcgaatggacc

SEQ ID NO: 222 - DNA, 85 bp, Artificial Sequence
  Synthetic
aacttcagcaaattctcaggatacaaaatcaatgtgcaaaaaccacaagcattcctatacaccaataatagacagtgagccaaat

SEQ ID NO: 223 - DNA, 111 bp, Artificial Sequence
  Synthetic
acatcaaacggaatcaaacggaattatcgaatggaatcgaaaagaatcatcgaacggactcgaatggaatcatctaatggaatggaatggaagaatccatggactcgaatg

SEQ ID NO: 224 - DNA, 76 bp, Artificial Sequence
  Synthetic
aatggaatcgaatgcaatcatcgaacggaatcgaatggcatcaccgaatggaatggaatggaatggaatggaatgg

SEQ ID NO: 225 - DNA, 67 bp, Artificial Sequence
  Synthetic
aatccagcatataaacagaaccaaagacaaaaaccacatgattatctcaatagatgcagaaaaggcc

SEQ ID NO: 226 - DNA, 131 bp, Artificial Sequence
  Synthetic
tgactaaacagagttgaacaagaacaaaaagcaaatttgcagaaatgaaatacatactaattgaaagtccatggacaggctcaacagatgatatagatacagctaaagagataattagtgaaatggatcag

SEQ ID NO: 227 - DNA, 75 bp, Artificial Sequence
  Synthetic
gatcatcagagaaacagagaaatgcaaattaaaaccacaatgagatactatctccacacaagtcagaatggctat

SEQ ID NO: 228 - DNA, 80 bp, Artificial Sequence
  Synthetic
aggatacaaaatcaatgtacaaaaatcacaaacattcttatacaccaacaacagacaaacagagagccaaatcatgggtg

SEQ ID NO: 229 - DNA, 99 bp, Artificial Sequence
  Synthetic
taagcaacttcagcaaagtctcaggatacaaaatcaatgtacaaaaatcacaagcattcttatacaccaacaacagacaaacaagagtgccaaatcatg

SEQ ID NO: 230 - DNA, 87 bp, Artificial Sequence
  Synthetic
agaattgattgaatccaagtggaattgaatggaatggaatggattagaaaggaatggaatggattggaatggattggaatggaaagg

SEQ ID NO: 231 - DNA, 76 bp, Artificial Sequence
  Synthetic
aatggaatgcaatcgaatggaatggaatcgaacggaatggaataaaatggaagaaaactggcaagaaatggaatcg

SEQ ID NO: 232 - DNA, 96 bp, Artificial Sequence
  Synthetic
aactgcatcaactaacaggcaaaataaccagctaatatcataatgacaggattaaattcacaaatgacaatattaaccgtaaatgtaaatgggcta

SEQ ID NO: 233 - DNA, 102 bp, Artificial Sequence
  Synthetic
tacaaagaactcaaacaaatcagcaagaacaaaaacaatcccaacaaaatgttggacaaagacatgaatagacaattctcgaaagaagatgtacaaatggct

SEQ ID NO: 234 - DNA, 99 bp, Artificial Sequence
  Synthetic
agagaaatgcaaatcaaaaccacaatggaataccatctcacgccagtcagaatggcaattattaaaaaatcacaacaattaatgatggcaaggctgtgg

SEQ ID NO: 235 - DNA, 108 bp, Artificial Sequence
  Synthetic
gtaaacaaacaatcaagcaagtaagaacagaaataacagcatttggcttttgagttaatgacaagaacactcggcatgggagcctgggtgagcaaatcacagatcttc

SEQ ID NO: 236 - DNA, 91 bp, Artificial Sequence
  Synthetic
gaatcaacccgagcggaaaggaatggaatggaatggaatcaacacgaatggaatggaacggaatggaatgggatgggatgaaatggaatgg

SEQ ID NO: 237 - DNA, 88 bp, Artificial Sequence
  Synthetic
agcaacttcagcaaagtctcaggagacaaaatcaatgtacaaaaatcacaagcattcttatacaccaataacagacaaacagagagcc

SEQ ID NO: 238 - DNA, 71 bp, Artificial Sequence
  Synthetic
aagaaatggaatcgaagagaatggaaacaaacggaatggaattgaatggaatggaattgaatggaatggga

SEQ ID NO: 239 - DNA, 101 bp, Artificial Sequence
  Synthetic
gacatgcaaacacaacacacagcacacatggaacatgcatcagacatgcaaacacaacacacataccacacatggcatatgcatcagacgtgcctcactac

SEQ ID NO: 240 - DNA, 91 bp, Artificial Sequence
  Synthetic
tacagataagaaaattgagactcaagagtattacataaattgtttcagctaccacagcaaaaaatggtatggttgggaatcaagctcaggg

SEQ ID NO: 241 - DNA, 135 bp, Artificial Sequence
  Synthetic
aaaggaatgcactcgaatggaatggacttgaatggaatgtctccgaatggaacagactcgtatgaaatggaatcgaatggaatggaatcaaatggaattgatttgagtgaaatggaatcaaatggaatggcaacg

SEQ ID NO: 242 - DNA, 95 bp, Artificial Sequence
  Synthetic
tgaaacaaatgataatgaaaatacaacataccaaacatacgagatacagtaaaagcagtactaagatgcaagtatatattgctacaagtgcctac

SEQ ID NO: 243 - DNA, 89 bp, Artificial Sequence
  Synthetic
ggaacaaaatgaaatcgaacggtaggaatcgtacagaacggaaagaaatggaacggaatggaatgcactcgaatggaaaggagtccaat

SEQ ID NO: 244 - DNA, 95 bp, Artificial Sequence
  Synthetic
aaattgattgaaatcatcataaaatggaatcgaagggaatcaacatcaaatggaatcaaatggaatcattgaacggaattgaatggaatcgtcat

SEQ ID NO: 245 - DNA, 96 bp, Artificial Sequence
  Synthetic
agaaaggattcgaatggaatgaaaaagaattgaatggaatagaacagaatggaatcaaatcgaatgaaatggaatggaatagaaaggaatggaatg

SEQ ID NO: 246 - DNA, 84 bp, Artificial Sequence
  Synthetic
agaatggaaagcaatagaatggaacgcactggattcgagtgcaatggaatcaattggaatggaatcgaatggaatggattggca

SEQ ID NO: 247 - DNA, 103 bp, Artificial Sequence
  Synthetic
aacaccaaacggaaaaaaacggaattatcgaatggaatcgaagagaatcttcgaacggacccgaatgggatcatctaatggaatggaatggaataatccatgg

SEQ ID NO: 248 - DNA, 94 bp, Artificial Sequence
  Synthetic
aatggagactaatgtaatagaatcaaatggaatggcatcgaatggaatggactggaatggaatgtgcatgaatggaatggaatcgaatggattg

SEQ ID NO: 249 - DNA, 84 bp, Artificial Sequence
  Synthetic
tgggatatgggtgaaagaacaagtttgcagaaaagatacagtgaattatggaccatgagttcgggaaagaagggtaggactgcg

SEQ ID NO: 250 - DNA, 151 bp, Artificial Sequence
  Synthetic
aaatcgaatggaacgcaatagaatagactcgaatgtaatggattgctatgtaattgattcgaatggaatggaatcgactggaatgcaatccaatggaatggaatgcaatgcaatggaatggaatcgaacggaatgcagtggaagggaatgg

SEQ ID NO: 251 - DNA, 107 bp, Artificial Sequence
  Synthetic
aatcaacaaggaactgaaacaagtaaacaagaaaacaaataacaccataaaacatgggcaaaggacataaacagacatttttcaaaaaagacatacaaatggccgag

SEQ ID NO: 252 - DNA, 89 bp, Artificial Sequence
  Synthetic
aatggaatcaacatcaaacggaaaaaaacggaattatcgaatggaatcgaagagaatcatcgaatggacccaggctggtcttgaactcc

SEQ ID NO: 253 - DNA, 82 bp, Artificial Sequence
  Synthetic
attgaatgggctagaatggaatcatctttgaacggaatcaaagggaatcatcatcgaatggaatcgaatggaaatgtcaacg

SEQ ID NO: 254 - DNA, 143 bp, Artificial Sequence
  Synthetic
aatggactcgaatggaatcaacatcaaatggaatcaagcggaattatcgaatgaaatcgaagagaatcatcgaatggactcgaaaggaatcatctaatggaatggaatggaataatccatggactcgaatgcaatcatcatcg

SEQ ID NO: 255 - DNA, 106 bp, Artificial Sequence
  Synthetic
acagacagagatttaaaacaataaacaagcagtaagcaaacacagataacaaaatgacatgatccaacaaatactcagaaggagacttagaaatgaattgagggtc

SEQ ID NO: 256 - DNA, 135 bp, Artificial Sequence
  Synthetic
aatgtaatccagcatataaacagagccaaagacaaaaaccacatgattatctcaatagatgcagaaaaagcctttgacaaaattcaacaacccttcatgctaaaaactctcaataaattaggtattgatgggacg

SEQ ID NO: 257 - DNA, 97 bp, Artificial Sequence
  Synthetic
aaacggaaaaaaacggaattattgaatggaatcgaagagaatcttcgaacggacccgaatggaatcatctaatggaatggaatggaataatccatgg

SEQ ID NO: 258 - DNA, 84 bp, Artificial Sequence
  Synthetic
gctcaaggaaataaaataggacacaaagaaatggaaaaacattccatactcatggatagaaagaatcaatatcatgaaatggcc

SEQ ID NO: 259 - DNA, 79 bp, Artificial Sequence
  Synthetic
actcgagtggaattgactgtaacaaaatggaaagtaacggattggaatcgaatggaacggaatggaatggaatggacat

SEQ ID NO: 260 - DNA, 120 bp, Artificial Sequence
  Synthetic
tacaaactttaaaaaatgatcaacagatacacagttagcaagaaagaattgagggcaaagaatatgccagacaaactcaagaggaagatgatggtagagataggtcacattggagtgtca

SEQ ID NO: 261 - DNA, 77 bp, Artificial Sequence
  Synthetic
ggaatcgaatggaatcaatatcaaacggaaaaaaacggaattatcgaatggaatcgaagagaatcatcgaatggacc

SEQ ID NO: 262 - DNA, 105 bp, Artificial Sequence
  Synthetic
aacggaatcaaacggaattatcgaatggaatcgaaaagaatcatcgaacggactcgaatggaatcatctaatgtaatggaatggaagaatccatggactcgaatg

SEQ ID NO: 263 - DNA, 84 bp, Artificial Sequence
  Synthetic
ggaaataacagagaacacaaacaaatgggaaaacattccatgttcatggataggaagaatcaatattgtgaaaatggccatact

SEQ ID NO: 264 - DNA, 105 bp, Artificial Sequence
  Synthetic
aacggaaaaaaacggaattatcgaatggaatcgaagagaatcatcgaatggaccagaatggaatcatctaatggaatggaatggaataatccatggactcgaatg

SEQ ID NO: 265 - DNA, 103 bp, Artificial Sequence
  Synthetic
caacatcaaacggaaaaaaacggaattatggaatggaatcgaagagaatcatcgaatggacccgaatggaatcatctgaaatataatagactcgaaaggaatg

SEQ ID NO: 266 - DNA, 78 bp, Artificial Sequence
  Synthetic
atggaatcgaatggaatggactggaatggaatggattcgaatggaatcgaatggaacaatatggaatggtaccaaatg

SEQ ID NO: 267 - DNA, 71 bp, Artificial Sequence
  Synthetic
gaatggaatcaacatcaaacggaaaaaaacggaattatcgaatggaatcgaagagaatcatcgaatggacc

SEQ ID NO: 268 - DNA, 105 bp, Artificial Sequence
  Synthetic
aaggaatttaagcaaatcaacaagcaaaaccaaaataatcccattaaaaagtgggtaaaggacatgaatacacacttgtcaatagaggacattcaagtggccaac

SEQ ID NO: 269 - DNA, 89 bp, Artificial Sequence
  Synthetic
aaatggactcgaatggaatcatcatagaatggaatcgaatgcaatggaatggaatcttccggaatggaatggaatggaatggaatggag

SEQ ID NO: 270 - DNA, 85 bp, Artificial Sequence
  Synthetic
gaatcancnnnnnnnggaatcgaatggaatcaacatcaaatggaatcaaatggaatcattgaacggaattgaatggaatcgtcat

SEQ ID NO: 271 - DNA, 92 bp, Artificial Sequence
  Synthetic
agcaacttcagcaaagtctcaggatacaaaatcaatgtacaaaaatcacaagcattcttatacaccaataacagacaaacagagagccaaaa

SEQ ID NO: 272 - DNA, 85 bp, Artificial Sequence
  Synthetic
tatgcaaatcaataaacataatccatcacataaacagaaacaaagacaaaatgacatgattatctcaatagatgcagaaaaggcc

SEQ ID NO: 273 - DNA, 69 bp, Artificial Sequence
  Synthetic
agtaaatcaccataaagaaggtaagagttcattcacaaaaacaacaaactgaagaatcaggccatagta

SEQ ID NO: 274 - DNA, 89 bp, Artificial Sequence
  Synthetic
agaaacagaaaacagtcaaaccaatgggcaatccatatcagatgcagtattatgaacagaagtgtaaagaatgcaccaggcacaatggc

SEQ ID NO: 275 - DNA, 98 bp, Artificial Sequence
  Synthetic
aggaaaaacaacaacaacaacaggaaaacaacctcagtatgaagacaagtacattgatttattcaacatttactgatcacttttcaggtggtaggcag

SEQ ID NO: 276 - DNA, 131 bp, Artificial Sequence
  Synthetic
gattggaacgaaatcgaatggaacggaatagaatagactcgaatgtaatggattgctatgtaattgattcgaatggaatggaatcgaatggaatgcaatccaatggaatggaatgcaatgcaatggaatgg

SEQ ID NO: 277 - DNA, 92 bp, Artificial Sequence
  Synthetic
aacatatggaaaaaaactcaacatcactgatcattagagaaatgcaaatcaaaaccacaatgagataccatctcacgccagtcagaatggcg

SEQ ID NO: 278 - DNA, 126 bp, Artificial Sequence
  Synthetic
atggaatggaataatcaacgtactcgaatgcaatcatcatcgtatagaatcgaatggaatcatcgaatggactcgaatggaataatcattgaacggagtcgaatggaatcatcatcggatggaaac

SEQ ID NO: 279 - DNA, 95 bp, Artificial Sequence
  Synthetic
aaanaantcnaatggaatcnntgncgaatggaatggaatggaatcgaanaattgaattgnnnanaatcnnangnaancnttgnatgggctcaaat

SEQ ID NO: 280 - DNA, 77 bp, Artificial Sequence
  Synthetic
agaaaagataactcgattaacaaatgaacaaacacctgaatacacaagtctcaaaagaagacataaaaatggccaac

SEQ ID NO: 281 - DNA, 102 bp, Artificial Sequence
  Synthetic
atggaatcaacatcaaacggaatcacacggaattatcgaatggaatcgaaaagaatcatcgaacggactcgaatggaatcatctaatggaatggaatggaag

SEQ ID NO: 282 - DNA, 103 bp, Artificial Sequence
  Synthetic
aatggaatcaacatcaaacggaatcaagcgaaattatcgaatggaatcgaagagaatcatcgaatggactcgaatggaatcatctaatggaatggaatgggat

SEQ ID NO: 283 - DNA, 82 bp, Artificial Sequence
  Synthetic
aaacacagtacaaatactaattcaaatcaaacttactcaaagtcataatcaaacatgccagacgggctgaggggcagcatta

SEQ ID NO: 284 - DNA, 76 bp, Artificial Sequence
  Synthetic
aaccactgctcaaggaaataagagagaacacaaacaaatgaaaaaacattccatgctcatggataggaagaatcag

SEQ ID NO: 285 - DNA, 100 bp, Artificial Sequence
  Synthetic
ggaatcgagtggaatcatcgaaagaaatcgaatggaatcattgtcgaatggaatggaatggaatcaaagaatggaatcgaagggaatcattggatgggct

SEQ ID NO: 286 - DNA, 139 bp, Artificial Sequence
  Synthetic
aaagaaagacagagaacaaacgtaattcaagatgactgtttacatatccaagaacattagatggtcaaagactttaagaaggaatacattcaaaggcaaaaagtcacttactgattttggtggagtttgccacatggac

SEQ ID NO: 287 - DNA, 90 bp, Artificial Sequence
  Synthetic
aagatagagttgaaacagtggacaattaaagagtaatttggaagaatggtgaaattacagccatgctttgaatcaggcgggttcactggc

SEQ ID NO: 288 - DNA, 77 bp, Artificial Sequence
  Synthetic
aagagtatcaacagtaaattacattagcagaagaatcaacaaacatgaaaatagaaattatggtagccaaagaacag

SEQ ID NO: 289 - DNA, 148 bp, Artificial Sequence
  Synthetic
gaaaggaatcatcattgaatgcaatcacatggaatcatcacagaatggaatcgtacggaatcatcatcgaatggaattgaatggaatcatcaattggactcgaatggaatcatcaaatggaatcgattggaagtgtcaaatggactcg

SEQ ID NO: 290 - DNA, 72 bp, Artificial Sequence
  Synthetic
cagcgcaccacagcacacacagtatacacatgacccacaatacacacaacacacaacacattcacacaccac

SEQ ID NO: 291 - DNA, 87 bp, Artificial Sequence
  Synthetic
gcaaacagaattcaacactacattagaacgatcattcatcacgacctagtaggatgtttttcctgggatgcaaggatggttcaacat

SEQ ID NO: 292 - DNA, 71 bp, Artificial Sequence
  Synthetic
caatcaaaacagcaatgagataccattttacaccaatcaaaatggctactaaaaagtcaaaagcaaatgcc

SEQ ID NO: 293 - DNA, 90 bp, Artificial Sequence
  Synthetic
agaaccatattgaagagacagagtgatatataaaactgctaactcaagcagcacaagaattaaatgaataccaagaaaatacttggccag

SEQ ID NO: 294 - DNA, 93 bp, Artificial Sequence
  Synthetic
tggaatagaatggaatcaatgttaagtggaatcgagtggaatcatcgaaagaaatcgaatggaatcattgtcgaatggtatggaatggaatca

SEQ ID NO: 295 - DNA, 124 bp, Artificial Sequence
  Synthetic
aatggaatggaatcatcgcatagaatngaatggaattatcatcgaattgaatcgaatggtatcaacatcaaacggaaaaaaacggaaatatcgaanggaatcgaagagaatcatcgaacggacc

SEQ ID NO: 296 - DNA, 95 bp, Artificial Sequence
  Synthetic
acatacgcaaatcaataaacataatccatcacataaacagaaccaaagacaaaaatcacatgattatctcaatagatgcagaaaaggccttcgac

SEQ ID NO: 297 - DNA, 83 bp, Artificial Sequence
  Synthetic
aaaaaatgttcaacatcactagtcagcagagaaatgcaaatcaaaatcacaatgagataacttctcacaccagacagcatggc

SEQ ID NO: 298 - DNA, 90 bp, Artificial Sequence
  Synthetic
gaaaaacaaaacaaaacaaacaaacaaacaatcaaaaaagtggtagcagaaaccagaaagtccatgtatatagctaattggcctggttgt

SEQ ID NO: 299 - DNA, 58 bp, Artificial Sequence
  Synthetic
aacagcaatgacaatgatcagtaacaacaagacttttaactttgaaaaaatcaggacc

SEQ ID NO: 300 - DNA, 91 bp, Artificial Sequence
  Synthetic
aagagcctgaatagctaaagtgatcataagcaaaaagaacaaagtcggaagcatcacattacctgacttcaaactatactcaaaggctatg

SEQ ID NO: 301 - DNA, 78 bp, Artificial Sequence
  Synthetic
aaaaggaaatacaagacaacaaacacagaaacacaaccatcgggcatcatgaaacctcgtgaagataatcatcagggt

SEQ ID NO: 302 - DNA, 91 bp, Artificial Sequence
  Synthetic
aagcaaagaaagaatgaagcagcaaaagaacgaaagcaggaatttattgaaaaccaaagtacactccacagtatgggagcggacccgagca

SEQ ID NO: 303 - DNA, 67 bp, Artificial Sequence
  Synthetic
gcaaatgattataagtgctgttatagaaacattcaaagaccagaaaaggaccacaatggctgaccac

SEQ ID NO: 304 - DNA, 87 bp, Artificial Sequence
  Synthetic
agagcagaaacaaatggaattgaaatgaagacaacaatcaaaagcatcaatgaaatgaaaagttgggttttggaagagagaaacaat

SEQ ID NO: 305 - DNA, 70 bp, Artificial Sequence
  Synthetic
acacaaacacacacacacacacacacacacacacacacacacacacacacacacacacacacacacatac

SEQ ID NO: 306 - DNA, 107 bp, Artificial Sequence
  Synthetic
aacaaacaaatgagatgatttcagatagtgataaacactataacataattaattcgtgccaatcagagcataacagtggtgtggtggctgtggaacagatagcagac

SEQ ID NO: 307 - DNA, 79 bp, Artificial Sequence
  Synthetic
aatggaatcgagtggaatggaaggcaatggaatagaatggaatggaatcgaaaggaacggaatggaatggaatggaatg

SEQ ID NO: 308 - DNA, 79 bp, Artificial Sequence
  Synthetic
agcagtgcaagaacaacataacatacaagtaaacaaacacatggggccaggtaataaaaagtcaggctcaagaggtcag

SEQ ID NO: 309 - DNA, 73 bp, Artificial Sequence
  Synthetic
agaaatggaatcggagagaatggaaacaaatggaatggaattgaatggaatggaattgaatggaatgggaacg

SEQ ID NO: 310 - DNA, 75 bp, Artificial Sequence
  Synthetic
gcactagtcagatcaagacagaaagtcaacgaacaaagaacagacttaaactacactctagaacaaatggaccta

SEQ ID NO: 311 - DNA, 87 bp, Artificial Sequence
  Synthetic
aagagaactgcaaaacactgctcaaagaaatcagagatgacaaaaacacatggaaaaacgtttcatgctcatggattggaagactta

SEQ ID NO: 312 - DNA, 83 bp, Artificial Sequence
  Synthetic
aatcaacacgaatagaatggaacggaatggaatggaatggaatggaatggaatggagtggaatggaacagaatggagtggaat

SEQ ID NO: 313 - DNA, 112 bp, Artificial Sequence
  Synthetic
aacatcaaacgaaatcaaacggaattatcaaattgaatcgaagagaatcatcgaattgccacgaatgcaaccatctaatggtatggaatggaataatccatggacccagatg

SEQ ID NO: 314 - DNA, 94 bp, Artificial Sequence
  Synthetic
cggaattatcatcgaatgtaatcgaatggaatcaacatcaaacggaaaaaaacggaattatcgaatggaatcgaagagaatcatcgaatggacc

SEQ ID NO: 315 - DNA, 125 bp, Artificial Sequence
  Synthetic
tggacacacacgaacacacacctacacacacgtggacacacacggacacatggacacacacgaacacatggacacacacacggggacacacacagacacacacagagacacacacggacacatgg

SEQ ID NO: 316 - DNA, 88 bp, Artificial Sequence
  Synthetic
agcaacttcagcaaagtctcaggatacaaaatcaatgtgcaaaaatcacaagcattcttatacaccaataacagacaaacagagagcc

SEQ ID NO: 317 - DNA, 96 bp, Artificial Sequence
  Synthetic
aaaatcaatatgaaaacaaacacaagcagacaaagaaaattgggcaaaaggtttgagcagacacttcaccaaagaagtacaaatggcaaatcagca

SEQ ID NO: 318 - DNA, 100 bp, Artificial Sequence
  Synthetic
atcaaacggaatcaaacggaattatcgaatggaatcgaagagaatcatcgaatggactcgaatggaatcatctaatggaatggaatggaagaatccatgg

SEQ ID NO: 319 - DNA, 101 bp, Artificial Sequence
  Synthetic
aacagatttaaacaaaccaacaagcaaaaaacgaacaactccattcaaacatggacaaaagacacgaacagacacttttcaaagaagacatacatgtggcc

SEQ ID NO: 320 - DNA, 99 bp, Artificial Sequence
  Synthetic
aaatggaatggaatgcacttgaaaggaatagactggaacaaaatgaaatcgaacggtaggaatcatacagaacagaaagaaatggaacggaatggaatg

SEQ ID NO: 321 - DNA, 68 bp, Artificial Sequence
  Synthetic
accacacacaaaatacaccacacaccacacacacaccacacactatacacacaccacacaccacacac

SEQ ID NO: 322 - DNA, 100 bp, Artificial Sequence
  Synthetic
aaagaaatagaagggagttgaacagaatcgaatggaatcgaatcaaatggaatcgaatggcatcaaatggaatcgaatggaatgtggtgaagtggattgg

SEQ ID NO: 323 - DNA, 86 bp, Artificial Sequence
  Synthetic
ggaatcatcataaaatggaatcgaatggaatcatcatcaaatggaatcaaatggaatcattgaacggaattgaatggaatcgtcat

SEQ ID NO: 324 - DNA, 77 bp, Artificial Sequence
  Synthetic
tggaatggaatggaatgaaataaacacgaatagaatggaacggaatggaacggaatggaatggaatggaatggaaag

SEQ ID NO: 325 - DNA, 87 bp, Artificial Sequence
  Synthetic
aagaattggacaaaacacacaaacaaagcaaggaaggaatgaaaggatttgttgaaaatgaaagtacactccacagtgtgggagcag

SEQ ID NO: 326 - DNA, 92 bp, Artificial Sequence
  Synthetic
taatcagcacaatcaactgtagtcacaaaacaaatagtaacgcaatgataaagaaacagagaactagttcaaataaacatgataagatgggg

SEQ ID NO: 327 - DNA, 78 bp, Artificial Sequence
  Synthetic
aagcggaattatcaaatggaatcgaagagaatggaaacaaatggaatggaattgaatggaatggaattgaatggaatg

SEQ ID NO: 328 - DNA, 97 bp, Artificial Sequence
  Synthetic
aagcaacttcagcaaagtctcaggacacaaaatcaatatgcgaaaatcacaagcattcctatacaccaataatagacaaacagagagccaaatcatg

SEQ ID NO: 329 - DNA, 62 bp, Artificial Sequence
  Synthetic
ttcacagcagcattacgcacaatagccagaaggtgggaacagacaaaatgccttttgatggg

SEQ ID NO: 330 - DNA, 88 bp, Artificial Sequence
  Synthetic
agaccctaatatcacagttaaacgaactagagaaggaagagcaaacaaattcaaaagctagcggaaagcaagaaataactaagaccag

SEQ ID NO: 331 - DNA, 86 bp, Artificial Sequence
  Synthetic
taaaagtgtgctcaacatcattgatcatcagagaaatgcaaatcaaaactacaatgagatatcatctcatcccagtcaaagtggct

SEQ ID NO: 332 - DNA, 92 bp, Artificial Sequence
  Synthetic
acttgaatcgaatggaaaggaatttaatgaacttaaatcgaatggaatataatggtatggaatggactcatggaatggaatggaaaggaatc

SEQ ID NO: 333 - DNA, 91 bp, Artificial Sequence
  Synthetic
tggaatcatcatcgaaagcaagcgaatggaatcatcaaatggaaacgaatggaatcatcgaatggactcggatggaattgttgaatggact

SEQ ID NO: 334 - DNA, 68 bp, Artificial Sequence
  Synthetic
tggaatcaacatcaaacggaaaaaaacggaattatcgaatggaatcgaagagaatcatcgaatggacc

SEQ ID NO: 335 - DNA, 86 bp, Artificial Sequence
  Synthetic
taagtgaattgaatagaatcaatctgaatgtaatgaaatggaatggaacggaatggaatggaatggaatggaatggaatggaatgg

SEQ ID NO: 336 - DNA, 98 bp, Artificial Sequence
  Synthetic
aggaaaatttaatcagcaggaatagaaacacacttgagaaatccatgtggaatgaaaagagaatggctgagcagcaacagattgtcaaaaaggaaatc

SEQ ID NO: 337 - DNA, 62 bp, Artificial Sequence
  Synthetic
aacatcaaacggaaaaaaaacggaattatcgaatggaatcgaagagaatcatcgaatggacc

SEQ ID NO: 338 - DNA, 82 bp, Artificial Sequence
  Synthetic
gaaaatgaacaatatgaacaaacaaacaaaattactacccttacgaaagtacgtgcattctagtatggtgacaaaaaggaaa

SEQ ID NO: 339 - DNA, 54 bp, Artificial Sequence
  Synthetic
agaaaacacacagacaacaaaaaacacagaacgacaatgacaaaatggccaagc

SEQ ID NO: 340 - DNA, 88 bp, Artificial Sequence
  Synthetic
agcaacttcagcaaagactcaggatacaaaatcaatgtgcaaaaatcacaagcattcttatacaccaataacagacagagagccaaat

SEQ ID NO: 341 - DNA, 97 bp, Artificial Sequence
  Synthetic
tgacatgcaagaaataaggaagtgcaaaaacaaacaaacaaacaacaacaacaacaacaacaacaacaacaaaaaacagtcccaaaaggatgggcag

SEQ ID NO: 342 - DNA, 132 bp, Artificial Sequence
  Synthetic
taattgagaataagcattccagtggaaaaaaaactaaacaatttgttgtaaaacatccttaaaagcatcagaaagttaatacagcaatgaagaattacaggaccaaattaagaatggtatggaagcctgtta

SEQ ID NO: 343 - DNA, 88 bp, Artificial Sequence
  Synthetic
tatcatcgaatggaatcgaatggaatcaacatcaaacggaaaaaaacggaattatcgaattgaatcgaagagaatcatcgaatggacc

SEQ ID NO: 344 - DNA, 74 bp, Artificial Sequence
  Synthetic
gaatggaatcaaatagaatggaatcgaaacaaatggaatggaatggaatgggagctgagattgtgtcactgcac

SEQ ID NO: 345 - DNA, 80 bp, Artificial Sequence
  Synthetic
agcaaaacaaacacaatctgtcgttcatggtactacgacatactgggagagatattcaaatgatcacacaaaacaacatg

SEQ ID NO: 346 - DNA, 93 bp, Artificial Sequence
  Synthetic
aaggattcgaatggaatgaaaaagaattgaatggaatagaacagaatggaatcaaatcgaatgaaatggagtggaatagaaaggaatggaatg

SEQ ID NO: 347 - DNA, 147 bp, Artificial Sequence
  Synthetic
aacggaatcaaacggaattatcgaatgnnntnnaagagaatcatcgaacggactcgaatggaatcatctaatggaatggaatggaagaatccatggactcgaatgcaatcatcatcgaatggaatcgaacggaatcatcgaatggcc

SEQ ID NO: 348 - DNA, 85 bp, Artificial Sequence
  Synthetic
aatcaactagatgtcaatggaatgcaatggaatagaatggaatggaattaacacgaatagaatggaatggaatggaatggaatgg

SEQ ID NO: 349 - DNA, 76 bp, Artificial Sequence
  Synthetic
tgtaacactgcaaaccataaaaaccgtagaagaaaacctagacaatactattcaggacataggcatgggcaaagac

SEQ ID NO: 350 - DNA, 88 bp, Artificial Sequence
  Synthetic
aatggactcgaatggaataatcattgaacggaatcgaatggaatcatcatcggatggaaatgaatggaatcatcatcgcatggaatcg

SEQ ID NO: 351 - DNA, 76 bp, Artificial Sequence
  Synthetic
gaatggaatgatacggaatagaatggaatggaacgaaatggaattgaaaggaaaggaatggaatggaatggaatgg

SEQ ID NO: 352 - DNA, 79 bp, Artificial Sequence
  Synthetic
aaaaatgaccagagcaatagaatgcattgaccagataaagaccttcacgtatgttgaactaaaatgtgtggtgcaggtg

SEQ ID NO: 353 - DNA, 84 bp, Artificial Sequence
  Synthetic
aatcatcatcgaatggaatcgaatggtatcattgantgnaatcgaatggaatcatcatcanatggaaatgaatggaatcgtcat

SEQ ID NO: 354 - DNA, 86 bp, Artificial Sequence
  Synthetic
acaaaatcaaactaacctcgataagaatgcaagtgaatcaaaatgagtttcaaggggttgtggctagtacacgctttctacagctg

SEQ ID NO: 355 - DNA, 65 bp, Artificial Sequence
  Synthetic
gaatcaaatcaatggaatcaaatcaaatggaatggaatggaattgtatggaatggaatggcatgg

SEQ ID NO: 356 - DNA, 125 bp, Artificial Sequence
  Synthetic
taatgcagtccaatagaatggaatcgaatggcatggaatataaagaaatggaatcgaagagaatgggaacaaatggaatggaattgagtggaatggaattgaatggaatgggaacgaatggagtg

SEQ ID NO: 357 - DNA, 91 bp, Artificial Sequence
  Synthetic
tgaatagacacacagaccaatggaacagaatagagaacacagaataaatctgcacacttatagccagctgatttttgacaaatttgccaag

SEQ ID NO: 358 - DNA, 61 bp, Artificial Sequence
  Synthetic
aacatcnnacggaaaaaaacggaattatcgaatggaatcgaagagaatcatcgaatggacc

SEQ ID NO: 359 - DNA, 99 bp, Artificial Sequence
  Synthetic
gccaacaatcatatgagaaaaagctcaacatcactgatcatttcaggaatgcaaatcaaaaccacaatgagatactatcacacatcaatcagaatggct

SEQ ID NO: 360 - DNA, 76 bp, Artificial Sequence
  Synthetic
gaatcgaatggaatcaacatcaaacggaaaaaaacggaattatcgaatggaatcgaagagaatcatcgaatggacc

SEQ ID NO: 361 - DNA, 99 bp, Artificial Sequence
  Synthetic
aatcaaatggaatgaaatcgaatggaattgaatcgaatggaatgcaatagaatgtcttcaaatggaatcgaatggaaattggtgaagtggacgggagtg

SEQ ID NO: 362 - DNA, 122 bp, Artificial Sequence
  Synthetic
taacagtaccaaaaaacagtcataatcttcaagagcttaaatttagcatgaaaggaagacattcatcaaagaatcacacaaaggaatgtaaaattaaatggagattagtgccaggaaagagc

SEQ ID NO: 363 - DNA, 71 bp, Artificial Sequence
  Synthetic
taatggaatcaacatcaaacggaaaaaaacggaattatcgaatgcaatcgaagagaatcatcgaatggacc

SEQ ID NO: 364 - DNA, 88 bp, Artificial Sequence
  Synthetic
agcaacttcagcaaagtctcagcatacaaaatcaatgtgcaaaaatcacacgcattcctatacaccaataacagacaaacagagagcc

SEQ ID NO: 365 - DNA, 70 bp, Artificial Sequence
  Synthetic
gaatcaaatggaatggactgtaatggaatggattcgaatggaatcgaatggagtggactcaaatggaatg

SEQ ID NO: 366 - DNA, 93 bp, Artificial Sequence
  Synthetic
aacaagtggacgaaggatatgaacagacacttctcaagacatttatgcagccaacagacacacgaaaaaatgctcatcatcactggccatcag

SEQ ID NO: 367 - DNA, 85 bp, Artificial Sequence
  Synthetic
aaacacacaaagcaacaaaagaacgaagcaacaaaagcatagatttattgaaatgaaagtacattctacagagtgggggcaggct

SEQ ID NO: 368 - DNA, 90 bp, Artificial Sequence
  Synthetic
atacaactaaagcaaatataagcaactaaagcaacagtacaactaaagcaaaacagaacaagactgccagggcctagaaaagccaagaac

SEQ ID NO: 369 - DNA, 68 bp, Artificial Sequence
  Synthetic
gcaatcgaatggaatggaatcgaacggaatggaataaaatggaagaaaactggcaagaaatggaatcg

SEQ ID NO: 370 - DNA, 72 bp, Artificial Sequence
  Synthetic
agcagccaacaagcatatgaaataatgctccacaacactcatcatcagagaaatgcaaatcaaaaccaaaat

SEQ ID NO: 371 - DNA, 105 bp, Artificial Sequence
  Synthetic
tggaaccgaacaaagtcatcaccgaatggaattgaaatgaatcataatcgaatggaatcaaatggcatcttcgaattgactcgaatgcaatcatccactgggctt

SEQ ID NO: 372 - DNA, 84 bp, Artificial Sequence
  Synthetic
aacggaatcacgcggaattatcgaatggaatcgaagagaatcatcgaatggactcgaatggaatcatctaatggaatggaatgg

SEQ ID NO: 373 - DNA, 90 bp, Artificial Sequence; identical to SEQ ID NO: 293
  Synthetic

SEQ ID NO: 374 - DNA, 68 bp, Artificial Sequence
  Synthetic
aaaacaaacaacaacgacaaatcatgagaccagagttaagaaacaatgagaccaggctgggtgtggtg

SEQ ID NO: 375 - DNA, 80 bp, Artificial Sequence
  Synthetic
aatcgaaaggaatgcaatattattgaacagaatcgaaaagaatggaatcaaatggaatggaacagagtggaatggactgc

SEQ ID NO: 376 - DNA, 79 bp, Artificial Sequence
  Synthetic
aaggaatcgaatggaagtgaatgaaattgaatcaacaggaatggaagggaatagaatagactgtaatggaatggactcg

SEQ ID NO: 377 - DNA, 85 bp, Artificial Sequence
  Synthetic
aatggactcgaatgaaatcatcatcaaacggaatcgaatggaatcattgaatggaatggaatggaatcatcatggaatggaaacg

SEQ ID NO: 378 - DNA, 78 bp, Artificial Sequence
  Synthetic
ttgaccagaacacattacacaatgctaatcaactgcaaaggagaatatgaacagagaggaggacatggatattttgtg

SEQ ID NO: 379 - DNA, 65 bp, Artificial Sequence
  Synthetic
aacccgagtgcaatagaatggaatcgaatggaatggaatggaatggaatggaatggaatggagtc

SEQ ID NO: 380 - DNA, 92 bp, Artificial Sequence
  Synthetic
aagagtattgaagttgacatatctagactgatcaagaacaaagacaaaaggtacagattatcaagaaaatgagcgggcaaagcaagatggcc

SEQ ID NO: 381 - DNA, 80 bp, Artificial Sequence
  Synthetic
gaatggaattgaaaggaatggaatgcaatggaatggaatgggatggaatggaatgcaatggaatcaactcgattgcaatg

SEQ ID NO: 382 - DNA, 101 bp, Artificial Sequence
  Synthetic
gaaaaaaacggaattatcnaattgaatcnaatanaatcatcnnnnngaccanantggaatcatctaatgnaatgnaatggaataatccatggactcnaatg

SEQ ID NO: 383 - DNA, 101 bp, Artificial Sequence
  Synthetic
gaaaaaaacggaattatcgaattgaatcgaatagaatcatcgaacggaccagaatggaatcatctaatggaatggaatggaataatccatggactcgaatg

SEQ ID NO: 384 - DNA, 91 bp, Artificial Sequence
  Synthetic
aaccactgcttaaggaaataagagagaacacaaacaaatggaaaaacgttccatgctcatggataggagaatcaatatcgtgaaaatggcc

SEQ ID NO: 385 - DNA, 95 bp, Artificial Sequence
  Synthetic
tatcgaatggaatggaaaggagtggagtagactcgaatagaatggactggaatgaaatagattcgaatggaatggaatggaatgaagtggactcg

SEQ ID NO: 386 - DNA, 84 bp, Artificial Sequence
  Synthetic
gtatcaacatcaaacggaaaaaaacggaattatcgaatggaatcatctaatggaatggaatggaataatccatggactcgaatg

SEQ ID NO: 387 - DNA, 87 bp, Artificial Sequence
  Synthetic
taaatggagacatcattgaatacaattgaatggaatcatcacatggaatcgaatggaatcatcgtaaatgcaatcaagtggaatcat

SEQ ID NO: 388 - DNA, 83 bp, Artificial Sequence
  Synthetic
gaatggaattgaaaggtatcaacaccaaacggaaaaaaaaacggaattatcgaatggaatcgaagagaatcatcgaacggacc

SEQ ID NO: 389 - DNA, 73 bp, Artificial Sequence
  Synthetic
agcaatttcagcaaagtctcaggatacaaaatcaatgtacaaattcacaagcattcttatggaccaacaacag

SEQ ID NO: 390 - DNA, 88 bp, Artificial Sequence
  Synthetic
aaccaaattagacaaattggaaatcattacacataacaaaagtaataaactgtcagcctcagtagtattcattgtacataaactggcc

SEQ ID NO: 391 - DNA, 109 bp, Artificial Sequence
  Synthetic
tattttaccagattattcaagcaatatatagacagcttaaagcatacaagaagacatgtatagatttacatgcaaacactgcaccactttacataagggacttgagcac

SEQ ID NO: 392 - DNA, 68 bp, Artificial Sequence
  Synthetic
ggaatcgaatggcatcaacatcaaacggaaaaaaacggaattatcgaatggaatcgaatggaatcatc

SEQ ID NO: 393 - DNA, 90 bp, Artificial Sequence
  Synthetic
aaacaaaacacagaaatgcaaagacaaaacataaaacgcagccataaaggacatattttagataactggggaaatttgtatgggctgtgt

SEQ ID NO: 394 - DNA, 84 bp, Artificial Sequence
  Synthetic
aggaaaagaaagaaatagaaaatgcgaaatggtaagaaaaaacagcataataaacatttgtatggtgttgatggacaatgcatt

SEQ ID NO: 395 - DNA, 103 bp, Artificial Sequence
  Synthetic
aatggaatcaacatcaaacggaatcaaacggaattatcgaatggaatcgaagagaatcatcgaacggactcgaatggaatcatctaatggaatggaatggaag

SEQ ID NO: 396 - DNA, 75 bp, Artificial Sequence
  Synthetic
aatcgaatggaatcagcatcaaacggaaaaaaacggaattatcgaatggaatcgaagagaatcatcgaatggacc

SEQ ID NO: 397 - DNA, 75 bp, Artificial Sequence
  Synthetic
aaagaaatggaatcgaagagaatggaaacaaatggaatggaattgaatggaatggaattgaatggaatgggaacg

SEQ ID NO: 398 - DNA, 90 bp, Artificial Sequence
  Synthetic
agaaagaatcaagaggaaatgcaagaaatccaaaacactgtaacagatatgatgaataatgaggtatgcactcatcagcagactcgacat

SEQ ID NO: 399 - DNA, 94 bp, Artificial Sequence
  Synthetic
aaacggaattatnnantggannnnaagnnaatcatcgaacggannnnannggaatcatntnnnngaanggaatggaacaatccatggtntnnnn

SEQ ID NO: 400 - DNA, 88 bp, Artificial Sequence
  Synthetic
agcaacttcagcaaagtttcaggatacaaaatcaatgtgcaaaaatcacaagcattcttatacaccaacaacagacaaacagagagcc

SEQ ID NO: 401 - DNA, 87 bp, Artificial Sequence
  Synthetic
agacagtcagacaatcacaaagaaacaagaatgaaaatgaatgaacaaaaccttcaagaaatatgggattatgaagaggccaaatgt

SEQ ID NO: 402 - DNA, 99 bp, Artificial Sequence
  Synthetic
atcataacgacangancaaattcacacacaacaatnnnnacnnnaaanncaaatgggttaaatnntncaattaaaggatgcagacgggcaaattggata

SEQ ID NO: 403 - DNA, 99 bp, Artificial Sequence
  Synthetic
atcataangacaagancaaattcacacacaacaatnnnnacnnnaaanncaaatgggttnaatgntncaattaaaggatgcagacggncaaattggata

SEQ ID NO: 404 - DNA, 95 bp, Artificial Sequence
  Synthetic
gaatggaatcgaatggattgatatcaactggaatggaatggaagggaatggaatggaatggaattgaaccaaatgtnnnngncttgaatggaatg

SEQ ID NO: 405 - DNA, 66 bp, Artificial Sequence
  Synthetic
gaatcaacatcaaacggaaaaaaacggaattatcgaatggaatcgaagagaatcatcgaatggacc

SEQ ID NO: 406 - DNA, 135 bp, Artificial Sequence
  Synthetic
atggaatcaacatcaaacggaatcaaacggaattatcgaatggaatcaaagagaatcatcgaacggactcgaatggaatcatctaatggaatggaatggaagaatccatggactcgaatgcaatcatcatcgaat

SEQ ID NO: 407 - DNA, 52 bp, Artificial Sequence
  Synthetic
ggaatggaatggaatggagcngaatngaanggannnnantcaaatggaatgc

SEQ ID NO: 408 - DNA, 90 bp, Artificial Sequence
  Synthetic
aacatacgcaaatcaataaatgtaatccagcatataaacagaaccaaagacaaaaaccacatgattatctcaatagatgcagaaaaggcc

SEQ ID NO: 409 - DNA, 124 bp, Artificial Sequence
  Synthetic
aaacgattggacaggaatggaatcaccatcgaatggaaacgaatggaatcttcgaatggaattgaatgaaattattgaacggaatcaaatagaatcatcattgaacagaatcaaattggatcat

SEQ ID NO: 410 - DNA, 79 bp, Artificial Sequence
  Synthetic
aaaagatgcaaaagtagcaaatgcaatgttaaaacaagcaaagaaagaatcaggtggaccacatagtgcagtgcttctc

SEQ ID NO: 411 - DNA, 85 bp, Artificial Sequence
  Synthetic
aacaataaacaaactccaactagacacaatagtcaaattgctgaaaatgaaatataaaggaacaatctcgatggtagcccaagga

SEQ ID NO: 412 - DNA, 90 bp, Artificial Sequence
  Synthetic
agtcaataacaagaagacaaacaacccaattacaaaatgggatatgaatttaatagatgttactccaaggaagatacacaaatggccaac

SEQ ID NO: 413 - DNA, 150 bp, Artificial Sequence
  Synthetic
aaaacacctaggaatacagataacaagggacattaactacctcttaaagagaactacaaaccactgctcaaggaaatgagagaggacacaaacacatggaaaaacattccatcctcatggataggaagaatcaatattgtgaaaatggcc

SEQ ID NO: 414 - DNA, 93 bp, Artificial Sequence
  Synthetic
gatatataaacaagaaaacaactaatcacaactcaatatcaaagtgcaatgatggtgcaaaatgcaagtatggtggggacagagaaaggatgc

SEQ ID NO: 415 - DNA, 66 bp, Artificial Sequence
  Synthetic
acacatatcaaacaaacaaaagcaattgactatctagaaatgtctgggaaatggcaagatattaca

SEQ ID NO: 416 - DNA, 86 bp, Artificial Sequence
  Synthetic
ggaatcatcatataatggaatcgaatggaatcaacatcaaatggaatcaaatggaatcattgaacggaattgaatggaatcgtcat

SEQ ID NO: 417 - DNA, 89 bp, Artificial Sequence
  Synthetic
cccaacttcaaattatactacaaggctacagtaatcaaaaaagcatagtactattacaaaaacagacacacaggccaatggaatacaat

SEQ ID NO: 418 - DNA, 103 bp, Artificial Sequence
  Synthetic
aaacgcagaaacaaatcaacgaaagaacgaagcaatgaaagacaaagcaacaaaagaatggagtaagaaagcacactccacaaagtggaagcaggctgggaca

SEQ ID NO: 419 - DNA, 95 bp, Artificial Sequence
  Synthetic
aactaacacaagaacagaaaaccaaacatcacatgttctcactcataagcgggagctgaacaatgagaacacacggacacagggagaggaacatg

SEQ ID NO: 420 - DNA, 89 bp, Artificial Sequence
  Synthetic
gccacaattttgaaacaaccataataatgagaatacacaagacaactccaataatgtgggaagacaaactttgcaattcacatcatggc

SEQ ID NO: 421 - DNA, 121 bp, Artificial Sequence
  Synthetic
aatggaatcaacatcaaacggaatcaaatggaattatcgaatggaatcgaagagaatcatcgaattgtcacgaatggaatcatctaatggaatggaatggaataatccatggcccctatgc

SEQ ID NO: 422 - DNA, 56 bp, Artificial Sequence
  Synthetic
taaacagaaccaaagacaaaaatcacatgattatctcaatagatgcagaaaaggcc

SEQ ID NO: 423 - DNA, 83 bp, Artificial Sequence
  Synthetic
atcaacagacaacagaaacaaatccacaaagcacttagttattagaactgtcatacagactgtacaacaaccacatttaccat

SEQ ID NO: 424 - DNA, 85 bp, Artificial Sequence
  Synthetic
aatggactcgaatgaaatcatcatcaaacagaatcgaatggaatcatctaatggaatggaatggcataatccatggactcgaatg

SEQ ID NO: 425 - DNA, 91 bp, Artificial Sequence
  Synthetic
taaaatgaaacaaatatacaacacgaaggttatcaccagaaatatgccaaaacttaaatatgagaataagacagtctcaggggccacagag

SEQ ID NO: 426 - DNA, 59 bp, Artificial Sequence
  Synthetic
aaaatacagcgttatgaaaagaatgaacacacacacacacacacacacacagaaaatgt

SEQ ID NO: 427 - DNA, 77 bp, Artificial Sequence
  Synthetic
tactctcagaagggaagcagatattcagcataaatcatattgtttgtacaaagagtctgggcatggtgaatgacact

SEQ ID NO: 428 - DNA, 138 bp, Artificial Sequence
  Synthetic
caaacaaataggtaccaaacaaataacaacataaacctgacaacacacttatttacaagagacatcccttatatgaaagggtacagaaaagtcgatggtaagatgatggggaaaggtataccaaccactagcagaagg

SEQ ID NO: 429 - DNA, 93 bp, Artificial Sequence
  Synthetic
tggaatcgaatggaatcaatatcaaacggaaaaaaacggaattatcgaatggaatcgaaaagaatcatcgaatgggcccgaatggaatcatct

SEQ ID NO: 430 - DNA, 85 bp, Artificial Sequence
  Synthetic
acaaatggaatcaacaacgaatggaatcgaatggaaacgccatcgaaaggaaacgaatggaattatcatgaaattgaaatggatg

SEQ ID NO: 431 - DNA, 91 bp, Artificial Sequence
  Synthetic
gacaagagttcagaaaggaagactacacagaaatacgcattttaaagtcactgacatggagatgacacttaaaaccatgaacatggatggg

SEQ ID NO: 432 - DNA, 94 bp, Artificial Sequence
  Synthetic
aaaataaacgcaaattaaaatcacaagataccaacacattcccacggctaagtacgaagaacaagggcgaatggtcagaattaagctcaaacct

SEQ ID NO: 433 - DNA, 89 bp, Artificial Sequence
  Synthetic
taaactgacacaaacacagacacacagatacacacatacatacagaaatacacattcacacacagacctggtctttggagccagagatg

SEQ ID NO: 434 - DNA, 81 bp, Artificial Sequence
  Synthetic
gatcaataaatgtaattcatcatataaacagagaactaaagacaaaaacacatgattatcgcaatacatgcagaaaaggcc

SEQ ID NO: 435 - DNA, 98 bp, Artificial Sequence
  Synthetic
aggacatgaatagacaattctcaaaagaagatacacaagtggcaaacaaacacatgaaaaaagactcaacattagtaatgaccatggaaatgcaaatc

SEQ ID NO: 436 - DNA, 60 bp, Artificial Sequence
  Synthetic
acatcaaacggaaaaaaacggaattatcgaatggaatcgaagagaatcatcgaatggacc

SEQ ID NO: 437 - DNA, 70 bp, Artificial Sequence
  Synthetic
aatggactcgaatagaattgactggaatggaatggactcgaatggaatggaatggaatggaagggactcg

SEQ ID NO: 438 - DNA, 138 bp, Artificial Sequence
  Synthetic
aagaaagacagagaacaaacgtaattcaagatgactgattacatatccaagaacattagatggtcaaagactttaagaaggaatacattcaaaggcaaaacgtcacttactgattttggtggagtttgccacatggac

SEQ ID NO: 439 - DNA, 75 bp, Artificial Sequence
  Synthetic
aacataatccatcaaataaacagaaccaaagacaaaaaccacatgattatctcaatagatgcagaaaaggccttc

SEQ ID NO: 440 - DNA, 83 bp, Artificial Sequence
  Synthetic
gaatggaatcgaatggaatgaacatcaaacggaaaaaaacggaattatcgaatggaatcaaagagaatcatcgaatggacccg

SEQ ID NO: 441 - DNA, 96 bp, Artificial Sequence
  Synthetic
atggactcgaatgtaataatcattgaacggaatcgaatggaatcatcatcggatggaaacgaatggaatcatcatcgaatggaatcgaatgggatc

SEQ ID NO: 442 - DNA, 113 bp, Artificial Sequence
  Synthetic
gaatggaatcaacatcaaacggaatcaaacggaattatcgaatggaatcgaagagaatcatcgaatggccacgaatggaatcatctaatggaatggaatggaataatccatgg

SEQ ID NO: 443 - DNA, 66 bp, Artificial Sequence
  Synthetic
gaaatggaatggaaaggaataaaatcaagtgaaattggatggaatggattggaatggattggaatg

SEQ ID NO: 444 - DNA, 98 bp, Artificial Sequence
  Synthetic
aaacggaaaaaaaacggaattatcgaatggaatcgaagagaatcatcgaacgaaccagaatggaatcatctaatggaatggaatggaataatccatgg

SEQ ID NO: 445 - DNA, 78 bp, Artificial Sequence
  Synthetic
attaacccgaatagaatggaatggaatggaatggaacggaacggaatggaatggaatggaatggaatggaatggatcg

SEQ ID NO: 446 - DNA, 82 bp, Artificial Sequence
  Synthetic
gcaaaacacaaacaacgccataaaaaactgggcaaaggatatgaacagacatttttcaaaacaaaacatacttatggccaac

SEQ ID NO: 447 - DNA, 61 bp, Artificial Sequence
  Synthetic
aacatcaaacggaaaaaaacggaattatcgtatggaatcgaagagaatcatcgaatggacc

SEQ ID NO: 448 - DNA, 84 bp, Artificial Sequence
  Synthetic
aaatcaataaatgtaattcagcatataaacagaaccaaagacaaaaaccacatgattatctcaatagatgcagaaaaggccttt

SEQ ID NO: 449 - DNA, 71 bp, Artificial Sequence
  Synthetic
agaatcaaatggaattgaatcgaatggaatcgaatggattggaaaggaatagaatggaatggaatggaatg

SEQ ID NO: 450 - DNA, 86 bp, Artificial Sequence
  Synthetic
gaatagaattgaatcatcattgaatggaatcgagtagaatcattgaaatcgaatggaatcatcatcgaatggaattgggtggaatc

SEQ ID NO: 451 - DNA, 148 bp, Artificial Sequence
  Synthetic
caccgaatagaatcgaatggaacaatcatcgaatggactcaaatggaattatcctcaaatggaatcgaatggaattatcgaatgcaatcgaatagaatcatcgaatagactcgaatggaatcatcgaatggaatggaatggaacagtc

SEQ ID NO: 452 - DNA, 85 bp, Artificial Sequence
  Synthetic
aaatcatcatcgaatggaatcgaatggtatcattgaatggaatcgaatggaatcatcatcagatggaaatgaatggaatcgtcat

SEQ ID NO: 453 - DNA, 95 bp, Artificial Sequence
  Synthetic
gaatggaatcgaaaggaatagaatggaatggatcgttatggaaagacatcgaatggaatggaattgactcgaatggaatggactggaatggaacg

SEQ ID NO: 454 - DNA, 86 bp, Artificial Sequence; identical to SEQ ID NO: 450
  Synthetic

SEQ ID NO: 455 - DNA, 110 bp, Artificial Sequence
  Synthetic
gaaaggaatagaatggaatggatcgttatggaaagacatcgaatgggatggaattgactcgaatggattggactggaatggaacggactcgaatggaatggactggaatg

SEQ ID NO: 456 - DNA, 88 bp, Artificial Sequence
  Synthetic
tggatttcagatatttaacacaaaatagtcaaagcagataaatactagcaacttatttttaatgggtaacatcatatgttcgtgcctt

SEQ ID NO: 457 - DNA, 88 bp, Artificial Sequence
  Synthetic
acagcagaaaacgaacatcagaaaatcactctacatgatgcttaaatacagagggcaagcaacccaagagaaaacaccacttcctaat

SEQ ID NO: 458 - DNA, 90 bp, Artificial Sequence
  Synthetic
aacatacacaaatcaataaacgtaatccagcttataaacagaaccaaagacaaaaaccacatgattatctcaatagatgcggaaaaggcc

SEQ ID NO: 459 - DNA, 93 bp, Artificial Sequence
  Synthetic
acatcaaacggaatcaaacggaattatcgaatggaatcgaaaagaatcatcgaacggactcgaatggaatcatctaatggaatggaatggaag

SEQ ID NO: 460 - DNA, 74 bp, Artificial Sequence
  Synthetic
atcgaatggaatcaacatcaaacggaaaaaaacggaattatcaaatggaatcgaagagaatcatcgaatggacc

SEQ ID NO: 461 - DNA, 86 bp, Artificial Sequence
  Synthetic
gaataatcattgaacggaatcgaatggaaacatcatcgaatggaaacgaatggaatcatcatcgaatggaaatgaaaggagtcatc

SEQ ID NO: 462 - DNA, 110 bp, Artificial Sequence
  Synthetic
catcaaacggaatcaaacggaattatcgaatggaatcgaaaagaatcatcgaacggactcgaatggaatcatctaatggaatggaatggaagaatccatggactcgaatg

SEQ ID NO: 463 - DNA, 92 bp, Artificial Sequence
  Synthetic
tccagtcgatcatcatatagtcagcacttatcatacaccaagccgtgtgcaaggaaagggaatacaaccatgaacatgatagatggatggtt

SEQ ID NO: 464 - DNA, 96 bp, Artificial Sequence
  Synthetic
acaaaccactgctcaaggaaataaggacacaaacaaatggaacaacattccgtgctcatggataggaagaatcaatatcgtgaaaatggccatact

SEQ ID NO: 465 - DNA, 87 bp, Artificial Sequence
  Synthetic
acaaaattgatagaccactagcaagactaataaagaagaaaagagagaagaatcattaccattcaggacataggcatgggcaaggac

SEQ ID NO: 466 - DNA, 97 bp, Artificial Sequence
  Synthetic
aaacggaatcaaacggaattatcgaatggaatcgaagagaatcatcgaatggactcgaatggaatcatctaatggaatggaatggaagaatccatgg

SEQ ID NO: 467 - DNA, 87 bp, Artificial Sequence
  Synthetic
atacacaaatcaataaatgtaatccagcatataaacagaaccaaagacaaaaaccatatgattatctcaatggatgcagaaaaggcc

SEQ ID NO: 468 - DNA, 61 bp, Artificial Sequence
  Synthetic
aatngaatagaatcatcgaatggactcgaatggaatcatcgannntantgatggaacagtc

SEQ ID NO: 469 - DNA, 124 bp, Artificial Sequence
  Synthetic
tggaatggaatcatcgcatagaatcgaatggaattaccatcgaatgggatcgaatggtatcaacatcaaacgcaaaaaaacggaattatcgaatggaatcgaagagaatcttcgaacggacccg

SEQ ID NO: 470 - DNA, 75 bp, Artificial Sequence
  Synthetic
gaattgaattgaatggaatggaatgcaatggaatctaatgaaacggaaaggaaaggaatggaatggaatggaatg

SEQ ID NO: 471 - DNA, 90 bp, Artificial Sequence
  Synthetic
aacagaatggaatcaaatcgaatgaaatggaatggaatagaaaggaatggaatgaaatggaatggaaaggattcgaatggaatgcaatcg

SEQ ID NO: 472 - DNA, 63 bp, Artificial Sequence
  Synthetic
atggaatggaatggaatggaattaaatggaatggaaaggaatggaatcgaatggaaaggaatc

SEQ ID NO: 473 - DNA, 102 bp, Artificial Sequence
  Synthetic
gtcgaaatgaatagaatgcaatcatcatcaaatggaatccaatggaatcatcatcaaatagaatcgaatggaatcatcaaatggaatcgaatggagtcattg

SEQ ID NO: 474 - DNA, 77 bp, Artificial Sequence
  Synthetic
tggaattatcgaaagcaaacgaatagaatcatcgaatggactcgaatggaatcatcgaatggaatggaatggaacag

SEQ ID NO: 475 - DNA, 69 bp, Artificial Sequence
  Synthetic
aaaggaatggaatgcaatggaatgcaatggaatgcacaggaatggaatggaatggaatggaaaggaatg

SEQ ID NO: 476 - DNA, 75 bp, Artificial Sequence
  Synthetic
aatctaatggaatcaacatcnaacggaaaaaaacggaattatcgaatggaatcnaagagaatcatcnaatggacc

SEQ ID NO: 477 - DNA, 100 bp, Artificial Sequence
  Synthetic
tacacaacaaaagaaatactcaacacagtaaacagacaaccttcagaacaggagaaaatatttgcaaatacatctaacaaagggctaatatccagaatct

SEQ ID NO: 478 - DNA, 70 bp, Artificial Sequence
  Synthetic
ngcaatcntagtntcagataaaacagacattaaaccaacaaagatcaaaagagacaaagaaggccantac

SEQ ID NO: 479 - DNA, 76 bp, Artificial Sequence
  Synthetic
gaatcgaatggaatcaacatcaaacggaaaaaaacggaattatcgaatggaatcnaaaagaatcatcnaatggacc

SEQ ID NO: 480 - DNA, 92 bp, Artificial Sequence
  Synthetic
acagttaacaaaaaccgaacaatctaattacgaaatgaacaaaagatatgaacagacatttcacccgagagtatacaggggccaggcatggt

SEQ ID NO: 481 - DNA, 85 bp, Artificial Sequence
  Synthetic
aatggaatcgaatggaatgcaatccaatggaatggaatgcaatgcaatggaatggaatcgaacggaatgcagtggaagggaatgg

SEQ ID NO: 482 - DNA, 102 bp, Artificial Sequence
  Synthetic
gaacacagaaaaatttcaaaggaataatcaacagggattgataactaactggatttagagagccaaggcaaagagaatcaaagcacagggcctgagtcggag

SEQ ID NO: 483 - DNA, 84 bp, Artificial Sequence
  Synthetic
tataccacacaaatgcaaaagattattagcaacaattatcaacagcaatatgtcaacaagttgacaaacctagaggacatggat

SEQ ID NO: 484 - DNA, 86 bp, Artificial Sequence
  Synthetic
caccatgagtcattaggtaaatgcaaatcaaaaccacaatgaaatacttcacacccatgaagatggctataataaaaaaacagaca

SEQ ID NO: 485 - DNA, 81 bp, Artificial Sequence
  Synthetic
agttgaatagaaccaatccgaatgaaatggaatggaatggaacggaatggaattgaatggaatggaatggaatgcaatgga

SEQ ID NO: 486 - DNA, 101 bp, Artificial Sequence
  Synthetic
aagtaataagactgaattagtaatacaaagtgtctcaacaaagaaaattgcgggactgttcatgctcatggacaggaagaatcaatatcatgaaaatggcc

SEQ ID NO: 487 - DNA, 90 bp, Artificial Sequence
  Synthetic
aactcgattgcaatggaatgtaatgtaatggaatggaatggaattaacgcgaatagaatggaatggaatgtaatggaacggaatggaatg

SEQ ID NO: 488 - DNA, 82 bp, Artificial Sequence
  Synthetic
aagcggaatagaattgaatcatcattgaatggaatcgagtagaatcattgaaatcgaatggaatcatagaatggaatccaat

SEQ ID NO: 489 - DNA, 91 bp, Artificial Sequence
  Synthetic
aaaggaaaactacaaaacactgctgaaagaaatcattgacaacacaaacaaatggaaacacatcccaagatcatgggtgggtggaatcaat

SEQ ID NO: 490 - DNA, 94 bp, Artificial Sequence
  Synthetic
aatggaatcnaaaggaatagaatggaatggatcgttatggaaagatatcgaatggaatggaattgactcgaatggaatggactggaatggaacg

SEQ ID NO: 491 - DNA, 91 bp, Artificial Sequence
  Synthetic
taacggaataatcatcgaacagaatcaaatggaatcatcattgaatggaattgaatggaatcttcgaatagacatgaatggaccatcatcg

SEQ ID NO: 492 - DNA, 105 bp, Artificial Sequence
  Synthetic
aaagaccgaaacaacaacagaaacagaaacaaacaacaataagaaaaaatgttaagcaaaacaaatgattgcacaacttacatgattactgagtgttctaatggt

SEQ ID NO: 493 - DNA, 92 bp, Artificial Sequence
  Synthetic
aagatttaaacataagacctaaaacgacaaaaatcctaggagaaaacctaagcaataccattcaggacataggcatgggcaaagacttcatg

SEQ ID NO: 494 - DNA, 99 bp, Artificial Sequence
  Synthetic
agaaacagccagaaaacaattattacctacagcattaaaactattcaaatgacagcatatttttcagcagaaatcatgaaggccagaaggacgtgtcat

SEQ ID NO: 495 - DNA, 92 bp, Artificial Sequence
  Synthetic
atgtacacaaatcaataaatgcagtccagcatataaacagaaccaaacacaaaaaccacatgattatctcaatagatgcagaaaaggccttt

SEQ ID NO: 496 - DNA, 88 bp, Artificial Sequence
  Synthetic
agcaacttcagcaaagtctcaggacacaaaatcaatgtgcaaaaatcacaagcattcttatacaccaataacagacaaacagagagcc

SEQ ID NO: 497 - DNA, 72 bp, Artificial Sequence
  Synthetic
ttgaatcgaatggaatcgaatggattggaaaggaatagaatggaatggaatggaattgactcaaatggaatg

SEQ ID NO: 498 - DNA, 87 bp, Artificial Sequence
  Synthetic
aacggaatcaaacggaattatcgaatggaatcgaatagaatcatcgaacggactcgaatggaatcatctaatggaatggaatggaag

SEQ ID NO: 499 - DNA, 76 bp, Artificial Sequence
  Synthetic
aacatcactgatcattagaaacacacaaatcaaaaccacaataagataccatctaacaccagtcacaatggctatt

SEQ ID NO: 500 - DNA, 94 bp, Artificial Sequence
  Synthetic
taagcaatttcagcagtctcaggatacaaaatcaatgtgcaaaaatcacaagcattcttatacaccaacaacagacaaacagagagccaaatcg

SEQ ID NO: 501 - DNA, 85 bp, Artificial Sequence
  Synthetic
agaaaaaaacaaacagcccattaaaaggtagacaaaggacatgaacacttttcaaaagaagacatacatgtggccaaacagcatg

SEQ ID NO: 502 - DNA, 84 bp, Artificial Sequence
  Synthetic
attggaatggaacggaacagaacggaatggaatggaatagaatggaatggaatggaatggtatggaatggaatggaatggtacg

SEQ ID NO: 503 - DNA, 80 bp, Artificial Sequence
  Synthetic
agagcatccacaaggcccaattcaaagaatctgaaataatgtattgttactgcaacagttgtgagtaccagtggcatcag

SEQ ID NO: 504 - DNA, 87 bp, Artificial Sequence
  Synthetic
aatccacaaagacaacagaagaaaagacaacagtagacaaggatgtcaaccacattttggaagagacaagtaatcaaacacatggca

SEQ ID NO: 505 - DNA, 106 bp, Artificial Sequence
  Synthetic
aaacagaaccacagatatctgtaaaggattacactatagtattcaacagagtatggaacagagtatagtattcaacagagtatgcaaagaaactaaggccagaaag

SEQ ID NO: 506 - DNA, 133 bp, Artificial Sequence
  Synthetic
agcaaacaaacaaacaaacaaacaaactatgacaggaacaaaacgtcacatatcaacattaacaaagaatgtaaacagcctaaatgcttcacttaaaagttatagacaggggctgggcatggtggctcacgcc

SEQ ID NO: 507 - DNA, 77 bp, Artificial Sequence
  Synthetic
aaaagtacagaagacaacaaaaaatgagagagagaaagataacagactatagcagcattggtgatcagagccaccag

SEQ ID NO: 508 - DNA, 137 bp, Artificial Sequence
  Synthetic
tacaagaaaatcacagtaacatttataaaacacagaagtgtgaacacacagctattgaccttgaaaacagtgaaagagggtcagctgtagaactaagacataagcaaagtttttcaatcaagaatacatgggtggcc

SEQ ID NO: 509 - DNA, 118 bp, Artificial Sequence
  Synthetic
gaatcgaatggaatcaacatcaaacggaaaaaaacggaattatcgaatggaatcgaaaagaatcatcgaacggactcgaatggaatcatctaatggaatggaatggaagaatccatgg

SEQ ID NO: 510 - DNA, 73 bp, Artificial Sequence
  Synthetic
aatggaatcgaatggaatcatcatcaaatggaatctaatggaatcattgaacggaattggatggaatcgtcat

SEQ ID NO: 511 - DNA, 105 bp, Artificial Sequence
  Synthetic
aacggaatcaaacggaattatcgaatggaatcgaagagaatcatcgaatggccacgaatggaatcatctaatggaatggaatggaataatccatggacccgaatg

SEQ ID NO: 512 - DNA, 62 bp, Artificial Sequence
  Synthetic
caacatcaaacggaaaaaaacggaattatcgaatggaatcgaagagaatcatcgaatggacc

SEQ ID NO: 513 - DNA, 82 bp, Artificial Sequence
  Synthetic
cacaaccaaagcaatgaaagaaaagcacagacttattgaaatgaaagtacacaccacagaatgggagcaggctcaagcaagc

SEQ ID NO: 514 - DNA, 103 bp, Artificial Sequence
  Synthetic
atcaaagggaatcaagcggaattatcgaatggaatcgaagagaatcatcgaatggactcgaatggaatcatgtgatggaatggaatggaataatccacggact

SEQ ID NO: 515 - DNA, 95 bp, Artificial Sequence
  Synthetic
aagaaacaatcaaaaggaagtgctagaaataaaacacactgtaatagaaaagaagaatgccttatgggcttatcaatagactagacatggccagg

SEQ ID NO: 516 - DNA, 84 bp, Artificial Sequence
  Synthetic
agataagaataaggcaaacatagtaatagggagttcatgaataacacacggaaagagaacttacagggctgtgatcaggaaacg

SEQ ID NO: 517 - DNA, 77 bp, Artificial Sequence
  Synthetic
ggaatcgaatggaatcaatatcaaacggagaaaaacggaattatcgaatggaatcgaagagaatcatcgaatggacc

SEQ ID NO: 518 - DNA, 61 bp, Artificial Sequence
  Synthetic
tcagaccatagcagataacatgcacattagcaatacgattgccatgacagagtggttggtg

SEQ ID NO: 519 - DNA, 73 bp, Artificial Sequence
  Synthetic
aggaatggacacgaacggaatgcaatcgaatggaatggaatctaatagaaaggaattgaatgaaatggactgg

SEQ ID NO: 520 - DNA, 82 bp, Artificial Sequence
  Synthetic
ggaagggaatcaaatgcaacagaatgtaatggaatggaatgcaatggaatgcaatggaatggaatggaatgcaatggaatgg

SEQ ID NO: 521 - DNA, 133 bp, Artificial Sequence
  Synthetic
aaattggattgaatcgaatcgaatggaaaaaatgaaatcaaatgaaattgaatggaatcgaaatgaatgtaaacaatggaatccaatggaatccaatggaatcgaatcaaatggttttgagtggcgtaaaatg

SEQ ID NO: 522 - DNA, 85 bp, Artificial Sequence
  Synthetic
aaggattcgaatggaatgcaatcgaatggaatggaatcgaacggaatggaataaaatggaagaaaactggcaagaaatggaatcg

SEQ ID NO: 523 - DNA, 86 bp, Artificial Sequence
  Synthetic
gaaaaatcattgaacggaatcgaatggaatcatcatcggatggaaacgaatggaatcatcatcgaatggaaatgaaaggagtcatc

SEQ ID NO: 524 - DNA, 92 bp, Artificial Sequence
  Synthetic
ggttcaacttacaatattttgacttgacaacagtgcaaaagcaatacacgattagtagaaacacacttccaatgcccataggaccattctgc

SEQ ID NO: 525 - DNA, 77 bp, Artificial Sequence
  Synthetic
ggaatcgaatggaatcaacatcaaacggagaaaaacggaattatcgaatggaatcgaagagaatcatcgaatggacc

SEQ ID NO: 526 - DNA, 96 bp, Artificial Sequence
  Synthetic
taacctgatttgccataatccacgatacgcttacaacagtgatatacaagttacatgagaaacacaaacattttgcaaggaaactgtggccagatg

SEQ ID NO: 527 - DNA, 102 bp, Artificial Sequence
  Synthetic
taactactcacagaactcaacaaaacactatacatgcatttaccagtttattataaagatacaagtcaggaacagccaaatggaagaaatgtaaatggcaag

SEQ ID NO: 528 - DNA, 95 bp, Artificial Sequence
  Synthetic
gctcaaagaaatcagaaatgacacaagcaaatggaaaaacatgccatgttcatgaatatgaagaatcaatattgttaaaatggccatactgctca

SEQ ID NO: 529 - DNA, 93 bp, Artificial Sequence
  Synthetic
aaagaaatgtcactgcgtatacacacacacgcacatacacacaccatggaatactactcagctatacaaaggaatgaaataatccacagccac

SEQ ID NO: 530 - DNA, 90 bp, Artificial Sequence
  Synthetic
gaatagaacagaatggaatcaaatcgaatgaaatggaatggaatagaaaggaatggaatgaaatggaatggaaaggattcgaatggaatg

SEQ ID NO: 531 - DNA, 76 bp, Artificial Sequence
  Synthetic
tgaacggaatcgaatggaatcatcatcggatggaaacgaatggaatcatcatcgaatggaaatgaaaggagtcatc

SEQ ID NO: 532 - DNA, 60 bp, Artificial Sequence
  Synthetic
gaatagaacgaaatggaatggaatggaatggaatggaaaggaatggaatggaatggaacg

SEQ ID NO: 533 - DNA, 74 bp, Artificial Sequence
  Synthetic
aacgtgacatacatacaaaaagtttttagagcaagtgaaattttagctgctatatgttaattggtggtaatccc

SEQ ID NO: 534 - DNA, 70 bp, Artificial Sequence
  Synthetic
ggaataacaacaacaacaaccaaaagacatatagaaaacaaacagcacgatggcagatgtaaagcctacc

SEQ ID NO: 535 - DNA, 124 bp, Artificial Sequence
  Synthetic
gacaaaaagaatcatcatcgaatagaatcaaatggaatctttgaatggactcaaaaggaatatcgtcaaatggaatcaaaagccatcatcgaatggactgaaatggaattatcaaatggactcg

SEQ ID NO: 536 - DNA, 91 bp, Artificial Sequence
  Synthetic
gtaacaaaacagactcatagaccaatagaacagaatagagaattcagaaataagactgcacttctatgaccatgtgatcttagacaaacct

SEQ ID NO: 537 - DNA, 77 bp, Artificial Sequence
  Synthetic
agataaaaagaacagcagccaaaatgacaaaagcaaaaagcaaaatcgtgttagagccaggtgtggtgatgtgtgct

SEQ ID NO: 538 - DNA, 82 bp, Artificial Sequence
  Synthetic
gcaatctcaggatacaaaatcaatgtgcaaaaatcacaagcattctcatacaccaataacagacaaacagagccaaatcatg

SEQ ID NO: 539 - DNA, 97 bp, Artificial Sequence
  Synthetic
aaccaaaccaagcaaacaaacaaacagtaaaaactcaataacaaccaacaaacaggaaataccaggtaattcagattatctagttatgtgccatagt

SEQ ID NO: 540 - DNA, 148 bp, Artificial Sequence
  Synthetic
gaatgaattgaatgcaaacatcgaatggtctcgaatggaatcatcttcaaatggaatggaatggaatcatcgcatagaatcgaatggaattatcaacgaatggaatcgaatggaatcatcatcagatggaaatgaatggaatcgtcat

SEQ ID NO: 541 - DNA, 97 bp, Artificial Sequence
  Synthetic
tggaatggaatcaaatcgcatggaatcgaatggaatagaaaagaatcaaacagagtggaatggaatggaatggaatggaatcatgccgaatggaatg

SEQ ID NO: 542 - DNA, 87 bp, Artificial Sequence
  Synthetic
gaatccatgttcatagcacaacaaccaaacagaagaaatcactgtgaaataagaaacaaagcaaaacacagatgtcgacacatggca

SEQ ID NO: 543 - DNA, 148 bp, Artificial Sequence
  Synthetic
aaatggaataatgaaatggaatcgaacggaatcatcatcaaaaggaaccgaatgaagtcattgaatggaatcaaaggcaatcatggtcgaatggaatcaaatggaaacagcattgaatagaattgaatggagtcatcacatggaatcg

SEQ ID NO: 544 - DNA, 80 bp, Artificial Sequence
  Synthetic
gaattaacccgaatagaatggaatggaatggaatggaacagaacggaacggaatggaatggaatggaatggaatggaatg

SEQ ID NO: 545 - DNA, 109 bp, Artificial Sequence
  Synthetic
aagatatacaagcagccaacaaacatacgaaagaatgctcaacatcactaatcctcagagaaatttaaatcaaaaccacaatgagttacaatctcataccagtcagaat

SEQ ID NO: 546 - DNA, 106 bp, Artificial Sequence
  Synthetic
agaattacaaaccactgctcaacaaaataaaagagtacacaaacaaatggaagaatattccatgcttatggataggaagaatcaatattgtgaaaatggccatact

SEQ ID NO: 547 - DNA, 93 bp, Artificial Sequence
  Synthetic
catcgaatggactcgaatggaataatcattgaacggaatcgaagggaatcatcatcggatggaaacgaatggaatcatcatcgaatggaaatg

SEQ ID NO: 548 - DNA, 133 bp, Artificial Sequence
  Synthetic
cacccatctgtaggaccaggaagcctgatgtgggagagaacagcaggctaaatccagggttggtctctacagcagagggaatcacaagcctgttagcaagtgaagaaccaacactggcaagagtgtgaaggcc

SEQ ID NO: 549 - DNA, 104 bp, Artificial Sequence
  Synthetic
taatgcaaactaaaacgacaatgagatatcaatacataactaccagaaaggctaacaaaaaaacagtcataacacaccaaaggctgatgagtgaggatgtgcag

SEQ ID NO: 550 - DNA, 105 bp, Artificial Sequence
  Synthetic
aaaggaatcaaacggaattatcgaatggaatcgaaaagaatcatcgaacggactcgaatggaatcatctaatggaatggaatggaagaatccatggactcgaatg

SEQ ID NO: 551 - DNA, 88 bp, Artificial Sequence
  Synthetic
agcaacttcagcaaagtctcaggatacaaaatcaatgagcaaaaatcacaagcattcttacacaccaataacagacaaacagagagcc

SEQ ID NO: 552 - DNA, 92 bp, Artificial Sequence
  Synthetic
ggatataaacaagaaaacaactaatcacaactcaatatcaaagtgcaatgatggtgcaaaatgcaagtatggtggggacagagaaaggatgc

SEQ ID NO: 553 - DNA, 80 bp, Artificial Sequence
  Synthetic
aatcagtaaacgtaatacagcatataaacagaaccaaagacaaaaaccacatgattatctcaatagatgcagaaaaggcc

SEQ ID NO: 554 - DNA, 61 bp, Artificial Sequence
  Synthetic
aacatcaaacggaaaaaaacggaaatatcgaatggaatcgaagagaatcatcgaatggacc

SEQ ID NO: 555 - DNA, 76 bp, Artificial Sequence
  Synthetic
taaaatggaatcgaatggaatcaacatcaaatggaatcaaatggaatcattgaacggaattgaatggaatcgtcat

SEQ ID NO: 556 - DNA, 84 bp, Artificial Sequence
  Synthetic
aatcatcatcgaatggaatcgaatggtatcattgaatggaatcgaatggaatcatcatcagatggaaatgaatggaatcgtcat

SEQ ID NO: 557 - DNA, 64 bp, Artificial Sequence
  Synthetic
caatgcgtcaagctcagacgtgcctcactacggcaatgcgtcaagctcaggcgtgcctcactat

SEQ ID NO: 558 - DNA, 74 bp, Artificial Sequence
  Synthetic
aagacagaacactgaaactcaacagagaagtaacaagaacacctaagacaaggaaggagagggaaggcaggcag

SEQ ID NO: 559 - DNA, 101 bp, Artificial Sequence
  Synthetic
taagctgataagcaactttagcaaagtctcaggatacaaaatcaatgtacaaaaatcacaagcattcttatacaccaacaacagacagacggagagccaaa

SEQ ID NO: 560 - DNA, 114 bp, Artificial Sequence
  Synthetic
atgaacacgaatgtaatgcaatccaatagaatggaatcgaatggcatggaatataaagaaatggaatcgaagagaatggaaacaaatggaatggaattgaatggaatggaattg

SEQ ID NO: 561 - DNA, 84 bp, Artificial Sequence
  Synthetic
aacaatcactagtccttaagtaagagacaacaccttttgtcacacacagtttgtcctaactttatcttggtaattggggagacc

SEQ ID NO: 562 - DNA, 87 bp, Artificial Sequence
  Synthetic
taatgagaagacacagacaacacaaagaatcacagaaacatgacacaggtgacaagaacaggcaaggacctgcagtgcacaggagcc

SEQ ID NO: 563 - DNA, 79 bp, Artificial Sequence
  Synthetic
tgttgagagaaattaaacaaagcacagataaatggaaaaacgtgttcatagattgaaagacttcatgttgtatggtgtc

SEQ ID NO: 564 - DNA, 88 bp, Artificial Sequence
  Synthetic
atcaaacggaatcaaacggaattatcgaatggaatcgaagagaatcatcgaacggactcgaatggaatcatctaatggaatgggatgg

SEQ ID NO: 565 - DNA, 87 bp, Artificial Sequence
  Synthetic
acacaacaaccaagaaacaaccccattaagaagtgggaaaaatacatgaataaacacatctcaaaagaagacaaacaagtggctaac

SEQ ID NO: 566 - DNA, 94 bp, Artificial Sequence
  Synthetic
aatggaaaggaatcaaatggaatataatggaatgcaatggactcgaatggaatggaatggaatggacccaaatggaatggaatggaatggaatg

SEQ ID NO: 567 - DNA, 91 bp, Artificial Sequence
  Synthetic
ggaatacaacggaatggaatcgaaaaaaatggaaaggaatgaaatgaatggaatggaatggaatggaatggatgggaatggaatggaatgg

SEQ ID NO: 568 - DNA, 95 bp, Artificial Sequence
  Synthetic
gaatcaagcggaattatcgaatggaatcgaagagaatcatcgaaaggactcgaatggaatcatctaatggaatggaatggaataatacacggacc

SEQ ID NO: 569 - DNA, 95 bp, Artificial Sequence
  Synthetic
aacaacaacaacaacaggaaaacaacctcagtatgaagacaagtacattgatttattcaacatttactgatcacttttcaggtggtaggcagacc

SEQ ID NO: 570 - DNA, 98 bp, Artificial Sequence
  Synthetic
aagataacctgtgcccaggagaaaaacaatcaatggcaacaaaagcagaaacaacacaaatgatacaattagcagacagaaacattgagattgctatt

SEQ ID NO: 571 - DNA, 88 bp, Artificial Sequence
  Synthetic
aatggactccaatggaataatcattgaacggaatcnaatggaatcatcatcggatggaaatgantggaatcntcntcnaatggaatcn

SEQ ID NO: 572 - DNA, 80 bp, Artificial Sequence
  Synthetic
anncnntaaacgtaatccatcacataaacangancnaanagnnnaaccgcnngattatctcnnnnnntgcnnaaaaggcc

SEQ ID NO: 573 - DNA, 95 bp, Artificial Sequence
  Synthetic
taattgattcgaaattaatggaattgaatggaatgcaatcaaatggaatggaatgtaatgcaatggaatgtaatagaatggaaagcaatggaatg

SEQ ID NO: 574 - DNA, 78 bp, Artificial Sequence
  Synthetic
aaaggaatggacttgaacaaaatgaaatcgaacgataggaatcgtacagaacggaaagaaatggaacggaatggaatg

SEQ ID NO: 575 - DNA, 88 bp, Artificial Sequence
  Synthetic
agcaacttcagcaaaatctcaggatacaaaatcaatgtacaaaaatcacaagcattcttatacaccaacaacagacaaacagagagcc

SEQ ID NO: 576 - DNA, 94 bp, Artificial Sequence
  Synthetic
tgagcagggaacaatgcggataaatttcacaaatacaatgttgagcaaaagaaagacacaaaanaatacacacatacacaccatatgggctagg

SEQ ID NO: 577 - DNA, 85 bp, Artificial Sequence
  Synthetic
aatggaatggaatgtacaagaaaggaatggaatgaaaccgaatggaatggaatggacgcaaaatgaatggaatggaagtcaatgg

SEQ ID NO: 578 - DNA, 129 bp, Artificial Sequence
  Synthetic
aagttcaaacatcagtattaaccttgaacatcaatggcctacatgcatcacttaaaacatacagacaggcaaattgggttaagaaaacaaacaagcaaacaaaacatgttccaaacatttgttggctat

SEQ ID NO: 579 - DNA, 87 bp, Artificial Sequence
  Synthetic
ggaataatcattgaacggaatcgaatggaatcatcatcggatggaaacgaatggaatcatcatcgaatggaaatgaaaggagtcatc

SEQ ID NO: 580 - DNA, 82 bp, Artificial Sequence
  Synthetic
ggaacgaaatcgaatggaacggaatagaatagactcgaatgtaatggattgctatgtaattgattcgaatggaatggaatcg

SEQ ID NO: 581 - DNA, 80 bp, Artificial Sequence
  Synthetic
tgaaaggaatagactggaacaaaatgaaatcgaatggtaggaatcatacagaacagaaagaaatggaacggaatggaatg

SEQ ID NO: 582 - DNA, 75 bp, Artificial Sequence
  Synthetic
aacccgaatagaatggaatggaatggaatggaacggaacggaatggaatggaatggattggaatggaatggaatg

SEQ ID NO: 583 - DNA, 90 bp, Artificial Sequence
  Synthetic
aaagagaatcaaatggaattgaatcgaatggaatcgaatggattggaaaggaatagaatggaatggaatggaatggaatggaatggaatg

SEQ ID NO: 584 - DNA, 106 bp, Artificial Sequence
  Synthetic
aaaacacacaaacatacatgtggatgcacatataaacatgcacatacacacacacataaatgcacaaacacacttaacacaagcacacatgcaaacaaacacatgg

SEQ ID NO: 585 - DNA, 70 bp, Artificial Sequence
  Synthetic
aatggaatcatcagtaatggaatggaaaggaatggaaaggactggaatggaatggaatggaatggaatgg

SEQ ID NO: 586 - DNA, 116 bp, Artificial Sequence
  Synthetic
ggaacaaaatgaaatcgaacggtaggaatcgtacagaacggaaagaaatggaacggaatggaatgcactcaaatggaaaggagtccaatggaatcgaaaggaatagaatggaatgg

SEQ ID NO: 587 - DNA, 132 bp, Artificial Sequence
  Synthetic
agaatgagatcaagcagtataataaaggaagaagtagcaaaattacaacagagcagtgaaatggatatgctttctggcaataattgtgaaaggtctggtaatgagaaagtagcaacagctagtggctgccac

SEQ ID NO: 588 - DNA, 98 bp, Artificial Sequence
  Synthetic
aacaaatggaatcaacatcgaatggaatcgaatggaaacaccatcgaattgaaacgaatggaattatcatgaaattgaaatggatggactcatcatcg

SEQ ID NO: 589 - DNA, 125 bp, Artificial Sequence
  Synthetic
taacatgcagcatgcacacacgaatacacaacacacaaacatgtatgcacgcacacgtgaatacacaacacacacaaacatgcatgcatgcatacatgaatacacagcacacaaatatccagcat

SEQ ID NO: 590 - DNA, 72 bp, Artificial Sequence
  Synthetic
gaatggaatcaacatcaaacggaaaaaaaacggaattatcgaatggaatcgaatagaatcatcgaatggacc

SEQ ID NO: 591 - DNA, 98 bp, Artificial Sequence
  Synthetic
aatcgaatgaaatggagtcaaaaggaatggaatcgaatggcaagaaatcgaatgtaatggaatcgcaaggaattgatgtgaacggaacggaatggaat

SEQ ID NO: 592 - DNA, 91 bp, Artificial Sequence
  Synthetic
aatggaattgaacggaaacatcagcgaatggaatcgaaaggaatcatcatggaatagattcgaatggaatggaaaggaatggaatggaatg

SEQ ID NO: 593 - DNA, 132 bp, Artificial Sequence
  Synthetic
atggaatcaacatcaaacagaatcaaacggaattatcgaatggaatcgaagacaatcatcgaatggactcgaatggaatcatctaatggaatggaatggaagaatccatggtctcgaatgcaatcatcatcg

SEQ ID NO: 594 - DNA, 86 bp, Artificial Sequence
  Synthetic
gaataatcattgaacggaatcgaatggaatcatcttcggatggaaacgaatggaatcatcatcgaatggaaatgaaaggagtcatc

SEQ ID NO: 595 - DNA, 88 bp, Artificial Sequence
  Synthetic
aatggactcgaatggaataatcattgaacggaatcgaatggaatcatcatcggatggaaatgagtggaatcatcatcgaatggaatcg

SEQ ID NO: 596 - DNA, 98 bp, Artificial Sequence
  Synthetic
aaatgaaatcgaacggtaggaatcgtacagaacggaaagaaatggaacggaatggaatgcaatcgaatggaaaggagtccaatggaagggaatcgaat

SEQ ID NO: 597 - DNA, 93 bp, Artificial Sequence
  Synthetic
taccaaacatttaaagaacaaatatcaatcctacgcaaaccattctgaaacacagagatggaggatatacagcgaaactcattctacatggcc

SEQ ID NO: 598 - DNA, 106 bp, Artificial Sequence
  Synthetic
tattggaatggaatggaatggagtcgaatggaacggaatgcactcgaatggaaggcaatgcaatggaatgcactcaacaggaatagaatggaatggaatggaatgg

SEQ ID NO: 599 - DNA, 86 bp, Artificial Sequence
  Synthetic
agagagtattcatcatgaggagtattactggacaaataattcacaaacgaacaaaccaaagcgatcatctttgtactggctggcta

SEQ ID NO: 600 - DNA, 64 bp, Artificial Sequence
  Synthetic
ggaatttaatagaatgtacccgaatggaacggaatggaatggaattgtatggcatggaatggaa

SEQ ID NO: 601 - DNA, 97 bp, Artificial Sequence
  Synthetic
gcaatccantanaatggaatcgaatggcatggaatataaagaaatggaatcgaagagaatggagacaaatggaatggaattgaatggaatggaattg

SEQ ID NO: 602 - DNA, 118 bp, Artificial Sequence
  Synthetic
aatggaatcgaatggaatcatcatcaaatggaatctaatggaatcattgaacggaattaaatggaatcgtcatcgaatgaattcaatgcaatcaacgaatggtctcgaatggaaccac

SEQ ID NO: 603 - DNA, 106 bp, Artificial Sequence
  Synthetic
aattgcaaaagaaacacacatatacacatataaaactcaagaaagacaaaactaacctatggtgatagaaatcagaaaagtacagtacattggttgtcttggtggg

SEQ ID NO: 604 - DNA, 89 bp, Artificial Sequence
  Synthetic
tgacatcattattatcaagaaacattcttaccactgttaccaacttcccaacacagactatggagagagagataagacagaatagcatt

SEQ ID NO: 605 - DNA, 81 bp, Artificial Sequence
  Synthetic
ggaatctataatacagctgtttatagccaagcactaaatcatatgatacagaaaacaaatgcagatggtttgaagggtggg

SEQ ID NO: 606 - DNA, 89 bp, Artificial Sequence
  Synthetic
aaagaattgaattgaatagaatcaccaatgaattgaatcgaatggaatcgtcatcgaatggaatcgaagggaatcattggatgggctca

SEQ ID NO: 607 - DNA, 87 bp, Artificial Sequence
  Synthetic
atcatcgaatggaatcgaatggaatcaatatcaaacggaaaaaaacggaattatcgaatggaatcgaatagaatcatcgaatggacc

SEQ ID NO: 608 - DNA, 83 bp, Artificial Sequence
  Synthetic
gaatgaaatcgtatagaatcatcgaatgcaactgaatggaatcattaaatggacttgaaaggaattattatggaatggaattg

SEQ ID NO: 609 - DNA, 90 bp, Artificial Sequence
  Synthetic
taagcaacttcagcaaagtctcaggatacaaaatcaatgtgcaaaaatctcaagcattcttatacacgaacaacagacaaacagagagct

SEQ ID NO: 610 - DNA, 79 bp, Artificial Sequence
  Synthetic
actcaaaaggaattgattcgaatggaatagaatggcaaggaatagtattgaattgaatggaatggaatggacccaaatg

SEQ ID NO: 611 - DNA, 95 bp, Artificial Sequence
  Synthetic
gaatggaatttaaaggaatagaatggaaggaatcggatggaatggaatggaatagaatggagtcgaatggaatagaatcgaatggaatggcattg

SEQ ID NO: 612 - DNA, 86 bp, Artificial Sequence
  Synthetic
aacaaaaaatgagtcaagccttaaataaaatcagagccaaaaaagaagacattacatctgataagacaaaaattcaaaggaccatc

SEQ ID NO: 613 - DNA, 96 bp, Artificial Sequence
  Synthetic
aacccagtggaattgaattgaatggaattgaatggaatggaaagaatcaatccgagtcgaatggaatggtatggaatggaatggcatggaatcaac

SEQ ID NO: 614 - DNA, 65 bp, Artificial Sequence
  Synthetic
atcaacatcaaacggaaaaaaaacggaattatcgaatggaatcgaagagaatcatcgaatggacc

SEQ ID NO: 615 - DNA, 92 bp, Artificial Sequence
  Synthetic
aaggaatggaatggtacggaatagaatggaatggaacgaattgtaatggaatggaatttaatggaacggaatggaatggaatggaatcaacg

SEQ ID NO: 616 - DNA, 66 bp, Artificial Sequence
  Synthetic
aacggaatggaaagcaatttaatcaaatgcaatacagtggaattgaagggaatggaatggaatggc

SEQ ID NO: 617 - DNA, 150 bp, Artificial Sequence
  Synthetic
aatcgaatggaacggaatagaatagactcgaatgtaatggattgctatgtaattgattcgaatggaatggaatcgaatggaatgcaatccaatggaatggaatgcaatgcaatggaatggaatcgaacggaatgcagtggaagggaatgg

SEQ ID NO: 618 - DNA, 87 bp, Artificial Sequence
  Synthetic
tagcaacattttagtaacatgatagaaacaaaacagcaacatagcaatgcaatagtaacacaacagcaacatcataacatggcagca

SEQ ID NO: 619 - DNA, 88 bp, Artificial Sequence
  Synthetic
ggacaaattgctagaaataaacaaattaccaaaaatgattcaagtagagacagagaatcaaaatagaactacacataagtgggccaag

SEQ ID NO: 620 - DNA, 86 bp, Artificial Sequence
  Synthetic
aaaatagaatgaaagagaatcaaatggaattgaatcgaatggaatcgaatggattggaaaggaatagaatggaatggaatggaatg

SEQ ID NO: 621 - DNA, 136 bp, Artificial Sequence
  Synthetic
agcaaacaagtgaataaacaagcaaacaagtgaacaagcaaacaagtgaataaacaagcaaacaagtgaacaagcaaacaagtgaataaacaagcaaacaagtgaacaaggaaacaagtgaataaacaaaggctct

SEQ ID NO: 622 - DNA, 94 bp, Artificial Sequence
  Synthetic
aatggaatcaacacgagtgcaattgaatggaatcgaatggaatggaatggaatggaatgaattcaacccgaatggaatggaaaggaatggaatc

SEQ ID NO: 623 - DNA, 90 bp, Artificial Sequence
  Synthetic
aatatacgcaaatcaataaatgtaatccagcatataaacagtactaaagacaaaaaccacatgattatctcaatagatgcagaaaaggcc

SEQ ID NO: 624 - DNA, 76 bp, Artificial Sequence
  Synthetic
gaatcgaatggaatcaacatcaaacggaaaaaaacggaattatcgaatggaatcgaagagnnnnnncgaatggacc

SEQ ID NO: 625 - DNA, 141 bp, Artificial Sequence
  Synthetic
aacacgaatgtaatgcaatccaatagaatggaatcgaatggcatggaatataaagaaatggaatcgaagagaatggaaacaaacggaatggaattgaatggaatggaattgaatggaatgggaacgaatggagtgaaattg

SEQ ID NO: 626 - DNA, 146 bp, Artificial Sequence
  Synthetic
gaatggaacggaatagaacagactcgaatgtaatggattgctatgtaattgattcgaatggaatggaatcgaatggaatgcaatccaatggaatggaatgcaatgcaatggaatggaatcgaatggaatgcagtggaagggaatgg

SEQ ID NO: 627 - DNA, 76 bp, Artificial Sequence
  Synthetic
gaatcgaatggaatcaatatcaaacggaaaaaaacggaattatcgaatggaatcgaagagaatcatcgaatggacc

SEQ ID NO: 628 - DNA, 82 bp, Artificial Sequence
  Synthetic
taaacaacgagaacacatgaacacaaagaggggaacaacagacaccaagaccttcttgagggtggaggatgggaggagggag

SEQ ID NO: 629 - DNA, 86 bp, Artificial Sequence
  Synthetic
agcaacttcagcagtctcagtatacaaaaacaatgtgcaaaaatcacaagcattcctatatgccaataacagacaaacagagagcc

SEQ ID NO: 630 - DNA, 95 bp, Artificial Sequence
  Synthetic
atcaaaagaaaagcaacctaacaaatacgggaagaatatttgaatagacatttcacaggaaaagatatatgaatggccaaaaagcaaatgaaaag

SEQ ID NO: 631 - DNA, 136 bp, Artificial Sequence
  Synthetic
ataaacatcaaacggaatcaaacggaattatcgaatggaatcgaagagaataatcgaatggactcaaatggagtcatctaatggaatggtatggaagaatccatggactccaacgcaatcatcagcgaatggaatc

SEQ ID NO: 632 - DNA, 101 bp, Artificial Sequence
  Synthetic
aaaagaaaagacaaaagacaccaattgccaatactgaaatgaaaaaacaggtaataactattgatcccatggacattaaaatgatgttgaaggaacaccac

SEQ ID NO: 633 - DNA, 92 bp, Artificial Sequence
  Synthetic
agcaataaccaaacaacctcattaaaaagtaggcaaaggacataaacagacacttttcaaaagaagacatacacgtggccaacaaacatatg

SEQ ID NO: 634 - DNA, 88 bp, Artificial Sequence
  Synthetic
agcaacttcagcaaagtctcaggatacaaaatcgatgtgcaaaaatcacaagcattcttatacaccaataacaggcaaacagagagcc

SEQ ID NO: 635 - DNA, 77 bp, Artificial Sequence
  Synthetic
gtcatatttgggatttatcatctgtttctattgttgttgttttagtacacacaaagccacaataaatattctaggct

SEQ ID NO: 636 - DNA, 87 bp, Artificial Sequence
  Synthetic
atcatcgaatggaatagaatggtatcaacatcaaacggagaaaaacggaattatcgaatggaatcgaagagaatcttcgaacggacc

SEQ ID NO: 637 - DNA, 97 bp, Artificial Sequence
  Synthetic
aaataagccaacggtcataaattgcaaagccttttacaatccaaacatgatggaaacgatatgccattttgaaggtgatttgaaaagcacatggttt

SEQ ID NO: 638 - DNA, 122 bp, Artificial Sequence
  Synthetic
gaatggaatcatcgcatagaatcggatggaattatcatcgaatggaatcgaatggtatcaacatcaaacggaaaaaaacggaattatcgaatggaatcgaattgaatcatcgaacggacccg

SEQ ID NO: 639 - DNA, 133 bp, Artificial Sequence
  Synthetic
aatggactcgaatggaataatcattgaacggaatcgaatggaatcatcatcggatggaaatgaatggaataatccatggactcgaatgcaatcatcatcgaatggaatcgaatggaatcatcgaatggactcg

SEQ ID NO: 640 - DNA, 70 bp, Artificial Sequence
  Synthetic
aatgcaatcatcaactggcttcgaatggaatcatcaagaatggaatcgaatggaatcatcgaatggactc

SEQ ID NO: 641 - DNA, 68 bp, Artificial Sequence
  Synthetic
aagagaccaataaggantangtaagcaacangaggaaggaganangggcaagagagatgaccagagtt

SEQ ID NO: 642 - DNA, 87 bp, Artificial Sequence
  Synthetic
tggaatcatcataaaatggaatcgaatggaatcaacatcaaatggaatcaaatggaatcattgaacggaattgaatggaatcgtcat

SEQ ID NO: 643 - DNA, 117 bp, Artificial Sequence
  Synthetic
ggaatcatcgcatagaatcgaatggaattatcatcgaatggaatcgaatggaatcaacatcaaacgaaaaaaaaccggaattatcgaatggaatcgaagagaatcatcgaacggacc

SEQ ID NO: 644 - DNA, 85 bp, Artificial Sequence
  Synthetic
aaatcatcatcgaatgggatcgaatggtatccttgaatggaatcgaatggaatcatcatcagatggaaatgaatggaatcgtcat

SEQ ID NO: 645 - DNA, 104 bp, Artificial Sequence
  Synthetic
ggaatgtaatagaacggaaagcaatggaatggaacgcactggattcgagtgcaatggaatctattggaatggaatcgaatggaatggtttggcatggaatggac

(19) United States
(12) Patent Application Publication
(10) Pub. No.: US 2013/0230884 A1
(43) Pub. Date: Sep. 5, 2013

(54) Methods to Identify Synthetic and Natural RNA Elements that Enhance Protein Translation

(71) Applicants: John Chaput, Phoenix, AZ (US); Sudhir Kumar, Tempe, AZ (US); Bertram Jacobs, Tempe, AZ (US)

(72) Inventors: John Chaput, Phoenix, AZ (US); Sudhir Kumar, Tempe, AZ (US); Bertram Jacobs, Tempe, AZ (US)

(21) Appl. No.: 13/703,561

(22) PCT Filed: Jul. 15, 2011
(86) PCT No.: PCT/US11/44198

§ 371 Date: Mar. 29, 2013

Related U.S. Application Data
(60) Provisional application No. 61/365,133, filed on Jul. 16, 2010.

(51) Int. Cl. C12N 15/10; C12N 15/113

(52) U.S. Cl. CPC C12N 15/1062; C12N 15/113
USPC 435/69.1; 506/14; 506/17; 506/9; 536/24.1; 435/320.1; 435/325; 435/366

(57) ABSTRACT

The present invention provides reagents and methods for identifying translation enhancing elements, as well as isolated translation enhancing elements and their use in protein expression reagents and methods.

### CROSS-REFERENCE

This application claims priority to U.S. Provisional Patent Application Ser. No. 61/365,133 filed Jul. 16, 2010, incorporated by reference herein in its entirety.

### STATEMENT OF U.S. GOVERNMENT INTEREST

This work was funded in part by NIH Eureka Award GM085530. The U.S. government has certain rights in the invention.

### BACKGROUND

Ribosomal initiation constitutes a critical step in the protein translation process, allowing the ribosome to locate the correct AUG start site in the RNA message and initiate the transfer of genetic information from RNA into proteins via the genetic code. In eukaryotes, recruitment of the 40S ribosomal subunit to the RNA message occurs by recognition of a 7-methylguanosine cap located at the 5′ end of the mRNA strand. Ribosomal recruitment can also occur by a less common cap-independent mechanism, an example of which is the internal ribosomal entry site (IRES). In many cases, the recruitment site is located some distance upstream of the initiation codon, which poses the question of how the ribosome is able to bypass the intervening sequence. While linear scanning is the dominant model used to explain this process, emerging evidence suggests that transient mRNA-rRNA base pairing may play an important role in the initiation of certain mRNAs. This possibility, and the fact that the genome is routinely and pervasively transcribed into RNA, raise many interesting questions about the role of RNA inside cells and the potential for many unknown protein coding regions.

Discovering translation initiation elements (TIEs), also known as translation enhancing elements (TEEs) in human and other higher order genomes is a challenging problem as computational methods are unable to locate these sequences at the DNA level. This limitation has created a pressing need for new functional tools that can be used to identify and map these sequences in known genomes.

### SUMMARY OF THE INVENTION

In a first aspect, the present invention provides nucleic acid libraries comprising a plurality of linear recombinant double stranded DNA constructs, wherein each double stranded DNA construct comprises

(a) a promoter;

(b) a heterologous coding region downstream from the promoter, wherein the coding region encodes a detectable polypeptide;

(c) a heterologous cross-linking region downstream of the coding region;

(d) a heterologous polynucleotide sequence of between 20-500 base pairs in length located downstream of the promoter and upstream of the coding region; and

(e) a first PCR primer binding site and a second PCR primer binding site, wherein the first PCR primer binding site is upstream of the polynucleotide sequence and the second PCR primer site is downstream of the polynucleotide sequence;

wherein at least 10¹³ different polynucleotide sequences are represented in the plurality of double stranded nucleic acid constructs, and wherein the first PCR primer and the second PCR primer are the same for each construct in the plurality of double stranded nucleic acid constructs.

In a second aspect, the present invention provides mRNA pools, comprising mRNA transcripts resulting from transcription of the nucleic acid libraries of the first aspect of the invention.

In a third aspect, the present invention provides methods for identifying translational enhancing elements (TEEs), comprising

(a) contacting the nucleic acid library of the first aspect of the invention with reagents for RNA transcription under conditions to promote transcription of RNA from the double stranded nucleic acid constructs, resulting in an RNA expression product;

(b) contacting the RNA expression product with reagents for ligating a linker containing a puromycin residue to the 3′ end of the RNA expression product, resulting in a labeled RNA expression product;

(c) contacting the labeled RNA expression product with reagents for protein expression under conditions to promote protein translation from the labeled RNA expression product, resulting in a RNA-polypeptide fusion product;

(d) isolating RNA-polypeptide fusion products;

(e) converting the isolated RNA-polypeptide fusion products to cDNA by reverse transcription-PCR using a primer to the 3′ end of the isolated RNA-polypeptide fusion products;

(f) amplifying the cDNA by PCR using primers to the 5′ and 3′ end of the cDNA; and

(g) repeating steps (a)-(f) a desired number of times, wherein the amplified polynucleotide sequence fragments comprise TEEs.

In a fourth aspect, the present invention provides isolated polynucleotides, comprising a nucleic acid sequence according to any one of SEQ ID NOS: 1-5 and 7-645. These polynucleotides have been identified as TEEs using the methods of the present invention.

In a fifth aspect, the present invention provides expression vectors comprising

(a) a promoter;

(b) a heterologous TEE downstream of the promoter, where the TEE comprises a polynucleotide according to the fourth aspect of the invention; and

(c) a cloning site suitable for cloning of an protein-encoding nucleic acid of interest located upstream of the TEE, and downstream of the promoter.

In a sixth aspect, the present invention provides recombinant host cells comprising the expression vector of the fifth aspect of the invention.

In a seventh aspect, the present invention provides methods for protein expression, comprising contacting an expression vector of the fifth aspect of the invention with reagents and under conditions suitable for promoting expression of a polypeptide cloned into the cloning site.

### DESCRIPTION OF THE FIGURES

FIG. 1. In vitro selection and characterization of RNA elements that mediate cap-independent. (A) Human genomic DNA fragments were inserted into a DNA cassette containing all of the sequence information necessary to perform an mRNA display selection. For each selection round, the dsDNA pool was in vitro transcribed into ssRNA, conjugated to a DNA-puromycin linker, and translated in vitro. Uncapped mRNA sequences that initiate translation of an intact ORF become covalently linked to a His-6 protein affinity tag encoded in the RNA message. Functional molecules are recovered, reverse transcribed, and amplified by PCR to generate the input for the next round of selection. (B) Generation of RNA-protein fusion molecule by the natural peptidyl transferase activity of the ribosome, which catalyzes the formation of a non-hydrolyzable amide bond between puromycin and the polypeptide chain. (C) The selection progress was monitored by measuring the fraction of S³⁵-labeled mRNA-peptide fusions that bound to an oligo-dT column and a Ni-NTA affinity column. Chromosomal distribution of in vitro selected sequences with 100% sequence similarity to the human reference genome (D) and their evolutionary conservation compared to the starting library (round 0) (E). (F) The distribution of individual repeat families in the starting library, random genomic sequences, and the in vitro selected sequences.

FIG. 2. Functional analysis of top nine sequences in human cells. (A) Schematic diagram showing the individual steps of a coupled transcription-translation assay for cytoplasmic RNA expression and analysis. A luciferase reporter plasmid carrying an insert and a promoter sequence specific to the vaccinia virus is transfected into HeLa cells that are immediately infected with vaccinia virus. Virus-infected cells synthesize a vaccinia RNA polymerase that enables cytoplasmic transcription of the reporter plasmid into RNA. The mRNA transcripts are translated by endogenous ribosomes and the cells are assayed for bioluminescence activity after 6 hours of infection. The translation efficiency of the top nine sequences identified in the cell-based screen in (B) HeLa cells and (C) in vitro in HeLa cell lysate.

FIG. 3. Functional analysis of the top nine sequences in the hairpin plasmid. (A) The sequences were inserted into a firefly reporter plasmid (F-luc-hp) containing a stable stem-loop structure. (B) The translation efficiency of the controls with no insert in vitro and in cell-based assays with and without the stable stem-loop structure. (C) The translation efficiency of the top nine sequences in vitro relative to the no insert control.
(D) The translation efficiency of the top nine sequences in HeLa cells relative to the no insert control after normalization for mRNA (E) No infection assay in HeLa cells demonstrating that HGL6.877, HGL6.1033, and HGL6.733 have weak promoter activity that is specific to vaccinia virus infection. No activity is observed for these sequences in the absence of the vaccinia virus (inset).

FIG. 4. Translation initiation efficiency of AUG triplet patterns. (A) In vitro translation efficiency of selected sequences with in-frame and out-of-frame AUG triplets. (B) Gel image illustrating start site usage of sequences in rabbit and human cell lysate. (C) In vitro translation efficiency of HGL6.877 and an unselected sequence (HGL0.53) with various combinations of AUG triplets.

### DETAILED DESCRIPTION OF THE INVENTION

In a first aspect, the present invention provides nucleic acid libraries comprising a plurality of linear recombinant double stranded DNA constructs, wherein each double stranded DNA construct comprises

(a) a promoter;

(b) a heterologous coding region downstream from the promoter, wherein the coding region encodes a detectable polypeptide;

(c) a heterologous cross-linking region downstream of the coding region;

(d) a heterologous polynucleotide sequence of between 20-500 base pairs in length located downstream of the promoter and upstream of the coding region; and

(e) a first PCR primer binding site and a second PCR primer binding site, wherein the first PCR primer binding site is upstream of the polynucleotide sequence and the second PCR primer site is downstream of the polynucleotide sequence;

wherein at least 10¹³ different polynucleotide sequences are represented in the plurality of double stranded nucleic acid constructs, and wherein the first PCR primer and the second PCR primer are the same for each construct in the plurality of double stranded nucleic acid constructs.

The nucleic acid libraries according to the present invention can be used, for example, in the methods of the invention for performing in vitro selection for the isolation of RNA elements (TEEs, including internal ribosome entry sites (IRESs)) that can mediate cap-independent protein translation. The libraries comprise a series of linear constructs, which, when used in in vitro selection methods as described herein, permit use of a library diversity of at least 10¹³ different polynucleotide sequences. As described in detail below, the inventors have used the libraries of the present invention to identify a large number of novel TEEs, including a number of IRESs. As used herein, a “library” is a collection of linear double stranded nucleic acid constructs.

As used herein, “heterologous” means that none of the promoter, coding region, genomic fragment, and cross-linking region are normally associated with each other (ie: they are not part of the same gene in vivo), but are recombinantly combined in the construct.

As used herein, a “promoter” is any DNA sequence that can be used to help drive RNA expression of a DNA sequence downstream of the promoter. Suitable promoters include, but are not limited to, the T7 promoter, SP6 promoter, CMV promoter, and vaccinia virus synthetic-late promoter. As will be understood by those of skill in the art, a given double stranded DNA construct may contain more than one promoter, as appropriate for a given proposed use.

As used herein, a “coding region” is any DNA sequence encoding a polypeptide product. As used herein, a “detectable polypeptide” is any polypeptide whose expression can be detected, including but not limited to a fluorescent polypeptide (GFP, BFP, etc.), a member of a binding pair, an affinity tag, etc. The ability to detect the polypeptide greatly facilitates the methods of the invention. Non-limiting examples of such detectable polypeptides include affinity tags, protein DX (Smith et al. (2007) PLoS ONE 2, e467), maltose-binding protein (MBP), streptavadin, glutathionine S-transferase (GST), flagellar protein FlaG (FLAG affinity tag), and myelocytomatosis and viral oncogene homologs (Myc affinity tag).

As used herein, a “cross linking region” is any nucleic acid sequence that can be expressed as RNA, where the expressed RNA can serve as a site for ligation/binding to a linker to form a stable complex between mRNA-ribosome-protein. In a preferred embodiment, expressed RNA from the cross-linking region can serve as a site for ligation to a linker containing a 3′-puromycin residue. In a non-limiting embodiment, the expressed RNA from the cross-linking region can serve as a site for photo-ligation of a psoralen-DNA-puromycin linker (5′-psoralen-(oligonucleotide complementary to linker)-(PEG₉)₂-A₁₅-ACC-puromycin). In a preferred embodiment, the linker is a DNA linker, and the mRNA expressed from the cross linking region is complementary to the DNA linker sequence to be used.

The polynucleotide sequence can be any suitable length, such as between 20-1000 base pairs. In a preferred embodiment, the polynucleotide sequence is between 20-500 base pairs, and may comprise genomic fragments, such as a representation of an entire or partial genome from an organism of interest, or may comprise synthetic sequences. In embodiments where genomic fragments are used, the genomic fragments may be generated by any appropriate means, including restriction enzyme digestion, shearing, polynucleotide synthesis, etc. Genomic fragments from any suitable organism of interest may be used, including but not limited to human, mammal, fish, reptile, plant, yeast, insect, prokaryotic, bacterial (E. coli, etc.), viral, fungal, and pathogenic organism genomic fragments. In another preferred embodiment, such genomic fragments are obtained from plurality of individual organisms of a single species; in a further embodiment, the plurality of individual organisms of a single species differ in ancestry, age, gender, and/or other characteristics.

The primer binding sites provide regions of known sequence around the polynucleotide sequence of unknown sequence to be tested for TEE activity. Additionally the primer binding sites provide a way to amplify only the polynucleotide sequence back out of the construct as desired. As will be understood by those of skill in the art, any suitable sequence can be used as a primer binding site so long as it can be used to bind a primer of interest. The primer binding site may be immediately adjacent to the polynucleotide sequence, or there may be additional nucleotides present between the primer binding site and the polynucleotide sequence as deemed appropriate for a given purpose.

As used herein, “at least 10¹³ different polynucleotide sequences are represented in the plurality of double stranded nucleic acid constructs” means that the library, in its entirety, contains at least 10¹³ different polynucleotide sequences that can be tested for TEE activity, while each different double stranded nucleic acid construct contains only a single polynucleotide sequence. In various embodiments, at least 10¹⁴ different polynucleotide sequences or at least 10¹⁵ different polynucleotide sequences are represented in the plurality of double stranded nucleic acid constructs.

It will be understood by those of skill in the art that the constructs of the invention may comprise further nucleotide elements as appropriate for a given intended use. In one preferred embodiment, the double stranded nucleic acid constructs further comprise one or more unique restriction sites upstream of the polynucleotide sequence and downstream of the promoter, and one or more unique restriction sites downstream of the polynucleotide sequence. This embodiment provides a further means by which to isolate polynucleotide sequences of interest from the constructs. In a further embodiment, the constructs do not include sequences encoding a 3′ poly(A) tail, or sequences that promote formation of a 5′ cap on the resulting transcript.

In another preferred embodiment, the second (3′) primer binding site is immediately upstream of the coding region in the double stranded nucleic acid construct. In this embodiment, the 3′ primer binding site abuts the coding region when the polynucleotide sequence is upstream of the promoter.

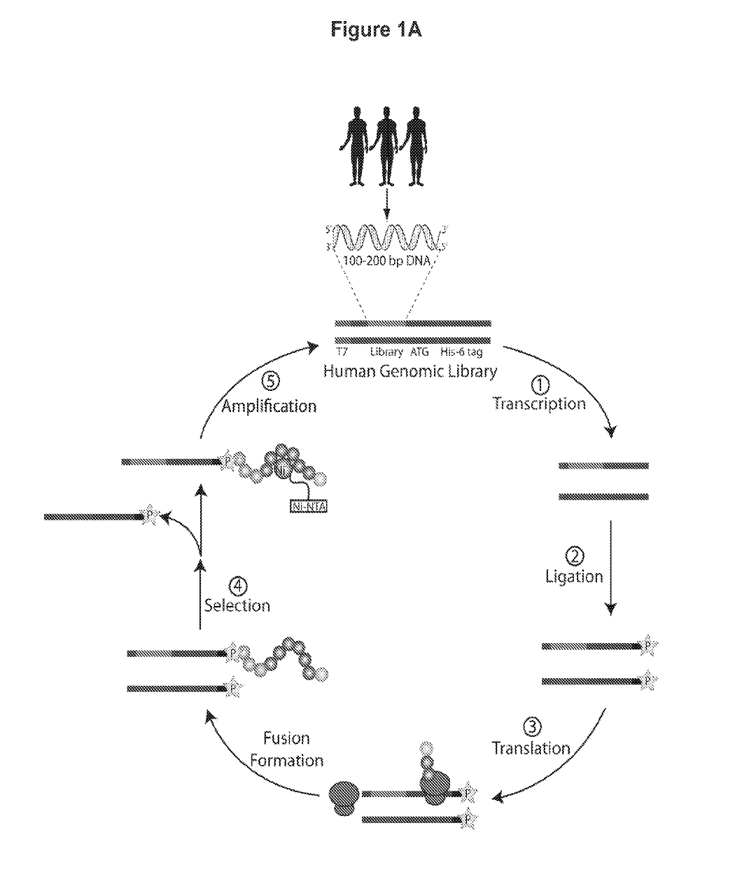

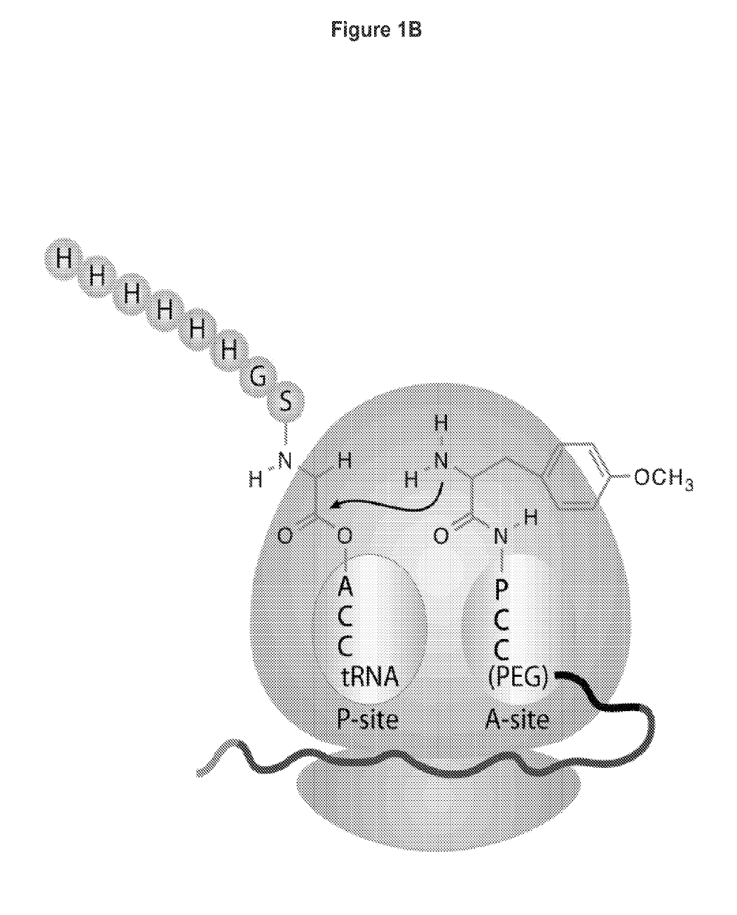

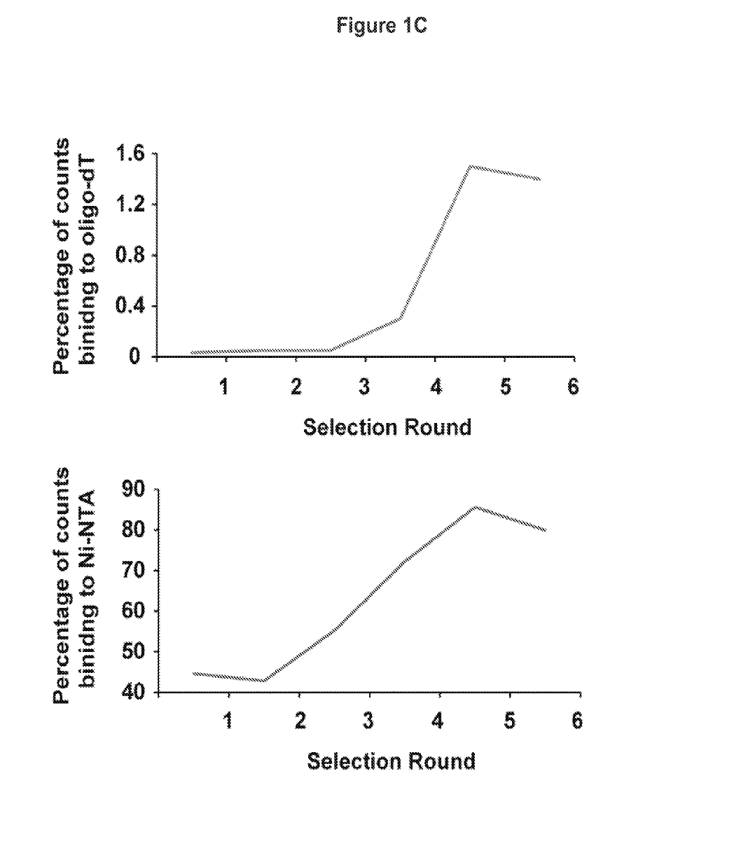

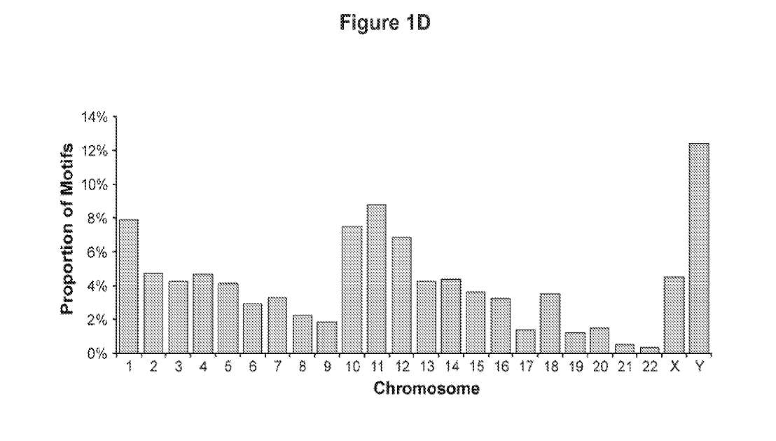

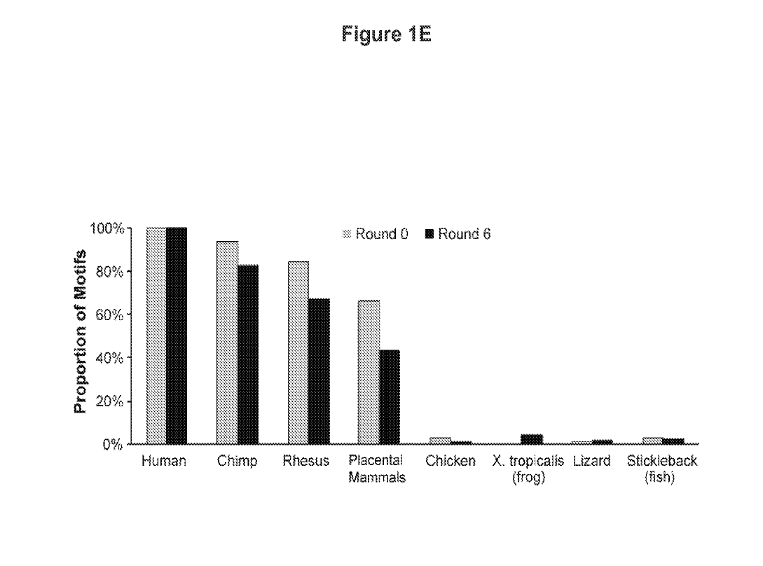

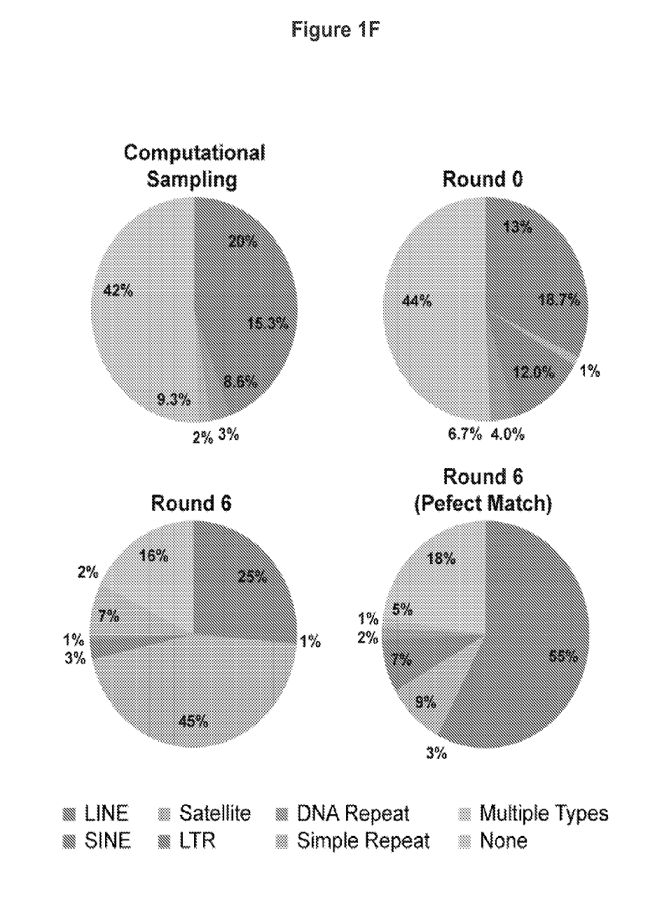

In a second aspect, the present invention provides an mRNA pool resulting from transcription of the library of any embodiment of the first aspect of the invention. Such mRNA pools can be used, for example, in the methods of the invention below. Any suitable technique for RNA transcription can be used. In one non-limiting embodiment, the double stranded DNA constructs each comprise a T7 RNA polymerase promoter, and the library is transcribed in vitro using T7 RNA polymerase, using standard techniques. It will be clear to those of skill in the art how to optimize transcription conditions in terms of buffers, nucleotides, salt conditions, etc., based on the general knowledge of in vitro transcription techniques in the art. The resulting mRNA pools will comprise single stranded RNA from all/almost all the double stranded DNA constructs in the library. In a further embodiment, the transcripts in the pooled mRNA comprise a DNA linker, containing a 3′ puromycin residue, ligated at the 3′ end of the transcript. In a further aspect, the invention provides pooled mRNA-peptide fusion molecules resulting from in vitro translation of the pooled mRNA. Methods for in vitro translation of RNA transcripts are well known to those of skill in the art. In one non-limiting embodiment, the methods comprise incubating the pooled mRNA with rabbit reticulocyte lysate and ³⁵S-methionine for a suitable time. The method may further comprise incubating the mixture overnight in the presence of suitable amounts of KCl and MgCl₂ to promote fusion formation. When the pool of RNA is translated in vitro, transcripts that contain a TEE (such as an IRES) in their 5′ UTR would initiate translation and produce an mRNA-peptide fusion molecule; thus, modifying TEE-containing RNAs with a selectable tag. The chemical bond forming step of mRNA display is due to the natural peptidyl transferase activity of the ribosome, which catalyzes the formation of a non-hydrolyzable amide bond between puromycin and the polypeptide chain (FIG. 1B). mRNA-peptide fusion molecules can be isolated by affinity purification, reverse-transcribed, and amplified to regenerate the pool of DNA for another selection cycle.

In a third aspect, the present invention provides in vitro methods for identifying translational enhancing elements (TEEs), comprising

(a) contacting the nucleic acid library of any embodiment or combination of embodiments of the first aspect of the invention with reagents for RNA transcription under conditions to promote transcription of RNA from the double stranded nucleic acid constructs, resulting in an RNA expression product;

(b) contacting the RNA expression product with reagents for ligating a linker containing a puromycin residue to the 3′ end of the RNA expression product, resulting in a labeled RNA expression product;

(c) contacting the labeled RNA expression product with reagents for protein expression under conditions to promote protein translation from the labeled RNA expression product, resulting in a RNA-polypeptide fusion product;

(d) isolating RNA-polypeptide fusion products;

(e) converting the isolated RNA-polypeptide fusion products to cDNA by reverse transcription-PCR using a primer to the 3′ end of the isolated RNA-polypeptide fusion products;

(f) amplifying the cDNA by PCR using primers to the 5′ and 3′ end of the cDNA; and

(g) repeating steps (a)-(f) a desired number of times, wherein the amplified polynucleotide sequence fragments comprise TEEs.

The methods of this aspect of the present invention serve to isolate RNA elements that could mediate cap-independent translation (ie: TEEs, including but not limited to IREs). The mechanism-based approach of mRNA display provides an efficient method to systematically and comprehensively survey nucleic acid sequences for all of the possible RNA elements that could initiate translation of uncapped mRNA transcripts. Since IRESs function by a cap-independent mechanism, this selection serves to identify IRESs as well as TEEs that promote cap-independent translation but do not initiate internally. All terms used in this third aspect have the same meaning as used elsewhere herein; similarly, all embodiments of the nucleic acid libraries and components thereof that are disclosed above, and combinations thereof, can be used in the methods of the invention. Thus, for example, each double stranded DNA construct comprises

(a) a promoter;

(b) a heterologous coding region downstream from the promoter, wherein the coding region encodes a detectable polypeptide;

(c) a heterologous cross-linking region downstream of the coding region;

(d) a heterologous polynucleotide sequence of between 20-1000 base pairs in length located downstream of the promoter and upstream of the coding region; and

(e) a first PCR primer binding site and a second PCR primer binding site, wherein the first PCR primer binding site is upstream of the polynucleotide sequence and the second PCR primer site is downstream of the polynucleotide sequence. In one non-limiting embodiment, the heterologous polynucleotide sequences are randomly digested fragments (in various non-limiting embodiments, ranging between 20-1000 nts, 20-750 nts, 20-500 nts; or about 150 nts) of total human DNA. Since the heterologous polynucleotide sequence is located downstream of the promoter and upstream of the coding region.

In the method, step (f) amplifying the cDNA by PCR using primers to the 5′ and 3′ end of the cDNA serves to add sequence information that was lost in steps (a) and (e). In one embodiment, primers to add a promoter (such as a T7 promoter) to the 5′ end and the cross-linking region (such as a photo-crosslinking) site (3′ end) back onto the DNA library are after each round of selection. The sequence of these PCR primers may vary depending on how each library is constructed. The result of this PCR is the fully constructed double stranded nucleic acid construct, which can be used to repeat steps (a)-(f) as desired.

Contacting the RNA expression product with reagents for ligating a linker containing a puromycin residue to the 3′ end of the RNA expression product, resulting in a labeled RNA expression product, can be carried out via any suitable method, including photo-crosslinking or Moore-Sharp splint-directed ligation.

Any suitable linker may be used. In a preferred embodiment the linker comprises a DNA linker complementary to the transcribed single stranded RNA. The DNA linker may comprise any suitable modifications, including but not limited non-natural residues and pegylation, as can be used in mRNA display.

In one preferred embodiment, the polynucleotide sequences in the library comprise genomic fragments; in a further preferred embodiment the starting pool of constructs used in the methods contains at least a 5×-1000× coverage of the genome of interest.

General conditions for in vitro transcription and translation, PCR, reverse transcription, and mRNA display techniques (including contacting an RNA expression product with reagents for ligating a linker containing a puromycin residue to the 3′ end of the RNA expression product), are well known to those of skill in the art. Exemplary such conditions are described above and in the examples that follow. To favor the selection of RNA elements that enhance ribosomal recruitment via a cap-independent mechanism, the pool of RNA transcripts is preferably devoid of a 5′ cap and 3′ poly(A) tail. As will be apparent to those of skill in the art, this can be accomplished, for example, by not including polyT sequences in the DNA template (to avoid poly(A) tail production) and by not providing capping enzymes required for 5′ cap production.

When the pool of RNA is translated in vitro, transcripts that contain a TEE in their 5′ UTR initiate translation and produce an mRNA-peptide fusion molecule; thus, modifying TEE-containing RNAs with a selectable tag. The chemical bond forming step of mRNA display is due to the natural peptidyl transferase activity of the ribosome, which catalyzes the formation of a non-hydrolyzable amide bond between puromycin and the polypeptide chain (FIG. 1B). mRNA-peptide fusion molecules can then be isolated by affinity purification, reverse-transcribed, and amplified to regenerate the pool of DNA for another selection cycle.

In one non-limiting embodiment, for each round of selection, the dsDNA library was transcribed with an RNA polymerase suitable for the promoter being used, photo-ligated to a psoralen-DNA-puromycin linker (5′-psoralen-oligonucleotide complementary to linker)-(PEG₉)₂-A₁₅-ACC-puromycin), and translated in vitro by incubating the library with rabbit reticulocyte lysate and ³⁵S-methionine under suitable conditions. mRNA-peptide fusion molecules are reverse transcribed, and can be purified by any suitable means, including but not limited to a two-step procedure on oligo (dT)-cellulose beads (NEB) and Ni-NTA agarose affinity resin (Qiagen). Functional TEEs are recovered by any suitable technique, including but not limited to eluting the column with imidazole, dialyzing the sample into water, and amplifying the cDNA by PCR. The selection progress can be monitored using any suitable technique, including but not limited to determining the fraction of S³⁵-labeled mRNA-peptide fusions that remained on the oligo (dT)/Ni-NTA affinity columns. After a desired number of rounds of selection and amplification, the TEEs can be identified by any suitable means, including but not limited to cloning and sequencing of the amplified DNA constructs.

The selection process (steps (a)-(f)) can be carried out any suitable number of times deemed appropriate to identify TEEs, such as 1, 2, 3, 4, 5, 6, 7, 8, 9, 10, or more times. In one preferred embodiment, at least three selection cycles are carried out, such that step (g) comprises repeating steps (a)-(f) at least two more times, and even more preferably at least 3, 4, 5, 6, 7, 8, 9, or more times.

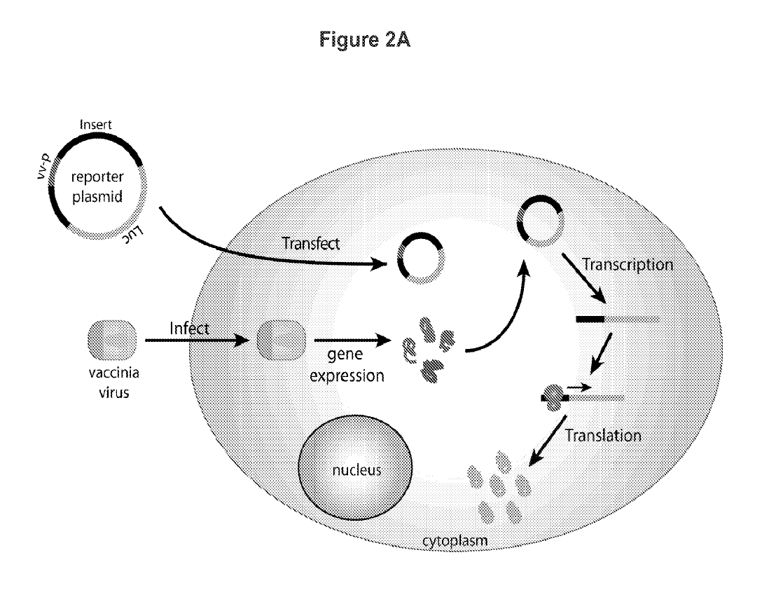

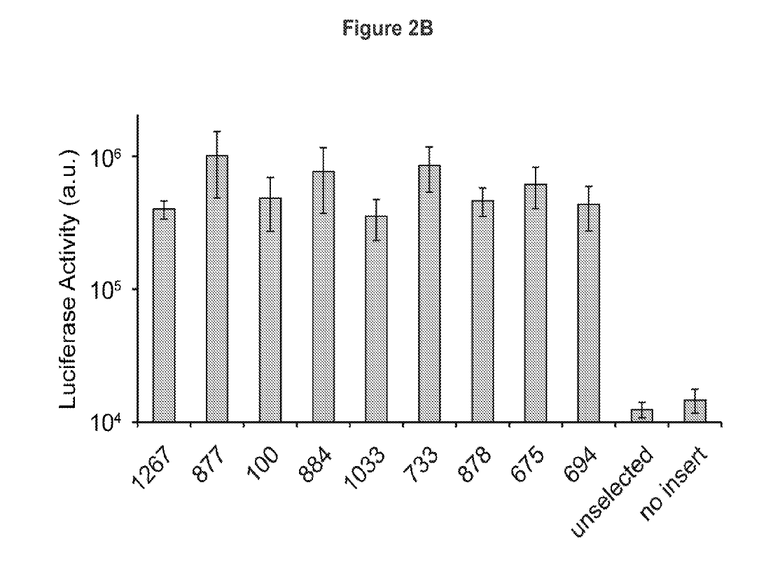

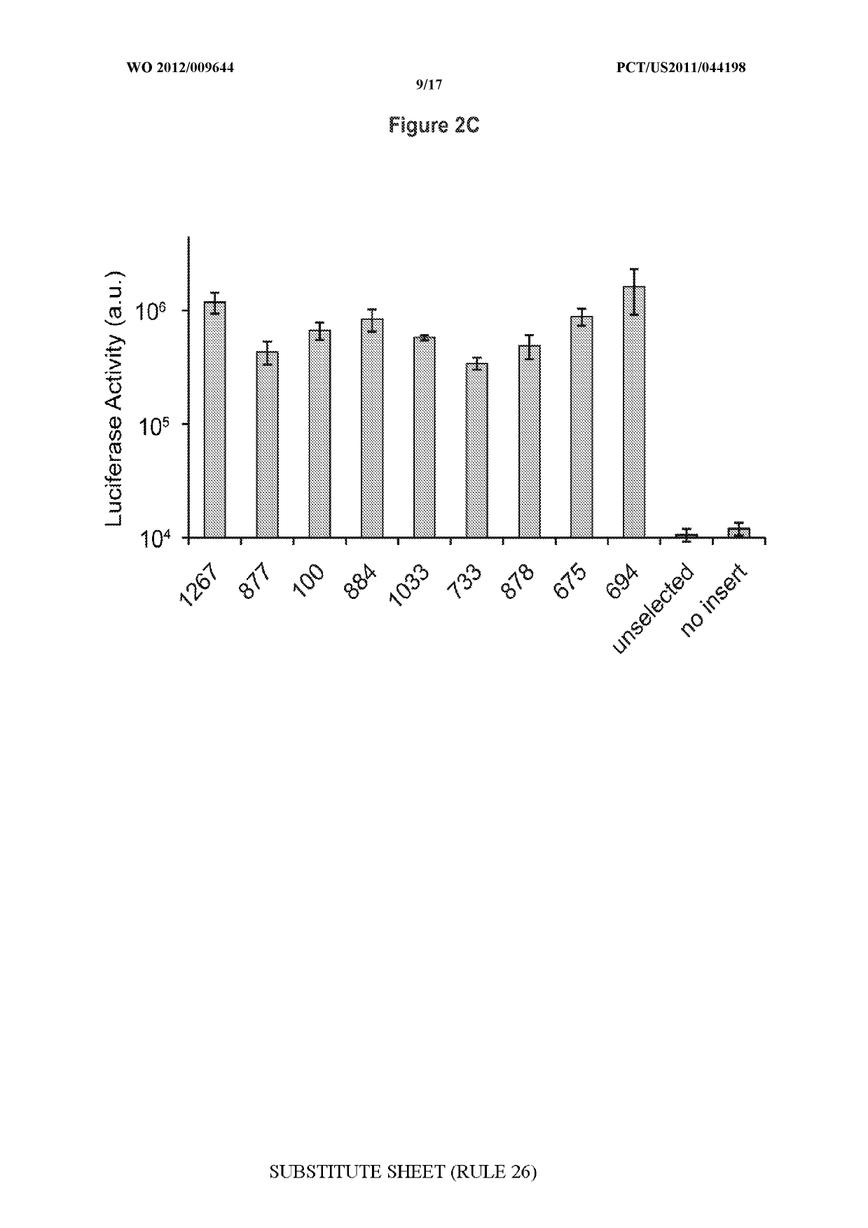

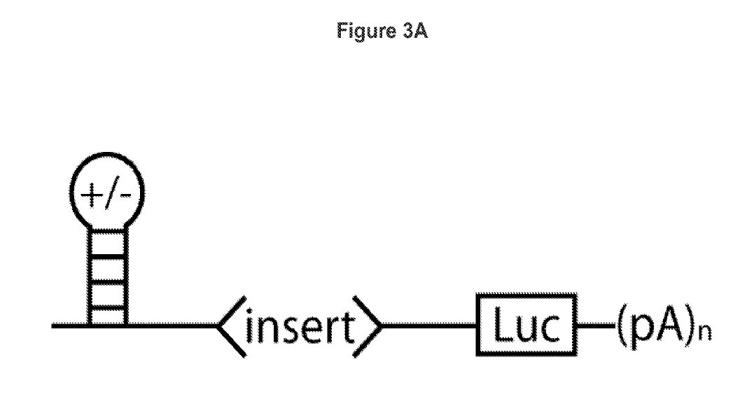

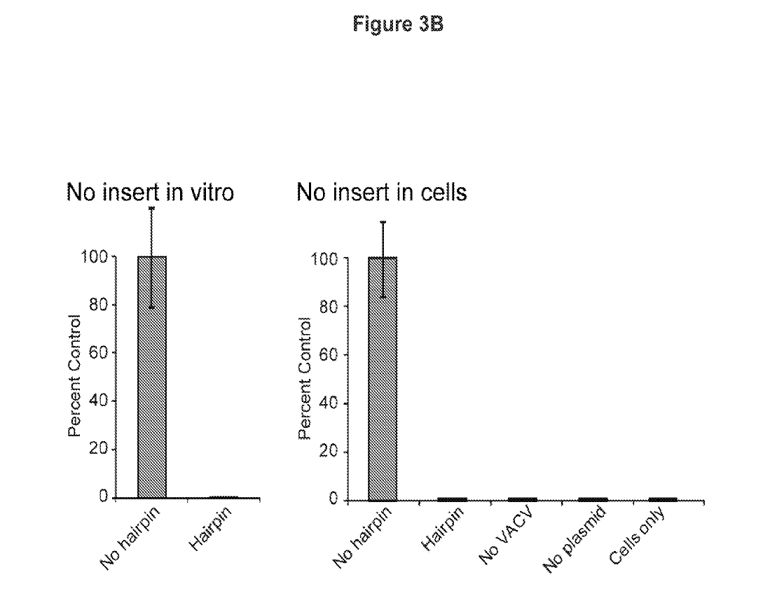

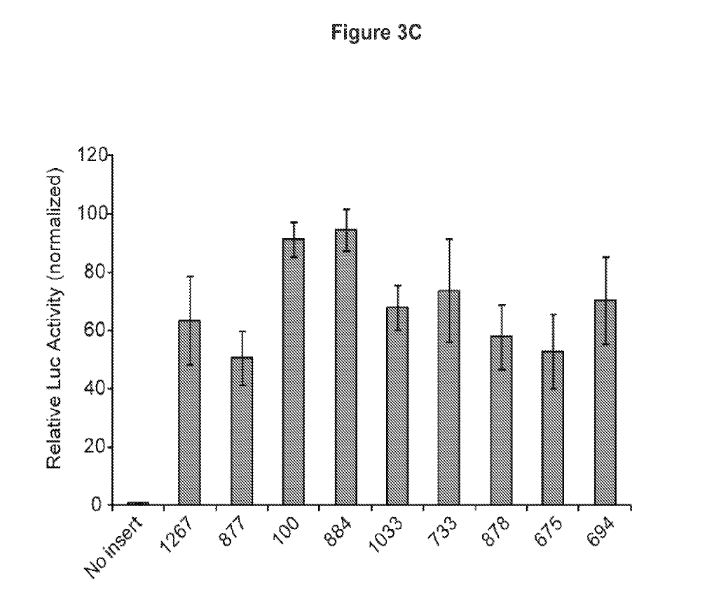

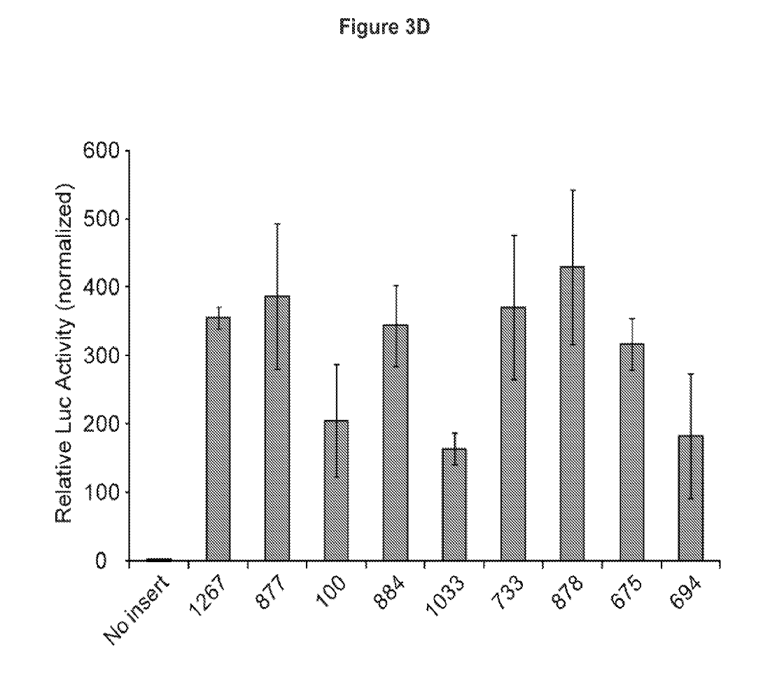

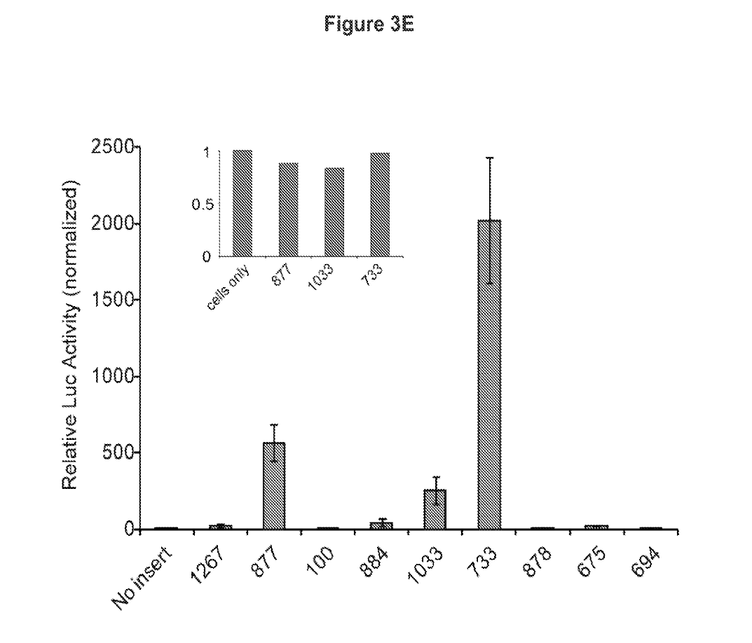

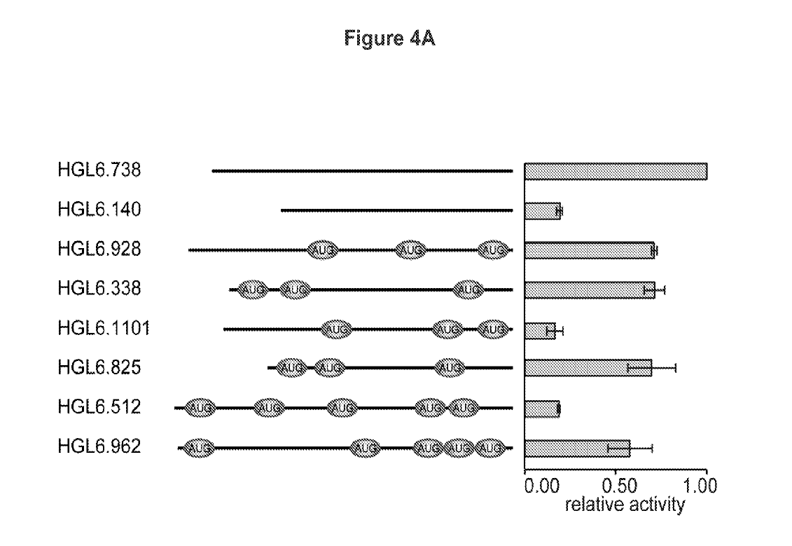

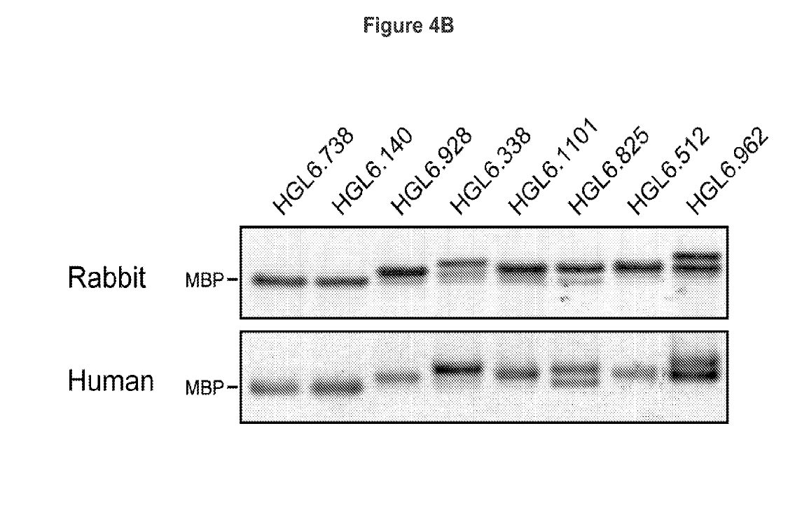

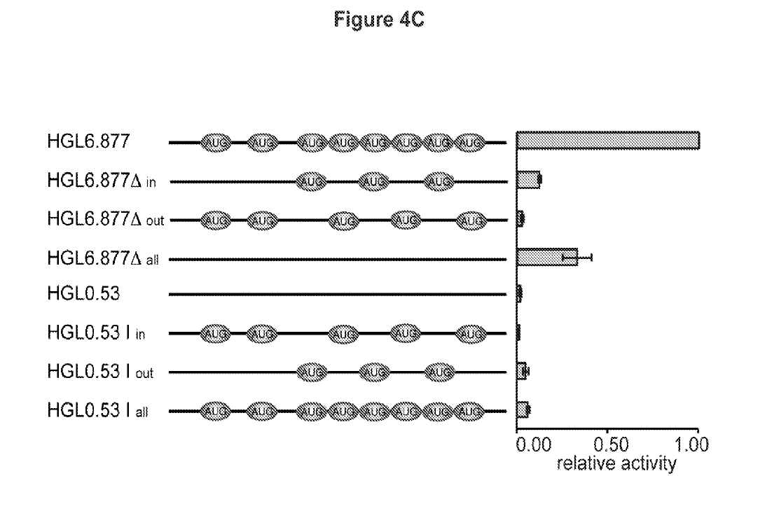

In one embodiment, the method further comprises testing polynucleotide sequences identified as TEEs for TEE activity in vivo using, for example, the vaccinia system described herein. Any suitable system may be used. In one non-limiting embodiment, a plasmid-based reporter assay that allows coupled transcription and translation to occur in the cytoplasm of human cells was developed (FIG. 2A), to test sequences under conditions that are not subject to nuclear processing. This system is based on an EMCV-driven system that relies on vaccinia virus (VACV) to circumvent nuclear expression (25). TEE candidate sequences are cloned into a monocistronic firefly luciferase reporter plasmid (F-luc-mono) containing a VACV-specific promoter. Transfected HeLa cells are infected with VACV, and after a brief incubation, cells are lysed and assayed for luciferase activity. Plasmids carrying no-insert or a randomly chosen sequence from the starting pool provided a basal level of activity.

In a further embodiment, TEE candidate sequences are tested for the ability to initiate internal translation initiation. Any suitable assay for testing internal translation initiation can be used, including but not limited to those disclosed herein. In one non-limiting embodiment, TEE candidate sequences are inserted into a firefly reporter plasmid (F-luc-hp) containing a stable stem-loop structure (ΔG=−58 kcal/mol) to prevent ribosomal scanning (FIG. 3A) (26).

In a fourth aspect, the present invention provides isolated polynucleotides, comprising a nucleic acid sequence according to any one of SEQ ID NOS: 1-5 and 7-645. In another embodiment, the isolated polynucleotides comprise or consist of a sequence according to one or more of SEQ ID NO: 7-645, listed in Table 1. The isolated polynucleotides listed in the recited tables were all identified as TEEs by the methods of the invention; all are human genomic sequences, and thus can be used, for example, in designing expression vectors for improved translational efficiency of one or more proteins encoded by the vector. In various preferred embodiments, the isolated polynucleotides are between 13-180, 13-170, 13-160, 13-150, 13-140, 13-130, 13-120, 13-110, 13-100, 13-90, 13-80, 13-70, 13-60, 13-50, 13-40, 13-30, or 13-20 nucleotides in length. In a preferred embodiment, the isolated polynucleotides consist of the recited sequence. In a further embodiment, the isolated polynucleotides comprise the sequence of SEQ ID NO:4 (A/-)(A/G)ATC(A/G)(A/G)TAAA(T/C)G, wherein the isolated polynucleotides is between 13-200 nucleotides in length. SEQ ID NO:4 is a consensus sequence found within a number of the TEES (Clones 985 (SEQ ID NO:448), 1092 (SEQ ID NO:495), 1347 (SEQ ID NO:623), 906 (SEQ ID NO:408), 12 (SEQ ID NO:12), 1200 (SEQ ID NO:553), 958 (SEQ ID NO:434), 1011 (SEQ ID NO:458), 459 (SEQ ID NO:214) in Table 1) identified using the methods of the invention. In a preferred embodiment, the isolated polynucleotides comprise the sequence of SEQ ID NO:55′-AAATCAATAAATG-3′, which is a conserved sequence found in the top-performing TEEs as described in the examples that follow. In various preferred embodiments, the isolated polynucleotides are between 13-180, 13-170, 13-160, 13-150, 13-140, 13-130, 13-120, 13-110, 13-100, 13-90, 13-80, 13-70, 13-60, 13-50, 13-40, 13-30, or 13-20 nucleotides in length.

In one embodiment, the polynucleotide is selected from the group consisting of SEQ ID NO:583 (clone 1267), SEQ ID NO:397 (clone 877), SEQ ID NO:54 (clone 100), SEQ ID NO:401 (clone 884), SEQ ID NO:471 (clone 1033), SEQ ID NO:327 (clone 733), SEQ ID NO:398 (clone 878), SEQ ID NO:301 (clone 675), and SEQ ID NO:310 (clone 694). These sequences have been identified as IRESs using the methods disclosed herein. In a further embodiment, the present invention provides isolated polynucleotides comprising a nucleic acid sequence according to SEQ ID NO:1. This sequence represents a consensus sequence of a subset of 733 (SEQ ID NO:327), 877 (SEQ ID NO:397), 1033 (SEQ ID NO:471), and 1267 (SEQ ID NO:583), and thus is strongly correlated with activity. In further embodiments, the isolated polynucleotides comprise a nucleic acid sequence according to SEQ ID NO:2 or SEQ ID NO:3, which are longer portions of the consensus sequence between 733 (SEQ ID NO:327), 877 (SEQ ID NO:397), 1033 (SEQ ID NO:471), 1267 (SEQ ID NO:583.

SEQ ID NO: 1:
5′AT(C/G)GAAT(C/G)(G/A)AA(G/T)(A/G/C)GAATGGA(A/T)
(A/T)(C/A/G)(A/G)AA(T/A)GGAAT(G/A)GAAT(T/G)(G/A)A
ATGGAATGGAA(T/A)(T/G)GA(A/T)T(G/C)GAATG-3′
SEQ ID NO: 2:
5′-( [symbol] --)( [symbol] --)( [symbol] --)( [symbol] --)( [symbol] --)
( [symbol] --)( [symbol] --)( [symbol] --)( [symbol] --)( [symbol] /--)(--/ [symbol] )
(- [symbol] )AT(C/G)
GAAT(C/G)(G/A)AA(G/T)(A/G/C)GAATGGA(AT)(A/T)
(C/A/G)(A/G)AA(T/A)GGAAT(G/A)GAAT(T/G)(G/A)
AATGGAATGGAA(T/A)(T/G)GA(A/T)T(G/C)GAATG-3′
SEQ ID NO; 3
5′-( [symbol] --)( [symbol] --)( [symbol] --)( [symbol] --)( [symbol] --)( [symbol] --)
( [symbol] --)( [symbol] --)( [symbol] --)( [symbol] --)(A/--)(A/--)(G/A/--)
(C/T/--)(G/--)(G/--)(A/--)(A/--)(T/--)(T/C/--)
(--/A/G)(--/A)AT(C/G)
GAAT(C/G)(G/A)AA(G/T)(A/G/C)GAATGGA(AT)(A/T)
(C/A/G)(A/G)AA(T/A)GGAAT(G/A)GAAT(T/G)(G/A)
AATGGAATGGAA(T/A)(T/G)GA(A/T)T(G/C)GAATG-3′

In a fifth aspect, the present invention provides expression constructs comprising:

(a) a promoter;

(b) a heterologous translational initiation element (TEE) downstream of the promoter, where the TEE comprises or consists of a sequence according to any one of SEQ ID NO:1-5 and 7-645; and

(c) a polylinker suitable for cloning of an open reading frame of interest located upstream or downstream of the TEE, and downstream of the promoter.

In this aspect, the invention provides constructs comprising the TEEs of the invention that are positioned relative to the polylinker (ie: one or more unique restriction sites to facilitate cloning) to increase translational efficiency of any polynucleotide coding region cloned into the polylinker. In a preferred embodiment, the TEE is between 13-500 nucleotides in length; in a more preferred embodiment, between 13 and 200 nucleotides in length. In a preferred embodiment, the polylinker is located downstream of the TEE. Any suitable coding region for which an increase in translational efficiency is desired can be cloned into the vector. Thus, in a further embodiment, the construct comprises a polynucleotide coding region cloned into the polylinker. In a further preferred embodiment, the TEE comprises or consists of the sequence of any one or more of SEQ ID NOS:1-5, 448, 495, 623, 408, 12, 553, 434, 458, 214, 327, 397, 471, and 583. In a further preferred embodiment, the TEE comprises or consists of the sequence of any one or more of 583 (clone 1267), SEQ ID NO:397 (clone 877), SEQ ID NO:54 (clone 100), SEQ ID NO:401 (clone 884), SEQ ID NO:471 (clone 1033), SEQ ID NO:327 (clone 733), SEQ ID NO:398 (clone 878), SEQ ID NO:301 (clone 675), and SEQ ID NO:310 (clone 694). These sequences have been identified as IRESs using the methods disclosed herein. Suitable promoters include, but are not limited to, the T7 promoter, SP6 promoter, CMV promoter, and vaccinia virus synthetic-late promoter. The constructs in this aspect of the invention may be linear constructs, or may be part of an expression vector, such as a plasmid or viral-based expression vector as are known in the art. As will be apparent to those of skill in the art, the constructs may contain any other components as desired by a user, such as origins of replication, selection markers, etc.

In a sixth aspect, the present invention provides recombinant host cell comprising an expression vector of any embodiment or combination of embodiments of the fifth aspect of the invention. Such host cells can be used, for example, to prepare large amounts of the expression vector and to provide for expression of the encoded proteins in the host cells. Any suitable host cell may be used, including but not limited to bacterial and eukaryotic host cells, including but not limited to mammalian and human cells.

In a seventh aspect, the present invention provides methods for protein expression, comprising contacting an expression construct according to any embodiment or combination of embodiments of the fifth aspect of the invention, wherein the construct comprises a polynucleotide coding region cloned into the polylinker, with reagents and under conditions suitable for promoting expression of the polypeptide encoded by the polynucleotide coding region. It is within the level of skill in the art to choose appropriate reagents and conditions for RNA expression from the expression construct, followed by translation of the encoded polypeptide. Exemplary reagents and conditions are described in the examples that follow. The methods of this aspect of the invention may be carried out in vitro or in vivo.

Unless clearly dictated otherwise by the context, all embodiments of any aspect of the invention may be combined with other embodiments of the same and different aspects.

### Example 1

### Genome-Wide Identification of Human Cap-Independent Translation Initiation Elements

Internal ribosomal entry sites (IRESs) are RNA elements located in the untranslated region of mRNA transcripts that initiate protein synthesis independent of the canonical 5′ cap. To date, only a handful of IRESs have been identified in higher order genomes. Here, we have applied a mechanism-based approach to search the entire human genome for RNA sequences with IRES activity. Starting from a library of >10¹³ human RNA fragments, we performed iterative cycles of mRNA display to capture leader sequences that mediate cap-independent translation. The selected sequences are distributed throughout the genome, and often occur in repetitive regions with high conservation to mammals. We observed strong cis-regulatory activity for more than 200 sequences tested in a monocistronic translation-enhancing assay. The most active sequences function as potent IRESs in vitro and in human cells. These results demonstrate the power of mRNA display as a genome-wide tool for identifying functional IRESs.

Initiation is a critical step in protein translation, allowing the ribosome to locate the translation start site in the RNA message and initiate the transfer of genetic information from RNA into protein via the genetic code. In eukaryotes, the 43S ribosomal pre-initiation complex (PIC) is recruited to the RNA message by recognition of the eIF4F cap-binding complex bound to a 7-methylguanosine cap located at the 5′ end of the mRNA strand (1, 2). A subset of leader sequences known as internal ribosomal entry sites (IRESs) can bypass the 5′ cap structure by recruiting the ribosome to internal positions in the 5′ untranslated region (5′ UTR) (3-7). IRESs play an important role in gene regulation by allowing essential proteins to be synthesized when normal cap-dependent translation is compromised (8). This can occur during regular cellular processes like mitosis and apoptosis (9, 10), as well as during hypoxia (11), viral infection (12), or during states of cellular dysregulation (13).

Ribosomal profiling, a technique that combines polysome fractioning with DNA microarrays, has been employed to profile cellular translation under conditions that impede normal cap-dependent translation (14). Data from these studies suggest that the human genome likely contains many more IRESs than previously thought; however, only a few human IRESs have been characterized in detail. These studies further suggest that cellular systems may possess mechanisms to support the coordinated regulation of specific IRES subtypes, as different physiological conditions gave rise to different IRES subsets. Despite a wealth of useful information gained by ribosomal profiling, this approach suffers from limited resolution and sequence accuracy, as well as an inability to distinguish stalled ribosomes from actively translating ribosomes. While continued technological advancement could circumvent some of these problems, thorough investigation of the human genome would require exhaustive sampling of countless conditions and cell types. This limitation has created a need for new molecular tools that can be used to identify human IRESs on a genome-wide scale (15).

To identify IRESs encoded in the human genome, we devised an in vitro selection strategy for the isolation of RNA elements that could mediate cap-independent translation. We reasoned that the mechanism-based approach of mRNA display provided an efficient method to systematically and comprehensively survey the entire human genome for all of the possible RNA elements that could initiate translation of uncapped mRNA transcripts (16). Since IRESs function by a cap-independent mechanism, it was hypothesized that this selection would lead to the discovery of human IRESs as well as human translation enhancing elements that promote cap-independent translation but do not initiate internally. In this scheme (FIG. 1A), a genomic library composed of randomly digested fragments (˜150 nts) of total human DNA was inserted into the 5′ UTR of a DNA cassette containing an open reading frame (ORF) encoding a peptide affinity tag. The library also contained all of the genetic information required for mRNA display. The library was converted to single-stranded RNA by in vitro transcription and photo-ligated at the 3′ end to a DNA linker containing a 3′ puromycin residue. To favor the selection of RNA elements that enhance ribosomal recruitment via a cap-independent mechanism, the pool of RNA transcripts was deprived of a 5′ cap and 3′ poly(A) tail. When the pool of RNA is translated in vitro, transcripts that contain an IRES in their 5′ UTR would initiate translation and produce an mRNA-peptide fusion molecule; thus, modifying IRES-containing RNAs with a selectable tag. The chemical bond forming step of mRNA display is due to the natural peptidyl transferase activity of the ribosome, which catalyzes the formation of a non-hydrolyzable amide bond between puromycin and the polypeptide chain (FIG. 1B) (17). mRNA-peptide fusion molecules could then be isolated by affinity purification, reverse-transcribed, and amplified to regenerate the pool of DNA for another selection cycle.

We started the selection with an RNA-DNA-puromycin library that contained >10¹³ sequences, which provided 100-1000-fold coverage of the human genome. We translated the library for 1 hour at 30° C. in nuclease treated reticulocyte lysate and fusion formation was promoted by incubating the mixture overnight at −20° C. in the presence of 600 mM KCl and 75 mM MgCl₂. mRNA-peptide fusions were isolated from the crude lysate by affinity purification on an oligo-(dT) resin, and the elution fractions were applied to Ni-NTA agarose beads. The Ni-NTA beads were thoroughly washed to remove RNA sequences that did not form mRNA-peptide fusions or did not initiate in the correct reading frame. mRNA-peptide fusions that remained bound to the column were selectively eluted with imidazole, exchanged into buffer, reverse-transcribed, and amplified by PCR to reinitiate the selection cycle described above. We monitored the selection progress by following the proportion of S³⁵-labeled mRNA-peptide fusions that remained in the pool after purification. The abundance of mRNA-peptide fusions increased up to round 5 and plateaued in round 6, indicating that the library became dominated by RNA elements that could enhance cap-independent translation (FIG. 1C).

We cloned and sequenced 712 members from round 6. Of these, 639 were non-redundant, indicating that the library contained significant sequence diversity even after six rounds of mRNA display (Table S1). Each non-redundant sequence was aligned to the human reference genome (hg18) using the UCSC BLAT web-tool (18). A subset of 229 sequences showed 100% identity to 1814 genomic locations. These sites are distributed across all 24 human chromosomes with ˜34% occurring in the intronic regions of known genes (FIG. 1D). The remaining 410 sequences have high homology (85-99% identity) to genomic sites, but contain small degrees of sequence variation that include single nucleotide polymorphisms in addition to small and large insertions and deletions. This level of variation is expected for individuals in a population (19), and it is known that gene regulatory sequences can differ between individual genomes (20). Since we could not distinguish between mutations that arose during the selection and those that occur naturally, we focused the remainder of our study on the set of 229 perfectly matched sequences.

We examined their evolutionary conservation using the 44-species UCSC alignments (FIG. 1E) (18). Of the 229 sequences, 82.5% are conserved in the chimpanzee genome (Pan troglodytes) and 43.2% are conserved in the genomes of other placental mammals (i.e., dogs, horses, and mice). The degree of sequence similarity ranged from 97-100% for chimpanzee and 96-100% for placental mammals. Sometimes significant sequence similarities (E-value<9e-13) were also observed in lower vertebrate genomes. For example, HGL6.634 homologs are found in lizards (Anolis carolinensis) and fish (Xenopus tropicalis and Gasterosteus aculeatus). This sequence overlaps the intron-exon junction for a chromatin modification-related protein. Similarly, HGL6.1305 shows high sequence similarity to the platypus and opossum genomes. This sequence is located in the intron of a neuronal PAS domain protein—a transcription factor expressed primarily in mammalian forebrains.

Because many of the perfectly matched sequences mapped to multiple genomic locations, we compared the distribution of repetitive elements found in the starting library to that of all round 6 sequences. The distribution of repetitive elements in the starting library is similar to the distribution obtained by random computational sampling (FIG. 1F). This is expected because our starting library contained an unbiased representation of the human genome (21-23). In contrast, round 6 was enriched in sequences that align to repetitive regions of the human genome. Of the 639 non-redundant sequences, most align to regions of LINE-1 (L1) retrotransposons and satellite DNA (25% and 45%, respectively). This distribution is comparable to what we observed for the set of 229 perfectly matched sequences with the difference that L1 elements are overrepresented at the expense of satellite DNA. This difference is not unexpected, as satellite DNA is known to contain large numbers of point mutations, which preclude their ability to map with 100% identity to the human reference genome (23).

We chose the set of 229 perfectly matched sequences and a set of 15 high homology sequences for functional characterization in human cells. Testing large numbers of sequences in cells presents a challenging problem as traditional assays are often complicated by splicing events that can occur during nuclear transcription and export (24). To test sequences under conditions that are not subject to nuclear processing, we developed a plasmid-based reporter assay that allows coupled transcription and translation to occur in the cytoplasm of human cells (FIG. 2A). We were inspired by an EMCV-driven system that relies on vaccinia virus (VACV) to circumvent nuclear expression (25). We inserted the sequences into a monocistronic firefly luciferase reporter plasmid (F-luc-mono) containing a VACV-specific promoter. Transfected HeLa cells were infected with VACV, and after a brief incubation, cells were lysed and assayed for luciferase activity. Plasmids carrying no-insert or a randomly chosen sequence from the starting pool provided a basal level of activity. We confirmed with no infection controls that luciferase activity was due to cytoplasmic expression by showing that uninfected cells have luciferase values equivalent to untreated cells. Plasmids carrying the selected sequences provided a range of activity (Fig. S1); the most active sequences enhanced translation ˜100-fold relative to the basal level after normalization for RNA (FIG. 2B). Analysis of the isolated RNA after six hours of expression demonstrated that the transcripts were intact and full-length. The top 9 sequences were validated in vitro in HeLa cell lysate to control for the strong capping mechanism associated with VACVs. The cell-free assay recapitulated the cell-based assay, confirming that activity did not depend on a 5′ cap or VACV infection (FIG. 2C).

To test whether the selected sequences were capable of internal translation initiation, we inserted the top 9 sequences from the monocistronic assay into a firefly reporter plasmid (F-luc-hp) containing a stable stem-loop structure (ΔG=−58 kcal/mol) to prevent ribosomal scanning (FIG. 3A) (26). Hairpin transcripts with no insert are poorly translated in vitro and in cells, yielding 0.3% and 1.5% of the respective activity observed for unobstructed transcripts after normalization for RNA (FIG. 3B). We confirmed that the RNA was stable in cells and in cell lysate, indicating that differences in activity were not due to selective degradation. We examined the top 9 sequences for IRES activity by first testing the reporter constructs in HeLa cell lysate. All nine sequences promote cap-independent translation initiation at levels consistent with known cellular IRESs (FIG. 3C) (26). We then examined the sequences in HeLa cells using cytoplasmic expression to avoid any possibility of nuclear splicing. In agreement with the in vitro assay, all of the sequences exhibit potent IRES activity (˜100-600-fold) when compared to the no insert control after normalization for RNA (FIG. 3D). To test for possible cryptic promoter activity, we repeated the cytoplasmic expression assay using a knock-out plasmid (F-luc-hp-ko) that deleted the VACV promoter. Under these conditions, HGL6.884 and HGL6.733 retain activity in VACV infected cells, indicating that a portion of their activity was due to monocistronic RNA that arose from a cryptic VACV promoter site. This prediction was confirmed by assaying HGL6.884 and HGL6.733 in uninfected cells, which yielded luciferase values equivalent to untreated cells (FIG. 3E). Direct transfection of the RNA-hairpin constructs into the cytoplasm of HeLa cells further corroborated our finding that all nine in vitro selected sequences mediate internal translation initiation (FIG. 3F).

Many well-characterized IRESs contain AUG triplets in their 5′ UTR that are expected to impede ribosomal scanning (2). Likewise, the human in vitro selected sequences identified in round 6 also have an abundance of AUG triplets. How is it then that a given AUG codon is selected as a start site when multiple options are present? One might expect a priori that AUGs in good sequence context would lead to more efficient translation initiation; however, only 1 out of 657 AUG codons observed in the 229 sequences contains a Kozak motif (Fig. S2) (27). To investigate this question, we selected ten sequences with a range of monocistronic activity and AUG triplet patterns, and examined their relative translation initiation efficiency and start site usage in vitro. This analysis revealed a number of striking observations (FIG. 4, A and B, and table S2). First, sequences with similar thermodynamic stability and no AUG codons can initiate translation with different levels of efficiency (HGL6.738 vs. HGL6.140). Second, out-of-frame start sites can be just as effective at translation initiation as in-frame start sites (HGL6.928 vs. HGL6.338). Third, AUG triplets near the 5′ end of the sequence are often bypassed in favor of downstream AUGs (e.g., HGL6.512, HGL6. 962, and HGL6.1155). Last, ribosomes that initiate translation at out-of-frame AUG codons can shift back into frame before reaching the designated ORF (e.g., HGL6.338, and HGL6.1155). Taken together, this data suggests that human cap-independent translation involves cis-regulatory elements in the 5′ UTR that function as ribosomal recruitment sites. This prediction is supported by the observation that many cellular IRESs have noncontiguous segments that retain IRES activity on their own (5, 28, 29).

To determine what role, if any, upstream AUG codons could play in ribosomal recruitment, we removed the in-frame, out-of-frame, and all AUG triplets from a high activity sequence (HGL6.877). Mutation of the AUG triplets had a strong negative impact on the translation initiation efficiency of all HGL6.877 variants (FIG. 4C). Even HGL6.877Δall devoid of all AUGs was less efficient than the parent sequence, indicating that AUGs can have a profound functional role in ribosomal recruitment. To determine whether translation initiation was due to the AUG codons or the surrounding sequence, we inserted the AUGs from HGL6.877 into an unselected sequence (HGL0.53) at the same locations that they appear in HGL6.877. Both HGL6.877 and HGL0.53 are identical in length, but HGL0.53 did not otherwise contain any AUG codons. HGL0.53 nor any of its AUG variants were capable of efficient translation initiation. This result demonstrates, at least for HG6.877, that presence of AUG triplets alone is not sufficient to initiate translation, but instead requires the ribosomal recruitment site in which the AUGs are imbedded for function.

Our results represent the first example of mRNA display as a genome-wide tool for identifying cap-independent translation initiation elements in the human genome. The in vitro selected IRESs characterized here represent novel regulatory elements that were previously hidden in the human genome. The general scheme used to identify these sequences is readily adaptable to other organisms and translation initiation mechanisms, and the versatility of the in vitro protocol makes it possible to explore ribosomal translation under a variety of conditions. We suggest that further discovery of additional cis-regulatory elements will advance our understanding of genome structure and function, and the biological role that IRESs play in the human genome.

### Materials and Methods

Library Assembly and mRNA Display Selection

The human DNA library was provided by the Szostak laboratory¹⁸. This library was modified by PCR to add the genetic information necessary for performing mRNA display³¹. For each round of selection, the dsDNA library was transcribed with T7 RNA polymerase, photo-ligated to a psoralen-DNA-puromycin linker (5′-psoralen-TAGCCGGTG-(PEG₉)₂-A₁₅-ACC-puromycin) (SEQ ID NO:6), and translated in vitro by incubating the library (1 nmol) with rabbit reticulocyte lysate and ³⁵S-methionine for 1 hour at 30° C. Fusion formation was promoted by incubating the mixture overnight at −20° C. in the presence of KCl (600 mM) and MgCl₂ (75 mM). The mRNA-peptide fusion molecules were reverse transcribed, and purified by a two-step procedure on oligo (dT)-cellulose beads (NEB) and Ni-NTA agarose affinity resin (Qiagen). Functional TEEs were recovered by eluting the column with imidazole, dialyzing the sample into water, and amplifying the cDNA by PCR. The selection progress was monitored by determining the fraction of S³⁵-labeled mRNA-peptide fusions that remained on the oligo (dT) Ni-NTA affinity columns. After 6 rounds of selection and amplification, the dsDNA library was cloned and sequenced.

### Luciferase Reporter Assay

A monocistronic luciferase reporter vector (pT7_v_<TEE>_FLuc) that contains both a T7 and a vaccinia virus synthetic late promoter was constructed by modifying a pT3-R-luc<IRES>F-luc(pA)₆₂ luciferase reporter plasmid provided by the Doudna laboratory (Gilbert et al., 2007)^{,32}. HeLa and HEK-293 cells were seeded at a density of 15,000 cells per well in white 96-well plates 18 hours prior to transfection. Cells were transfected with a complex of the reporter plasmid (200 ng) and Lipofectamine 2000 (0.5 μl) in Opti-MEM (Invitrogen), and immediately infected with the Copenhagen strain (VC-2) of WT vaccinia virus at a multiplicity of infection (m.o.i) of 5 PFU/cell (FIG. 4). Cells were lysed (6.5 hours post-infection) in the 96-well plates and the luciferase activity was measured using the Promega Luciferase Assay System with a Glomax microplate luminometer (Promega). Cell-free characterization of the top TEEs was performed using the Human In Vitro Protein Expression Kit (Pierce). Luciferase expression was achieved following manufacturer's protocols using 300 ng of linear template for a two-hour transcription at 32° C. followed by a 90 min translation at 30° C.

### RNA Characterization

A portion of the cells used in the transfect-infect study was separately lysed to evaluate the quality of the cellular RNA. Isolated RNA was reverse transcribed with Superscript II (Invitrogen), and realtime PCR was used to determine the mRNA levels of luciferase relative to the housekeeping gene hypoxanthine-guanine phospho-ribosyltransferase (HPRT). Using the ΔΔCt method, the amount of luciferase mRNA was normalized to HPRT mRNA levels. In addition, the length of luciferase mRNA was determined using PCR to analyze the relative proportion of the 5′- and 3′-ends of representative cDNA molecules.

### Mutagenesis Study.

The 13-nucleotide core motif was assayed for activity by constructing five luciferase reporter constructs in which the 13-mer motif was either added to the 5′ end of a low activity TEE (clones 499, 646 and 347) or deleted from the 5′ end of a high activity TEE (clones 1092 and 1347). HGL sequences 1092 and 1347 were regenerated with the 13-nucleotide deletion by Klenow DNA polymerase extension followed by a restriction enzyme digest with BamHI and NcoI. The digested fragments were then ligated into the luciferase reporter plasmid pT7_v_<TEE>_FLuc. The insertion constructs were generated by overlap PCR, and then digested and ligated into the reporter plasmid. Translation enhancement of the modified sequences was assessed using the transfect/infect assay in HeLa cells. Sequences 1347 and 499 were additionally characterized in BSC40, RK13, BHK and 129SV cells.

### Bioinformatics Analysis

Bioinformatics analysis was used to analyze 143 sequences from the naïve library, and 709 sequences isolated after six rounds of in vitro selection. The genomic locations of all non-redundant sequences were determined using the BLAT webtool to map each sequence to the human reference genome (hg18)²¹. This analysis revealed that 75 sequences from the naïve pool and 227 sequences from the round 6 pool matched with perfect sequence identity to the human reference genome. The program RepeatMasker was used to classify the selected sequences into specific repeat families³³. By randomly selecting 10,000 genomic locations, we generated the null expectation for the fraction of sequence motifs of length 200 nucleotides to overlap a repeat family. This number was 45.7% and was not statistically-significantly different from that observed for Round-0 sequences. However, the null hypothesis for TEEs is rejected at P<10-6 indicating that TEEs are significantly enriched in their involvement with repeat families.

TABLE 1
Clones sequenced for characterization after six rounds of mRNA display selection.

| Entry | Clone | Duplicates | Sequence |
|---|---|---|---|
| 1 | HGL6.1 | HGL6.346, | AATCGAATGGAATCAACATCAAACGGAAAAAAACGGAATTATCGAATGGAATCG |
| | | HGL6.670, | AAGAGAATCATCGAATGGACC (SEQ ID NO: 7) |
| | | HGL6.676, | |
| | | HGL6.715, | |
| | | HGL6.961, | |
| | | HGL6.1106, | |
| | | HGL6.1182, | |
| | | HGL6.1338 | |
| 2 | HGL6.5 | | TGGAATCGAATGGAATCAACATCAAACGGAAAAAAACGGAATTATCGAATGGA |
| | | | ATCGAAGAGAATCATCGAATGGACC (SEQ ID NO: 8) |
| 3 | HGL6.7 | | AGCATTCATATCTTGCAGTGTTGGGAAAGAGTGAGAGGTTGTGATGTCAAGAAG |
| | | | GATAGGTCAGAAGTGGAAGGTATGGGGGATTGTGCCTGCTGTCATGGCT (SEQ ID |
| | | | NO: 9) |
| 4 | HGL6.8 | | GGAACGAAATCGAATGGAACGGAATAGAATAGACTCGAATGTAATGGATTGCTA |
| | | | TGTAATTGATTCGAATGGAATGGAATCGAATGGAATGCAATCCAATGGAATGGA |
| | | | ATGCAATGCAATGAATGGAATGGAATGGAATGGAATGGAA (SEQ ID NO: 10) |
| 5 | HGL6.9 | | GGAACGAAATCGAATGGAACGGAATAGAATAGACTCGAATGTAATGGATTGCTA |
| | | | TGTAATTGATTCGAATGGAATGGAATCGAATGGAATGCAATCCAATGGAATGGA |
| | | | ATGCAATGCAATGAATGGAATGGAATGGAATGGAATGGA (SEQ ID NO: 11) |
| 6 | HGL6.12 | | TACGCAAATCGATAAATGTAATCCAGCATATAAACAGAACCAAAGACAAAAACC |
| | | | ACATGATTATCTCAATAGATGCAGAAAAGGCC (SEQ ID NO: 12) |
| 7 | HGL6.14 | | ACTCGAATGCAATCAACATCAAACGGAATCAAACGGAATTATCGAATGGAATCG |
| | | | AAGAGAATCATCGAACGGACTCGAATGGAATCATCTAATGGAATGGAATGG |
| | | | (SEQ ID NO: 13) |
| 8 | HGL6.18 | | GAAATTCCAATTAAAATGAAATCGACTTATCTTAACAAATATAGCAATGCTGACA |
| | | | ACACTTCTCCGGATATGGGTACTGCT (SEQ ID NO: 14) |
| 9 | HGL6.20 | | AAGGAAAAGTAAAAGGAACTTAACACCTTCAAGAAAAGACAGACAAATAACAA |
| | | | AACAGCAGTTTGATAGAATGAGATATCAGGGGATGGCA (SEQ ID NO: 15) |
| 10 | HGL6.21 | | ATCAACATCAAACGGAAAAAACGGAATTATCGAATGGAATCGAAGAGAATCATC |
| | | | GAACGGACC (SEQ ID NO: 16) |
| 11 | HGL6.22 | | AAAGAAAGACAGAGAACAAACGTAATTCAAGATGACTGATTACATATCCAAGAA |
| | | | CATTAGATGGTCAAAGACTTTAAGAAGGAATACATTCAAAGGCAAAAAGTCACT |
| | | | TACTGATTTTGGTGGAGTTTGCCACATGGAC (SEQ ID NO: 17) |
| 12 | HGL6.23 | | AAGGGAATTGAATAGAATGAATCCGAATGGAATGGAATGGAATGGAATGGAAT |
| | | | GGAATGGAATGGAATGGAATGGAATG (SEQ ID NO: 18) |
| 13 | HGL6.24 | | GAATGGAATCGAATCAAATTAAATCAAATGGAATGCAATAGAAGGGAATACAAT |
| | | | GGAATAGAATGGAATGGAATGGAATGGACT (SEQ ID NO: 19) |
| 14 | HGL6.25 | | ACAGCAAGAGAGAAATAAAACGACAAGAAAACTACAAAATGCCTATCAATAGT |
| | | | TACTTTAAATATCAGTGGACCAAATCAGTGAAACAAAAGACACAGAGTGGC |
| | | | (SEQ ID NO: 20) |
| 15 | HGL6.27 | | TAGCAGGAAACAGCAAACTCAAATTAAGTAATTTCAAGAGCGTATCATCAATGA |
| | | | ACTATTTTCAAAGATGTGGGCAAGAT (SEQ ID NO: 21) |
| 16 | HGL6.28 | | AAACGGAATTATCAAATGGAATCGAAGAGAATCATCGAACGGACTCGAATGGAA |
| | | | TCATCTAATGGAATGGAATGGAAG (SEQ ID NO: 22) |
| 17 | HGL6.30 | | GAATGAAATGAAATCAAATNGAATGTACATGAATGGAATAGAAAAGAATGCATC |
| | | | TTTCTCGAACGGAAGTGCATTGAATGGAAAGGAATCTACTGGAATGGATTCGAA |
| | | | TGGAATGGAANGGGATGGAATGGTATGG (SEQ ID NO: 23) |
| 18 | HGL6.32 | | AATGGACTCGAATGAAATCATCATCAAACGGAATCGAATGGAATCATTGAATGG |
| | | | AAAGGATGGGATCATCATGGAATGGAAACGAATGGAATCACTG (SEQ ID NO: 24) |
| 19 | HGL6.34 | | AATGGAATCATTGAATGGAATGGAATGGAATCATCAAAGAAAGGAATCGAAGG |
| | | | GAATCATCGAATGGAATCAAACGGAATCATCGAATGGAATGGAATGGAATG |
| | | | (SEQ ID NO: 25) |
| 20 | HGL6.38 | HGL6.537 | AGCAGAAGAAATAACTGAAATCAGAGTGAAACTGAATCAAATTGAGATGCAAA |
| | | | AATACATACGAAATGGCCAG (SEQ ID NO: 26) |
| 21 | HGL6.40 | | AGTTAATCCGAATAGAATGGAATGGAATGCAATGGAACGGAATGGAACGGAAT |
| | | | GGAATGGAATGGAATGGAATGGAATG (SEQ ID NO: 27) |
| 22 | HGL6.42 | | ATGGAATCAACATCAAACGGAATCAAACGGAATTATCGAATGGAATCGAAGAGA |
| | | | ATCATCGAACGGATTCGAATGGAATCATCTAATGGAATGGAATGGAAGAATCCA |
| | | | TGGACTCGAATGCAATCATCAGCGAATGGAATCGAATGGAATCATCGAATGGAC |
| | | | TCG (SEQ ID NO: 28) |
| 23 | HGL6.44 | | AAAGGAATGGACTGGAACAAAATGAAATCGAACGGTAGGAATCGTACAGAACG |
| | | | GACAGAAATGGAACGGCATGGAATGCACTCG (SEQ ID NO: 29) |
| 24 | HGL6.47 | | AAATCAACAACAAACGGAAAAAAAAGGAATTATCGAATGGAATCAAAGAGAAT |
| | | | CATCGAATGGACC (SEQ ID NO: 30) |
| 25 | HGL6.50 | | AAATGAACAAAACTAGAGGAATGACATTACCTGACTTCAAATTATACTACAGAG |
| | | | CTATAGTAACCAAAACAGCATGGTACAGGCAT (SEQ ID NO: 31) |
| 26 | HGL6.51 | | GTAATGGAATGGAATGGAAAGGAATCGAAACGAAAGGAATGGAGACAGATGGA |
| | | | ATGGAATGGAACAGAG (SEQ ID NO: 32) |
| 27 | HGL6.52 | HGL6.496, | ATCGAATGGAATCAACATCAAACGGAAAAAAACGGAATTATCGAATGGAATCGA |
| | | HGL6.881, | AGAGAATCATCGAATGGACC (SEQ ID NO: 33) |
| | | HGL6.1207 | |
| 28 | HGL6.57 | | CAATCAGAGCGGACACAAACAAATTGCATGGGAAGAATCAATATCGTGAAAATG |
| | | | GCC (SEQ ID NO: 34) |
| 29 | HGL6.59 | | AGACCTTTCTCAGAAGACACACAAATTGCCAACAGGTATATGAAAAAATGTTCA |
| | | | ATATCACTAATCATCAGGGCGATGCC (SEQ ID NO: 35) |
| 30 | HGL6.61 | | CATGGAATCGAATGGAATTATCATCGAATGGAATCGAATGGTACCAACACCAAA |
| | | | CGGAAAAAAACGGAATTATCGAATGGAATCGAAGAGAATCTTCGAACGGACC |
| | | | (SEQ ID NO: 36) |
| 31 | HGL6.63 | | GAACGATTTATCACTGAAAATTAATACTCATGCAAGTAGTAAACGAATGTAATG |
| | | | ACCATGATAAGGAGACGGACGGTGGTGATAGT (SEQ ID NO: 37) |
| 32 | HGL6.65 | | AAAGATCAANGNNCAAAAATCAGCAGCATTTCTATAAACCAACAATGTCCAGGC |
| | | | TGAGAGNGAAATCAAGAAANCAATTC (SEQ ID NO: 38) |
| 33 | HGL6.66 | | ACACACATACCAACAGAACATGACAAAAGAACAAAACCAGCCGCATGCATACTC |
| | | | GATGGAGACAAAGGTAACACTGCAGAATGGTGAAGGAAGAACAGTCATTTTAAT |
| | | | GACAGTGTTGGCT (SEQ ID NO: 39) |
| 34 | HGL6.67 | HGL6.463, | AATGGAATCAACATCAAACGGAAAAAAACGGAATTATCGAATGGAATCGAAGA |
| | | HGL6.775, | GAATCATCGAATGGACC (SEQ ID NO: 40) |
| | | HGL6.936 | |
| 35 | HGL6.68 | | ATCAAAAGGAACGGAATGGAATGGAATGGAATGGAATGGAATGGAATGGAATG |
| | | | GAATGAAATCAACCCGAATGGAATGGATTGGCATAGAGTGGAATGG (SEQ ID |
| | | | NO: 41) |
| 36 | HGL6.70 | HGL6.71 | TAAAGAAAAACAAACAAACAGAAATCAATGAAAATCCCATTCAAAGGTCAGCA |
| | | | ACCTCAAAGACTGAAGGTAGATAAGCCCACAAGGATG (SEQ ID NO: 42) |
| 37 | HGL6.73 | | AAACGGAAAAAAACGGAATTATCGAATGGAATCGAATAGAATCATCGAATGGA |
| | | | CC (SEQ ID NO: 43) |
| 38 | HGL6.74 | | GGAATCAACTCGATTGCAATGGAATGCAATGGAAAGGAATGGAATGCAATTAAA |
| | | | GCGAATAGAATGGAATGGAATGGAATGGAACGGAATGGAATG (SEQ ID NO: 44) |
| 39 | HGL6.76 | | GAAGAAGAAAAAACATGGATATACAATGTCAACAGAAATCAAGGAGAAACGGA |
| | | | ATTTCACCAATCAATTTAGTGATCTGGGTT (SEQ ID NO: 45) |
| 40 | HGL6.82 | | TGGAATCATCTAATGGAATGGAATGGAATAATCCATGGACTCGAATGCAATCAT |
| | | | CATAAAATGGAATCGAATGGAATCAACATCAAATGGAATCAAATGGGATCATTG |
| | | | AACGGAATTGAATGGAATCGTCAT (SEQ ID NO: 46) |
| 41 | HGL6.83 | | TGAACAGAGAATTGGACAAAACGCACAAAGTAAAGAAAAAGAATGAAGCAACA |
| | | | AAAGCAGAGATTTATTGAAAACAAAAGTACACACCACACAGGGTGGGAGTGG |
| | | | (SEQ ID NO: 47) |
| 42 | HGL6.85 | HGL6.980, | GGAATCAACATCAAACGGAAAAAAACGGAATTATCGAATGGAATCGAAGAGAA |
| | | HGL6.1002 | TCATCGAATGGACC (SEQ ID NO: 48) |
| 43 | HGL6.88 | | AACACGACTTTGAGAAGAGTAAGTGATTGTTAATTAAAGCAAGAGAATTATTGA |
| | | | TGTATCACAGTCATGAGAAATATTGGAAGGAATATGGTCCATAC (SEQ ID NO: 49) |
| 44 | HGL6.91 | | TGAAAAGAAGAATGACCATAAGCAAGCAGATGAAAAACAAAACAGAATTTTTA |
| | | | CAGACGTCTTGGACTGATATCTTGGGC (SEQ ID NO: 50) |
| 45 | HGL6.92 | | AATCAATAAATGTAAACCAGCATATAAACAGAACCAACGACAAAAACCACATGA |
| | | | TTATCTCAATAGATGCAGAAAAGGCC (SEQ ID NO: 51) |
| 46 | HGL6.95 | | CAACATCAAACGGAATCAAACGGAATTATCGAATGGAATCGAAGAGAATCATCG |
| | | | AATGGACTCGAATGGAATCATCTAATGGAATGGAATGGAAG (SEQ ID NO: 52) |
| 47 | HGL6.96 | | AATGGAAGGGAATGGAATGGAATCGAATCGAATGGAACAGAATTCAATGGAAT |
| | | | GGAATGGAATGGAATGGAATCGAATGGAATGG (SEQ ID NO: 53) |
| 48 | HGL6.100 | | AAAGACTTAAACATAAGACCTAAAACCATAAAAACCACAGAAGAAAACATAGG |
| | | | CAATGCCATTCAGGACATAGGCATGGGCAAAGACTTC (SEQ ID NO: 54) |
| 49 | HGL6.101 | | AGACTTGAAAAGCACAGACAACGAAAGCAAAAATGGACAAATGGAATCACATC |
| | | | AAGCTAAAAGGTTTTGCATGGCAAAGG (SEQ ID NO: 55) |
| 50 | HGL6.1l2 | HGL6.952, | AGCAACTTCAGCAAAGTCTCAGGATACAAAATCAATGTGCAAAAATCACAAGCA |
| | | HGL6.955 | TTCTTATACACCAACAACAGACAAACAGAGAGCC (SEQ ID NO: 56) |
| 51 | HGL6.113 | | TGAATGCTATAGAGCAGTAAAAACAAATAAATGAACTACATTACAGCTACTTAC |
| | | | AACCATATGAAAGAATATAACCATAACAATGATGAGTGGACAAAAGCTAAGTGT |
| | | | GAAAGAATGCATAGTGCTACAGCAGCCAACATTTACAGC (SEQ ID NO: 57) |
| 52 | HGL6.115 | | AACAAAATTGAACAACATGCAAAGAAACATAAACGAAGCAATGAAAGTGTGCA |
| | | | GATCCACTGAAATGAAAGTGCTGTCCAGAGTGGGAGCCAGCTCGAGA (SEQ ID |
| | | | NO: 58) |
| 53 | HGL6.116 | | TGGAATTATCGTCGAATAGAATCGAATGGTATCAACATCAAACGGAAAAAAACG |
| | | | GAATTATCGAATGGAATCGAAGAGAATCATCGAACGGACTCGAATGGAATCATC |
| | | | TAATGGAATGGAATGGAATAATCCATGG (SEQ ID NO: 59) |
| 54 | HGL6.117 | | AGATAAGTGGATGAACAGATGGACAGATGGATGGATGGATGGATGGATGGATG |
| | | | GATGCCTGGAAGAAAGAAGAATGGATAGTAAGCTGGGTATA (SEQ ID NO: 60) |
| 55 | HGL6.119 | | AATCAAAGAATTGAATCGAATGGAATCATCTAATGTACTCGAATGGAATCACCA |
| | | | T (SEQ ID NO: 61) |
| 56 | HGL6.121 | | AATGGAATCGAACGGAATCATCATCAAACGGAACCGAATGGAATCATTGAATGG |
| | | | AATCAAAGGCAATCATGGTCGAATG (SEQ ID NO: 62) |
| 57 | HGL6.122 | | AGGAATCTATAATACAGCTGTTTATAGCCAAGCACTAAATCATATGATACAGAA |
| | | | AACAAATGCAGATGGTTTGAAGGGTGGG (SEQ ID NO: 63) |
| 58 | HGL6.125 | | AACGGAAAAAAACGGAATTATCGAATGGAATCGAAGAGAATCATCGAATGGAC |
| | | | C (SEQ ID NO: 64) |
| 59 | HGL6.126 | | TGAGAAAATGATGGAAAAGAGGAATAANACGAAACAAAACCACAGGAACACAG |
| | | | GTGCATGTGAATGTGCACAGACAAAGATACAGGGCGGACTGGGAAGGAAGTTTC |
| | | | TGCACCAGAATTTGGGG (SEQ ID NO: 65) |
| 60 | HGL6.132 | | AATGGAATCGAAGAGAATGGAAACAAATGGAATGGAATTGAATGGAATGGAAT |
| | | | TGAATGGAATGGGAAGGAATGGAGTG (SEQ ID NO: 66) |
| 61 | HGL6.134 | | AATGTCAAGTGGAATCGAGTGGAATCATCGAAAGAAATCGAATGGAATCGAAGG |
| | | | GAATCATTGGATGGGCTCAAAT (SEQ ID NO: 67) |
| 62 | HGL6.137 | | AAACAATGGAAGATAATGGAAAGATATCGAATGGAATAGAATGGAATGGAATG |
| | | | GACTCAAATGGAATGGACTTTAATGGAATGG (SEQ ID NO: 68) |
| 63 | HGL6.138 | | GAACAATCAATGGAAGCAGAAACAAATAAACCAAGGTGTGCATCAAGGAATAC |
| | | | ATTCACGCATGATGGCTGTATGAGTAAAATG (SEQ ID NO: 69) |
| 64 | HGL6.139 | | AAACCGAATGGAATGGAATGGACGCAAAATGAATGGAATGGAAGTCAATGGAC |
| | | | TCGAAATGAATGGAATGGAATGGAATGGAATG (SEQ ID NO: 70) |
| 65 | HGL6.140 | | AGGATACAAAATCAAAGTGCAAAAATCACAAGCATTCTTATACACCAATAACAG |
| | | | ACAAACAGAGAGCC (SEQ ID NO: 71) |
| 66 | HGL6.147 | | GGAATCGAATGGAATCAACATCAAACGGAAAAAAACAGAATTATCGTATGGAAT |
| | | | CGAATAGAATCATCGAATGGACC (SEQ ID NO: 72) |
| 67 | HGL6.148 | | CAACCCGAGTGGAATAAAATGGAATGGAATGGAATGAAATGGAATGGATCGGA |
| | | | ATGGAATCCAATGGAATCAACTGGAATGGAATGGAATGGAATG (SEQ ID NO: 73) |
| 68 | HGL6.149 | | TATCATCGAATGGAATCGAATGGAATCAACATCAAACGGAAAAAAACGGAATTA |
| | | | TCGAATGGAATCGAAGAGAATCATCGAATGGACC (SEQ ID NO: 74) |
| 69 | HGL6.150 | | CGGAATAATCATTGAACGGAATCGAATGGAATCATCATCGGATGGAAACGAATG |
| | | | GAATCATCATCGAATGGAAATGAAAGGAGTCATC (SEQ ID NO: 75) |
| 70 | HGL6.151 | | CAACACACAGAGATTAAAACAAACAAACAAACAATCCAGCCCTGACATTTATGA |
| | | | GTTTACAGACTGGTGGAGAGGCAGAGAAG (SEQ ID NO: 76) |
| 71 | HGL6.152 | | GGAATGGAATGAACACGAATGTAATGCAACCCAATAGAATGGAATCGAATGGCA |
| | | | TGGAATATAAAGAAATGGAATCGAAGAGAATGGAAACAAATGGAATGGAATTG |
| | | | (SEQ ID NO: 77) |
| 72 | HGL6.153 | | CACTACAAACCACGCTCAAGGCAATAAAAGAACACAAACAAATGGAAAAACAT |
| | | | TCCATGCTCATGGATGGG (SEQ ID NO: 78) |
| 73 | HGL6.158 | | AATCGAATGGAATTAACATCAAACGGAAAAAAACGGAATTATCGAATGGAATCG |
| | | | AAGAGAATCATCGAATGGACC (SEQ ID NO: 79) |
| 74 | HGL6.161 | | TGGAAAAGAATCAAATTGAATGGCATCGAACGGAATGGGATGGAATGGAATAG |
| | | | ACCCAGATGTAATGGACTCGAATGGAATG (SEQ ID NO: 80) |
| 75 | HGL6.163 | | AATCAGTCTAGATCTTAAAGGAACACCAGAGGGAGTATTTAAATGTGCCCAATA |
| | | | AGCAAGAATTATGGTGATGTGGAAGTA (SEQ ID NO: 81) |
| 76 | HGL6.164 | | CCATAACACAATTAAAAACAACCTAAATGTCTAATAGAAGAACACTGTTCAGAC |
| | | | CGGGCATGGTGGCTTATACC (SEQ ID NO: 82) |
| 77 | HGL6.165 | | GACTAATATTCAGAATATACAAGGAACTCAAACAACTCAACAGTAGAAAAAAAA |
| | | | ACCTGAATAGACATTTCTCAAAAGAAGACATACAAATGGCC (SEQ ID NO :33) |
| 78 | HGL6.171 | HGL6.1149 | AACAGACCATAAATAAACACAGAAGACACACGAGTGTAAAGTCAGTGCCCCGCT |
| | | | GCGAATTAAATCGGGGTGATGTGATGGCGAGTGAGTGGGTAGTT (SEQ ID NO: |
| | | | 84) |
| 79 | HGL6.174 | | ATCATTGAATGCAATCACATGGAATCATCACAGAATGGAATCGTACGGAATCAT |
| | | | CATCGAATGGAATTGAATGGAATCATCAATTGGACTCGAATGGAAACATCAAAT |
| | | | GGAATCGATTGGAAGTGTCGAATGGACTCG (SEQ ID NO: 85) |
| 80 | HGL6.175 | | GGTCCATTCGATGATTCTCTTCGATTCCATTCGATAATTCCGTTTTTTCCCGTTTG |
| | | | ATGTTGATTCC (SEQ ID NO: 86) |
| 81 | HGL6.178 | | AGCAACTTCAGTAAAGTGTCAGGATACAAAATCAATGTGCAAAAATCACAAGCA |
| | | | TTCTTATACATCAATAACAGACAAACAGAGAGCCAAA (SEQ ID NO: 87) |
| 82 | HGL6.180 | | AGCAACTTCAGCAAAGTCTCAGGATACAAAATCAATGTGCAAAAATCACAAGCA |
| | | | TTCCTATACACCAACAACAGACAAACAGAGAGCC (SEQ ID NO: 88) |
| 83 | HGL6.181 | | GAATAATCATTGAACGGAATCGAATGGAATCATCATCGGATGGAAACGAATGGA |
| | | | ATCATCATCGAATGGAAATGAAAGGAGTCATC (SEQ ID NO: 89) |
| 84 | HGL6.182 | HGL6.902 | TAATCAT CT TCGAATTGAAAACAAAGCAATCATTAAATGTACTCTAACGGAATCA |
| | | | TCGAATGGACC (SEQ ID NO: 90) |
| 85 | HGL6.184 | HGL6.1215 | GGAATCGAATGGAATCAACATCAAACGGAAAAAAACGGAATTATCGAATGGAA |
| | | | TCGAAGAGAATCATCGAATGGACC (SEQ ID NO: 91) |
| 86 | HGL6.186 | | GATCAGCTTAGAATACAATGGAACAGAACAGATTAGAACAATGTGATTTTATTA |
| | | | GGGGCCACAGCACTGTTGACTCAAGTACAAGTTCTGACTCATGTAGAACTAACA |
| | | | CTTTT (SEQ ID NO: 92) |
| 87 | HGL6.187 | | AGAGAAAAGATGATCATGTAACCATTGAAAAGACAATGTACAAAACTAATACTA |
| | | | ATCACACAGGACCAGAAAGCAATTTAGACCAT (SEQ ID NO: 93) |
| 88 | HGL6.190 | | AATGGAATCGAATGGAATCAACATCAAACGGAAAAAACGGAATTATCGAATGG |
| | | | AATCAAAGAGAATCATCGAATGGACC (SEQ ID NO: 94) |
| 89 | HGL6.191 | | AATGGAATTATCATCGAATGGAATCGAATGGAATCAACATCAAACGGAAAAAAA |
| | | | CGGAATTATCGAATGGAATCGAAGAGAATCATCGAATGGACC (SEQ ID NO: 95) |
| 90 | HGL6.197 | | GTCAACACAGGACCAACATAGGACCAACACAGGGTCAACACAGGACCAACATA |
| | | | GGACCAACACAGGGTCAACACAAGACCAACATGGGACCAACACAGGGTCAACA |
| | | | TAGGACCAACATGGGACCAACACAGGGTCAACACAGGACCAAC (SEQ ID NO: 96) |
| 91 | HGL6.198 | | TATAGTTGAATGAACACACATACACACACACATGCCACAAAACAAAAACAAAGT |
| | | | TATCCTCACACACAGGATAGAAACCAAACCAAATCCCAACACATGGCAAGATGA |
| | | | T (SEQ ID NO: 97) |
| 92 | HGL6.206 | | GAATCAACTCGATTGCAATCGAATGGAATGGAATGGTATTAACAGAATAGAATG |
| | | | GAATGGAATGGAATGGAACGGAACG (SEQ ID NO: 98) |
| 93 | HGL6.208 | | AATGGAATGGAATAATCGACGGACCCGAATGCAATCATCATCGTACAGAATCGA |
| | | | ATGGAATCATCGAATGGACTGGAATGGAATGG (SEQ ID NO: 99) |
| 94 | HGL6.210 | | AATACAAACCACTGCTCAACGAAATAAAAGAGGATACAAACAAATGGAAGAAC |
| | | | ATTCTATGCTCATGGGTAGGATGAATTCATATCGTGAAAATGGCCATACTGCC |
| | | | (SEQ ID NO: 100) |
| 95 | HGL6.215 | | AAACACGCAAACACACACACAAGCACACTACCACACAAGCGGACACACATGCA |
| | | | AACACGCGAACACACACACATATACACACAAGCACATTACAAAACACAAGCAA |
| | | | ACACCAGCAGACACACAAACACACAAACATACATGG (SEQ ID NO: 101) |
| 96 | HGL6.219 | | AATCGAACGGAATCAACATCAAACGGAAAAAAAACGGAATTATCGAATGGAAT |
| | | | CGAAGAGAATCATCGAATGGACC (SEQ ID NO: 102) |
| 97 | HGL6.220 | HGL6.301, | ACACATTTCAAGGAAGGAAACAAGAACAGACAGAAACACAACATACTTCATGA |
| | | HGL6.1353 | AACCACATTTTAGCATCCTGGCCGAGTATTCATCA (SEQ ID NO: 103) |
| 98 | HGL6.222 | | GGATACAAAATCAATGTACAAAAATCACAAGCATTCTTATACACCAATAACAGA |
| | | | CAAACAGAGAGCC (SEQ ID NO: 104) |
| 99 | HGL6.223 | | TAATTGATTCGAATGGAATGGAATAGAATGGAATTGAATGGAATGGACCATAAT |
| | | | GGATTGGACTTTAATAGAAAGGGCATG (SEQ ID NO: 105) |
| 100 | HGL6.225 | | AGCAACTTCAGCAAAGTCTCAGGATACAAAATCAATGTACAAAAGTCACAAGCA |
| | | | TTCTTATACACCAACAAAAGACAAACAGAGAGCC (SEQ ID NO: 106) |
| 101 | HGL6.228 | | ACATCAAACGGAAAAAAAAAACAAAACGGAATTATCGAATGGAATCGAAGAGA |
| | | | ATCATCGAATGGACC (SEQ ID NO: 107) |
| 102 | HGL6.229 | | ACATCTCACTTTTAGTAATGAACAGATCATTCAGACAGAAAATTAGCAAAGAAA |
| | | | CATCAGAGTTAAACTACACTCTAAACCAAATGGACCTA (SEQ ID NO: 108) |
| 103 | HGL6.231 | | GAAGAAAGCATTCATTCAAGACATCTAACTCGTTGATATAATGCATACAGTTCAA |
| | | | AATGATTACACTATCATTACATCTAGGGCTTTC (SEQ ID NO: 109) |
| 104 | HGL6.232 | | GCAAAAGAAACAATCAGTAGAGTAAACAGACAACTCATAGAATGCAAGAAAAT |
| | | | CATCGCAATCTGTACATCCAACAAAGGGCT (SEQ ID NO: 110) |
| 105 | HGL6.235 | | ACACACACATTCAAAGCAGCAATATTTACAACAGCCAAAAGGTGGAAACAATTG |
| | | | AGCAATTG (SEQ ID NO: 111) |
| 106 | HGL6.237 | | ATCATCGAATAGAATCGAATGGTATCAACACCAAACGGAAAAAAACGGAATTAT |
| | | | CGAATGGAATCGAAGAGAATCTTCGAACGGACC (SEQ ID NO: 112) |
| 107 | HGL6.238 | | TGAAAATACAAATGACCATGCAAGTAATTCCGCAGGGAGAGAGCGGATATGAAC |
| | | | AAACAGAAGAAATCAGATGGGATAGTGCTGGCGGGAAGTCA (SEQ ID NO: 113) |
| 108 | HGL6.239 | | AATCGAAAGGAATGTCATCGAATGGAATGGACTCAAATGGAATAGAATCGGATG |
| | | | GAATGGCATCGAATGGAATGGAATGGAATTGGATGGAC (SEQ ID NO: 114) |
| 109 | HGL6.241 | | AACATGAACAGTGGAACAATCAGTGAACCAATACAAGGGTTAAATAAGCTAGCA |
| | | | ATTAAAAGCTGTATCACTGGTCTAAAGATAGAAGATCAAGTAGAAAATCAGCGC |
| | | | AAGAGGAAAGATATACGAAAACTAATGGCC (SEQ ID NO: 115) |
| 110 | HGL6.243 | | CGAATGGAATCATTATGGAATGGAATGAAATGGAATAATCAAATGGAATTGAAT |
| | | | GGAATCATCGAATGGAATCGAACAAAATCCTCTTTGAATGGAATAAGATGGAAT |
| | | | CACCAAATGGAATTG (SEQ ID NO: 116) |
| 111 | HGL6.246 | | AAACGGAATCAAACGGAATTATCGAATGGAATCGAAGAGAATCATCGAACGGA |
| | | | CTCGAATGGAATCATCTAATGGAATGGAATGGAAGAATCCATGGACT (SEQ ID |
| | | | NO: 117) |
| 112 | HGL6.247 | | GCTAGTTCAACATATGCAAATCAATAAACGTAATCCATCACATAAACAGAACCA |
| | | | ATGACAAAAACCACGATTATCTCAATAGATGCAGAAAAGGCC (SEQ ID NO: 118) |
| 113 | HGL6.256 | | ACCAATCAAGAAAACAATGCAACCCACAGAGAATGGACAAAAGCAAGGCAGGA |
| | | | CAATGGCT (SEQ ID NO: 119) |
| 114 | HGL6.26 | | ATCGAATGGAATCAACATCAGACGGAAAAAAACGGAATTATCAAATGGAATCGA |
| | | | AGAGAATCATCGAATGGACC (SEQ ID NO: 120) |
| 115 | HGL6.260 | | ATGGAATCAACATCAAACGGAAAAAAAAACGGAATTATCGAATGGAATCGAAG |
| | | | AGAATCATCGAATGGACCAGAATGGAATCATCTAATGGAATGGAATGG (SEQ ID |
| | | | NO: 121) |
| 116 | HGL6.261 | HGL6.1088 | AATGGAATCATCATCGAATGGAATCGAATGGAATCATGGAATGGAATCAAATGG |
| | | | AATCAAATGGAATCGAATGGAATGGAATGGAATG (SEQ ID NO: 122) |
| 117 | HGL6.262 | | AACGGAATCAAACGGAATTACCGAATGGAATCGAATAGAATCATCGAACGGACT |
| | | | CGAATGGAATCATCTAATGGAATGGAATGGAAG (SEQ ID NO: 123) |
| 118 | HGL6.263 | | AAACGGAATCAAACGGAATTATCGAATGGAATCGAAAAGAATCATCGAACGGA |
| | | | CTCGAATGGAATCATCTAATGGAATGGAATGGAAGAATCCATGG (SEQ ID NO: |
| | | | 124) |
| 119 | HGL6.266 | | AGCAACTTCAGCAAAGTCTCAGGATACAAAATCAATGTACAAAAATCACAAGCA |
| | | | TTCTTATACACCAATAACAGACAAACAGAGAGCC (SEQ ID NO: 125) |
| 120 | HGL6.267 | | GAATGATACGGANTANNNNGNAATGGAACGAAATGAAATGGAATGGAATGGAA |
| | | | TGGAATGGAATGGAATGG (SEQ ID NO: 126) |
| 121 | HGL6.268 | | AATGGACTCGAATGGATTAATCATTGAACGGAATCGAATGGAATCATCATCGGA |
| | | | TGGTAATGAATGGAATCATCATCGAATGGAATCGG (SEQ ID NO: 127) |
| 122 | HGL6.271 | | GAATGGAATCGAAAGGAATGTCATCGAATGGAATGGAATGGAACGGAATGGAA |
| | | | TCGAATGGAATGGACTCGAATGGAATAGAATCGAATGCAATGGCATCG (SEQ ID |
| | | | NO: 128) |
| 123 | HGL6.274 | HGL6.466, | GAATAGAATAGAATGGAATCATCGAATGGAATCGAATGGAATCATCATGATATG |
| | | HGL6.883 | GAATTGAGTGGAATC (SEQ ID NO: 129) |
| 124 | HGL6.276 | | TAAGCCGATAAGCAACTTCAGCAAAGTCTCAGGAGACAAAATCAATGTGCAAAA |
| | | | AATCACAAGCATTCTTATACACTAATAACAGACAAACAGAGAGCCAAATCATG |
| | | | (SEQ ID NO: 130) |
| 125 | HGL6.277 | | AGCAACTTCAGCAAAGTCTCAGGATACAAAATCAATGTGCAAAAATCAAAAGCA |
| | | | TTCTTATGCACCAATAACAGACACAGAGCCAAAT (SEQ ID NO: 131) |
| 126 | HGL6.278 | | AGGAAAGTTTTCAATATGAGAAAGATACAAACCAACAGAATAAGCAAACTGGAT |
| | | | AAACAGAAAATACAGAGAGAGCCAAGG (SEQ ID NO: 132) |
| 127 | HGL6.280 | | AATGGAATGGAACGCAATTGAATGGAATGGAATGGAACGGAATCAACCTGAGTC |
| | | | AAATGGAATGGAATGGAATGGAATG (SEQ ID NO: 133) |
| 128 | HGL6.289 | | AGGAAAATGCAAATCAGAACGACTATAACACACCATCTCAAACTCGTTAGGATG |
| | | | GCTATTATCAAAAAGTCAAGAGATAACAAATGTGGGCAAGGG (SEQ ID NO: 134) |
| 129 | HGL6.290 | | GGAACGAAATCGAATGGAACGGAATAGAATAGACTCGAATGTCATGGATTGCTA |
| | | | TGTAATTGATTGGAATGGAATGGAATCG (SEQ ID NO: 135) |
| 130 | HGL6.291 | | GAATTGAAAGGAATGTATTGGAATAAAATGGAATCGAATAGGTTGAAATACCAT |
| | | | AGGTTCGAATTGAATGGAATGGGAGGGACACCAATGGAATTG (SEQ ID NO: 136) |
| 131 | HGL6.292 | | AACAAAACAAAAACCCAACTCAATAACAAGAAGACAAACAACCCAATTTAAAA |
| | | | TGAGCAAAGAACTTGATAAACATGTCTCCAAAGAAGATACGGCCAAAGAGCAC |
| | | | (SEQ ID NO: 137) |
| 132 | HGL6.295 | | ATGGTTAAAACTCAACAATGAAAACACAAACAGCGCAATTTAAAAATGGGCAAA |
| | | | ATGACAGGCCAGACCCAGTGGCTCATGCG (SEQ ID NO: 138) |
| 133 | HGL6.300 | | AAGCAACTTCAGCAAAGTCTCGGGATACAAAATCAATGTGCAAAAATCACAAGC |
| | | | ATTCTTATACACCACTAACAGACAAATGGAGAGTC (SEQ ID NO: 139) |
| 134 | HGL6.302 | | GAATGGAATCAACATCAAACGGAAAAAAACGGAATTATCGAATGGAATCGAAG |
| | | | AGAATCATCGAATGGACCAGAATGGAATCATCTAATGGAATGGAATGGAATAAT |
| | | | CCATGG (SEQ ID NO: 140) |
| 135 | HGL6.305 | | TAGAAGGAATTTGATACATGCTCAGAAATACAGGCAAAGGAAGTAGGTGCCTGC |
| | | | CAGTGAACACAGGGGAACTATGGCTCCTA (SEQ ID NO: 141) |
| 136 | HGL6.310 | | GGAATCGAATGGAATCAACATCAAACGGAAAAAAACGGAATTATCGAATGGAA |
| | | | TCGAAGAGAATCATCGAATGGACC (SEQ ID NO: 142) |
| 137 | HGL6.311 | | AACTAAGACAACAGATTGATTTACACTACTATTTTCACACAGCCAAAAATATCAC |
| | | | TATGGCAATCGTCAAAAGGTCAATTCAAAGATGGGACAGT (SEQ ID NO: 143) |
| 138 | HGL6.315 | | AAAAGCAATTGGACTGATTTTAAATATACGTGGCAACAAGGATAAACTGCTAAT |
| | | | GATGGGTTTGCAAATACAGATCG (SEQ ID NO: 144) |
| 139 | HGL6.317 | HGL6.1189 | AATGGAATCAACATCGAACGGAAAAAAACGGAATTATCGAATGGAATCGAAGA |
| | | | GAATCATCGAATGGACC (SEQ ID NO: 145) |
| 140 | HGL6.319 | | TGCAAGATAACACATTTTAGTTGACACCATTGAAAACAGTTTTAACCAAGAATAT |
| | | | TAGAACCAATGAAGCAGAGAAATCAAAAGGGTGGATGGAACTGCCAAAGGATG |
| | | | (SEQ ID NO: 146) |
| 141 | HGL6.321 | | TAGAACAGAATTGAATGGAATGGCATCAAATGGAATGGAAACGAAAGGAATGG |
| | | | AATTGAATGGACTCAAATGTTATGGAATCAAAGGGAATGGACTC (SEQ ID NO: |
| | | | 147) |
| 142 | HGL6.323 | | AAGAGAATCATCGAATGGAATCGAATGGAATCAACATCAAACGGAAAAAAACG |
| | | | GAATTATCGAATGGAATCGAAGAGAATCATCGAATGGACC (SEQ ID NO: 148) |
| 143 | HGL6.324 | HGL6.431, | ATCAACATCAAACGGAAAAAAACGGAATTATCGAATGGAATCGAAGAGAATCAT |
| | | HGL6.1071 | CGAATGGACC (SEQ ID NO: 149) |
| 144 | HGL6.326 | | GAATCAACATCAAACGGAAAAAAACCGAATTATCGAATGGAATCGAAGAGAAT |
| | | | CATCGAATGGACC (SEQ ID NO: 150) |
| 145 | HGL6.327 | | ATCAACATCAAACGGAATCAAACGGAATTATCGAATGGAATCGAAGAGAATCAT |
| | | | CAAATGGACTCGAATGGAATCATCTAATGGAATGGAATGGAAGAATCCATGG |
| | | | (SEQ ID NO: 151) |
| 146 | HGL6.330 | HGL6.1005 | AAACAGTTCAAAAATTATTGCAACAAAATGAGAGAGATGAGTTTATCTTGCAAA |
| | | | CTAATGGATGGTAGCAGTGACAGTGGCAAAACGTGGTTTGATTCT (SEQ ID NO: |
| | | | 152) |
| 147 | HGL6.334 | | ATCGAATGGAATCATTGAATGGAAAGGAATGGAATCATCATGGAATGGAAACGA |
| | | | ATGGAATCACTGAATGGACTCGAATGGGATCATCA (SEQ ID NO: 153) |
| 148 | HGL6.335 | | ATTCAGCCTTTAAAAAAAGAAGACAGTCCTGTCATTTGTGACAATATGAATGAA |
| | | | ACAGACATCACATTAAATGAAATGAGCCAGGCGCAG (SEQ ID NO: 154) |
| 149 | HGL6.336 | | AGGAGAATAGCAGTAGAATGACAAAATTAGATTTTCACATGAAACTTGATGACA |
| | | | GTGTAGGAAATGGACTGAAAGGACAAGAC (SEQ ID NO: 155) |
| 150 | HGL6.337 | HGL6.1095, | AACCCACAAAGACAACAGAAGAAAAGACAACAGTAGACAAGGATGTCAACCAC |
| | | HGL6.1367 | ATTTTGGAAGAGACAAGTAATCAAACACATGGCA (SEQ ID NO: 156) |
| 151 | HGL6.338 | | GAAAATGAACAATATGAACAAACAAACAAAATTACTACCCTTACGAAAGTACGT |
| | | | GCATTCTAGTATGGTGACAAAAAGGAAAG (SEQ ID NO: 157) |
| 152 | HGL6.339 | | AACATCAAACGGAATCAAACGGAATTATCGAATGGAATCGAAGAGAATCATCGA |
| | | | ACGGACTCGAATGGAATCATCTAATGGAATGGAATGGAAGAATCCATGGACTCG |
| | | | AATGCAATCATCATCGAATGAAATCGAATGGAATCATCGAATGGACTCG (SEQ ID |
| | | | NO: 158) |
| 153 | HGL6.340 | | ACCAACATAAGACAAAGAAACATCCAGCAGCTGCCTATGGCAAAAGATTACAAT |
| | | | GTGTCAAACAAGAGGGCAATG (SEQ ID NO: 159) |
| 154 | HGL6.342 | | ATGGAATTCAATGGAATGGACATGANTGNAATGNACTTCAATGGAATGGNATCN |
| | | | AATGGAATGNAATTCANT (SEQ ID NO: 160) |
| 155 | HGL6.343 | | TATGACTTTCACAAATTACAGAAAAAGACACCCATTTGACAAGGGAACTGAAGG |
| | | | TGGTGAAGACATACTGGCAGGCTAC (SEQ ID NO: 161) |
| 156 | HGL6.344 | | AATGGAAAGGAATCGAATGGAAGGGAATGAAATTGAATCAACAGGAATGGAAG |
| | | | GGAATAGAATAGACGGCAATGGAATGGACTCG (SEQ ID NO: 162) |
| 157 | HGL6.347 | | AGCCTATCAAAAAGTGGGCTAAGAATATGAATACACAATTCTCAAAAGAAGATA |
| | | | TACAAATGGGCAACAAACATATGAAAACATACTCAACATCACTAATGATCAGGG |
| | | | AAATG (SEQ ID NO: 163) |
| 158 | HGL6.352 | HGL6.710 | AGCAACTTCAGCAAAGTATCAGGATACAAAATCAATGTACAAAAATCCCAAGCA |
| | | | TTCTTATACACCAACAACAGACAAACAGAGAGCC (SEQ ID NO: 164) |
| 159 | HGL6.353 | | AAAGACAATATACAAATGGCCAATAAGCACATGAAAAGACGCTCAACATCCTTA |
| | | | GTCGTTAAGGCAATGCAAATCAAAACCACAATG (SEQ ID NO: 165) |
| 160 | HGL6.354 | | AGCAACTTCAGCAAAGTCTCAGGATACAAAATCGATGTGCAAAAATCACAAGCA |
| | | | TTCTTATACACCAACAACAGATAAACAGAGAGCC (SEQ ID NO: 166) |
| 161 | HGL6.356 | | AACGGAAAAAAAACGGAATTATCGAATGGAATCGAAGAGAATCATCGAATGGA |
| | | | CCAGAATGGAATCATCTAATGGAATGGAATGGAATAATCCATGGACTCGAATG |
| | | | (SEQ ID NO: 167) |
| 162 | HGL6.357 | | AACAGCAATAGACACAAAGTCAGCACTTACAGTACAAAAACTAATGGCAAAAGC |
| | | | ACATGAAGTGGGACAT (SEQ ID NO: 168) |
| 163 | HGL6.358 | | GGAATCAAACGGAATTATCGAATGGAATCGAAGAGAATCATAGAACGGACTCAA |
| | | | ATGGAATCATCTAATGGAATGGAATGGGAGAATCCATGGACTCGAATG (SEQ ID |
| | | | NO: 169) |
| 164 | HGL6.360 | HGL6.1105 | AATGGAATCAATATCAAACGGAAAAAAACGGAATTATCGAATGGAATCGAAGA |
| | | | GAATCATCGAATGGACC (SEQ ID NO: 170) |
| 165 | HGL6.362 | | AAAATGATCATGAGAAAATTCAGCAACAAAACCATGAAATTGCAAAGATATTAC |
| | | | TTTTGGGATGGAACAGAGCTGGAAGGCAAAGAG (SEQ ID NO: 171) |
| 166 | HGL6.364 | | AACGGAATCAAACGGAATTATCGAATGGAATCGAAAAGAATCATCGAACGGACT |
| | | | CGAATGGAATCATCTAATGGAATGGAATGGAAGAATCCATGG (SEQ ID NO: 172) |
| 167 | HGL6.367 | | AAACGGAATTATCGAANGGAATCAAAGAGAATCATCGAANNNNNACGAATGGA |
| | | | ATCATATAATGGAATGGAATGGAATAATCCATGGACC (SEQ ID NO: 173) |
| 168 | HGL6.369 | | AATGGAATCGAATGGATTGATATCAAATGGAATGGAATGGAAGGGAATGGAATG |
| | | | GAATGGAATTGAACCAAATGTAATGGATTTG (SEQ ID NO: 174) |
| 169 | HGL6.371 | | TAAAAGACGGAACAGATAGAAAGCAGAAAGGAAAGGTGAATTGCATTACCACT |
| | | | ATTCATACTGCCACACACATGACATTAGGCCAAGTC (SEQ ID NO: 175) |
| 170 | HGL6.372 | | ACAAACAATCCAATTCGAAAATGGGCAAGATATTTCACCAAAGACATGAGCTGA |
| | | | TATTTCAC (SEQ ID NO: 176) |
| 171 | HGL6.373 | | AATGGAATCGAATGGAACAATCAAATGGACTCCAATGGAGTCATCTAATGGAAT |
| | | | CGAGTGGAATCATCGAATGGACTCG (SEQ ID NO: 177) |
| 172 | HGL6.374 | | TAACACATAAACAAACACAGAGACAAAATCTCCGAGATGTTAATCTGCTCCAGC |
| | | | AATACAGAACAATTTCTATTACCAACAGAATGCTTAATTTTTCTGCCT (SEQ ID |
| | | | NO: 178) |
| 173 | HGL6.379 | | GGAATCGAATGGAATCAACATCAAACGGAAAAAAACGGAATTATCGAATGGAA |
| | | | TCAAAGAGAATCATCGAATGGACC (SEQ ID NO: 179) |
| 174 | HGL6.382 | | AGAATGGAAAGGAATCGAAACGAAAGGAATGGAGACAGATGGAATGGAATG |
| | | | (SEQ ID NO: 180) |
| 175 | HGL6.383 | | GAATGGAATGGAAAGGAATCGAAACGAAAGGAATGGAGACAGATGGAATGGAA |
| | | | TGGAACAGAGAGCAATGG (SEQ ID NO: 181) |
| 176 | HGL6.387 | | GAATCATCATAAAATGGAATCGAATGGAATCAACATCAAATGGAATCAAATGGT |
| | | | CTCGAATGGAATCATCTTCAAATGGAATGGAATGG (SEQ ID NO: 182) |
| 177 | HGL6.389 | | AACAACAATGACAAACAAACAACAACGACAAAGACATTTATTTGGTTCACAAAT |
| | | | CTCCAGGGTGTACAAGAAGCATGGTGCCAGCATCTGCTCAGCTTCTGATGAGGG |
| | | | CTCTGGGAAGCTTTTACTC (SEQ ID NO: 183) |
| 178 | HGL6.390 | | AACGGACTCGAACGGAATATAATGGAATGGAATGGATTCGAAAGGAATGGAAT |
| | | | GGAATGGACAGGAAAAGAATTGAATGGGATTGGAATGGAATCG (SEQ ID NO: |
| | | | 184) |
| 179 | HGL6.393 | | AGGAAATAAAAGAAGACACAAACAAATGGAAGAACATTCCATGCTTATGGATA |
| | | | GGGAGAATCAGTATCGTGAAAATGGCCATACT (SEQ ID NO: 185) |
| 180 | HGL6.394 | HGL6.1136 | AACATCAAACGAAATCAAACGGAATTATCAAATTGAATCGAAGAGAATCATCGA |
| | | | ATTGCCACGAATGCAATCATCTAATGGTATGGAATGGAATAATCCATGGACCCA |
| | | | GATG (SEQ ID NO: 186) |
| 181 | HGL6.395 | | AGAAATTAACAGCAAAAGAAGGATGCAGTGCAACTCAGGACAACACATACAATT |
| | | | CAAGCAACAAATGTATAGTGGCTGGGCACCAAGGATACAG (SEQ ID NO: 187) |
| 182 | HGL6.396 | | GCAATAAAATCGACTCAGATAGAGAAGAATGCAATGGAATGGAATGGAATGGA |
| | | | ATGGAATGGGATGGAATGGTATGGAATGG (SEQ ID NO: 188) |
| 183 | HGL6.397 | | CCACATAAAACAAAACTACAAGACAATGATAAAGTTCACAACATTAACACAATC |
| | | | AGTAATGGAAAAGCCTAGTCAATGGCAG (SEQ ID NO: 189) |
| 184 | HGL6.399 | | GGACAACATACACAAATCAGTCAAGATACATCATTTCAACAGAATGAAAGACAA |
| | | | AAACCATTTGATCACTTCAATCGATGATGAAAAAGCA (SEQ ID NO: 190) |
| 185 | HGL6.400 | | GAAATCATCATCAAACGGAATCGAATGGAATCATTGAATGGAATGGAATGGAAT |
| | | | CATCATGGAATGGAAACG (SEQ ID NO: 191) |
| 186 | HGL6.405 | | TGGAATGGANTGGAATGNAATCNAATCNNNTGGTAATGAATCAAATGGAATCAA |
| | | | ATCGAATGGNAATAATGGAATCNANNGGAAACGAATGGNATCGAATTGCACTGA |
| | | | TTCTACTGACTTCGAGGAAAATGAAATGAAATGCGGTGAAGTGGAATGG (SEQ ID |
| | | | NO: 192) |
| 187 | HGL6.409 | | AGCAACTTCAGCAAAGTCTCAGGATACAAAATCAATGGGAAAAAATCACAAGCA |
| | | | TTCCTATACATCAATAACAGACAAACAGAGAGCC (SEQ ID NO: 193) |
| 188 | HGL6.410 | | GAATGTTATGAAATCAACTCGAACGGAATGCAATAGAATGGAATGGAATGGAAT |
| | | | GGAATGGAATGGAATGG (SEQ ID NO: 194) |
| 189 | HGL6.412 | | AGTAGAATTGCAATTGCAAATTTCACACATATACTCACACACAAGTACACACATC |
| | | | CACTTTTACAACTAAAAAAACTAGCACCCAGGACAGGTGCAGTGGCT (SEQ ID |
| | | | NO: 195) |
| 190 | HGL6.416 | | GGAATCAACATCAAACGGAAAAAAAACGGAATTATCGAATGGAATCGAAGAGA |
| | | | ATCATCGAATGGACC (SEQ ID NO: 196) |
| 191 | HGL6.420 | | GGAATAATCATCATCAAACAGAACCAAATGGAATCATTGAATGGAATCAAAGGC |
| | | | AATCATGGTCGAATG (SEQ ID NO: 197) |
| 192 | HGL6.422 | | ACTCAGGAAAAATAACGAATCCAACTCACAGGAGAAAGAAGTACAAACCAGAA |
| | | | ACCAATTTCAAATTACAAGGACCAGAATACTCATGTTGGCTGGCCAGT (SEQ ID |
| | | | NO: 198) |
| 193 | HGL6.424 | | AAACGCACAAACAAAGCAAGGAAAGAATGAAGCAACAAAAGCAGAGATTTATT |
| | | | GAAAATGAAAAATACACTCCACAGGGTGGG (SEQ ID NO: 199) |
| 194 | HGL6.429 | | GCATAGAATCGAATGGAATTATCATTGAATGGAATCGAATGGAATCAACATCAA |
| | | | ACGGAAAAAAACGGAATTATCGAATGGAATCGAAGAGAATCATCGAATGGACC |
| | | | C (SEQ ID NO: 200) |
| 195 | HGL6.430 | | AATGGAATCGAANAGAATCATCGAACGGACTCGAATGGAATCATCTAATGGAAT |
| | | | GGAATGGAATAATCCATGGACCCGAATG (SEQ ID NO: 201) |
| 196 | HGL6.433 | | AAATGAATCGAATGGAATTGAATGGAATCAAATAGAACAAATGGAATCGAAATG |
| | | | AATCAAATGGAATCGAATCGAATGGAATTGAATGGCATGGAATTG (SEQ ID NO: |
| | | | 202) |
| 197 | HGL6.436 | | NTCACAATCACACAACACATTGCACATGNNNANNATGCACTCACAATACACACA |
| | | | CAACACATACACAACACACATGCAATACAACACAAAACGCAACACAACATATAC |
| | | | ACNACACACAGCACACANATGCC (SEQ ID NO: 203) |
| 198 | HGL6.442 | | GAATGGAATCAAATCGAATGAAATGGAATGGAATAGAAAGGAATGGAATGAAA |
| | | | TGGAATGGAAAGGATTCGAAT (SEQ ID NO: 204) |
| 199 | HGL6.445 | | AAAGACTTAAACGTTAGACCTAAAACCATAAAAACCCTAGAGGAAAACCTAGGC |
| | | | ATTACCATTCAGGACTTAGGCATGGGCAAGGAC (SEQ ID NO: 205) |
| 200 | HGL6.446 | | GTTTACAGTCAAGTGTACAAACAGAATATAAGCAAACAAAAGAGAACATATACT |
| | | | TACAAACTATGCTAAGTGCCATGAAGGAAAAG (SEQ ID NO: 206) |
| 201 | HGL6.447 | | AAAGTCCAAAGATGAACAAAATATCCAGAAGGAAAACAAATGCACTTGGGGAG |
| | | | TGGGAAAGAAAACCAAGACTGAGCAATGCGTCAAGCTCAGACGTGCCTCACTAC |
| | | | G (SEQ ID NO: 207) |
| 202 | HGL6.448 | | AAACGGAATCAAACGGAATTATCGAATGGAGTCGAAAAGAATCATCGAACGGA |
| | | | CTCGAATGGAATCATCTAATGGAATGGAATGGAAGAATCCATGG (SEQ ID NO: |
| | | | 208) |
| 203 | HGL6.450 | HGL6.1296 | AATTGATTCGAAATTAATGGAATTGAATGGAATGCAATCAAATGGAATGGAATG |
| | | | TAATGCAATGGAATGTAATAGAATGGAAAGCAATGGAATG (SEQ ID NO: 209) |
| 204 | HGL6.453 | | TACAGAACACATGACTCAACAACAGCAGAAAGCATATTCTTTTCAAATGCACAT |
| | | | GAAACATTATCATGATGGACCAAAT (SEQ ID NO: 210) |
| 205 | HGL6.454 | | TAAGACACATAGAAAACATAAAGCAAAATGGCAGATGTAAATGCAACCTATCAA |
| | | | TCAAAACATTACGAATGGCTT (SEQ ID NO: 211) |
| 206 | HGL6.456 | | GGAACAAAATGAAATCGAACGGTAGGAATCATACAGAACAGAAAGAAATGGAA |
| | | | CGGAATGGAATG (SEQ ID NO: 212) |
| 207 | HGL6.457 | | AACGGAAAAAACGGAATTATCGAATGGAATCGAAGAGAATCATCGAATGGAAT |
| | | | CGAATGGAGTCATCG (SEQ ID NO: 213) |
| 208 | HGL6.459 | HGL6.806 | AACATACGAAAATCAATAAACGTAATCCAGCATATAAACAGAACCAAAGACAA |
| | | | AAACCACATGATTATCTCAATAGATGCAGAAAAGGCCTTT (SEQ ID NO: 214) |
| 209 | HGL6.460 | HGL6.1163 | AATCGAACGGAATCAACATCAAACGGAAAAAAACGGAATTATCGAATGGAATC |
| | | | GAAGAGAATCATCGAATGGACC (SEQ ID NO: 215) |
| 210 | HGL6.461 | | AGAATGGAATGCAATAGAATGGAATGCAATGGAATGGAGTCATCCGTAATGGAA |
| | | | TGGAAAGGAATGCAATGGAATGGAATGGAATGG (SEQ ID NO: 216) |
| 211 | HGL6.462 | | GGAATAAAACGGACTCAATAGTAATGGATTGCAATGTAATTGATTCGATTTCGA |
| | | | ATGGAATCGCATGGAATGTAATGGAATGGAATGGAATGGAAGGC (SEQ ID NO: |
| | | | 217) |
| 212 | HGL6.467 | | AGCAACTTCAGCAAAGTCTCAGGATACAAAATCAATGTACAAAAATCACAAGCA |
| | | | TTCTTATACACCAACAACAGACAAACAGAGAGCC (SEQ ID NO: 218) |
| 213 | HGL6.476 | | TAAGCAGAGAAAATATCAACACGAAAATAATGCAAGGAGAAAAATACAGAACA |
| | | | ATCCAAAATGTGGCC (SEQ ID NO: 219) |
| 214 | HGL6.487 | | AATGGAATCAACATCAAACGGAAAAAAACGGAATTATCGTATGGAATCGAAAA |
| | | | GAATTATCGAATGGACC (SEQ ID NO: 220) |
| 215 | HGL6.489 | HGL6.587 | TCAAACGGAAAAAAACGGAATTATCGAATGGAATCGAAGAGAATCATCGAATG |
| | | | GACC (SEQ ID NO: 221) |
| 216 | HGL6.490 | | AACTTCAGCAAATTCTCAGGATACAAAATCAATGTGCAAAAACCACAAGCATTC |
| | | | CTATACACCAATAATAGACAGTGAGCCAAAT (SEQ ID NO: 222) |
| 217 | HGL6.494 | HGL6.1131 | ACATCAAACGGAATCAAACGGAATTATCGAATGGAATCGAAAAGAATCATCGAA |
| | | | CGGACTCGAATGGAATCATCTAATGGAATGGAATGGAAGAATCCATGGACTCGA |
| | | | ATG (SEQ ID NO: 223) |
| 218 | HGL6.497 | | AATGGAATCGAATGCAATCATCGAACGGAATCGAATGGCATCACCGAATGGAAT |
| | | | GGAATGGAATGGAATGGAATGG (SEQ ID NO: 224) |
| 219 | HGL6.499 | | AATCCAGCATATAAACAGAACCAAAGACAAAAACCACATGATTATCTCAATAGA |
| | | | TGCAGAAAAGGCC (SEQ ID NO: 225) |
| 220 | HGL6.500 | | TGACTAAACAGAGTTGAACAAGAACAAAAAGCAAATTTGCAGAAATGAAATAC |
| | | | ATACTAATTGAAAGTCCATGGACAGGCTCAACAGATGATATAGATACAGCTAAA |
| | | | GAGATAATTAGTGAAATGGATCAG (SEQ ID NO: 226) |
| 221 | HGL6.501 | | GATCATCAGAGAAACAGAGAAATGCAAATTAAAACCACAATGAGATACTATCTC |
| | | | CACACAAGTCAGAATGGCTAT (SEQ ID NO: 227) |
| 222 | HGL6.503 | | AGGATACAAAATCAATGTACAAAAATCACAAACATTCTTATACACCAACAACAG |
| | | | ACAAACAGAGAGCCAAATCATGGGTG (SEQ ID NO: 228) |
| 223 | HGL6.505 | | TAAGCAACTTCAGCAAAGTCTCAGGATACAAAATCAATGTACAAAAATCACAAG |
| | | | CATTCTTATACACCAACAACAGACAAACAAGAGTGCCAAATCATG (SEQ ID NO: |
| | | | 229) |
| 224 | HGL6.506 | | AGAATTGATTGAATCCAAGTGGAATTGAATGGAATGGAATGGATTAGAAAGGAA |
| | | | TGGAATGGATTGGAATGGATTGGAATGGAAAGG (SEQ ID NO: 230) |
| 225 | HGL6.508 | | AATGGAATGCAATCGAATGGAATGGAATCGAACGGAATGGAATAAAATGGAAG |
| | | | AAAACTGGCAAGAAATGGAATCG (SEQ ID NO: 231) |
| 226 | HGL6.509 | | AACTGCATCAACTAACAGGCAAAATAACCAGCTAATATCATAATGACAGGATTA |
| | | | AATTCACAAATGACAATATTAACCGTAAATGTAAATGGGCTA (SEQ ID NO: 232) |
| 227 | HGL6.510 | | TACAAAGAACTCAAACAAATCAGCAAGAACAAAAACAATCCCAACAAAATGTTG |
| | | | GACAAAGACATGAATAGACAATTCTCGAAAGAAGATGTACAAATGGCT (SEQ ID |
| | | | NO: 233) |
| 228 | HGL6.512 | | AGAGAAATGCAAATCAAAACCACAATGGAATACCATCTCACGCCAGTCAGAATG |
| | | | GCAATTATTAAAAAATCACAACAATTAATGATGGCAAGGCTGTGG (SEQ ID NO: |
| | | | 234) |
| 229 | HGL6.513 | | GTAAACAAACAATCAAGCAAGTAAGAACAGAAATAACAGCATTTGGCTTTTGAG |
| | | | TTAATGACAAGAACACTCGGCATGGGAGCCTGGGTGAGCAAATCACAGATCTTC |
| | | | (SEQ ID NO: 235) |
| 230 | HGL6.514 | | GAATCAACCCGAGCGGAAAGGAATGGAATGGAATGGAATCAACACGAATGGAA |
| | | | TGGAACGGAATGGAATGGGATGGGATGAAATGGAATGG (SEQ ID NO: 236) |
| 231 | HGL6.516 | | AGCAACTTCAGCAAAGTCTCAGGAGACAAAATCAATGTACAAAAATCACAAGCA |
| | | | TTCTTATACACCAATAACAGACAAACAGAGAGCC (SEQ ID NO: 237) |
| 232 | HGL6.520 | | AAGAAATGGAATCGAAGAGAATGGAAACAAACGGAATGGAATTGAATGGAATG |
| | | | GAATTGAATGGAATGGGA (SEQ ID NO: 238) |
| 233 | HGL6.522 | | GACATGCAAACACAACACACAGCACACATGGAACATGCATCAGACATGCAAACA |
| | | | CAACACACATACCACACATGGCATATGCATCAGACGTGCCTCACTAC (SEQ ID |
| | | | NO: 239) |
| 234 | HGL6.528 | | TACAGATAAGAAAATTGAGACTCAAGAGTATTACATAAATTGTTTCAGCTACCAC |
| | | | AGCAAAAAATGGTATGGTTGGGAATCAAGCTCAGGG (SEQ ID NO: 240) |
| 235 | HGL6.529 | | AAAGGAATGCACTCGAATGGAATGGACTTGAATGGAATGTCTCCGAATGGAACA |
| | | | GACTCGTATGAAATGGAATCGAATGGAATGGAATCAAATGGAATTGATTTGAGT |
| | | | GAAATGGAATCAAATGGAATGGCAACG (SEQ ID NO: 241) |
| 236 | HGL6.530 | | TGAAACAAATGATAATGAAAATACAACATACCAAACATACGAGATACAGTAAAA |
| | | | GCAGTACTAAGATGCAAGTATATATTGCTACAAGTGCCTAC (SEQ ID NO: 242) |
| 237 | HGL6.531 | | GGAACAAAATGAAATCGAACGGTAGGAATCGTACAGAACGGAAAGAAATGGAA |
| | | | CGGAATGGAATGCACTCGAATGGAAAGGAGTCCAAT (SEQ ID NO: 243) |
| 238 | HGL6.532 | | AAATTGATTGAAATCATCATAAAATGGAATCGAAGGGAATCAACATCAAATGGA |
| | | | ATCAAATGGAATCATTGAACGGAATTGAATGGAATCGTCAT (SEQ ID NO: 244) |
| 239 | HGL6.533 | | AGAAAGGATTCGAATGGAATGAAAAAGAATTGAATGGAATAGAACAGAATGGA |
| | | | ATCAAATCGAATGAAATGGAATGGAATAGAAAGGAATGGAATG (SEQ ID NO: |
| | | | 245) |
| 240 | HGL6.534 | | AGAATGGAAAGCAATAGAATGGAACGCACTGGATTCGAGTGCAATGGAATCAAT |
| | | | TGGAATGGAATCGAATGGAATGGATTGGCA (SEQ ID NO: 246) |
| 241 | HGL6.535 | | AACACCAAACGGAAAAAAACGGAATTATCGAATGGAATCGAAGAGAATCTTCG |
| | | | AACGGACCCGAATGGGATCATCTAATGGAATGGAATGGAATAATCCATGG (SEQ |
| | | | ID NO: 247) |
| 242 | HGL6.536 | | AATGGAGACTAATGTAATAGAATCAAATGGAATGGCATCGAATGGAATGGACTG |
| | | | GAATGGAATGTGCATGAATGGAATGGAATCGAATGGATTG (SEQ ID NO: 248) |
| 243 | HGL6.539 | | TGGGATATGGGTGAAAGAACAAGTTTGCAGAAAAGATACAGTGAATTATGGACC |
| | | | ATGAGTTCGGGAAAGAAGGGTAGGACTGCG (SEQ ID NO: 249) |
| 244 | HGL6.540 | | AAATCGAATGGAACGCAATAGAATAGACTCGAATGTAATGGATTGCTATGTAAT |
| | | | TGATTCGAATGGAATGGAATCGACTGGAATGCAATCCAATGGAATGGAATGCAA |
| | | | TGCAATGGAATGGAATCGAACGGAATGCAGTGGAAGGGAATGG (SEQ ID NO: |
| | | | 250) |
| 245 | HGL6.541 | | AATCAACAAGGAACTGAAACAAGTAAACAAGAAAACAAATAACACCATAAAAC |
| | | | ATGGGCAAAGGACATAAACAGACATTTTTCAAAAAAGACATACAAATGGCCGAG |
| | | | (SEQ ID NO: 251) |
| 246 | HGL6.542 | | AATGGAATCAACATCAAACGGAAAAAAACGGAATTATCGAATGGAATCGAAGA |
| | | | GAATCATCGAATGGACCCAGGCTGGTCTTGAACTCC (SEQ ID NO: 252) |
| 247 | HGL6.545 | | ATTGAATGGGCTAGAATGGAATCATCTTTGAACGGAATCAAAGGGAATCATCAT |
| | | | CGAATGGAATCGAATGGAAATGTCAACG (SEQ ID NO: 253) |
| 248 | HGL6.547 | | AATGGACTCGAATGGAATCAACATCAAATGGAATCAAGCGGAATTATCGAATGA |
| | | | AATCGAAGAGAATCATCGAATGGACTCGAAAGGAATCATCTAATGGAATGGAAT |
| | | | GGAATAATCCATGGACTCGAATGCAATCATCATCG (SEQ ID NO: 254) |
| 249 | HGL6.549 | | ACAGACAGAGATTTAAAACAATAAACAAGCAGTAAGCAAACACAGATAACAAA |
| | | | ATGACATGATCCAACAAATACTCAGAAGGAGACTTAGAAATGAATTGAGGGTC |
| | | | (SEQ ID NO: 255) |
| 250 | HGL6.553 | | AATGTAATCCAGCATATAAACAGAGCCAAAGACAAAAACCACATGATTATCTCA |
| | | | ATAGATGCAGAAAAAGCCTTTGACAAAATTCAACAACCCTTCATGCTAAAAACT |
| | | | CTCAATAAATTAGGTATTGATGGGACG (SEQ ID NO: 256) |
| 251 | HGL6.555 | | AAACGGAAAAAAACGGAATTATTGAATGGAATCGAAGAGAATCTTCGAACGGA |
| | | | CCCGAATGGAATCATCTAATGGAATGGAATGGAATAATCCATGG (SEQ ID NO: |
| | | | 257) |
| 252 | HGL6.557 | HGL6.1238 | GCTCAAGGAAATAAAATAGGACACAAAGAAATGGAAAAACATTCCATACTCATG |
| | | | GATAGAAAGAATCAATATCATGAAATGGCC (SEQ ID NO: 258) |
| 253 | HGL6.560 | | ACTCGAGTGGAATTGACTGTAACAAAATGGAAAGTAACGGATTGGAATCGAATG |
| | | | GAACGGAATGGAATGGAATGGACAT (SEQ ID NO: 259) |
| 254 | HGL6.561 | | TACAAACTTTAAAAAATGATCAACAGATACACAGTTAGCAAGAAAGAATTGAGG |
| | | | GCAAAGAATATGCCAGACAAACTCAAGAGGAAGATGATGGTAGAGATAGGTCA |
| | | | CATTGGAGTGTCA (SEQ ID NO: 260) |
| 255 | HGL6.562 | HGL6.154, | GGAATCGAATGGAATCAATATCAAACGGAAAAAAACGGAATTATCGAATGGAAT |
| | | HGL6.114 | CGAAGAGAATCATCGAATGGACC (SEQ ID NO: 261) |
| 256 | HGL6.564 | | AACGGAATCAAACGGAATTATCGAATGGAATCGAAAAGAATCATCGAACGGACT |
| | | | CGAATGGAATCATCTAATGTAATGGAATGGAAGAATCCATGGACTCGAATG |
| | | | (SEQ ID NO: 262) |
| 257 | HGL6.565 | | GGAAATAACAGAGAACACAAACAAATGGGAAAACATTCCATGTTCATGGATAGG |
| | | | AAGAATCAATATTGTGAAAATGGCCATACT (SEQ ID NO: 263) |
| 258 | HGL6.570 | | AACGGAAAAAAACGGAATTATCGAATGGAATCGAAGAGAATCATCGAATGGAC |
| | | | CAGAATGGAATCATCTAATGGAATGGAATGGAATAATCCATGGACTCGAATG |
| | | | (SEQ ID NO: 264) |
| 259 | HGL6.581 | | CAACATCAAACGGAAAAAAACGGAATTATGGAATGGAATCGAAGAGAATCATC |
| | | | GAATGGACCCGAATGGAATCATCTGAAATATAATAGACTCGAAAGGAATG (SEQ |
| | | | ID NO: 265) |
| 260 | HGL6.589 | | ATGGAATCGAATGGAATGGACTGGAATGGAATGGATTCGAATGGAATCGAATGG |
| | | | AACAATATGGAATGGTACCAAATG (SEQ ID NO: 266) |
| 261 | HGL6.595 | HGL6.1293 | GAATGGAATCAACATCAAACGGAAAAAAACGGAATTATCGAATGGAATCGAAG |
| | | | AGAATCATCGAATGGACC (SEQ ID NO: 267) |
| 262 | HGL6.606 | | AAGGAATTTAAGCAAATCAACAAGCAAAACCAAAATAATCCCATTAAAAAGTGG |
| | | | GTAAAGGACATGAATACACACTTGTCAATAGAGGACATTCAAGTGGCCAAC |
| | | | (SEQ ID NO: 268) |
| 263 | HGL6.608 | | AAATGGACTCGAATGGAATCATCATAGAATGGAATCGAATGCAATGGAATGGAA |
| | | | TCTTCCGGAATGGAATGGAATGGAATGGAATGGAG (SEQ ID NO: 269) |
| 264 | HGL6.609 | | GAATCANCNNNNNNNGGAATCGAATGGAATCAACATCAAATGGAATCAAATGG |
| | | | AATCATTGAACGGAATTGAATGGAATCGTCAT (SEQ ID NO: 270) |
| 265 | HGL6.610 | | AGCAACTTCAGCAAAGTCTCAGGATACAAAATCAATGTACAAAAATCACAAGCA |
| | | | TTCTTATACACCAATAACAGACAAACAGAGAGCCAAAA (SEQ ID NO: 271) |
| 266 | HGL6.611 | | TATGCAAATCAATAAACATAATCCATCACATAAACAGAAACAAAGACAAAATGA |
| | | | CATGATTATCTCAATAGATGCAGAAAAGGCC (SEQ ID NO: 272) |
| 267 | HGL6.615 | | AGTAAATCACCATAAAGAAGGTAAGAGTTCATTCACAAAAACAACAAACTGAAG |
| | | | AATCAGGCCATAGTA (SEQ ID NO: 273) |
| 268 | HGL6.617 | | AGAAACAGAAAACAGTCAAACCAATGGGCAATCCATATCAGATGCAGTATTATG |
| | | | AACAGAAGTGTAAAGAATGCACCAGGCACAATGGC (SEQ ID NO: 274) |
| 269 | HGL6.619 | | AGGAAAAACAACAACAACAACAGGAAAACAACCTCAGTATGAAGACAAGTACA |
| | | | TTGATTTATTCAACATTTACTGATCACTTTTCAGGTGGTAGGCAG (SEQ ID NO: |
| | | | 275) |
| 270 | HGL6.623 | | GATTGGAACGAAATCGAATGGAACGGAATAGAATAGACTCGAATGTAATGGATT |
| | | | GCTATGTAATTGATTCGAATGGAATGGAATCGAATGGAATGCAATCCAATGGAA |
| | | | TGGAATGCAATGCAATGGAATGG (SEQ ID NO: 276) |
| 271 | HGL6.624 | | AACATATGGAAAAAAACTCAACATCACTGATCATTAGAGAAATGCAAATCAAAA |
| | | | CCACAATGAGATACCATCTCACGCCAGTCAGAATGGCG (SEQ ID NO: 277) |
| 272 | HGL6.625 | | ATGGAATGGAATAATCAACGTACTCGAATGCAATCATCATCGTATAGAATCGAA |
| | | | TGGAATCATCGAATGGACTCGAATGGAATAATCATTGAACGGAGTCGAATGGAA |
| | | | TCATCATCGGATGGAAAC (SEQ ID NO: 278) |
| 273 | HGL6.627 | | AAANAANTCNAATGGAATCNNTGNCGAATGGAATGGAATGGAATCGAANAATT |
| | | | GAATTGNNNANAATCNNANGNAANCNTTGNATGGGCTCAAAT (SEQ ID NO: 279) |
| 274 | HGL6.629 | | AGAAAAGATAACTCGATTAACAAATGAACAAACACCTGAATACACAAGTCTCAA |
| | | | AAGAAGACATAAAAATGGCCAAC (SEQ ID NO: 280) |
| 275 | HGL6.632 | | ATGGAATCAACATCAAACGGAATCACACGGAATTATCGAATGGAATCGAAAAGA |
| | | | ATCATCGAACGGACTCGAATGGAATCATCTAATGGAATGGAATGGAAG (SEQ ID |
| | | | NO: 281) |
| 276 | HGL6.633 | HGL6.1135 | AATGGAATCAACATCAAACGGAATCAAGCGAAATTATCGAATGGAATCGAAGAG |
| | | | AATCATCGAATGGACTCGAATGGAATCATCTAATGGAATGGAATGGGAT (SEQ ID |
| | | | NO: 282) |
| 277 | HGL6.634 | | AAACACAGTACAAATACTAATTCAAATCAAACTTACTCAAAGTCATAATCAAAC |
| | | | ATGCCAGACGGGCTGAGGGGCAGCATTA (SEQ ID NO: 283) |
| 278 | HGL6.638 | | AACCACTGCTCAAGGAAATAAGAGAGAACACAAACAAATGAAAAAACATTCCA |
| | | | TGCTCATGGATAGGAAGAATCAG (SEQ ID NO: 284) |
| 279 | HGL6.641 | | GGAATCGAGTGGAATCATCGAAAGAAATCGAATGGAATCATTGTCGAATGGAAT |
| | | | GGAATGGAATCAAAGAATGGAATCGAAGGGAATCATTGGATGGGCT (SEQ ID |
| | | | NO: 285) |
| 280 | HGL6.642 | | AAAGAAAGACAGAGAACAAACGTAATTCAAGATGACTGTTTACATATCCAAGAA |
| | | | CATTAGATGGTCAAAGACTTTAAGAAGGAATACATTCAAAGGCAAAAAGTCACT |
| | | | TACTGATTTTGGTGGAGTTTGCCACATGGAC (SEQ ID NO: 286) |
| 281 | HGL6.645 | | AAGATAGAGTTGAAACAGTGGACAATTAAAGAGTAATTTGGAAGAATGGTGAAA |
| | | | TTACAGCCATGCTTTGAATCAGGCGGGTTCACTGGC (SEQ ID NO: 287) |
| 282 | HGL6.646 | | AAGAGTATCAACAGTAAATTACATTAGCAGAAGAATCAACAAACATGAAAATAG |
| | | | AAATTATGGTAGCCAAAGAACAG (SEQ ID NO: 288) |
| 283 | HGL6.647 | | GAAAGGAATCATCATTGAATGCAATCACATGGAATCATCACAGAATGGAATCGT |
| | | | ACGGAATCATCATCGAATGGAATTGAATGGAATCATCAATTGGACTCGAATGGA |
| | | | ATCATCAAATGGAATCGATTGGAAGTGTCAAATGGACTCG (SEQ ID NO: 289) |
| 284 | HGL6.651 | | CAGCGCACCACAGCACACACAGTATACACATGACCCACAATACACACAACACAC |
| | | | AACACATTCACACACCAC (SEQ ID NO: 290) |
| 285 | HGL6.655 | | GCAAACAGAATTCAACACTACATTAGAACGATCATTCATCACGACCTAGTAGGA |
| | | | TGTTTTTCCTGGGATGCAAGGATGGTTCAACAT (SEQ ID NO: 291) |
| 286 | HGL6.656 | | CAATCAAAACAGCAATGAGATACCATTTTACACCAATCAAAATGGCTACTAAAA |
| | | | AGTCAAAAGCAAATGCC (SEQ ID NO: 292) |
| 287 | HGL6.658 | HGL6.830 | AGAACCATATTGAAGAGACAGAGTGATATATAAAACTGCTAACTCAAGCAGCAC |
| | | | AAGAATTAAATGAATACCAAGAAAATACTTGGCCAG (SEQ ID NO: 293) |
| 288 | HGL6.660 | | TGGAATAGAATGGAATCAATGTTAAGTGGAATCGAGTGGAATCATCGAAAGAAA |
| | | | TCGAATGGAATCATTGTCGAATGGTATGGAATGGAATCA (SEQ ID NO: 294) |
| 289 | HGL6.661 | | AATGGAATGGAATCATCGCATAGAATNGAATGGAATTATCATCGAATTGAATCG |
| | | | AATGGTATCAACATCAAACGGAAAAAAACGGAAATATCGAANGGAATCGAAGA |
| | | | GAATCATCGAACGGACC (SEQ ID NO: 295) |
| 290 | HGL6.662 | | ACATACGCAAATCAATAAACATAATCCATCACATAAACAGAACCAAAGACAAAA |
| | | | ATCACATGATTATCTCAATAGATGCAGAAAAGGCCTTCGAC (SEQ ID NO: 296) |
| 291 | HGL6.663 | | AAAAAATGTTCAACATCACTAGTCAGCAGAGAAATGCAAATCAAAATCACAATG |
| | | | AGATAACTTCTCACACCAGACAGCATGGC (SEQ ID NO: 297) |
| 292 | HGL6.668 | | GAAAAACAAAACAAAACAAACAAACAAACAATCAAAAAAGTGGTAGCAGAAAC |
| | | | CAGAAAGTCCATGTATATAGCTAATTGGCCTGGTTGT (SEQ ID NO: 298) |
| 293 | HGL6.671 | | AACAGCAATGACAATGATCAGTAACAACAAGACTTTTAACTTTGAAAAAATCAG |
| | | | GACC (SEQ ID NO: 299) |
| 294 | HGL6.672 | | AAGAGCCTGAATAGCTAAAGTGATCATAAGCAAAAAGAACAAAGTCGGAAGCA |
| | | | TCACATTACCTGACTTCAAACTATACTCAAAGGCTATG (SEQ ID NO: 300) |
| 295 | HGL6.675 | | AAAAGGAAATACAAGACAACAAACACAGAAACACAACCATCGGGCATCATGAA |
| | | | ACCTCGTGAAGATAATCATCAGGGT (SEQ ID NO: 301) |
| 296 | HGL6.677 | | AAGCAAAGAAAGAATGAAGCAGCAAAAGAACGAAAGCAGGAATTTATTGAAAA |
| | | | CCAAAGTACACTCCACAGTATGGGAGCGGACCCGAGCA (SEQ ID NO: 302) |
| 297 | HGL6.679 | | GCAAATGATTATAAGTGCTGTTATAGAAACATTCAAAGACCAGAAAAGGACCAC |
| | | | AATGGCTGACCAC (SEQ ID NO: 303) |
| 298 | HGL6.681 | | AGAGCAGAAACAAATGGAATTGAAATGAAGACAACAATCAAAAGCATCAATGA |
| | | | AATGAAAAGTTGGGTTTTGGAAGAGAGAAACAAT (SEQ ID NO: 304) |
| 299 | HGL6.683 | | ACACAAACACACACACACACACACACACACACACACACACACACACACACACAC |
| | | | ACACACACACACATAC (SEQ ID NO: 305) |
| 300 | HGL6.686 | | AACAAACAAATGAGATGATTTCAGATAGTGATAAACACTATAACATAATTAATT |
| | | | CGTGCCAATCAGAGCATAACAGTGGTGTGGTGGCTGTGGAACAGATAGCAGAC |
| | | | (SEQ ID NO: 306) |
| 301 | HGL6.688 | | AATGGAATCGAGTGGAATGGAAGGCAATGGAATAGAATGGAATGGAATCGAAA |
| | | | GGAACGGAATGGAATGGAATGGAATG (SEQ ID NO: 307) |
| 302 | HGL6.689 | | AGCAGTGCAAGAACAACATAACATACAAGTAAACAAACACATGGGGCCAGGTA |
| | | | ATAAAAAGTCAGGCTCAAGAGGTCAG (SEQ ID NO: 308) |
| 303 | HGL6.690 | | AGAAATGGAATCGGAGAGAATGGAAACAAATGGAATGGAATTGAATGGAATGG |
| | | | AATTGAATGGAATGGGAACG (SEQ ID NO: 309) |
| 304 | HGL6.694 | | GCACTAGTCAGATCAAGACAGAAAGTCAACGAACAAAGAACAGACTTAAACTAC |
| | | | ACTCTAGAACAAATGGACCTA (SEQ ID NO: 310) |
| 305 | HGL6.704 | | AAGAGAACTGCAAAACACTGCTCAAAGAAATCAGAGATGACAAAAACACATGG |
| | | | AAAAACGTTTCATGCTCATGGATTGGAAGACTTA (SEQ ID NO: 311) |
| 306 | HGL6.705 | | AATCAACACGAATAGAATGGAACGGAATGGAATGGAATGGAATGGAATGGAAT |
| | | | GGAGTGGAATGGAACAGAATGGAGTGGAAT (SEQ ID NO: 312) |
| 307 | HGL6.707 | | AACATCAAACGAAATCAAACGGAATTATCAAATTGAATCGAAGAGAATCATCGA |
| | | | ATTGCCACGAATGCAACCATCTAATGGTATGGAATGGAATAATCCATGGACCCA |
| | | | GATG (SEQ ID NO: 313) |
| 308 | HGL6.714 | | CGGAATTATCATCGAATGTAATCGAATGGAATCAACATCAAACGGAAAAAAACG |
| | | | GAATTATCGAATGGAATCGAAGAGAATCATCGAATGGACC (SEQ ID NO: 314) |
| 309 | HGL6.719 | | TGGACACACACGAACACACACCTACACACACGTGGACACACACGGACACATGGA |
| | | | CACACACGAACACATGGACACACACACGGGGACACACACAGACACACACAGAG |
| | | | ACACACACGGACACATGG (SEQ ID NO: 315) |
| 310 | HGL6.720 | HGL6.1044 | AGCAACTTCAGCAAAGTCTCAGGATACAAAATCAATGTGCAAAAATCACAAGCA |
| | | | TTCTTATACACCAATAACAGACAAACAGAGAGCC (SEQ ID NO: 316) |
| 311 | HGL6.721 | HGL6.1020 | AAAATCAATATGAAAACAAACACAAGCAGACAAAGAAAATTGGGCAAAAGGTT |
| | | | TGAGCAGACACTTCACCAAAGAAGTACAAATGGCAAATCAGCA (SEQ ID NO: |
| | | | 317) |
| 312 | HGL6.724 | | ATCAAACGGAATCAAACGGAATTATCGAATGGAATCGAAGAGAATCATCGAATG |
| | | | GACTCGAATGGAATCATCTAATGGAATGGAATGGAAGAATCCATGG (SEQ ID NO: |
| | | | 318) |
| 313 | HGL6.725 | | AACAGATTTAAACAAACCAACAAGCAAAAAACGAACAACTCCATTCAAACATGG |
| | | | ACAAAAGACACGAACAGACACTTTTCAAAGAAGACATACATGTGGCC (SEQ ID |
| | | | NO: 319) |
| 314 | HGL6.726 | | AAATGGAATGGAATGCACTTGAAAGGAATAGACTGGAACAAAATGAAATCGAA |
| | | | CGGTAGGAATCATACAGAACAGAAAGAAATGGAACGGAATGGAATG (SEQ ID |
| | | | NO: 320) |
| 315 | HGL6.727 | | ACCACACACAAAATACACCACACACCACACACACACCACACACTATACACACAC |
| | | | CACACACCACACAC (SEQ ID NO: 321) |
| 316 | HGL6.728 | | AAAGAAATAGAAGGGAGTTGAACAGAATCGAATGGAATCGAATCAAATGGAAT |
| | | | CGAATGGCATCAAATGGAATCGAATGGAATGTGGTGAAGTGGATTGG (SEQ ID |
| | | | NO: 322) |
| 317 | HGL6.729 | | GGAATCATCATAAAATGGAATCGAATGGAATCATCATCAAATGGAATCAAATGG |
| | | | AATCATTGAACGGAATTGAATGGAATCGTCAT (SEQ ID NO: 323) |
| 318 | HGL6.730 | | TGGAATGGAATGGAATGAAATAAACACGAATAGAATGGAACGGAATGGAACGG |
| | | | AATGGAATGGAATGGAATGGAAAG (SEQ ID NO: 324) |
| 319 | HGL6.731 | | AAGAATTGGACAAAACACACAAACAAAGCAAGGAAGGAATGAAAGGATTTGTT |
| | | | GAAAATGAAAGTACACTCCACAGTGTGGGAGCAG (SEQ ID NO: 325) |
| 320 | HGL6.732 | | TAATCAGCACAATCAACTGTAGTCACAAAACAAATAGTAACGCAATGATAAAGA |
| | | | AACAGAGAACTAGTTCAAATAAACATGATAAGATGGGG (SEQ ID NO: 326) |
| 321 | HGL6.733 | | AAGCGGAATTATCAAATGGAATCGAAGAGAATGGAAACAAATGGAATGGAATT |
| | | | GAATGGAATGGAATTGAATGGAATG (SEQ ID NO: 327) |
| 322 | HGL6.734 | | AAGCAACTTCAGCAAAGTCTCAGGACACAAAATCAATATGCGAAAATCACAAGC |
| | | | ATTCCTATACACCAATAATAGACAAACAGAGAGCCAAATCATG (SEQ ID NO: 328) |
| 323 | HGL6.736 | | TTCACAGCAGCATTACGCACAATAGCCAGAAGGTGGGAACAGACAAAATGCCTT |
| | | | TTGATGGG (SEQ ID NO: 329) |
| 324 | HGL6.738 | | AGACCCTAATATCACAGTTAAACGAACTAGAGAAGGAAGAGCAAACAAATTCAA |
| | | | AAGCTAGCGGAAAGCAAGAAATAACTAAGACCAG (SEQ ID NO: 330) |
| 325 | HGL6.739 | | TAAAAGTGTGCTCAACATCATTGATCATCAGAGAAATGCAAATCAAAACTACAA |
| | | | TGAGATATCATCTCATCCCAGTCAAAGTGGCT (SEQ ID NO: 331) |
| 326 | HGL6.740 | | ACTTGAATCGAATGGAAAGGAATTTAATGAACTTAAATCGAATGGAATATAATG |
| | | | GTATGGAATGGACTCATGGAATGGAATGGAAAGGAATC (SEQ ID NO: 332) |
| 327 | HGL6.742 | | TGGAATCATCATCGAAAGCAAGCGAATGGAATCATCAAATGGAAACGAATGGAA |
| | | | TCATCGAATGGACTCGGATGGAATTGTTGAATGGACT (SEQ ID NO: 333) |
| 328 | HGL6.743 | | TGGAATCAACATCAAACGGAAAAAAACGGAATTATCGAATGGAATCGAAGAGA |
| | | | ATCATCGAATGGACC (SEQ ID NO: 334) |
| 329 | HGL6.745 | | TAAGTGAATTGAATAGAATCAATCTGAATGTAATGAAATGGAATGGAACGGAAT |
| | | | GGAATGGAATGGAATGGAATGGAATGGAATGG (SEQ ID NO: 335) |
| 330 | HGL6.747 | | AGGAAAATTTAATCAGCAGGAATAGAAACACACTTGAGAAATCCATGTGGAATG |
| | | | AAAAGAGAATGGCTGAGCAGCAACAGATTGTCAAAAAGGAAATC (SEQ ID NO: |
| | | | 336) |
| 331 | HGL6.749 | HGL6.897 | AACATCAAACGGAAAAAAAACGGAATTATCGAATGGAATCGAAGAGAATCATC |
| | | | GAATGGACC (SEQ ID NO: 337) |
| 332 | HGL6.756 | | GAAAATGAACAATATGAACAAACAAACAAAATTACTACCCTTACGAAAGTACGT |
| | | | GCATTCTAGTATGGTGACAAAAAGGAAA (SEQ ID NO: 338) |
| 333 | HGL6.757 | | AGAAAACACACAGACAACAAAAAACACAGAACGACAATGACAAAATGGCCAAG |
| | | | C (SEQ ID NO: 339) |
| 334 | HGL6.758 | HGL6.1040 | AGCAACTTCAGCAAAGACTCAGGATACAAAATCAATGTGCAAAAATCACAAGCA |
| | | | TTCTTATACACCAATAACAGACAGAGAGCCAAAT (SEQ ID NO: 340) |
| 335 | HGL6.759 | | TGACATGCAAGAAATAAGGAAGTGCAAAAACAAACAAACAAACAACAACAACA |
| | | | ACAACAACAACAACAACAAAAAACAGTCCCAAAAGGATGGGCAG (SEQ ID NO: |
| | | | 341) |
| 336 | HGL6.760 | | TAATTGAGAATAAGCATTCCAGTGGAAAAAAAACTAAACAATTTGTTGTAAAAC |
| | | | ATCCTTAAAAGCATCAGAAAGTTAATACAGCAATGAAGAATTACAGGACCAAAT |
| | | | TAAGAATGGTATGGAAGCCTGTTA (SEQ ID NO: 342) |
| 337 | HGL6.762 | | TATCATCGAATGGAATCGAATGGAATCAACATCAAACGGAAAAAAACGGAATTA |
| | | | TCGAATTGAATCGAAGAGAATCATCGAATGGACC (SEQ ID NO: 343) |
| 338 | HGL6.764 | | GAATGGAATCAAATAGAATGGAATCGAAACAAATGGAATGGAATGGAATGGGA |
| | | | GCTGAGATTGTGTCACTGCAC (SEQ ID NO: 344) |
| 339 | HGL6.765 | | AGCAAAACAAACACAATCTGTCGTTCATGGTACTACGACATACTGGGAGAGATA |
| | | | TTCAAATGATCACACAAAACAACATG (SEQ ID NO: 345) |
| 340 | HGL6.766 | | AAGGATTCGAATGGAATGAAAAAGAATTGAATGGAATAGAACAGAATGGAATC |
| | | | AAATCGAATGAAATGGAGTGGAATAGAAAGGAATGGAATG (SEQ ID NO: 346) |
| 341 | HGL6.768 | | AACGGAATCAAACGGAATTATCGAATGNNNTNNAAGAGAATCATCGAACGGACT |
| | | | CGAATGGAATCATCTAATGGAATGGAATGGAAGAATCCATGGACTCGAATGCAA |
| | | | TCATCATCGAATGGAATCGAACGGAATCATCGAATGGCC (SEQ ID NO: 347) |
| 342 | HGL6.771 | | AATCAACTAGATGTCAATGGAATGCAATGGAATAGAATGGAATGGAATTAACAC |
| | | | GAATAGAATGGAATGGAATGGAATGGAATGG (SEQ ID NO: 348) |
| 343 | HGL6.772 | | TGTAACACTGCAAACCATAAAAACCGTAGAAGAAAACCTAGACAATACTATTCA |
| | | | GGACATAGGCATGGGCAAAGAC (SEQ ID NO: 349) |
| 344 | HGL6.773 | | AATGGACTCGAATGGAATAATCATTGAACGGAATCGAATGGAATCATCATCGGA |
| | | | TGGAAATGAATGGAATCATCATCGCATGGAATCG (SEQ ID NO: 350) |
| 345 | HGL6.776 | | GAATGGAATGATACGGAATAGAATGGAATGGAACGAAATGGAATTGAAAGGAA |
| | | | AGGAATGGAATGGAATGGAATGG (SEQ ID NO: 351) |
| 346 | HGL6.777 | | AAAAATGACCAGAGCAATAGAATGCATTGACCAGATAAAGACCTTCACGTATGT |
| | | | TGAACTAAAATGTGTGGTGCAGGTG (SEQ ID NO: 352) |
| 347 | HGL6.781 | | AATCATCATCGAATGGAATCGAATGGTATCATTGANTGNAATCGAATGGAATCA |
| | | | TCATCANATGGAAATGAATGGAATCGTCAT (SEQ ID NO: 353) |
| 348 | HGL6.785 | | ACAAAATCAAACTAACCTCGATAAGAATGCAAGTGAATCAAAATGAGTTTCAAG |
| | | | GGGTTGTGGCTAGTACACGCTTTCTACAGCTG (SEQ ID NO: 354) |
| 349 | HGL6.787 | | GAATCAAATCAATGGAATCAAATCAAATGGAATGGAATGGAATTGTATGGAATG |
| | | | GAATGGCATGG (SEQ ID NO: 355) |
| 350 | HGL6.789 | | TAATGCAGTCCAATAGAATGGAATCGAATGGCATGGAATATAAAGAAATGGAAT |
| | | | CGAAGAGAATGGGAACAAATGGAATGGAATTGAGTGGAATGGAATTGAATGGA |
| | | | ATGGGAACGAATGGAGTG (SEQ ID NO: 356) |
| 351 | HGL6.792 | | TGAATAGACACACAGACCAATGGAACAGAATAGAGAACACAGAATAAATCTGC |
| | | | ACACTTATAGCCAGCTGATTTTTGACAAATTTGCCAAG (SEQ ID NO: 357) |
| 352 | HGL6.797 | HGL6.810, | AACATCNNACGGAAAAAAACGGAATTATCGAATGGAATCGAAGAGAATCATCG |
| | | HGL6.1172, | AATGGACC (SEQ ID NO: 358) |
| | | HGL6.1223 | |
| 353 | HGL6.801 | | GCCAACAATCATATGAGAAAAAGCTCAACATCACTGATCATTTCAGGAATGCAA |
| | | | ATCAAAACCACAATGAGATACTATCACACATCAATCAGAATGGCT (SEQ ID NO: |
| | | | 359) |
| 354 | HGL6.802 | HGL6.118, | GAATCGAATGGAATCAACATCAAACGGAAAAAAACGGAATTATCGAATGGAATC |
| | | HGL6.590, | GAAGAGAATCATCGAATGGACC (SEQ ID NO: 360) |
| | | HGL6.1051, | |
| | | HGL6.1170, | |
| | | HGL6.1248, | |
| | | HGL6.1372 | |
| 355 | HGL6.804 | | AATCAAATGGAATGAAATCGAATGGAATTGAATCGAATGGAATGCAATAGAATG |
| | | | TCTTCAAATGGAATCGAATGGAAATTGGTGAAGTGGACGGGAGTG (SEQ ID NO: |
| | | | 361) |
| 356 | HGL6.805 | | TAACAGTACCAAAAAACAGTCATAATCTTCAAGAGCTTAAATTTAGCATGAAAG |
| | | | GAAGACATTCATCAAAGAATCACACAAAGGAATGTAAAATTAAATGGAGATTAG |
| | | | TGCCAGGAAAGAGC (SEQ ID NO: 362) |
| 357 | HGL6.808 | | TAATGGAATCAACATCAAACGGAAAAAAACGGAATTATCGAATGCAATCGAAGA |
| | | | GAATCATCGAATGGACC (SEQ ID NO: 363) |
| 358 | HGL6.813 | | AGCAACTTCAGCAAAGTCTCAGCATACAAAATCAATGTGCAAAAATCACACGCA |
| | | | TTCCTATACACCAATAACAGACAAACAGAGAGCC (SEQ ID NO: 364) |
| 359 | HGL6.815 | | GAATCAAATGGAATGGACTGTAATGGAATGGATTCGAATGGAATCGAATGGAGT |
| | | | GGACTCAAATGGAATG (SEQ ID NO: 365) |
| 360 | HGL6.816 | | AACAAGTGGACGAAGGATATGAACAGACACTTCTCAAGACATTTATGCAGCCAA |
| | | | CAGACACACGAAAAAATGCTCATCATCACTGGCCATCAG (SEQ ID NO: 366) |
| 361 | HGL6.819 | | AAACACACAAAGCAACAAAAGAACGAAGCAACAAAAGCATAGATTTATTGAAA |
| | | | TGAAAGTACATTCTACAGAGTGGGGGCAGGCT (SEQ ID NO: 367) |
| 362 | HGL6.820 | | ATACAACTAAAGCAAATATAAGCAACTAAAGCAACAGTACAACTAAAGCAAAA |
| | | | CAGAACAAGACTGCCAGGGCCTAGAAAAGCCAAGAAC (SEQ ID NO: 368) |
| 363 | HGL6.822 | | GCAATCGAATGGAATGGAATCGAACGGAATGGAATAAAATGGAAGAAAACTGG |
| | | | CAAGAAATGGAATCG (SEQ ID NO: 369) |
| 364 | HGL6.825 | | AGCAGCCAACAAGCATATGAAATAATGCTCCACAACACTCATCATCAGAGAAAT |
| | | | GCAAATCAAAACCAAAAT (SEQ ID NO: 370) |
| 365 | HGL6.826 | | TGGAACCGAACAAAGTCATCACCGAATGGAATTGAAATGAATCATAATCGAATG |
| | | | GAATCAAATGGCATCTTCGAATTGACTCGAATGCAATCATCCACTGGGCTT (SEQ |
| | | | ID NO: 371) |
| 366 | HGL6.827 | HGL6.829 | AACGGAATCACGCGGAATTATCGAATGGAATCGAAGAGAATCATCGAATGGACT |
| | | | CGAATGGAATCATCTAATGGAATGGAATGG (SEQ ID NO: 372) |
| 367 | HGL6.830 | | AGAACCATATTGAAGAGACAGAGTGATATATAAAACTGCTAACTCAAGCAGCACAAGAATTA |
| | | | AATGAATACCAAGAAAATACTTGGCCAG (SEQ ID NO: 373) |
| 368 | HGL6.831 | | AAAACAAACAACAACGACAAATCATGAGACCAGAGTTAAGAAACAATGAGACC |
| | | | AGGCTGGGTGTGGTG (SEQ ID NO: 374) |
| 369 | HGL6.833 | | AATCGAAAGGAATGCAATATTATTGAACAGAATCGAAAAGAATGGAATCAAATG |
| | | | GAATGGAACAGAGTGGAATGGACTGC (SEQ ID NO: 375) |
| 370 | HGL6.836 | | AAGGAATCGAATGGAAGTGAATGAAATTGAATCAACAGGAATGGAAGGGAATA |
| | | | GAATAGACTGTAATGGAATGGACTCG (SEQ ID NO: 376) |
| 371 | HGL6.837 | | AATGGACTCGAATGAAATCATCATCAAACGGAATCGAATGGAATCATTGAATGG |
| | | | AATGGAATGGAATCATCATGGAATGGAAACG (SEQ ID NO: 377) |
| 372 | HGL6.838 | | TTGACCAGAACACATTACACAATGCTAATCAACTGCAAAGGAGAATATGAACAG |
| | | | AGAGGAGGACATGGATATTTTGTG (SEQ ID NO: 378) |
| 373 | HGL6.839 | | AACCCGAGTGCAATAGAATGGAATCGAATGGAATGGAATGGAATGGAATGGAA |
| | | | TGGAATGGAGTC (SEQ ID NO: 379) |
| 374 | HGL6.843 | | AAGAGTATTGAAGTTGACATATCTAGACTGATCAAGAACAAAGACAAAAGGTAC |
| | | | AGATTATCAAGAAAATGAGCGGGCAAAGCAAGATGGCC (SEQ ID NO: 380) |
| 375 | HGL6.847 | | GAATGGAATTGAAAGGAATGGAATGCAATGGAATGGAATGGGATGGAATGGAA |
| | | | TGCAATGGAATCAACTCGATTGCAATG (SEQ ID NO: 381) |
| 376 | HGL6.849 | | GAAAAAAACGGAATTATCNAATTGAATCNAATANAATCATCNNNNNGACCANA |
| | | | NTGGAATCATCTAATGNAATGNAATGGAATAATCCATGGACTCNAATG (SEQ ID |
| | | | NO: 382) |
| 377 | HGL6.850 | | GAAAAAAACGGAATTATCGAATTGAATCGAATAGAATCATCGAACGGACCAGAA |
| | | | TGGAATCATCTAATGGAATGGAATGGAATAATCCATGGACTCGAATG (SEQ ID |
| | | | NO: 383) |
| 378 | HGL6.853 | | AACCACTGCTTAAGGAAATAAGAGAGAACACAAACAAATGGAAAAACGTTCCAT |
| | | | GCTCATGGATAGGAGAATCAATATCGTGAAAATGGCC (SEQ ID NO: 384) |
| 379 | HGL6.854 | | TATCGAATGGAATGGAAAGGAGTGGAGTAGACTCGAATAGAATGGACTGGAATG |
| | | | AAATAGATTCGAATGGAATGGAATGGAATGAAGTGGACTCG (SEQ ID NO: 385) |
| 380 | HGL6.855 | | GTATCAACATCAAACGGAAAAAAACGGAATTATCGAATGGAATCATCTAATGGA |
| | | | ATGGAATGGAATAATCCATGGACTCGAATG (SEQ ID NO: 386) |
| 381 | HGL6.856 | | TAAATGGAGACATCATTGAATACAATTGAATGGAATCATCACATGGAATCGAAT |
| | | | GGAATCATCGTAAATGCAATCAAGTGGAATCAT (SEQ ID NO: 387) |
| 382 | HGL6.857 | | GAATGGAATTGAAAGGTATCAACACCAAACGGAAAAAAAAACGGAATTATCGA |
| | | | ATGGAATCGAAGAGAATCATCGAACGGACC (SEQ ID NO: 388) |
| 383 | HGL6.858 | | AGCAATTTCAGCAAAGTCTCAGGATACAAAATCAATGTACAAATTCACAAGCAT |
| | | | TCTTATGGACCAACAACAG (SEQ ID NO: 389) |
| 384 | HGL6.860 | | AACCAAATTAGACAAATTGGAAATCATTACACATAACAAAAGTAATAAACTGTC |
| | | | AGCCTCAGTAGTATTCATTGTACATAAACTGGCC (SEQ ID NO: 390) |
| 385 | HGL6.861 | | TATTTTACCAGATTATTCAAGCAATATATAGACAGCTTAAAGCATACAAGAAGAC |
| | | | ATGTATAGATTTACATGCAAACACTGCACCACTTTACATAAGGGACTTGAGCAC |
| | | | (SEQ ID NO: 391) |
| 386 | HGL6.863 | | GGAATCGAATGGCATCAACATCAAACGGAAAAAAACGGAATTATCGAATGGAAT |
| | | | CGAATGGAATCATC (SEQ ID NO: 392) |
| 387 | HGL6.864 | | AAACAAAACACAGAAATGCAAAGACAAAACATAAAACGCAGCCATAAAGGACA |
| | | | TATTTTAGATAACTGGGGAAATTTGTATGGGCTGTGT (SEQ ID NO: 393) |
| 388 | HGL6.866 | HGL6.867 | AGGAAAAGAAAGAAATAGAAAATGCGAAATGGTAAGAAAAAACAGCATAATAA |
| | | | ACATTTGTATGGTGTTGATGGACAATGCATT (SEQ ID NO: 394) |
| 389 | HGL6.869 | | AATGGAATCAACATCAAACGGAATCAAACGGAATTATCGAATGGAATCGAAGAG |
| | | | AATCATCGAACGGACTCGAATGGAATCATCTAATGGAATGGAATGGAAG (SEQ |
| | | | ID NO: 395) |
| 390 | HGL6.872 | HGL6.1072, | AATCGAATGGAATCAGCATCAAACGGAAAAAAACGGAATTATCGAATGGAATCG |
| | | HGL6.1301 | AAGAGAATCATCGAATGGACC (SEQ ID NO: 396) |
| 391 | HGL6.877 | | AAAGAAATGGAATCGAAGAGAATGGAAACAAATGGAATGGAATTGAATGGAAT |
| | | | GGAATTGAATGGAATGGGAACG (SEQ ID NO: 397) |
| 392 | HGL6.878 | | AGAAAGAATCAAGAGGAAATGCAAGAAATCCAAAACACTGTAACAGATATGAT |
| | | | GAATAATGAGGTATGCACTCATCAGCAGACTCGACAT (SEQ ID NO: 398) |
| 393 | HGL6.879 | | AAACGGAATTATNNANTGGANNNNAAGNNAATCATCGAACGGANNNNANNGGA |
| | | | ATCATNTNNNNGAANGGAATGGAACAATCCATGGTNTNNNN (SEQ ID NO: 399) |
| 394 | HGL6.882 | HGL6.971 | AGCAACTTCAGCAAAGTTTCAGGATACAAAATCAATGTGCAAAAATCACAAGCA |
| | | | TTCTTATACACCAACAACAGACAAACAGAGAGCC (SEQ ID NO: 400) |
| 395 | HGL6.884 | | AGACAGTCAGACAATCACAAAGAAACAAGAATGAAAATGAATGAACAAAACCT |
| | | | TCAAGAAATATGGGATTATGAAGAGGCCAAATGT (SEQ ID NO: 401) |
| 396 | HGL6.885 | | ATCATAACGACANGANCAAATTCACACACAACAATNNNNACNNNAAANNCAAA |
| | | | TGGGTTAAATNNTNCAATTAAAGGATGCAGACGGGCAAATTGGATA (SEQ ID NO: |
| | | | 402) |
| 397 | HGL6.891 | | ATCATAANGACAAGANCAAATTCACACACAACAATNNNNACNNNAAANNCAAA |
| | | | TGGGTTNAATGNTNCAATTAAAGGATGCAGACGGNCAAATTGGATA (SEQ ID NO: |
| | | | 403) |
| 398 | HGL6.895 | | GAATGGAATCGAATGGATTGATATCAACTGGAATGGAATGGAAGGGAATGGAAT |
| | | | GGAATGGAATTGAACCAAATGTNNNNGNCTTGAATGGAATG (SEQ ID NO: 404) |
| 399 | HGL6.898 | | GAATCAACATCAAACGGAAAAAAACGGAATTATCGAATGGAATCGAAGAGAAT |
| | | | CATCGAATGGACC (SEQ ID NO: 405) |
| 400 | HGL6.904 | | ATGGAATCAACATCAAACGGAATCAAACGGAATTATCGAATGGAATCAAAGAGA |
| | | | ATCATCGAACGGACTCGAATGGAATCATCTAATGGAATGGAATGGAAGAATCCA |
| | | | TGGACTCGAATGCAATCATCATCGAAT (SEQ ID NO: 406) |
| 401 | HGL6.905 | | GGAATGGAATGGAATGGAGCNGAATNGAANGGANNNNANTCAAATGGAATGC |
| | | | (SEQ ID NO: 407) |
| 402 | HGL6.906 | | AACATACGCAAATCAATAAATGTAATCCAGCATATAAACAGAACCAAAGACAA |
| | | | AAACCACATGATTATCTCAATAGATGCAGAAAAGGCC (SEQ ID NO: 408) |
| 403 | HGL6.911 | | AAACGATTGGACAGGAATGGAATCACCATCGAATGGAAACGAATGGAATCTTCG |
| | | | AATGGAATTGAATGAAATTATTGAACGGAATCAAATAGAATCATCATTGAACAG |
| | | | AATCAAATTGGATCAT (SEQ ID NO: 409) |
| 404 | HGL6.912 | | AAAAGATGCAAAAGTAGCAAATGCAATGTTAAAACAAGCAAAGAAAGAATCAG |
| | | | GTGGACCACATAGTGCAGTGCTTCTC (SEQ ID NO: 410) |
| 405 | HGL6.914 | | AACAATAAACAAACTCCAACTAGACACAATAGTCAAATTGCTGAAAATGAAATA |
| | | | TAAAGGAACAATCTCGATGGTAGCCCAAGGA (SEQ ID NO: 411) |
| 406 | HGL6.915 | HGL6.916 | AGTCAATAACAAGAAGACAAACAACCCAATTACAAAATGGGATATGAATTTAAT |
| | | | AGATGTTACTCCAAGGAAGATACACAAATGGCCAAC (SEQ ID NO: 412) |
| 407 | HGL6.919 | | AAAACACCTAGGAATACAGATAACAAGGGACATTAACTACCTCTTAAAGAGAAC |
| | | | TACAAACCACTGCTCAAGGAAATGAGAGAGGACACAAACACATGGAAAAACAT |
| | | | TCCATCCTCATGGATAGGAAGAATCAATATTGTGAAAATGGCC (SEQ ID NO: 413) |
| 408 | HGL6.921 | | GATATATAAACAAGAAAACAACTAATCACAACTCAATATCAAAGTGCAATGATG |
| | | | GTGCAAAATGCAAGTATGGTGGGGACAGAGAAAGGATGC (SEQ ID NO: 414) |
| 409 | HGL6.923 | | ACACATATCAAACAAACAAAAGCAATTGACTATCTAGAAATGTCTGGGAAATGG |
| | | | CAAGATATTACA (SEQ ID NO: 415) |
| 410 | HGL6.924 | | GGAATCATCATATAATGGAATCGAATGGAATCAACATCAAATGGAATCAAATGG |
| | | | AATCATTGAACGGAATTGAATGGAATCGTCAT (SEQ ID NO: 416) |
| 411 | HGL6.926 | | CCCAACTTCAAATTATACTACAAGGCTACAGTAATCAAAAAAGCATAGTACTATT |
| | | | ACAAAAACAGACACACAGGCCAATGGAATACAAT (SEQ ID NO: 417) |
| 412 | HGL6.927 | | AAACGCAGAAACAAATCAACGAAAGAACGAAGCAATGAAAGACAAAGCAACAA |
| | | | AAGAATGGAGTAAGAAAGCACACTCCACAAAGTGGAAGCAGGCTGGGACA (SEQ |
| | | | ID NO: 418) |
| 413 | HGL6.928 | | AACTAACACAAGAACAGAAAACCAAACATCACATGTTCTCACTCATAAGCGGGA |
| | | | GCTGAACAATGAGAACACACGGACACAGGGAGAGGAACATG (SEQ ID NO: 419) |
| 414 | HGL6.929 | | GCCACAATTTTGAAACAACCATAATAATGAGAATACACAAGACAACTCCAATAA |
| | | | TGTGGGAAGACAAACTTTGCAATTCACATCATGGC (SEQ ID NO: 420) |
| 415 | HGL6.933 | | AATGGAATCAACATCAAACGGAATCAAATGGAATTATCGAATGGAATCGAAGAG |
| | | | AATCATCGAATTGTCACGAATGGAATCATCTAATGGAATGGAATGGAATAATCC |
| | | | ATGGCCCCTATGC (SEQ ID NO: 421) |
| 416 | HGL6.934 | HGL6.935 | TAAACAGAACCAAAGACAAAAATCACATGATTATCTCAATAGATGCAGAAAAGG |
| | | | CC (SEQ ID NO: 422) |
| 417 | HGL6.937 | | ATCAACAGACAACAGAAACAAATCCACAAAGCACTTAGTTATTAGAACTGTCAT |
| | | | ACAGACTGTACAACAACCACATTTACCAT (SEQ ID NO: 423) |
| 418 | HGL6.938 | | AATGGACTCGAATGAAATCATCATCAAACAGAATCGAATGGAATCATCTAATGG |
| | | | AATGGAATGGCATAATCCATGGACTCGAATG (SEQ ID NO: 424) |
| 419 | HGL6.939 | | TAAAATGAAACAAATATACAACACGAAGGTTATCACCAGAAATATGCCAAAACT |
| | | | TAAATATGAGAATAAGACAGTCTCAGGGGCCACAGAG (SEQ ID NO: 425) |
| 420 | HGL6.940 | | AAAATACAGCGTTATGAAAAGAATGAACACACACACACACACACACACACAGA |
| | | | AAATGT (SEQ ID NO: 426) |
| 421 | HGL6.942 | | TACTCTCAGAAGGGAAGCAGATATTCAGCATAAATCATATTGTTTGTACAAAGA |
| | | | GTCTGGGCATGGTGAATGACACT (SEQ ID NO: 427) |
| 422 | HGL6.943 | | CAAACAAATAGGTACCAAACAAATAACAACATAAACCTGACAACACACTTATTT |
| | | | ACAAGAGACATCCCTTATATGAAAGGGTACAGAAAAGTCGATGGTAAGATGATG |
| | | | GGGAAAGGTATACCAACCACTAGCAGAAGG (SEQ ID NO: 428) |
| 423 | HGL6.944 | | TGGAATCGAATGGAATCAATATCAAACGGAAAAAAACGGAATTATCGAATGGAA |
| | | | TCGAAAAGAATCATCGAATGGGCCCGAATGGAATCATCT (SEQ ID NO: 429) |
| 424 | HGL6.945 | | ACAAATGGAATCAACAACGAATGGAATCGAATGGAAACGCCATCGAAAGGAAA |
| | | | CGAATGGAATTATCATGAAATTGAAATGGATG (SEQ ID NO: 430) |
| 425 | HGL6.947 | | GACAAGAGTTCAGAAAGGAAGACTACACAGAAATACGCATTTTAAAGTCACTGA |
| | | | CATGGAGATGACACTTAAAACCATGAACATGGATGGG (SEQ ID NO: 431) |
| 426 | HGL6.956 | | AAAATAAACGCAAATTAAAATCACAAGATACCAACACATTCCCACGGCTAAGTA |
| | | | CGAAGAACAAGGGCGAATGGTCAGAATTAAGCTCAAACCT (SEQ ID NO: 432) |
| 427 | HGL6.957 | | TAAACTGACACAAACACAGACACACAGATACACACATACATACAGAAATACACA |
| | | | TTCACACACAGACCTGGTCTTTGGAGCCAGAGATG (SEQ ID NO: 433) |
| 0 | HGL6.958 | | GATCAATAAATGTAATTCATCATATAAACAGAGAACTAAAGACAAAAACACATG |
| | | | ATTATCGCAATACATGCAGAAAAGGCC (SEQ ID NO: 434) |
| 429 | HGL6.962 | | AGGACATGAATAGACAATTCTCAAAAGAAGATACACAAGTGGCAAACAAACAC |
| | | | ATGAAAAAAGACTCAACATTAGTAATGACCATGGAAATGCAAATC (SEQ ID NO: |
| | | | 435) |
| 430 | HGL6.963 | | ACATCAAACGGAAAAAAACGGAATTATCGAATGGAATCGAAGAGAATCATCGA |
| | | | ATGGACC (SEQ ID NO: 436) |
| 431 | HGL6.965 | | AATGGACTCGAATAGAATTGACTGGAATGGAATGGACTCGAATGGAATGGAATG |
| | | | GAATGGAAGGGACTCG (SEQ ID NO: 437) |
| 432 | HGL6.966 | | AAGAAAGACAGAGAACAAACGTAATTCAAGATGACTGATTACATATCCAAGAAC |
| | | | ATTAGATGGTCAAAGACTTTAAGAAGGAATACATTCAAAGGCAAAACGTCACTT |
| | | | ACTGATTTTGGTGGAGTTTGCCACATGGAC (SEQ ID NO: 438) |
| 433 | HGL6.967 | | AACATAATCCATCAAATAAACAGAACCAAAGACAAAAACCACATGATTATCTCA |
| | | | ATAGATGCAGAAAAGGCCTTC (SEQ ID NO: 439) |
| 434 | HGL6.969 | | GAATGGAATCGAATGGAATGAACATCAAACGGAAAAAAACGGAATTATCGAAT |
| | | | GGAATCAAAGAGAATCATCGAATGGACCCG (SEQ ID NO: 440) |
| 435 | HGL6.972 | | ATGGACTCGAATGTAATAATCATTGAACGGAATCGAATGGAATCATCATCGGAT |
| | | | GGAAACGAATGGAATCATCATCGAATGGAATCGAATGGGATC (SEQ ID NO: 441) |
| 436 | HGL6.974 | | GAATGGAATCAACATCAAACGGAATCAAACGGAATTATCGAATGGAATCGAAGA |
| | | | GAATCATCGAATGGCCACGAATGGAATCATCTAATGGAATGGAATGGAATAATC |
| | | | CATGG (SEQ ID NO: 442) |
| 437 | HGL6.975 | | GAAATGGAATGGAAAGGAATAAAATCAAGTGAAATTGGATGGAATGGATTGGA |
| | | | ATGGATTGGAATG (SEQ ID NO: 443) |
| 438 | HGL6.978 | | AAACGGAAAAAAAACGGAATTATCGAATGGAATCGAAGAGAATCATCGAACGA |
| | | | ACCAGAATGGAATCATCTAATGGAATGGAATGGAATAATCCATGG (SEQ ID NO: |
| | | | 444) |
| 439 | HGL6.981 | | ATTAACCCGAATAGAATGGAATGGAATGGAATGGAACGGAACGGAATGGAATG |
| | | | GAATGGAATGGAATGGAATGGATCG (SEQ ID NO: 445) |
| 440 | HGL6.982 | | GCAAAACACAAACAACGCCATAAAAAACTGGGCAAAGGATATGAACAGACATT |
| | | | TTTCAAAACAAAACATACTTATGGCCAAC (SEQ ID NO: 446) |
| 441 | HGL6.984 | | AACATCAAACGGAAAAAAACGGAATTATCGTATGGAATCGAAGAGAATCATCGA |
| | | | ATGGACC (SEQ ID NO: 447) |
| 442 | HGL6.985 | | AAATCAATAAATGTAATTCAGCATATAAACAGAACCAAAGACAAAAACCACAT |
| | | | GATTATCTCAATAGATGCAGAAAAGGCCTTT (SEQ ID NO: 448) |
| 443 | HGL6.986 | | AGAATCAAATGGAATTGAATCGAATGGAATCGAATGGATTGGAAAGGAATAGA |
| | | | ATGGAATGGAATGGAATG (SEQ ID NO: 449) |
| 444 | HGL6.988 | | GAATAGAATTGAATCATCATTGAATGGAATCGAGTAGAATCATTGAAATCGAAT |
| | | | GGAATCATCATCGAATGGAATTGGGTGGAATC (SEQ ID NO: 450) |
| 445 | HGL6.989 | | CACCGAATAGAATCGAATGGAACAATCATCGAATGGACTCAAATGGAATTATCC |
| | | | TCAAATGGAATCGAATGGAATTATCGAATGCAATCGAATAGAATCATCGAATAG |
| | | | ACTCGAATGGAATCATCGAATGGAATGGAATGGAACAGTC (SEQ ID NO: 451) |
| 446 | HGL6.992 | HGL6.1286 | AAATCATCATCGAATGGAATCGAATGGTATCATTGAATGGAATCGAATGGAATC |
| | | | ATCATCAGATGGAAATGAATGGAATCGTCAT (SEQ ID NO: 452) |
| 447 | HGL6.997 | | GAATGGAATCGAAAGGAATAGAATGGAATGGATCGTTATGGAAAGACATCGAAT |
| | | | GGAATGGAATTGACTCGAATGGAATGGACTGGAATGGAACG (SEQ ID NO: 453) |
| 448 | HGL6.998 | | GAATAGAATTGAATCATCATTGAATGGAATCGAGTAGAATCATTGAAATCGAAT |
| | | | GGAATCATCATCGAATGGAATTGGGTGGAATC (SEQ ID NO: 454) |
| 449 | HGL6.1001 | | GAAAGGAATAGAATGGAATGGATCGTTATGGAAAGACATCGAATGGGATGGAA |
| | | | TTGACTCGAATGGATTGGACTGGAATGGAACGGACTCGAATGGAATGGACTGGA |
| | | | ATG (SEQ ID NO: 455) |
| 450 | HGL6.1003 | | TGGATTTCAGATATTTAACACAAAATAGTCAAAGCAGATAAATACTAGCAACTT |
| | | | ATTTTTAATGGGTAACATCATATGTTCGTGCCTT (SEQ ID NO: 456) |
| 451 | HGL6.1004 | | ACAGCAGAAAACGAACATCAGAAAATCACTCTACATGATGCTTAAATACAGAGG |
| | | | GCAAGCAACCCAAGAGAAAACACCACTTCCTAAT (SEQ ID NO: 457) |
| 452 | HGL6.1011 | | AACATACACAAATCAATAAACGTAATCCAGCTTATAAACAGAACCAAAGACAAA |
| | | | AACCACATGATTATCTCAATAGATGCGGAAAAGGCC (SEQ ID NO: 458) |
| 453 | HGL6.1012 | | ACATCAAACGGAATCAAACGGAATTATCGAATGGAATCGAAAAGAATCATCGAA |
| | | | CGGACTCGAATGGAATCATCTAATGGAATGGAATGGAAG (SEQ ID NO: 459) |
| 454 | HGL6.1013 | | ATCGAATGGAATCAACATCAAACGGAAAAAAACGGAATTATCAAATGGAATCGA |
| | | | AGAGAATCATCGAATGGACC (SEQ ID NO: 460) |
| 455 | HGL6.1014 | | GAATAATCATTGAACGGAATCGAATGGAAACATCATCGAATGGAAACGAATGGA |
| | | | ATCATCATCGAATGGAAATGAAAGGAGTCATC (SEQ ID NO: 461) |
| 456 | HGL6.1015 | | CATCAAACGGAATCAAACGGAATTATCGAATGGAATCGAAAAGAATCATCGAAC |
| | | | GGACTCGAATGGAATCATCTAATGGAATGGAATGGAAGAATCCATGGACTCGAA |
| | | | TG (SEQ ID NO: 462) |
| 457 | HGL6.1016 | | TCCAGTCGATCATCATATAGTCAGCACTTATCATACACCAAGCCGTGTGCAAGGA |
| | | | AAGGGAATACAACCATGAACATGATAGATGGATGGTT (SEQ ID NO: 463) |
| 458 | HGL6.1017 | | ACAAACCACTGCTCAAGGAAATAAGGACACAAACAAATGGAACAACATTCCGTG |
| | | | CTCATGGATAGGAAGAATCAATATCGTGAAAATGGCCATACT (SEQ ID NO: 464) |
| 459 | HGL6.1019 | | ACAAAATTGATAGACCACTAGCAAGACTAATAAAGAAGAAAAGAGAGAAGAAT |
| | | | CATTACCATTCAGGACATAGGCATGGGCAAGGAC (SEQ ID NO: 465) |
| 460 | HGL6.1024 | | AAACGGAATCAAACGGAATTATCGAATGGAATCGAAGAGAATCATCGAATGGAC |
| | | | TCGAATGGAATCATCTAATGGAATGGAATGGAAGAATCCATGG (SEQ ID NO: 466) |
| 461 | HGL6.1026 | | ATACACAAATCAATAAATGTAATCCAGCATATAAACAGAACCAAAGACAAAAA |
| | | | CCATATGATTATCTCAATGGATGCAGAAAAGGCC (SEQ ID NO: 467) |
| 462 | HGL6.1027 | | AATNGAATAGAATCATCGAATGGACTCGAATGGAATCATCGANNNTANTGATGG |
| | | | AACAGTC (SEQ ID NO: 468) |
| 463 | HGL6.1030 | | TGGAATGGAATCATCGCATAGAATCGAATGGAATTACCATCGAATGGGATCGAA |
| | | | TGGTATCAACATCAAACGCAAAAAAACGGAATTATCGAATGGAATCGAAGAGAA |
| | | | TCTTCGAACGGACCCG (SEQ ID NO: 469) |
| 464 | HGL6.1031 | | GAATTGAATTGAATGGAATGGAATGCAATGGAATCTAATGAAACGGAAAGGAA |
| | | | AGGAATGGAATGGAATGGAATG (SEQ ID NO: 470) |
| 465 | HGL6.1033 | | AACAGAATGGAATCAAATCGAATGAAATGGAATGGAATAGAAAGGAATGGAAT |
| | | | GAAATGGAATGGAAAGGATTCGAATGGAATGCAATCG (SEQ ID NO: 471) |
| 466 | HGL6.1034 | | ATGGAATGGAATGGAATGGAATTAAATGGAATGGAAAGGAATGGAATCGAATG |
| | | | GAAAGGAATC (SEQ ID NO: 472) |
| 467 | HGL6.1037 | HGL6.1245 | GTCGAAATGAATAGAATGCAATCATCATCAAATGGAATCCAATGGAATCATCAT |
| | | | CAAATAGAATCGAATGGAATCATCAAATGGAATCGAATGGAGTCATTG (SEQ ID |
| | | | NO: 473) |
| 468 | HGL6.1039 | | TGGAATTATCGAAAGCAAACGAATAGAATCATCGAATGGACTCGAATGGAATCA |
| | | | TCGAATGGAATGGAATGGAACAG (SEQ ID NO: 474) |
| 469 | HGL6.1045 | | AAAGGAATGGAATGCAATGGAATGCAATGGAATGCACAGGAATGGAATGGAAT |
| | | | GGAATGGAAAGGAATG (SEQ ID NO: 475) |
| 470 | HGL6.1046 | | AATCTAATGGAATCAACATCNAACGGAAAAAAACGGAATTATCGAATGGAATCN |
| | | | AAGAGAATCATCNAATGGACC (SEQ ID NO: 476) |
| 471 | HGL6.1047 | | TACACAACAAAAGAAATACTCAACACAGTAAACAGACAACCTTCAGAACAGGA |
| | | | GAAAATATTTGCAAATACATCTAACAAAGGGCTAATATCCAGAATCT (SEQ ID |
| | | | NO: 477) |
| 472 | HGL6.1048 | | NGCAATCNTAGTNTCAGATAAAACAGACATTAAACCAACAAAGATCAAAAGAG |
| | | | ACAAAGAAGGCCANTAC (SEQ ID NO: 478) |
| 473 | HGL6.1052 | | GAATCGAATGGAATCAACATCAAACGGAAAAAAACGGAATTATCGAATGGAATC |
| | | | NAAAAGAATCATCNAATGGACC (SEQ ID NO: 479) |
| 474 | HGL6.1055 | | ACAGTTAACAAAAACCGAACAATCTAATTACGAAATGAACAAAAGATATGAACA |
| | | | GACATTTCACCCGAGAGTATACAGGGGCCAGGCATGGT (SEQ ID NO: 480) |
| 475 | HGL6.1056 | | AATGGAATCGAATGGAATGCAATCCAATGGAATGGAATGCAATGCAATGGAATG |
| | | | GAATCGAACGGAATGCAGTGGAAGGGAATGG (SEQ ID NO: 481) |
| 476 | HGL6.1057 | | GAACACAGAAAAATTTCAAAGGAATAATCAACAGGGATTGATAACTAACTGGAT |
| | | | TTAGAGAGCCAAGGCAAAGAGAATCAAAGCACAGGGCCTGAGTCGGAG (SEQ ID |
| | | | NO: 482) |
| 477 | HGL6.1058 | | TATACCACACAAATGCAAAAGATTATTAGCAACAATTATCAACAGCAATATGTC |
| | | | AACAAGTTGACAAACCTAGAGGACATGGAT (SEQ ID NO: 483) |
| 478 | HGL6.1061 | | CACCATGAGTCATTAGGTAAATGCAAATCAAAACCACAATGAAATACTTCACAC |
| | | | CCATGAAGATGGCTATAATAAAAAAACAGACA (SEQ ID NO: 484) |
| 479 | HGL6.1067 | | AGTTGAATAGAACCAATCCGAATGAAATGGAATGGAATGGAACGGAATGGAATT |
| | | | GAATGGAATGGAATGGAATGCAATGGA (SEQ ID NO: 485) |
| 480 | HGL6.1069 | | AAGTAATAAGACTGAATTAGTAATACAAAGTGTCTCAACAAAGAAAATTGCGGG |
| | | | ACTGTTCATGCTCATGGACAGGAAGAATCAATATCATGAAAATGGCC (SEQ ID |
| | | | NO: 486) |
| 481 | HGL6.1070 | | AACTCGATTGCAATGGAATGTAATGTAATGGAATGGAATGGAATTAACGCGAAT |
| | | | AGAATGGAATGGAATGTAATGGAACGGAATGGAATG (SEQ ID NO: 487) |
| 482 | HGL6.1074 | | AAGCGGAATAGAATTGAATCATCATTGAATGGAATCGAGTAGAATCATTGAAAT |
| | | | CGAATGGAATCATAGAATGGAATCCAAT (SEQ ID NO: 488) |
| 483 | HGL6.1076 | | AAAGGAAAACTACAAAACACTGCTGAAAGAAATCATTGACAACACAAACAAAT |
| | | | GGAAACACATCCCAAGATCATGGGTGGGTGGAATCAAT (SEQ ID NO: 489) |
| 484 | HGL6.1077 | | AATGGAATCNAAAGGAATAGAATGGAATGGATCGTTATGGAAAGATATCGAATG |
| | | | GAATGGAATTGACTCGAATGGAATGGACTGGAATGGAACG (SEQ ID NO: 490) |
| 485 | HGL6.1078 | | TAACGGAATAATCATCGAACAGAATCAAATGGAATCATCATTGAATGGAATTGA |
| | | | ATGGAATCTTCGAATAGACATGAATGGACCATCATCG (SEQ ID NO: 491) |
| 486 | HGL6.1084 | | AAAGACCGAAACAACAACAGAAACAGAAACAAACAACAATAAGAAAAAATGTT |
| | | | AAGCAAAACAAATGATTGCACAACTTACATGATTACTGAGTGTTCTAATGGT |
| | | | (SEQ ID NO: 492) |
| 487 | HGL6.1085 | | AAGATTTAAACATAAGACCTAAAACGACAAAAATCCTAGGAGAAAACCTAAGCA |
| | | | ATACCATTCAGGACATAGGCATGGGCAAAGACTTCATG (SEQ ID NO: 493) |
| 488 | HGL6.1090 | | AGAAACAGCCAGAAAACAATTATTACCTACAGCATTAAAACTATTCAAATGACA |
| | | | GCATATTTTTCAGCAGAAATCATGAAGGCCAGAAGGACGTGTCAT (SEQ ID NO: |
| | | | 494) |
| 489 | HGL6.1092 | | ATGTACACAAATCAATAAATGCAGTCCAGCATATAAACAGAACCAAACACAAA |
| | | | AACCACATGATTATCTCAATAGATGCAGAAAAGGCCTTT (SEQ ID NO: 495) |
| 490 | HGL6.1093 | | AGCAACTTCAGCAAAGTCTCAGGACACAAAATCAATGTGCAAAAATCACAAGCA |
| | | | TTCTTATACACCAATAACAGACAAACAGAGAGCC (SEQ ID NO: 496) |
| 491 | HGL6.1094 | | TTGAATCGAATGGAATCGAATGGATTGGAAAGGAATAGAATGGAATGGAATGGA |
| | | | ATTGACTCAAATGGAATG (SEQ ID NO: 497) |
| 492 | HGL6.1097 | HGL6.1241 | AACGGAATCAAACGGAATTATCGAATGGAATCGAATAGAATCATCGAACGGACT |
| | | | CGAATGGAATCATCTAATGGAATGGAATGGAAG (SEQ ID NO: 498) |
| 493 | HGL6.1098 | | AACATCACTGATCATTAGAAACACACAAATCAAAACCACAATAAGATACCATCT |
| | | | AACACCAGTCACAATGGCTATT (SEQ ID NO: 499) |
| 494 | HGL6.1100 | | TAAGCAATTTCAGCAGTCTCAGGATACAAAATCAATGTGCAAAAATCACAAGCA |
| | | | TTCTTATACACCAACAACAGACAAACAGAGAGCCAAATCG (SEQ ID NO: 500) |
| 495 | HGL6.1101 | | AGAAAAAAACAAACAGCCCATTAAAAGGTAGACAAAGGACATGAACACTTTTCA |
| | | | AAAGAAGACATACATGTGGCCAAACAGCATG (SEQ ID NO: 501) |
| 496 | HGL6.1103 | | ATTGGAATGGAACGGAACAGAACGGAATGGAATGGAATAGAATGGAATGGAAT |
| | | | GGAATGGTATGGAATGGAATGGAATGGTACG (SEQ ID NO: 502) |
| 497 | HGL6.1104 | | AGAGCATCCACAAGGCCCAATTCAAAGAATCTGAAATAATGTATTGTTACTGCA |
| | | | ACAGTTGTGAGTACCAGTGGCATCAG (SEQ ID NO: 503) |
| 498 | HGL6.1107 | | AATCCACAAAGACAACAGAAGAAAAGACAACAGTAGACAAGGATGTCAACCAC |
| | | | ATTTTGGAAGAGACAAGTAATCAAACACATGGCA (SEQ ID NO: 504) |
| 499 | HGL6.1109 | | AAACAGAACCACAGATATCTGTAAAGGATTACACTATAGTATTCAACAGAGTAT |
| | | | GGAACAGAGTATAGTATTCAACAGAGTATGCAAAGAAACTAAGGCCAGAAAG |
| | | | (SEQ ID NO: 505) |
| 500 | HGL6.1110 | | AGCAAACAAACAAACAAACAAACAAACTATGACAGGAACAAAACGTCACATAT |
| | | | CAACATTAACAAAGAATGTAAACAGCCTAAATGCTTCACTTAAAAGTTATAGAC |
| | | | AGGGGCTGGGCATGGTGGCTCACGCC (SEQ ID NO: 506) |
| 501 | HGL6.1111 | | AAAAGTACAGAAGACAACAAAAAATGAGAGAGAGAAAGATAACAGACTATAGC |
| | | | AGCATTGGTGATCAGAGCCACCAG (SEQ ID NO: 507) |
| 502 | HGL6.1114 | | TACAAGAAAATCACAGTAACATTTATAAAACACAGAAGTGTGAACACACAGCTA |
| | | | TTGACCTTGAAAACAGTGAAAGAGGGTCAGCTGTAGAACTAAGACATAAGCAAA |
| | | | GTTTTTCAATCAAGAATACATGGGTGGCC (SEQ ID NO: 508) |
| 503 | HGL6.1116 | | GAATCGAATGGAATCAACATCAAACGGAAAAAAACGGAATTATCGAATGGAATC |
| | | | GAAAAGAATCATCGAACGGACTCGAATGGAATCATCTAATGGAATGGAATGGAA |
| | | | GAATCCATGG (SEQ ID NO: 509) |
| 504 | HGL6.1117 | | AATGGAATCGAATGGAATCATCATCAAATGGAATCTAATGGAATCATTGAACGG |
| | | | AATTGGATGGAATCGTCAT (SEQ ID NO: 510) |
| 505 | HGL6.1118 | | AACGGAATCAAACGGAATTATCGAATGGAATCGAAGAGAATCATCGAATGGCCA |
| | | | CGAATGGAATCATCTAATGGAATGGAATGGAATAATCCATGGACCCGAATG |
| | | | (SEQ ID NO: 511) |
| 506 | HGL6.1121 | | CAACATCAAACGGAAAAAAACGGAATTATCGAATGGAATCGAAGAGAATCATC |
| | | | GAATGGACC (SEQ ID NO: 512) |
| 507 | HGL6.1122 | | CACAACCAAAGCAATGAAAGAAAAGCACAGACTTATTGAAATGAAAGTACACA |
| | | | CCACAGAATGGGAGCAGGCTCAAGCAAGC (SEQ ID NO: 513) |
| 508 | HGL6.1123 | HGL6.1229 | ATCAAAGGGAATCAAGCGGAATTATCGAATGGAATCGAAGAGAATCATCGAATG |
| | | | GACTCGAATGGAATCATGTGATGGAATGGAATGGAATAATCCACGGACT (SEQ ID |
| | | | NO: 514) |
| 509 | HGL6.1125 | | AAGAAACAATCAAAAGGAAGTGCTAGAAATAAAACACACTGTAATAGAAAAGA |
| | | | AGAATGCCTTATGGGCTTATCAATAGACTAGACATGGCCAGG (SEQ ID NO: 515) |
| 510 | HGL6.1127 | | AGATAAGAATAAGGCAAACATAGTAATAGGGAGTTCATGAATAACACACGGAA |
| | | | AGAGAACTTACAGGGCTGTGATCAGGAAACG (SEQ ID NO: 516) |
| 511 | HGL6.1128 | | GGAATCGAATGGAATCAATATCAAACGGAGAAAAACGGAATTATCGAATGGAAT |
| | | | CGAAGAGAATCATCGAATGGACC (SEQ ID NO: 517) |
| 512 | HGL6.1130 | | TCAGACCATAGCAGATAACATGCACATTAGCAATACGATTGCCATGACAGAGTG |
| | | | GTTGGTG (SEQ ID NO: 518) |
| 513 | HGL6.1132 | | AGGAATGGACACGAACGGAATGCAATCGAATGGAATGGAATCTAATAGAAAGG |
| | | | AATTGAATGAAATGGACTGG (SEQ ID NO: 519) |
| 514 | HGL6.1133 | | GGAAGGGAATCAAATGCAACAGAATGTAATGGAATGGAATGCAATGGAATGCA |
| | | | ATGGAATGGAATGGAATGCAATGGAATGG (SEQ ID NO: 520) |
| 515 | HGL6.1138 | | AAATTGGATTGAATCGAATCGAATGGAAAAAATGAAATCAAATGAAATTGAATG |
| | | | GAATCGAAATGAATGTAAACAATGGAATCCAATGGAATCCAATGGAATCGAATC |
| | | | AAATGGTTTTGAGTGGCGTAAAATG (SEQ ID NO: 521) |
| 516 | HGL6.1139 | | AAGGATTCGAATGGAATGCAATCGAATGGAATGGAATCGAACGGAATGGAATA |
| | | | AAATGGAAGAAAACTGGCAAGAAATGGAATCG (SEQ ID NO: 522) |
| 517 | HGL6.1141 | | GAAAAATCATTGAACGGAATCGAATGGAATCATCATCGGATGGAAACGAATGGA |
| | | | ATCATCATCGAATGGAAATGAAAGGAGTCATC (SEQ ID NO: 523) |
| 518 | HGL6.1147 | | GGTTCAACTTACAATATTTTGACTTGACAACAGTGCAAAAGCAATACACGATTAG |
| | | | TAGAAACACACTTCCAATGCCCATAGGACCATTCTGC (SEQ ID NO: 524) |
| 519 | HGL6.1150 | | GGAATCGAATGGAATCAACATCAAACGGAGAAAAACGGAATTATCGAATGGAA |
| | | | TCGAAGAGAATCATCGAATGGACC (SEQ ID NO: 525) |
| 520 | HGL6.1152 | | TAACCTGATTTGCCATAATCCACGATACGCTTACAACAGTGATATACAAGTTACA |
| | | | TGAGAAACACAAACATTTTGCAAGGAAACTGTGGCCAGATG (SEQ ID NO: 526) |
| 521 | HGL6.1153 | | TAACTACTCACAGAACTCAACAAAACACTATACATGCATTTACCAGTTTATTATA |
| | | | AAGATACAAGTCAGGAACAGCCAAATGGAAGAAATGTAAATGGCAAG (SEQ ID |
| | | | NO: 527) |
| 522 | HGL6.1155 | | GCTCAAAGAAATCAGAAATGACACAAGCAAATGGAAAAACATGCCATGTTCATG |
| | | | AATATGAAGAATCAATATTGTTAAAATGGCCATACTGCTCA (SEQ ID NO: 528) |
| 523 | HGL6.1157 | | AAAGAAATGTCACTGCGTATACACACACACGCACATACACACACCATGGAATAC |
| | | | TACTCAGCTATACAAAGGAATGAAATAATCCACAGCCAC (SEQ ID NO: 529) |
| 524 | HGL6.1159 | | GAATAGAACAGAATGGAATCAAATCGAATGAAATGGAATGGAATAGAAAGGAA |
| | | | TGGAATGAAATGGAATGGAAAGGATTCGAATGGAATG (SEQ ID NO: 530) |
| 525 | HGL6.1162 | | TGAACGGAATCGAATGGAATCATCATCGGATGGAAACGAATGGAATCATCATCG |
| | | | AATGGAAATGAAAGGAGTCATC (SEQ ID NO: 531) |
| 526 | HGL6.1165 | | GAATAGAACGAAATGGAATGGAATGGAATGGAATGGAAAGGAATGGAATGGAA |
| | | | TGGAACG (SEQ ID NO: 532) |
| 527 | HGL6.1166 | | AACGTGACATACATACAAAAAGTTTTTAGAGCAAGTGAAATTTTAGCTGCTATAT |
| | | | GTTAATTGGTGGTAATCCC (SEQ ID NO: 533) |
| 528 | HGL6.1169 | | GGAATAACAACAACAACAACCAAAAGACATATAGAAAACAAACAGCACGATGG |
| | | | CAGATGTAAAGCCTACC (SEQ ID NO: 534) |
| 529 | HGL6.1174 | | GACAAAAAGAATCATCATCGAATAGAATCAAATGGAATCTTTGAATGGACTCAA |
| | | | AAGGAATATCGTCAAATGGAATCAAAAGCCATCATCGAATGGACTGAAATGGAA |
| | | | TTATCAAATGGACTCG (SEQ ID NO: 535) |
| 530 | HGL6.1175 | | GTAACAAAACAGACTCATAGACCAATAGAACAGAATAGAGAATTCAGAAATAA |
| | | | GACTGCACTTCTATGACCATGTGATCTTAGACAAACCT (SEQ ID NO: 536) |
| 531 | HGL6.1176 | | AGATAAAAAGAACAGCAGCCAAAATGACAAAAGCAAAAAGCAAAATCGTGTTA |
| | | | GAGCCAGGTGTGGTGATGTGTGCT (SEQ ID NO: 537) |
| 532 | HGL6.1178 | | GCAATCTCAGGATACAAAATCAATGTGCAAAAATCACAAGCATTCTCATACACC |
| | | | AATAACAGACAAACAGAGCCAAATCATG (SEQ ID NO: 538) |
| 533 | HGL6.1179 | | AACCAAACCAAGCAAACAAACAAACAGTAAAAACTCAATAACAACCAACAAAC |
| | | | AGGAAATACCAGGTAATTCAGATTATCTAGTTATGTGCCATAGT (SEQ ID NO: |
| | | | 539) |
| 534 | HGL6.1181 | | GAATGAATTGAATGCAAACATCGAATGGTCTCGAATGGAATCATCTTCAAATGG |
| | | | AATGGAATGGAATCATCGCATAGAATCGAATGGAATTATCAACGAATGGAATCG |
| | | | AATGGAATCATCATCAGATGGAAATGAATGGAATCGTCAT (SEQ ID NO: 540) |
| 535 | HGL6.1183 | | TGGAATGGAATCAAATCGCATGGAATCGAATGGAATAGAAAAGAATCAAACAG |
| | | | AGTGGAATGGAATGGAATGGAATGGAATCATGCCGAATGGAATG (SEQ ID NO: |
| | | | 541) |
| 536 | HGL6.1184 | | GAATCCATGTTCATAGCACAACAACCAAACAGAAGAAATCACTGTGAAATAAGA |
| | | | AACAAAGCAAAACACAGATGTCGACACATGGCA (SEQ ID NO: 542) |
| 537 | HGL6.1185 | | AAATGGAATAATGAAATGGAATCGAACGGAATCATCATCAAAAGGAACCGAAT |
| | | | GAAGTCATTGAATGGAATCAAAGGCAATCATGGTCGAATGGAATCAAATGGAAA |
| | | | CAGCATTGAATAGAATTGAATGGAGTCATCACATGGAATCG (SEQ ID NO: 543) |
| 538 | HGL6.1186 | | GAATTAACCCGAATAGAATGGAATGGAATGGAATGGAACAGAACGGAACGGAA |
| | | | TGGAATGGAATGGAATGGAATGGAATG (SEQ ID NO: 544) |
| 539 | HGL6.1188 | | AAGATATACAAGCAGCCAACAAACATACGAAAGAATGCTCAACATCACTAATCC |
| | | | TCAGAGAAATTTAAATCAAAACCACAATGAGTTACAATCTCATACCAGTCAGAA |
| | | | T (SEQ ID NO: 545) |
| 540 | HGL6.1190 | | AGAATTACAAACCACTGCTCAACAAAATAAAAGAGTACACAAACAAATGGAAG |
| | | | AATATTCCATGCTTATGGATAGGAAGAATCAATATTGTGAAAATGGCCATACT |
| | | | (SEQ ID NO: 546) |
| 541 | HGL6.1192 | | CATCGAATGGACTCGAATGGAATAATCATTGAACGGAATCGAAGGGAATCATCA |
| | | | TCGGATGGAAACGAATGGAATCATCATCGAATGGAAATG (SEQ ID NO: 547) |
| 542 | HGL6.1194 | | CACCCATCTGTAGGACCAGGAAGCCTGATGTGGGAGAGAACAGCAGGCTAAATC |
| | | | CAGGGTTGGTCTCTACAGCAGAGGGAATCACAAGCCTGTTAGCAAGTGAAGAAC |
| | | | CAACACTGGCAAGAGTGTGAAGGCC (SEQ ID NO: 548) |
| 543 | HGL6.1195 | | TAATGCAAACTAAAACGACAATGAGATATCAATACATAACTACCAGAAAGGCTA |
| | | | ACAAAAAAACAGTCATAACACACCAAAGGCTGATGAGTGAGGATGTGCAG (SEQ |
| | | | ID NO: 549) |
| 544 | HGL6.1196 | | AAAGGAATCAAACGGAATTATCGAATGGAATCGAAAAGAATCATCGAACGGACT |
| | | | CGAATGGAATCATCTAATGGAATGGAATGGAAGAATCCATGGACTCGAATG |
| | | | (SEQ ID NO: 550) |
| 545 | HGL6.1198 | | AGCAACTTCAGCAAAGTCTCAGGATACAAAATCAATGAGCAAAAATCACAAGCA |
| | | | TTCTTACACACCAATAACAGACAAACAGAGAGCC (SEQ ID NO: 551) |
| 546 | HGL6.1199 | | GGATATAAACAAGAAAACAACTAATCACAACTCAATATCAAAGTGCAATGATGG |
| | | | TGCAAAATGCAAGTATGGTGGGGACAGAGAAAGGATGC (SEQ ID NO: 552) |
| 547 | HGL6.1200 | | AATCAGTAAACGTAATACAGCATATAAACAGAACCAAAGACAAAAACCACATG |
| | | | ATTATCTCAATAGATGCAGAAAAGGCC (SEQ ID NO: 553) |
| 548 | HGL6.1202 | | AACATCAAACGGAAAAAAACGGAAATATCGAATGGAATCGAAGAGAATCATCG |
| | | | AATGGACC (SEQ ID NO: 554) |
| 549 | HGL6.1203 | | TAAAATGGAATCGAATGGAATCAACATCAAATGGAATCAAATGGAATCATTGAA |
| | | | CGGAATTGAATGGAATCGTCAT (SEQ ID NO: 555) |
| 550 | HGL6.1204 | | AATCATCATCGAATGGAATCGAATGGTATCATTGAATGGAATCGAATGGAATCA |
| | | | TCATCAGATGGAAATGAATGGAATCGTCAT (SEQ ID NO: 556) |
| 551 | HGL6.1205 | | CAATGCGTCAAGCTCAGACGTGCCTCACTACGGCAATGCGTCAAGCTCAGGCGT |
| | | | GCCTCACTAT (SEQ ID NO: 557) |
| 552 | HGL6.1206 | | AAGACAGAACACTGAAACTCAACAGAGAAGTAACAAGAACACCTAAGACAAGG |
| | | | AAGGAGAGGGAAGGCAGGCAG (SEQ ID NO: 558) |
| 553 | HGL6.1209 | | TAAGCTGATAAGCAACTTTAGCAAAGTCTCAGGATACAAAATCAATGTACAAAA |
| | | | ATCACAAGCATTCTTATACACCAACAACAGACAGACGGAGAGCCAAA (SEQ ID |
| | | | NO: 559) |
| 554 | HGL6.1212 | | ATGAACACGAATGTAATGCAATCCAATAGAATGGAATCGAATGGCATGGAATAT |
| | | | AAAGAAATGGAATCGAAGAGAATGGAAACAAATGGAATGGAATTGAATGGAAT |
| | | | GGAATTG (SEQ ID NO: 560) |
| 555 | HGL6.1216 | | AACAATCACTAGTCCTTAAGTAAGAGACAACACCTTTTGTCACACACAGTTTGTC |
| | | | CTAACTTTATCTTGGTAATTGGGGAGACC (SEQ ID NO: 561) |
| 556 | HGL6.1217 | | TAATGAGAAGACACAGACAACACAAAGAATCACAGAAACATGACACAGGTGAC |
| | | | AAGAACAGGCAAGGACCTGCAGTGCACAGGAGCC (SEQ ID NO: 562) |
| 557 | HGL6.1218 | | TGTTGAGAGAAATTAAACAAAGCACAGATAAATGGAAAAACGTGTTCATAGATT |
| | | | GAAAGACTTCATGTTGTATGGTGTC (SEQ ID NO: 563) |
| 558 | HGL6.1219 | | ATCAAACGGAATCAAACGGAATTATCGAATGGAATCGAAGAGAATCATCGAACG |
| | | | GACTCGAATGGAATCATCTAATGGAATGGGATGG (SEQ ID NO: 564) |
| 559 | HGL6.1222 | | ACACAACAACCAAGAAACAACCCCATTAAGAAGTGGGAAAAATACATGAATAA |
| | | | ACACATCTCAAAAGAAGACAAACAAGTGGCTAAC (SEQ ID NO: 565) |
| 560 | HGL6.1225 | | AATGGAAAGGAATCAAATGGAATATAATGGAATGCAATGGACTCGAATGGAATG |
| | | | GAATGGAATGGACCCAAATGGAATGGAATGGAATGGAATG (SEQ ID NO: 566) |
| 561 | HGL6.1226 | | GGAATACAACGGAATGGAATCGAAAAAAATGGAAAGGAATGAAATGAATGGAA |
| | | | TGGAATGGAATGGAATGGATGGGAATGGAATGGAATGG (SEQ ID NO: 567) |
| 562 | HGL6.1227 | | GAATCAAGCGGAATTATCGAATGGAATCGAAGAGAATCATCGAAAGGACTCGAA |
| | | | TGGAATCATCTAATGGAATGGAATGGAATAATACACGGACC (SEQ ID NO: 568) |
| 563 | HGL6.1232 | | AACAACAACAACAACAGGAAAACAACCTCAGTATGAAGACAAGTACATTGATTT |
| | | | ATTCAACATTTACTGATCACTTTTCAGGTGGTAGGCAGACC (SEQ ID NO: 569) |
| 564 | HGL6.1233 | | AAGATAACCTGTGCCCAGGAGAAAAACAATCAATGGCAACAAAAGCAGAAACA |
| | | | ACACAAATGATACAATTAGCAGACAGAAACATTGAGATTGCTATT (SEQ ID NO: |
| | | | 570) |
| 565 | HGL6.1234 | | AATGGACTCCAATGGAATAATCATTGAACGGAATCNAATGGAATCATCATCGGA |
| | | | TGGAAATGANTGGAATCNTCNTCNAATGGAATCN (SEQ ID NO: 571) |
| 566 | HGL6.1237 | | ANNCNNTAAACGTAATCCATCACATAAACANGANCNAANAGNNNAACCGCNNG |
| | | | ATTATCTCNNNNNNTGCNNAAAAGGCC (SEQ ID NO: 572) |
| 567 | HGL6.1240 | HGL6.1277 | TAATTGATTCGAAATTAATGGAATTGAATGGAATGCAATCAAATGGAATGGAAT |
| | | | GTAATGCAATGGAATGTAATAGAATGGAAAGCAATGGAATG (SEQ ID NO: 573) |
| 568 | HGL6.1242 | | AAAGGAATGGACTTGAACAAAATGAAATCGAACGATAGGAATCGTACAGAACG |
| | | | GAAAGAAATGGAACGGAATGGAATG (SEQ ID NO: 574) |
| 569 | HGL6.1243 | | AGCAACTTCAGCAAAATCTCAGGATACAAAATCAATGTACAAAAATCACAAGCA |
| | | | TTCTTATACACCAACAACAGACAAACAGAGAGCC (SEQ ID NO: 575) |
| 570 | HGL6.1247 | | TGAGCAGGGAACAATGCGGATAAATTTCACAAATACAATGTTGAGCAAAAGAAA |
| | | | GACACAAAANAATACACACATACACACCATATGGGCTAGG (SEQ ID NO: 576) |
| 571 | HGL6.1254 | | AATGGAATGGAATGTACAAGAAAGGAATGGAATGAAACCGAATGGAATGGAAT |
| | | | GGACGCAAAATGAATGGAATGGAAGTCAATGG (SEQ ID NO: 577) |
| 572 | HGL6.1260 | | AAGTTCAAACATCAGTATTAACCTTGAACATCAATGGCCTACATGCATCACTTAA |
| | | | AACATACAGACAGGCAAATTGGGTTAAGAAAACAAACAAGCAAACAAAACATG |
| | | | TTCCAAACATTTGTTGGCTAT (SEQ ID NO: 578) |
| 573 | HGL6.1262 | | GGAATAATCATTGAACGGAATCGAATGGAATCATCATCGGATGGAAACGAATGG |
| | | | AATCATCATCGAATGGAAATGAAAGGAGTCATC (SEQ ID NO: 579) |
| 574 | HGL6.1264 | | GGAACGAAATCGAATGGAACGGAATAGAATAGACTCGAATGTAATGGATTGCTA |
| | | | TGTAATTGATTCGAATGGAATGGAATCG (SEQ ID NO: 580) |
| 575 | HGL6.1265 | | TGAAAGGAATAGACTGGAACAAAATGAAATCGAATGGTAGGAATCATACAGAA |
| | | | CAGAAAGAAATGGAACGGAATGGAATG (SEQ ID NO: 581) |
| 576 | HGL6.1266 | | AACCCGAATAGAATGGAATGGAATGGAATGGAACGGAACGGAATGGAATGGAA |
| | | | TGGATTGGAATGGAATGGAATG (SEQ ID NO: 582) |
| 577 | HGL6.1267 | | AAAGAGAATCAAATGGAATTGAATCGAATGGAATCGAATGGATTGGAAAGGAA |
| | | | TAGAATGGAATGGAATGGAATGGAATGGAATGGAATG (SEQ ID NO: 583) |
| 578 | HGL6.1269 | | AAAACACACAAACATACATGTGGATGCACATATAAACATGCACATACACACACA |
| | | | CATAAATGCACAAACACACTTAACACAAGCACACATGCAAACAAACACATGG |
| | | | (SEQ ID NO: 584) |
| 579 | HGL6.1270 | | AATGGAATCATCAGTAATGGAATGGAAAGGAATGGAAAGGACTGGAATGGAAT |
| | | | GGAATGGAATGGAATGG (SEQ ID NO: 585) |
| 580 | HGL6.1271 | | GGAACAAAATGAAATCGAACGGTAGGAATCGTACAGAACGGAAAGAAATGGAA |
| | | | CGGAATGGAATGCACTCAAATGGAAAGGAGTCCAATGGAATCGAAAGGAATAG |
| | | | AATGGAATGG (SEQ ID NO: 586) |
| 581 | HGL6.1272 | | AGAATGAGATCAAGCAGTATAATAAAGGAAGAAGTAGCAAAATTACAACAGAG |
| | | | CAGTGAAATGGATATGCTTTCTGGCAATAATTGTGAAAGGTCTGGTAATGAGAA |
| | | | AGTAGCAACAGCTAGTGGCTGCCAC (SEQ ID NO: 587) |
| 582 | HGL6.1273 | | AACAAATGGAATCAACATCGAATGGAATCGAATGGAAACACCATCGAATTGAAA |
| | | | CGAATGGAATTATCATGAAATTGAAATGGATGGACTCATCATCG (SEQ ID NO: |
| | | | 588) |
| 583 | HGL6.1278 | | TAACATGCAGCATGCACACACGAATACACAACACACAAACATGTATGCACGCAC |
| | | | ACGTGAATACACAACACACACAAACATGCATGCATGCATACATGAATACACAGC |
| | | | ACACAAATATCCAGCAT (SEQ ID NO: 589) |
| 584 | HGL6.1279 | | GAATGGAATCAACATCAAACGGAAAAAAAACGGAATTATCGAATGGAATCGAA |
| | | | TAGAATCATCGAATGGACC (SEQ ID NO: 590) |
| 585 | HGL6.1281 | | AATCGAATGAAATGGAGTCAAAAGGAATGGAATCGAATGGCAAGAAATCGAAT |
| | | | GTAATGGAATCGCAAGGAATTGATGTGAACGGAACGGAATGGAAT (SEQ ID NO: |
| | | | 591) |
| 586 | HGL6.1282 | | AATGGAATTGAACGGAAACATCAGCGAATGGAATCGAAAGGAATCATCATGGA |
| | | | ATAGATTCGAATGGAATGGAAAGGAATGGAATGGAATG (SEQ ID NO: 592) |
| 587 | HGL6.1283 | | ATGGAATCAACATCAAACAGAATCAAACGGAATTATCGAATGGAATCGAAGACA |
| | | | ATCATCGAATGGACTCGAATGGAATCATCTAATGGAATGGAATGGAAGAATCCA |
| | | | TGGTCTCGAATGCAATCATCATCG (SEQ ID NO: 593) |
| 588 | HGL6.1284 | | GAATAATCATTGAACGGAATCGAATGGAATCATCTTCGGATGGAAACGAATGGA |
| | | | ATCATCATCGAATGGAAATGAAAGGAGTCATC (SEQ ID NO: 594) |
| 589 | HGL6.1288 | | AATGGACTCGAATGGAATAATCATTGAACGGAATCGAATGGAATCATCATCGGA |
| | | | TGGAAATGAGTGGAATCATCATCGAATGGAATCG (SEQ ID NO: 595) |
| 590 | HGL6.1290 | | AAATGAAATCGAACGGTAGGAATCGTACAGAACGGAAAGAAATGGAACGGAAT |
| | | | GGAATGCAATCGAATGGAAAGGAGTCCAATGGAAGGGAATCGAAT (SEQ ID NO: |
| | | | 596) |
| 591 | HGL6.1291 | | TACCAAACATTTAAAGAACAAATATCAATCCTACGCAAACCATTCTGAAACACA |
| | | | GAGATGGAGGATATACAGCGAAACTCATTCTACATGGCC (SEQ ID NO: 597) |
| 592 | HGL6.1292 | | TATTGGAATGGAATGGAATGGAGTCGAATGGAACGGAATGCACTCGAATGGAAG |
| | | | GCAATGCAATGGAATGCACTCAACAGGAATAGAATGGAATGGAATGGAATGG |
| | | | (SEQ ID NO: 598) |
| 593 | HGL6.1294 | | AGAGAGTATTCATCATGAGGAGTATTACTGGACAAATAATTCACAAACGAACAA |
| | | | ACCAAAGCGATCATCTTTGTACTGGCTGGCTA (SEQ ID NO: 599) |
| 594 | HGL6.1295 | | GGAATTTAATAGAATGTACCCGAATGGAACGGAATGGAATGGAATTGTATGGCA |
| | | | TGGAATGGAA (SEQ ID NO: 600) |
| 595 | HGL6.1298 | | GCAATCCANTANAATGGAATCGAATGGCATGGAATATAAAGAAATGGAATCGAA |
| | | | GAGAATGGAGACAAATGGAATGGAATTGAATGGAATGGAATTG (SEQ ID NO: |
| | | | 601) |
| 596 | HGL6.1299 | | AATGGAATCGAATGGAATCATCATCAAATGGAATCTAATGGAATCATTGAACGG |
| | | | AATTAAATGGAATCGTCATCGAATGAATTCAATGCAATCAACGAATGGTCTCGA |
| | | | ATGGAACCAC (SEQ ID NO: 602) |
| 597 | HGL6.1300 | | AATTGCAAAAGAAACACACATATACACATATAAAACTCAAGAAAGACAAAACTA |
| | | | ACCTATGGTGATAGAAATCAGAAAAGTACAGTACATTGGTTGTCTTGGTGGG |
| | | | (SEQ ID NO: 603) |
| 598 | HGL6.1303 | | TGACATCATTATTATCAAGAAACATTCTTACCACTGTTACCAACTTCCCAACACA |
| | | | GACTATGGAGAGAGAGATAAGACAGAATAGCATT (SEQ ID NO: 604) |
| 599 | HGL6.1305 | | GGAATCTATAATACAGCTGTTTATAGCCAAGCACTAAATCATATGATACAGAAA |
| | | | ACAAATGCAGATGGTTTGAAGGGTGGG (SEQ ID NO: 605) |
| 600 | HGL6.1308 | | AAAGAATTGAATTGAATAGAATCACCAATGAATTGAATCGAATGGAATCGTCAT |
| | | | CGAATGGAATCGAAGGGAATCATTGGATGGGCTCA (SEQ ID NO: 606) |
| 601 | HGL6.1311 | | ATCATCGAATGGAATCGAATGGAATCAATATCAAACGGAAAAAAACGGAATTAT |
| | | | CGAATGGAATCGAATAGAATCATCGAATGGACC (SEQ ID NO: 607) |
| 602 | HGL6.1314 | | GAATGAAATCGTATAGAATCATCGAATGCAACTGAATGGAATCATTAAATGGAC |
| | | | TTGAAAGGAATTATTATGGAATGGAATTG (SEQ ID NO: 608) |
| 603 | HGL6.1316 | | TAAGCAACTTCAGCAAAGTCTCAGGATACAAAATCAATGTGCAAAAATCTCAAG |
| | | | CATTCTTATACACGAACAACAGACAAACAGAGAGCT (SEQ ID NO: 609) |
| 604 | HGL6.1317 | | ACTCAAAAGGAATTGATTCGAATGGAATAGAATGGCAAGGAATAGTATTGAATT |
| | | | GAATGGAATGGAATGGACCCAAATG (SEQ ID NO: 610) |
| 605 | HGL6.1319 | | GAATGGAATTTAAAGGAATAGAATGGAAGGAATCGGATGGAATGGAATGGAAT |
| | | | AGAATGGAGTCGAATGGAATAGAATCGAATGGAATGGCATTG (SEQ ID NO: 611) |
| 606 | HGL6.1323 | | AACAAAAAATGAGTCAAGCCTTAAATAAAATCAGAGCCAAAAAAGAAGACATT |
| | | | ACATCTGATAAGACAAAAATTCAAAGGACCATC (SEQ ID NO: 612) |
| 607 | HGL6.1324 | | AACCCAGTGGAATTGAATTGAATGGAATTGAATGGAATGGAAAGAATCAATCCG |
| | | | AGTCGAATGGAATGGTATGGAATGGAATGGCATGGAATCAAC (SEQID NO: 613) |
| 608 | HGL6.1327 | | ATCAACATCAAACGGAAAAAAAACGGAATTATCGAATGGAATCGAAGAGAATC |
| | | | ATCGAATGGACC (SEQ ID NO: 614) |
| 609 | HGL6.1331 | | AAGGAATGGAATGGTACGGAATAGAATGGAATGGAACGAATTGTAATGGAATG |
| | | | GAATTTAATGGAACGGAATGGAATGGAATGGAATCAACG (SEQ ID NO: 615) |
| 610 | HGL6.1334 | | AACGGAATGGAAAGCAATTTAATCAAATGCAATACAGTGGAATTGAAGGGAATG |
| | | | GAATGGAATGGC (SEQ ID NO: 616) |
| 611 | HGL6.1335 | | AATCGAATGGAACGGAATAGAATAGACTCGAATGTAATGGATTGCTATGTAATT |
| | | | GATTCGAATGGAATGGAATCGAATGGAATGCAATCCAATGGAATGGAATGCAAT |
| | | | GCAATGGAATGGAATCGAACGGAATGCAGTGGAAGGGAATGG (SEQ ID NO: 617) |
| 612 | HGL6.1336 | | TAGCAACATTTTAGTAACATGATAGAAACAAAACAGCAACATAGCAATGCAATA |
| | | | GTAACACAACAGCAACATCATAACATGGCAGCA (SEQ ID NO: 618) |
| 613 | HGL6.1337 | | GGACAAATTGCTAGAAATAAACAAATTACCAAAAATGATTCAAGTAGAGACAGA |
| | | | GAATCAAAATAGAACTACACATAAGTGGGCCAAG (SEQ ID NO: 619) |
| 614 | HGL6.1340 | | AAAATAGAATGAAAGAGAATCAAATGGAATTGAATCGAATGGAATCGAATGGA |
| | | | TTGGAAAGGAATAGAATGGAATGGAATGGAATG (SEQ ID NO: 620) |
| 615 | HGL6.1342 | | AGCAAACAAGTGAATAAACAAGCAAACAAGTGAACAAGCAAACAAGTGAATAA |
| | | | ACAAGCAAACAAGTGAACAAGCAAACAAGTGAATAAACAAGCAAACAAGTGAA |
| | | | CAAGGAAACAAGTGAATAAACAAAGGCTCT (SEQ ID NO: 621) |
| 616 | HGL6.1346 | | AATGGAATCAACACGAGTGCAATTGAATGGAATCGAATGGAATGGAATGGAATG |
| | | | GAATGAATTCAACCCGAATGGAATGGAAAGGAATGGAATC (SEQ ID NO: 622) |
| 617 | HGL6.1347 | | AATATACGCAAATCAATAAATGTAATCCAGCATATAAACAGTACTAAAGACAAA |
| | | | AACCACATGATTATCTCAATAGATGCAGAAAAGGCC (SEQ ID NO: 623) |
| 618 | HGL6.1352 | | GAATCGAATGGAATCAACATCAAACGGAAAAAAACGGAATTATCGAATGGAATC |
| | | | GAAGAGNNNNNNCGAATGGACC (SEQ ID NO: 624) |
| 619 | HGL6.1354 | | AACACGAATGTAATGCAATCCAATAGAATGGAATCGAATGGCATGGAATATAAA |
| | | | GAAATGGAATCGAAGAGAATGGAAACAAACGGAATGGAATTGAATGGAATGGA |
| | | | ATTGAATGGAATGGGAACGAATGGAGTGAAATTG (SEQ ID NO: 625) |
| 620 | HGL6.1355 | | GAATGGAACGGAATAGAACAGACTCGAATGTAATGGATTGCTATGTAATTGATT |
| | | | CGAATGGAATGGAATCGAATGGAATGCAATCCAATGGAATGGAATGCAATGCAA |
| | | | TGGAATGGAATCGAATGGAATGCAGTGGAAGGGAATGG (SEQ ID NO: 626) |
| 621 | HGL6.1356 | | GAATCGAATGGAATCAATATCAAACGGAAAAAAACGGAATTATCGAATGGAATC |
| | | | GAAGAGAATCATCGAATGGACC (SEQ ID NO: 627) |
| 622 | HGL6.1359 | | TAAACAACGAGAACACATGAACACAAAGAGGGGAACAACAGACACCAAGACCT |
| | | | TCTTGAGGGTGGAGGATGGGAGGAGGGAG (SEQ ID NO: 628) |
| 623 | HGL6.1360 | | AGCAACTTCAGCAGTCTCAGTATACAAAAACAATGTGCAAAAATCACAAGCATT |
| | | | CCTATATGCCAATAACAGACAAACAGAGAGCC (SEQ ID NO: 629) |
| 624 | HGL6.1361 | | ATCAAAAGAAAAGCAACCTAACAAATACGGGAAGAATATTTGAATAGACATTTC |
| | | | ACAGGAAAAGATATATGAATGGCCAAAAAGCAAATGAAAAG (SEQ ID NO: 630) |
| 625 | HGL6.1364 | | ATAAACATCAAACGGAATCAAACGGAATTATCGAATGGAATCGAAGAGAATAAT |
| | | | CGAATGGACTCAAATGGAGTCATCTAATGGAATGGTATGGAAGAATCCATGGAC |
| | | | TCCAACGCAATCATCAGCGAATGGAATC (SEQ ID NO: 631) |
| 626 | HGL6.1365 | | AAAAGAAAAGACAAAAGACACCAATTGCCAATACTGAAATGAAAAAACAGGTA |
| | | | ATAACTATTGATCCCATGGACATTAAAATGATGTTGAAGGAACACCAC (SEQ ID |
| | | | NO: 632) |
| 627 | HGL6.1368 | | AGCAATAACCAAACAACCTCATTAAAAAGTAGGCAAAGGACATAAACAGACACT |
| | | | TTTCAAAAGAAGACATACACGTGGCCAACAAACATATG (SEQ ID NO: 633) |
| 628 | HGL6.1370 | | AGCAACTTCAGCAAAGTCTCAGGATACAAAATCGATGTGCAAAAATCACAAGCA |
| | | | TTCTTATACACCAATAACAGGCAAACAGAGAGCC (SEQ ID NO: 634) |
| 629 | HGL6.1371 | | GTCATATTTGGGATTTATCATCTGTTTCTATTGTTGTTGTTTTAGTACACACAAAG |
| | | | CCACAATAAATATTCTAGGCT (SEQ ID NO: 635) |
| 630 | HGL6.1373 | | ATCATCGAATGGAATAGAATGGTATCAACATCAAACGGAGAAAAACGGAATTAT |
| | | | CGAATGGAATCGAAGAGAATCTTCGAACGGACC (SEQ ID NO: 636) |
| 631 | HGL6.1374 | | AAATAAGCCAACGGTCATAAATTGCAAAGCCTTTTACAATCCAAACATGATGGA |
| | | | AACGATATGCCATTTTGAAGGTGATTTGAAAAGCACATGGTTT (SEQ ID NO: 637) |
| 632 | HGL6.1375 | | GAATGGAATCATCGCATAGAATCGGATGGAATTATCATCGAATGGAATCGAATG |
| | | | GTATCAACATCAAACGGAAAAAAACGGAATTATCGAATGGAATCGAATTGAATC |
| | | | ATCGAACGGACCCG (SEQ ID NO: 638) |
| 633 | HGL6.1378 | | AATGGACTCGAATGGAATAATCATTGAACGGAATCGAATGGAATCATCATCGGA |
| | | | TGGAAATGAATGGAATAATCCATGGACTCGAATGCAATCATCATCGAATGGAAT |
| | | | CGAATGGAATCATCGAATGGACTCG (SEQ ID NO: 639) |
| 634 | HGL6.1379 | | AATGCAATCATCAACTGGCTTCGAATGGAATCATCAAGAATGGAATCGAATGGA |
| | | | ATCATCGAATGGACTC (SEQ ID NO: 640) |
| 635 | HGL6.1380 | | AAGAGACCAATAAGGANTANGTAAGCAACANGAGGAAGGAGANANGGGCAAG |
| | | | AGAGATGACCAGAGTT (SEQ ID NO: 641) |
| 636 | HGL6.1382 | | TGGAATCATCATAAAATGGAATCGAATGGAATCAACATCAAATGGAATCAAATG |
| | | | GAATCATTGAACGGAATTGAATGGAATCGTCAT (SEQ ID NO: 642) |
| 637 | HGL6.1383 | | GGAATCATCGCATAGAATCGAATGGAATTATCATCGAATGGAATCGAATGGAAT |
| | | | CAACATCAAACGAAAAAAAACCGGAATTATCGAATGGAATCGAAGAGAATCATC |
| | | | GAACGGACC (SEQ ID NO: 643) |
| 638 | HGL6.1384 | | AAATCATCATCGAATGGGATCGAATGGTATCCTTGAATGGAATCGAATGGAATC |
| | | | ATCATCAGATGGAAATGAATGGAATCGTCAT (SEQ ID NO: 644) |
| 639 | HGL6.1386 | | GGAATGTAATAGAACGGAAAGCAATGGAATGGAACGCACTGGATTCGAGTGCA |
| | | | ATGGAATCTATTGGAATGGAATCGAATGGAATGGTTTGGCATGGAATGGAC (SEQ |
| | | | ID NO: 645) |

### REFERENCES

1. R. J. Jackson, C. U. T. Hellen, T. V. Pestova, Nat. Rev. Mol. Cell. Biol. 10, 113 (2010).
2. N. Sonenberg, A. G. Hinnebusch, Cell 136, 731 (2009).
3. C. U. T. Hellen, P. Sarnow, Genes Dev. 15, 1593 (2001).
4. S. Vagner, B. Galy, S. Pyronnet, EMBO Rep. 2, 893 (2001).
5. M. Stoneley, A. E. Willis, Oncogene 23, 3200 (2004).
6. J. S. Kieft, A. Grech, P. Adams, J. A. Doudna, Cold Spring Harbor Symp. on Quant. Biol. 66, 277 (2001).
7. N. Malys, J. E. G. McCarthy, Cell. Mol. Life. Sci. 68, 991 (2011).
8. T. A. Hughes, Trends Genet. 22, 119 (2006).
9. X. Qin, P. Sarnow, J. Biol. Chem. 279, 13721 (2004).
10. M. Bushell et al., Mol. Cell 23, 401 (2006).
11. J. D. Thomas, G. J. Johannes, RNA 13, 1116 (2007).
12. G. Johannes, M. S. Carter, M. B. Eisen, P. O. Brown, P. Sarnow, Proc. Natl. Acad. Sci. USA 96, 13118 (1999).
13. S. Braunstein et al., Mol. Cell. 28, 501 (2007).
14. K. A. Spriggs, M. Stoneley, M. Bushell, A. E. Willis, Biol. Cell 100, 27 (2008).
15. S. D. Baird, M. Turcotte, R. G. Korneluk, M. Holcik, RNA 12, 1755 (2006).
16. R. W. Roberts, J. W. Szostak, Proc. Natl. Acad. Sci. USA 94, 12297 (1997).
17. T. T. Takahashi, R. J. Austin, R. W. Roberts, Trends Biochem. Sci. 28, 159 (2003).
18. W. J. Kent, Genome Res. 12, 656 (2002).
19. J. O. Korbel et al., Science 318, 420 (2007).
20. M. Kasowski et al., Science 328, 232 (2010).
21. K. Salehi-Ashtiani, A. Luptak, A. Litovchick, J. W. Szostak, Science 313, 1788 (2006).
22. E. S. Lander et al., Nature 409, 860 (2001).
23. R. Cordaux, M. A. Batzer, Nat. Rev. Genet. 10, 691 (2009).
24. B. T. Baranick et al., Proc. Natl. Acad. Sci. USA 105, 4733 (2008).
25. T. R. Fuerst, E. G. Niles, F. W. Studier, B. Moss, Proc. Natl. Acad. Sci. USA 83, 8122 (1986).
26. W. V. Gilbert, K. H. Zhou, T. K. Butler, J. A. Doudna, Science 317, 1224 (2007).
27. M. Kozak, Nuc. Acids Res. 15, 8125 (1987).
28. Q. Yang, P. Sarnow, Nuc. Acids Res. 25, 2800 (1997).
29. I. Huez et al., Mol. Cell. Biol. 18, 6178 (1998).

We claim:

1. A nucleic acid library comprising a plurality of linear recombinant double stranded DNA constructs, wherein each double stranded DNA construct comprises
(a) a promoter;
(b) a heterologous coding region downstream from the promoter, wherein the coding region encodes a detectable polypeptide;
(c) a heterologous cross-linking region downstream of the coding region;
(d) a heterologous polynucleotide sequence of between 20-500 base pairs in length located downstream of the promoter and upstream of the coding region; and
(e) a first PCR primer binding site and a second PCR primer binding site, wherein the first PCR primer binding site is upstream of the polynucleotide sequence and the second PCR primer site is downstream of the polynucleotide sequence;
wherein at least 10¹³ different polynucleotide sequences are represented in the plurality of double stranded nucleic acid constructs, and wherein the first PCR primer and the second PCR primer are the same for each construct in the plurality of double stranded nucleic acid constructs.

2. The nucleic acid library of claim 1, wherein expressed RNA from the cross-linking region can serve as a site for ligation to a linker containing a 3′-puromycin residue.

3. The nucleic acid library of claim 2, wherein mRNA expressed from the cross linking region is complementary to a DNA linker sequence to be used.

4. The nucleic acid library of claim 1, wherein the polynucleotide sequence of between 20-500 base pairs are genomic fragments.

5. The nucleic acid library of claim 1, wherein the polynucleotide sequence of between 20-500 base pairs are synthetic sequences.

6. The nucleic acid library of claim 1, wherein the polynucleotide sequence is between 20-400 base pairs in length.

7. The nucleic acid library of claim 1, wherein the library comprises at least 10¹⁴ different polynucleotide sequences.

8. The nucleic acid library of claim 1, wherein the double stranded nucleic acid constructs further comprise
(a) one or more unique restriction sites upstream of the polynucleotide sequence and downstream of the promoter, and
(b) one or more unique restriction sites downstream of the polynucleotide sequence.

9. The nucleic acid library of claim 1, wherein the first (5′) or second (3′) primer binding site is upstream of the coding region in the double stranded nucleic acid construct.

10. An mRNA pool resulting from transcription of the library of claim 1.

11. A method for identifying translational enhancing elements (TEEs), comprising
(a) contacting the nucleic acid library of claim 1 with reagents for RNA transcription under conditions to promote transcription of RNA from the double stranded nucleic acid constructs, resulting in an RNA expression product;
(b) contacting the RNA expression product with reagents for ligating a linker containing a puromycin residue to the 3′ end of the RNA expression product, resulting in a labeled RNA expression product;
(c) contacting the labeled RNA expression product with reagents for protein expression under conditions to promote protein translation from the labeled RNA expression product, resulting in a RNA-polypeptide fusion product;
(d) isolating RNA-polypeptide fusion products;
(e) converting the isolated RNA-polypeptide fusion products to cDNA by reverse transcription-PCR using a primer to the 3′ end of the isolated RNA-polypeptide fusion products;
(f) amplifying the cDNA by PCR using primers to the 5′ and 3′ end of the cDNA; and
(g) repeating steps (a)-(f) a desired number of times, wherein the amplified polynucleotide sequence fragments comprise TEEs.

12. The method of claim 11, wherein the primers used in step (f) add a promoter to the 5′ end and a cross-linking region to the 3′ end of the cDNA after each round of selection.

13. The method of claim 11, wherein the linker comprises a DNA linker complementary to the RNA expression product.

14. The method of claim 11, wherein the polynucleotide sequences in the library comprise genomic fragments, and wherein a starting pool of library constructs contains at least a five-fold coverage of the genome of interest.

15. The method of claim 11, wherein the method further comprises testing polynucleotide sequences identified as TEEs for TEE activity in vivo.

16. An isolated polynucleotide, comprising a nucleic acid sequence according to any one of SEQ ID NOS: 1-5 and 7-645.

17. The isolated polynucleotide of claim 16, wherein the polynucleotide is selected from the group consisting of SEQ ID NO:1-5, 448, 495, 623, 408, 12, 54, 401, 553, 434, 458, 214, 327, 397, 471, 398, 301, 310 and 583.

18. An isolated polynucleotide comprising a nucleic acid sequence according to SEQ ID NO:1.

19. The isolated polynucleotide of claim 18, comprising a nucleic acid sequence according to SEQ ID NO:2.

20. The isolated polynucleotide of claim 18, comprising a nucleic acid sequence according to SEQ ID NO:3.

21. An isolated polynucleotide comprising a nucleic acid sequence according to SEQ ID NO:4.

22. The isolated polynucleotide of claim 16, wherein the polynucleotide is 200 nucleotides or less in length.

23. An expression vector comprising
(a) a promoter;
(b) a heterologous TEE downstream of the promoter, where the TEE comprises a polynucleotide according to claim 16; and
(c) a cloning site suitable for cloning of an protein-encoding nucleic acid of interest located upstream of the TEE, and downstream of the promoter.

24. The expression vector of claim 23, further comprising a protein-encoding nucleic acid cloned into the cloning site.

25. A recombinant host cell comprising the expression vector of claim 23.

26. A method for protein expression, comprising contacting the expression vector of claim 24 with reagents and under conditions suitable for promoting expression of the polypeptide encoded by the protein-encoding nucleic acid.

28. The method of claim 26, wherein the protein expression is carried out in vitro.

29. The method of claim 26, wherein the protein expression is carried out in a recombinant host cell.
